(19) United States
(12) Patent Application Publication
(10) Pub. No.: US 2020/0239530 A1
(43) Pub. Date: Jul. 30, 2020

(54) COMPOUNDS AND METHODS OF MODULATING PROTEIN DEGRADATION

(71) Applicant: Vividion Therapeutics, Inc., San Diego, CA (US)

(72) Inventors: Matthew PATRICELLI, San Diego, CA (US); Dean STAMOS, San Diego, CA (US); Gabe SIMON, San Diego, CA (US); Benjamin HORNING, San Diego, CA (US)

(21) Appl. No.: 16/650,765

(22) PCT Filed: Sep. 27, 2018
(86) PCT No.: PCT/US2018/053146

§ 371 Date: Mar. 25, 2020

Related U.S. Application Data
(60) Provisional application No. 62/564,184, filed on Sep. 27, 2017.

(51) Int. Cl. C07K 14/47; C12N 9/10

(52) U.S. Cl. CPC C07K 14/47; C12N 9/104

(57) ABSTRACT

Disclosed herein are protein-probe adducts and synthetic ligands that inhibit protein-probe adduct formation, in which the protein is part of the E3 ligase complex and the protein is modified to alter the substrate recognition of the E3 ligase complex. In some instances, also provided herein are protein-probe adducts and synthetic ligands that inhibit protein-probe adduct formation, in which the protein is modified or tagged for degradation. In some instances, additionally provided herein are cysteine-containing protein binding domains that interact with a probe and/or a ligand described herein.

### CROSS-REFERENCE

This application claims the benefit of U.S. Provisional Application No. 62/564,184, filed Sep. 27, 2017, which application is incorporated herein by reference in its entirety.

### BACKGROUND OF THE DISCLOSURE

Protein biosynthesis and degradation is a dynamic process which sustains normal cell metabolism. In some instances, production of new proteins modulate proliferation and differentiation of cells and upon completion, these protein are degraded through one of two proteolytic mechanisms, the lysosome degradation system or the ubiquitin proteasome pathway. In some cases, a majority of cellular proteins are degraded by the proteasome pathway, and the process is initiated via tagging of a ubiquitin.

### SUMMARY OF THE DISCLOSURE

In certain embodiments, described herein are compositions that comprise cysteine-containing proteins that are conjugated with a probe of Formula (I) or with a ligand disclosed herein. In some embodiments, disclosed herein is a protein-probe adduct wherein the probe binds to a cysteine residue illustrated in Table 1A or Table 2A; wherein the probe has a structure represented by Formula (I):

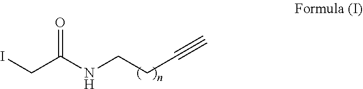

wherein,
n is 0-8.

In some embodiments, disclosed herein is a synthetic ligand that inhibits a covalent interaction between a protein and a probe, wherein in the absence of the synthetic ligand, the probe binds to a cysteine residue illustrated in Table 1A or Table 2A; and wherein the probe has a structure represented by Formula (I):

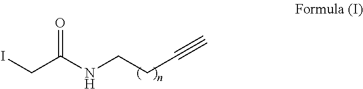

wherein,
n is 0-8.

In some embodiments, disclosed herein is a protein binding domain wherein said protein binding domain comprises a cysteine residue illustrated in Table 1A or Table 2A, wherein said cysteine forms an adduct with a compound of Formula I,

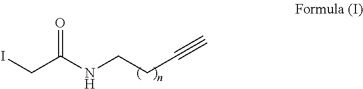

and wherein a compound of Formula IIA or Formula IIB interferes with the formation of the cysteine adduct by the compound of Formula I, wherein Formula (IIA) or Formula (IIB) have the structure:

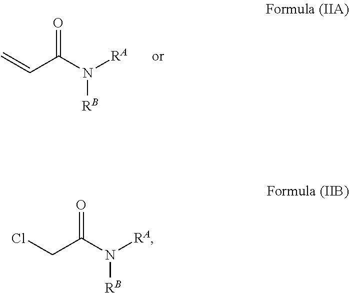

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl; or
or R^{A} and R^{B} together with the nitrogen to which they are attached form a 5, 6, 7 or 8-membered heterocyclic ring A, optionally having one additional heteroatom moiety independently selected from NR¹, O, or S; wherein A is optionally substituted; and
R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

### DETAILED DESCRIPTION OF THE DISCLOSURE

Ubiquitin-proteasome system is characterized by the E1, E2, and E3 enzyme. First, a ubiquitin molecule is chemically activated in an ATP-dependent manner by an E1-activating enzyme forming a thioester bond between the C-terminal glycine residue of ubiquitin and a conserved cysteine residue of the E1. Then, ubiquitin is transferred on to an E2-conjugated enzyme via a trans-thiolation reaction. Next, an isopeptide bond between the ε-amino group of a substrate lysine residue and the C-terminal glycine residue of ubiquitin is formed via E3 ligase-mediated catalysis and then between ubiquitin molecules to form poly-ubiquitin chains. Upon completion of the ubiquination process, the tagged substrate is subsequently recognized and degraded by the 26S proteasome in an ATP-dependent manner.

In some cases, the E3 ubiquitin ligase family is divided into three families, the HECT (homologous with E6-associated protein C-terminus) family, the RING finger family, and the RBR (RING-between RING_RING) family. HECT E3 enzyme forms a covalent thioester intermediate by accepting a ubiquitin molecule from the E2-ubiquitin via a conserved cysteine residue prior to transferring the ubiquitin molecule to a substrate. RING E3 enzyme directly transfers a ubiquitin molecule to a substrate by bringing both the E2-ubiquitin and the substrate in close proximity to each other. The RBR family recruit E3-ubiquitin conjugated by an N-terminal RING domain and then transfer ubiquitin on to a HECT-type C-terminal catalytic cysteine residue of the E3 before transferring on to the substrate.

In some instances, the RING finger family is further categorized into two subgroups, CRL and APC/C (anaphase-promoting complex/cyclosome). In some cases, the CRL and APC/C subfamilies comprise multi-subunit complexes comprising an adaptor, a substrate receptor subunit, a Cullin scaffold, and a RING-box subunit.

In some embodiments, the CUL4-RBX1-DDB1-CRBN complex (CRL4^{CRBN}) is an E3 ligase that falls under the CRL subgroup of the RING finger family. The CRL4^{CRBN} complex comprises the adaptor protein DDB1, which connects the substrate receptor cereblon (CRBN) to the Cullin 4 (CUL4) scaffold. The Cullin 4 scaffold further binds to RBX1. Upon substrate binding, the CUL4-RBX1-DDB1-CRBN complex bridges the substrate to the E2-ubiquitin to initiate a direct transfer of ubiquitin molecule onto the substrate.

In some instances, thalidomide and related immunomodulatory (IMiD) compounds such as lenalidomide and pomalidomide promote and modulate cereblon recruitment of neosubstrates. For example, a cereblon modulator CC-220 has been shown to improve degradation of Ikaros and Aiolos, two zinc finger transcription factors that have been implicated in lymphoid development and differentiation (Matyskiela, et al., “A cereblon modulator (CC-220) with improved degradation of Ikaros and Aiolos,” J Med Chem. Apr. 20, 2017). Further, dBET1, a bifunctional phthalimide-conjugated ligand which is a substrate for cereblon, selectively targets BRD4, a transcriptional coactivator, for degradation.

In some embodiments, provided herein are protein-probe adducts and synthetic ligands that inhibit protein-probe adduct formation, in which the protein is part of the E3 ligase complex and the protein is modified to alter the substrate recognition of the E3 ligase complex. In some instances, also provided herein are protein-probe adducts and synthetic ligands that inhibit protein-probe adduct formation, in which the protein is modified or tagged for degradation. In some instances, additionally provided herein are cysteine-containing protein binding domains that interact with a probe and/or a ligand described herein.

In some embodiments, provided herein are protein-probe adducts illustrated in Table 2B, wherein the percent inhibition is greater than 98%. In some embodiments, provided herein are protein-probe adducts illustrated in Table 2B, wherein the percent inhibition is greater than 97%. In some embodiments, provided herein are protein-probe adducts illustrated in Table 2B, wherein the percent inhibition is greater than 96%. In some embodiments, provided herein are protein-probe adducts illustrated in Table 2B, wherein the percent inhibition is greater than 95%. In some embodiments, provided herein are protein-probe adducts illustrated in Table 2B, wherein the percent inhibition is greater than 94%. In some embodiments, provided herein are protein-probe adducts illustrated in Table 2B, wherein the percent inhibition is greater than 93%. In some embodiments, provided herein are protein-probe adducts illustrated in Table 2B, wherein the percent inhibition is greater than 92%. In some embodiments, provided herein are protein-probe adducts illustrated in Table 2B, wherein the percent inhibition is greater than 91%. In some embodiments, provided herein are protein-probe adducts illustrated in Table 2B, wherein the percent inhibition is greater than 90%. In some embodiments, provided herein are protein-probe adducts illustrated in Table 2B, wherein the percent inhibition is greater than 85%. In some embodiments, provided herein are protein-probe adducts illustrated in Table 2B, wherein the percent inhibition is greater than 80%. In some embodiments, provided herein are protein-probe adducts illustrated in Table 2B, wherein the percent inhibition is greater than 75%. In some embodiments, provided herein are protein-probe adducts illustrated in Table 2B, wherein the percent inhibition is greater than 70%. In some embodiments, provided herein are protein-probe adducts illustrated in Table 2B, wherein the percent inhibition is greater than 65%.

In some embodiments, further described herein is a method of modulating or altering recruitment of neosubstrates to the ubiquitin proteasome pathway. In some instances, the method comprises covalent binding of a reactive residue on one or more proteins described below for modulation of substrate interaction. In some cases, the method comprises covalent binding of a reactive cysteine residue on one or more proteins described below for substrate modulation.

### Cysteine-Containing Proteins and Protein Conjugates

In some embodiments, described herein are cysteine-containing proteins that upon interaction with a probe or a ligand described herein, alters the recruitment of neosubstrates to the ubiquitin proteasome pathway. In some instance, the cysteine-containing protein is a member of the E3 ubiquitin ligase family. In other instances, the cysteine-containing protein is a member of the E3 ligase complex, such as for example, an adaptor, a substrate receptor subunit, a Cullin scaffold, or a RING-box subunit protein. In additional instances, the cysteine-containing protein is a target to be recruited by the ubiquitin proteasome pathway as a neosubstrate.

In some embodiments, the cysteine-containing protein is a protein illustrated in Table 1 (e.g., Table 1A). In some embodiments, Table 1 further illustrates one or more cysteine residues for interaction with a probe and/or a ligand described herein. In some cases, the cysteine residue number of a cysteine-containing protein is in reference to the respective UNIPROT identifier.

In some embodiments, the cysteine-containing protein is a protein illustrated in Table 2 (e.g., Table 2A). In some embodiments, Table 2 further illustrates one or more cysteine residues for interaction with a probe and/or a ligand described herein. In some cases, the cysteine residue number of a cysteine-containing protein is in reference to the respective UNIPROT identifier.

In some instances, a cysteine residue illustrated in Table 1 (e.g, Table 1A) or Table 2 (e.g., Table 2A) is located from 10 Å to 60 Å away from an active site residue of the respective cysteine-containing protein. In some instances, the cysteine residue is located at least 10 Å, 12 Å, 15 Å, 20 Å, 25 Å, 30 Å, 35 Å, 40 Å, 45 Å, or 50 Å away from an active site residue of the respective cysteine-containing protein. In some instances, the cysteine residue is located about 10 Å, 12 Å, 15 Å, 20 Å, 25 Å, 30 Å, 35 Å, 40 Å, 45 Å, or 50 Å away from an active site residue of the respective cysteine-containing protein.

In some instances, the probe binds to a cysteine residue of a member of the E3 ligase complex. In some cases, the probe binds to a cysteine residue of Anaphase-promoting complex subunit 16, Anaphase-promoting complex subunit 7, Apoptosis-resistant E3 ubiquitin protein ligase 1, Transcription regulator protein BACH1, Transcription regulator protein BACH2, Baculoviral IAP repeat-containing protein 2, Baculoviral IAP repeat-containing protein 3, DDB1- and CUL4-associated factor 17, Denticleless protein homolog, F-box only protein 11, F-box only protein 30, E3 ubiquitin-protein ligase HECTD1, Probable E3 ubiquitin-protein ligase HERC1, Probable E3 ubiquitin-protein ligase HERC4, E3 ISG15-protein ligase HERC5, E3 ubiquitin-protein ligase HUWE1, Kelch repeat and BTB domain-containing protein 8, Kelch-like ECH-associated protein 1, MYCBP2 Probable E3 ubiquitin-protein ligase MYCBP2, E3 ubiquitin-protein ligase MYCBP2, Polycomb group RING finger protein 2, E3 SUMO-protein ligase PIAS4, Protein PML, E3 ubiquitin-protein ligase RING1, E3 ubiquitin-protein ligase RING2, E3 ubiquitin-protein ligase BRE1A, E3 ubiquitin-protein ligase RNF213, RING finger protein 214, E3 ubiquitin-protein ligase RNF25, E3 ubiquitin-protein ligase BRE1B, RING-box protein 2, Tumor necrosis factor alpha-induced protein 3, E3 ubiquitin-protein ligase TRIM33, E3 ubiquitin-protein ligase TRIM56, Tripartite motif-containing protein 65, E3 ubiquitin-protein ligase TRIM71, E3 ubiquitin-protein ligase TRIP12, (E3-independent) E2 ubiquitin-conjugating enzyme, UBE4A Ubiquitin conjugation factor E4 A, Ubiquitin conjugation factor E4 A, E3 ubiquitin-protein ligase UBR2, E3 ubiquitin-protein ligase UBR4, E3 ubiquitin-protein ligase UBR5, Protein VPRBP, Vacuolar protein sorting-associated protein 18 homolog, or Nuclear-interacting partner of ALK.

In some instances, the protein is Ankyrin repeat and BTB/POZ domain-containing protein 1, Ankyrin repeat and BTB/POZ domain-containing protein 2, Activating molecule in BECN1-regulated autophagy protein 1, Anaphase-promoting complex subunit 11, Anaphase-promoting complex subunit 15, Anaphase-promoting complex subunit 16, Anaphase-promoting complex subunit 2, Anaphase-promoting complex subunit 7, Rabankyrin-5, Ankyrin repeat and IBR domain-containing protein 1, Amyloid protein-binding protein 2, Apoptosis-resistant E3 ubiquitin protein ligase 1, E3 ubiquitin-protein ligase ARIH1, E3 ubiquitin-protein ligase ARIH2, Armadillo repeat-containing protein 5, Ankyrin repeat and SOCS box protein 2, Ankyrin repeat and SOCS box protein 6, Transcriptional regulator ATRX, Transcription regulator protein BACH1, Transcription regulator protein BACH2, Baculoviral IAP repeat-containing protein 2, Baculoviral IAP repeat-containing protein 3, Baculoviral IAP repeat-containing protein 6, Breast cancer type 1 susceptibility protein, F-box/WD repeat-containing protein 1A, Cullin-associated NEDD8-dissociated protein 1, Cullin-associated NEDD8-dissociated protein 2, E3 ubiquitin-protein ligase CBL, E3 ubiquitin-protein ligase CBL-B, E3 ubiquitin-protein ligase CBL-C, Cyclin-F, Cell division cycle protein 20 homolog, Cell division cycle protein 23 homolog, Cell division cycle protein 27 homolog, Cell growth regulator with RING finger domain protein 1, E3 ubiquitin-protein ligase CHFR, Clusterin, CCR4-NOT transcription complex subunit 4, COMM domain-containing protein 2, COMM domain-containing protein 9, Cullin-4A, Cullin-5, Cullin-7, Cullin-9, DDB1- and CUL4-associated factor 1, DDB1- and CUL4-associated factor 10, DDB1- and CUL4-associated factor 13, DDB1- and CUL4-associated factor 16, DDB1- and CUL4-associated factor 17, DDB1- and CUL4-associated factor 5, DDB1- and CUL4-associated factor 6, DDB1- and CUL4-associated factor 7, DNA damage-binding protein 2, Zinc finger protein neuro-d4, Denticleless protein homolog, E3 ubiquitin-protein ligase DTX1, Probable E3 ubiquitin-protein ligase DTX3, E3 ubiquitin-protein ligase DTX3L, E3 SUMO-protein ligase EGR2, Ectoderm-neural cortex protein 1, DNA excision repair protein ERCC-8, E3 ubiquitin-protein ligase FANCL, F-box DNA helicase 1, F-box/LRR-repeat protein 12, F-box/LRR-repeat protein 17, F-box/LRR-repeat protein 18, F-box/LRR-repeat protein 20, F-box/LRR-repeat protein 3, F-box/LRR-repeat protein 6, F-box only protein 11, F-box only protein 30, F-box only protein 31, F-box only protein 38, F-box only protein 42, F-box/SPRY domain-containing protein 1, F-box only protein 5, F-box only protein 7, F-box only protein 9, F-box/WD repeat-containing protein 11, F-box/WD repeat-containing protein 4, F-box/WD repeat-containing protein 8, F-box/WD repeat-containing protein 9, Protein fem-1 homolog A, Protein fem-1 homolog B, Gigaxonin, General transcription factor IIH subunit 2, E3 ubiquitin-protein ligase HACE1, E3 ubiquitin-protein ligase HECTD1, Probable E3 ubiquitin-protein ligase HECTD4, E3 ubiquitin-protein ligase HECW1, E3 ubiquitin-protein ligase HECW2, Probable E3 ubiquitin-protein ligase HERC1, E3 ubiquitin-protein ligase HERC2, Probable E3 ubiquitin-protein ligase HERC3, Probable E3 ubiquitin-protein ligase HERC4, E3 ISG15-protein ligase HERC5, Helicase-like transcription factor, E3 ubiquitin-protein ligase HUWE1, Actin-binding protein IPP, Interferon regulatory factor 2-binding protein 1, Interferon regulatory factor 2-binding protein 2, Interferon regulatory factor 2-binding protein-like, E3 ubiquitin-protein ligase Itchy homolog, Influenza virus NS1A-binding protein, Kelch repeat and BTB domain-containing protein 11, Kelch repeat and BTB domain-containing protein 4, Kelch repeat and BTB domain-containing protein 6, Kelch repeat and BTB domain-containing protein 7, Kelch repeat and BTB domain-containing protein 8, E3 ubiquitin-protein ligase KCMF1, BTB/POZ domain-containing protein KCTD3, BTB/POZ domain-containing protein KCTD7, Kelch-like ECH-associated protein 1, Kelch domain-containing protein 10, Kelch domain-containing protein 3, Kelch-like protein 20, Kelch-like protein 24, Kelch-like protein 26, Kelch-like protein 36, E3 ubiquitin-protein ligase LNX, LON peptidase N-terminal domain and RING finger protein 2, Leucine-rich repeat protein 1, Leucine-rich repeat-containing protein 41, E3 ubiquitin-protein ligase LRSAM1, E3 ubiquitin-protein ligase listerin, E3 ubiquitin-protein ligase MARCH1, E3 ubiquitin-protein ligase MARCH3, E3 ubiquitin-protein ligase MARCH5, E3 ubiquitin-protein ligase MARCH6, E3 ubiquitin-protein ligase MARCH7, E3 ubiquitin-protein ligase Mdm2, E3 ubiquitin-protein ligase MGRN1, E3 ubiquitin-protein ligase MIB2, Probable E3 ubiquitin-protein ligase MID2, E3 ubiquitin-protein ligase makorin-1, Probable E3 ubiquitin-protein ligase makorin-2, Male-specific lethal 1 homolog, E3 ubiquitin-protein ligase MYCBP2, E3 ubiquitin-protein ligase NEDD4, E3 ubiquitin-protein ligase NEDD4-like, Transcriptional repressor NF-X1, NF-X1-type zinc finger protein NFXL1, Nitric oxide synthase-interacting protein, Histone-lysine N-methyltransferase NSD2, OTU domain-containing protein 7B, POZ-, AT hook-, and zinc finger-containing protein 1, Polycomb group RING finger protein 2, E3 ubiquitin-protein ligase PDZRN3, E3 ubiquitin-protein ligase pellino homolog 1, E3 ubiquitin-protein ligase pellino homolog 2, Peroxisome biogenesis factor 10, PHD and RING finger domain-containing protein 1, E3 SUMO-protein ligase PIAS2, E3 SUMO-protein ligase PIAS4, E3 ubiquitin-protein ligase Praja-2, Protein PML, RING-type E3 ubiquitin-protein ligase PPIL2, E3 ubiquitin-protein ligase parkin, Pre-mRNA-processing factor 19, E3 SUMO-protein ligase RanBP2, E3 ubiquitin-protein ligase RBX1, RCC1 and BTB domain-containing protein 1, RING finger and CHY zinc finger domain-containing protein 1, E3 ubiquitin-protein ligase RFWD3, E3 ubiquitin-protein ligase RING1, E3 ubiquitin-protein ligase RLIM, RING finger protein 10, E3 ubiquitin-protein ligase RNF114, E3 ubiquitin-protein ligase RNF126, E3 ubiquitin-protein ligase RNF128, E3 ubiquitin-protein ligase RNF135, E3 ubiquitin-protein ligase RNF14, E3 ubiquitin-protein ligase RNF144B, RING finger protein 148, E3 ubiquitin-protein ligase RNF149, RING finger protein 150, E3 ubiquitin ligase RNF157, E3 ubiquitin-protein ligase RNF168, E3 ubiquitin-protein ligase RNF187, E3 ubiquitin-protein ligase RNF19B, E3 ubiquitin-protein ligase RING2, E3 ubiquitin-protein ligase BRE1A, E3 ubiquitin-protein ligase RNF213, RING finger protein 214, E3 ubiquitin-protein ligase RNF216, Probable E3 ubiquitin-protein ligase RNF217, RING finger protein 219, E3 ubiquitin-protein ligase RNF25, E3 ubiquitin-protein ligase RNF31, E3 ubiquitin-protein ligase BRE1B, E3 ubiquitin-protein ligase RNF43, E3 ubiquitin-protein ligase RNF5, RING-box protein 2, E3 ubiquitin-protein ligase RNF8, RUN and FYVE domain-containing protein 1, E3 ubiquitin-protein ligase SH3RF2, SH3KBP1-binding protein 1, Structure-specific endonuclease subunit SLX4, E3 ubiquitin-protein ligase SMURF1, E3 ubiquitin-protein ligase SMURF2, Suppressor of cytokine signaling 2, Suppressor of cytokine signaling 3, Suppressor of cytokine signaling 6, Suppressor of cytokine signaling 7, Speckle-type POZ protein, Tumor necrosis factor alpha-induced protein 3, E3 ubiquitin-protein ligase Topors, TNF receptor-associated factor 1, TNF receptor-associated factor 2, TNF receptor-associated factor 3, E3 ubiquitin-protein ligase TRIM11, E3 ubiquitin-protein ligase TRIM22, E3 ubiquitin/ISG15 ligase TRIM25, Transcription intermediary factor 1-beta, Tripartite motif-containing protein 3, E3 ubiquitin-protein ligase TRIM32, E3 ubiquitin-protein ligase TRIM33, E3 ubiquitin-protein ligase TRIM36, E3 ubiquitin-protein ligase TRIM4, Tripartite motif-containing protein 47, E3 ubiquitin-protein ligase TRIM56, Tripartite motif-containing protein 59, Tripartite motif-containing protein 65, E3 ubiquitin-protein ligase TRIM7, E3 ubiquitin-protein ligase TRIM71, Tripartite motif-containing protein 72, E3 ubiquitin-protein ligase TRIM8, E3 ubiquitin-protein ligase TRIP12, Short transient receptor potential channel 4-associated protein, E3 ubiquitin-protein ligase TTC3, (E3-independent) E2 ubiquitin-conjugating enzyme, Ubiquitin-protein ligase E3 Å, Ubiquitin-protein ligase E3B, Ubiquitin-protein ligase E3C, E3 ubiquitin-protein ligase E3D, Ubiquitin conjugation factor E4 A, Ubiquitin conjugation factor E4 B, E3 ubiquitin-protein ligase UBR1, E3 ubiquitin-protein ligase UBR2, E3 ubiquitin-protein ligase UBR3, E3 ubiquitin-protein ligase UBR4, E3 ubiquitin-protein ligase UBR5, Putative E3 ubiquitin-protein ligase UBR7, RING finger protein unkempt homolog, Putative E3 ubiquitin-protein ligase UNKL, Vacuolar protein sorting-associated protein 11 homolog, Vacuolar protein sorting-associated protein 18 homolog, Vacuolar protein sorting-associated protein 41 homolog, Vacuolar protein sorting-associated protein 8 homolog, WD repeat-containing protein 26, WD and tetratricopeptide repeats protein 1, NEDD4-like E3 ubiquitin-protein ligase WWP1, NEDD4-like E3 ubiquitin-protein ligase WWP2, Nuclear-interacting partner of ALK, Zinc finger protein-like 1, E3 ubiquitin-protein ligase ZNF598, E3 ubiquitin-protein ligase ZNRF3.

In some embodiments, the cysteine-containing protein is a member of the E3-RING family. In some instances, the probe binds to a cysteine residue of a member of the E3-RING family. In some instances, the members comprise Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, and TNF receptor-associated factor 6. In some cases, the probe binds to a cysteine residue of Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, or TNF receptor-associated factor 6.

In some instances, the cysteine-containing protein is a member of the Cullin RING ligase (CRL) family. In some cases, the members comprise Elongin-B and Elongin-C. In some cases, the probe binds to a cysteine residue of Elongin-B or Elongin-C.

In some instances, the cysteine-containing protein is B-cell lymphoma 6 protein. In some cases, the probe binds to a cysteine residue of B-cell lymphoma 6 protein.

In some instances, the cysteine-containing protein is (E3-independent) E2 ubiquitin-conjugating enzyme. In some cases, the probe binds to a cysteine residue of (E3-independent) E2 ubiquitin-conjugating enzyme.

In some embodiments, described herein include a protein-probe adduct wherein the probe binds to a cysteine residue illustrated in Table 1A or Table 2A; wherein the probe has a structure represented by Formula (I):

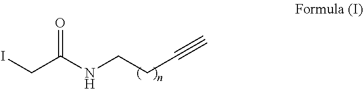

wherein,

n is 0-8.

In some instances, n is 0, 1, 2, 3, 4, 5, 6, 7, or 8. In some instances, n is 1. In some instances, n is 2. In some instances, n is 3. In some instances, n is 4. In some instances, n is 5. In some instances, n is 6. In some instances, n is 7. In some instances, n is 8.

In some instances, the protein is Ankyrin repeat and BTB/POZ domain-containing protein 1, Ankyrin repeat and BTB/POZ domain-containing protein 2, Activating molecule in BECN1-regulated autophagy protein 1, Anaphase-promoting complex subunit 11, Anaphase-promoting complex subunit 15, Anaphase-promoting complex subunit 16, Anaphase-promoting complex subunit 2, Anaphase-promoting complex subunit 7, Rabankyrin-5, Ankyrin repeat and IBR domain-containing protein 1, Amyloid protein-binding protein 2, Apoptosis-resistant E3 ubiquitin protein ligase 1, E3 ubiquitin-protein ligase ARIH1, E3 ubiquitin-protein ligase ARIH2, Armadillo repeat-containing protein 5, Ankyrin repeat and SOCS box protein 2, Ankyrin repeat and SOCS box protein 6, Transcriptional regulator ATRX, Transcription regulator protein BACH1, Transcription regulator protein BACH2, Baculoviral IAP repeat-containing protein 2, Baculoviral IAP repeat-containing protein 3, Baculoviral IAP repeat-containing protein 6, Breast cancer type 1 susceptibility protein, F-box/WD repeat-containing protein 1A, Cullin-associated NEDD8-dissociated protein 1, Cullin-associated NEDD8-dissociated protein 2, E3 ubiquitin-protein ligase CBL, E3 ubiquitin-protein ligase CBL-B, E3 ubiquitin-protein ligase CBL-C, Cyclin-F, Cell division cycle protein 20 homolog, Cell division cycle protein 23 homolog, Cell division cycle protein 27 homolog, Cell growth regulator with RING finger domain protein 1, E3 ubiquitin-protein ligase CHFR, Clusterin, CCR4-NOT transcription complex subunit 4, COMM domain-containing protein 2, COMM domain-containing protein 9, Cullin-4A, Cullin-5, Cullin-7, Cullin-9, DDB1- and CUL4-associated factor 1, DDB1- and CUL4-associated factor 10, DDB1- and CUL4-associated factor 13, DDB1- and CUL4-associated factor 16, DDB1- and CUL4-associated factor 17, DDB1- and CUL4-associated factor 5, DDB1- and CUL4-associated factor 6, DDB1- and CUL4-associated factor 7, DNA damage-binding protein 2, Zinc finger protein neuro-d4, Denticleless protein homolog, E3 ubiquitin-protein ligase DTX1, Probable E3 ubiquitin-protein ligase DTX3, E3 ubiquitin-protein ligase DTX3L, E3 SUMO-protein ligase EGR2, Ectoderm-neural cortex protein 1, DNA excision repair protein ERCC-8, E3 ubiquitin-protein ligase FANCL, F-box DNA helicase 1, F-box/LRR-repeat protein 12, F-box/LRR-repeat protein 17, F-box/LRR-repeat protein 18, F-box/LRR-repeat protein 20, F-box/LRR-repeat protein 3, F-box/LRR-repeat protein 6, F-box only protein 11, F-box only protein 30, F-box only protein 31, F-box only protein 38, F-box only protein 42, F-box/SPRY domain-containing protein 1, F-box only protein 5, F-box only protein 7, F-box only protein 9, F-box/WD repeat-containing protein 11, F-box/WD repeat-containing protein 4, F-box/WD repeat-containing protein 8, F-box/WD repeat-containing protein 9, Protein fem-1 homolog A, Protein fem-1 homolog B, Gigaxonin, General transcription factor IIH subunit 2, E3 ubiquitin-protein ligase HACE1, E3 ubiquitin-protein ligase HECTD1, Probable E3 ubiquitin-protein ligase HECTD4, E3 ubiquitin-protein ligase HECW1, E3 ubiquitin-protein ligase HECW2, Probable E3 ubiquitin-protein ligase HERC1, E3 ubiquitin-protein ligase HERC2, Probable E3 ubiquitin-protein ligase HERC3, Probable E3 ubiquitin-protein ligase HERC4, E3 ISG15-protein ligase HERC5, Helicase-like transcription factor, E3 ubiquitin-protein ligase HUWE1, Actin-binding protein IPP, Interferon regulatory factor 2-binding protein 1, Interferon regulatory factor 2-binding protein 2, Interferon regulatory factor 2-binding protein-like, E3 ubiquitin-protein ligase Itchy homolog, Influenza virus NS1A-binding protein, Kelch repeat and BTB domain-containing protein 11, Kelch repeat and BTB domain-containing protein 4, Kelch repeat and BTB domain-containing protein 6, Kelch repeat and BTB domain-containing protein 7, Kelch repeat and BTB domain-containing protein 8, E3 ubiquitin-protein ligase KCMF1, BTB/POZ domain-containing protein KCTD3, BTB/POZ domain-containing protein KCTD7, Kelch-like ECH-associated protein 1, Kelch domain-containing protein 10, Kelch domain-containing protein 3, Kelch-like protein 20, Kelch-like protein 24, Kelch-like protein 26, Kelch-like protein 36, E3 ubiquitin-protein ligase LNX, LON peptidase N-terminal domain and RING finger protein 2, Leucine-rich repeat protein 1, Leucine-rich repeat-containing protein 41, E3 ubiquitin-protein ligase LRSAM1, E3 ubiquitin-protein ligase listerin, E3 ubiquitin-protein ligase MARCH1, E3 ubiquitin-protein ligase MARCH3, E3 ubiquitin-protein ligase MARCH5, E3 ubiquitin-protein ligase MARCH6, E3 ubiquitin-protein ligase MARCH7, E3 ubiquitin-protein ligase Mdm2, E3 ubiquitin-protein ligase MGRN1, E3 ubiquitin-protein ligase MIB2, Probable E3 ubiquitin-protein ligase MID2, E3 ubiquitin-protein ligase makorin-1, Probable E3 ubiquitin-protein ligase makorin-2, Male-specific lethal 1 homolog, E3 ubiquitin-protein ligase MYCBP2, E3 ubiquitin-protein ligase NEDD4, E3 ubiquitin-protein ligase NEDD4-like, Transcriptional repressor NF-X1, NF-X1-type zinc finger protein NFXL1, Nitric oxide synthase-interacting protein, Histone-lysine N-methyltransferase NSD2, OTU domain-containing protein 7B, POZ-, AT hook-, and zinc finger-containing protein 1, Polycomb group RING finger protein 2, E3 ubiquitin-protein ligase PDZRN3, E3 ubiquitin-protein ligase pellino homolog 1, E3 ubiquitin-protein ligase pellino homolog 2, Peroxisome biogenesis factor 10, PHD and RING finger domain-containing protein 1, E3 SUMO-protein ligase PIAS2, E3 SUMO-protein ligase PIAS4, E3 ubiquitin-protein ligase Praja-2, Protein PML, RING-type E3 ubiquitin-protein ligase PPIL2, E3 ubiquitin-protein ligase parkin, Pre-mRNA-processing factor 19, E3 SUMO-protein ligase RanBP2, E3 ubiquitin-protein ligase RBX1, RCC1 and BTB domain-containing protein 1, RING finger and CHY zinc finger domain-containing protein 1, E3 ubiquitin-protein ligase RFWD3, E3 ubiquitin-protein ligase RING1, E3 ubiquitin-protein ligase RLIM, RING finger protein 10, E3 ubiquitin-protein ligase RNF114, E3 ubiquitin-protein ligase RNF126, E3 ubiquitin-protein ligase RNF128, E3 ubiquitin-protein ligase RNF135, E3 ubiquitin-protein ligase RNF14, E3 ubiquitin-protein ligase RNF144B, RING finger protein 148, E3 ubiquitin-protein ligase RNF149, RING finger protein 150, E3 ubiquitin ligase RNF157, E3 ubiquitin-protein ligase RNF168, E3 ubiquitin-protein ligase RNF187, E3 ubiquitin-protein ligase RNF19B, E3 ubiquitin-protein ligase RING2, E3 ubiquitin-protein ligase BRE1A, E3 ubiquitin-protein ligase RNF213, RING finger protein 214, E3 ubiquitin-protein ligase RNF216, Probable E3 ubiquitin-protein ligase RNF217, RING finger protein 219, E3 ubiquitin-protein ligase RNF25, E3 ubiquitin-protein ligase RNF31, E3 ubiquitin-protein ligase BRE1B, E3 ubiquitin-protein ligase RNF43, E3 ubiquitin-protein ligase RNF5, RING-box protein 2, E3 ubiquitin-protein ligase RNF8, RUN and FYVE domain-containing protein 1, E3 ubiquitin-protein ligase SH3RF2, SH3KBP1-binding protein 1, Structure-specific endonuclease subunit SLX4, E3 ubiquitin-protein ligase SMURF1, E3 ubiquitin-protein ligase SMURF2, Suppressor of cytokine signaling 2, Suppressor of cytokine signaling 3, Suppressor of cytokine signaling 6, Suppressor of cytokine signaling 7, Speckle-type POZ protein, Tumor necrosis factor alpha-induced protein 3, E3 ubiquitin-protein ligase Topors, TNF receptor-associated factor 1, TNF receptor-associated factor 2, TNF receptor-associated factor 3, E3 ubiquitin-protein ligase TRIM11, E3 ubiquitin-protein ligase TRIM22, E3 ubiquitin/ISG15 ligase TRIM25, Transcription intermediary factor 1-beta, Tripartite motif-containing protein 3, E3 ubiquitin-protein ligase TRIM32, E3 ubiquitin-protein ligase TRIM33, E3 ubiquitin-protein ligase TRIM36, E3 ubiquitin-protein ligase TRIM4, Tripartite motif-containing protein 47, E3 ubiquitin-protein ligase TRIM56, Tripartite motif-containing protein 59, Tripartite motif-containing protein 65, E3 ubiquitin-protein ligase TRIM7, E3 ubiquitin-protein ligase TRIM71, Tripartite motif-containing protein 72, E3 ubiquitin-protein ligase TRIM8, E3 ubiquitin-protein ligase TRIP12, Short transient receptor potential channel 4-associated protein, E3 ubiquitin-protein ligase TTC3, (E3-independent) E2 ubiquitin-conjugating enzyme, Ubiquitin-protein ligase E3A, Ubiquitin-protein ligase E3B, Ubiquitin-protein ligase E3C, E3 ubiquitin-protein ligase E3D, Ubiquitin conjugation factor E4 A, Ubiquitin conjugation factor E4 B, E3 ubiquitin-protein ligase UBR1, E3 ubiquitin-protein ligase UBR2, E3 ubiquitin-protein ligase UBR3, E3 ubiquitin-protein ligase UBR4, E3 ubiquitin-protein ligase UBR5, Putative E3 ubiquitin-protein ligase UBR7, RING finger protein unkempt homolog, Putative E3 ubiquitin-protein ligase UNKL, Vacuolar protein sorting-associated protein 11 homolog, Vacuolar protein sorting-associated protein 18 homolog, Vacuolar protein sorting-associated protein 41 homolog, Vacuolar protein sorting-associated protein 8 homolog, WD repeat-containing protein 26, WD and tetratricopeptide repeats protein 1, NEDD4-like E3 ubiquitin-protein ligase WWP1, NEDD4-like E3 ubiquitin-protein ligase WWP2, Nuclear-interacting partner of ALK, Zinc finger protein-like 1, E3 ubiquitin-protein ligase ZNF598, E3 ubiquitin-protein ligase ZNRF3.

In some embodiments, the probe with a structure represented by Formula (I) binds to a cysteine residue of a member of the E3-RING family. In some instances, the members comprise Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, and TNF receptor-associated factor 6. In some cases, the probe with a structure represented by Formula (I) binds to a cysteine residue of Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, or TNF receptor-associated factor 6.

In some instances, the probe with a structure represented by Formula (I) binds to a cysteine residue of Elongin-B or Elongin-C.

In some instances, the probe with a structure represented by Formula (I) binds to a cysteine residue of B-cell lymphoma 6 protein.

In some instances, the probe with a structure represented by Formula (I) binds to a cysteine residue of (E3-independent) E2 ubiquitin-conjugating enzyme.

In some instances, the protein is E3 ubiquitin-protein ligase TRIP12 (TRIP12). In some cases, the cysteine residue is C1959, wherein the numberings of the amino acid positions correspond to the amino acid positions with the UniProt Identifier Q14669. In some cases, the probe binds to C1959 of TRIP12.

In some instances, the protein is anaphase-promoting complex subunit 16 (ANAPC16). In some cases, the cysteine residue is C55, wherein the numbering of the amino acid position corresponds to the amino acid position with the UniProt Identifier Q96DE5. In some cases, the probe binds to C55 of ANAPC16.

In some instances, the protein is probable E3 ubiquitin-protein ligase MYCBP2 (MYCBP2). In some cases, the cysteine residue is C1131 or C3152, wherein the numberings of the amino acid positions correspond to the amino acid positions with the UniProt Identifier 075592. In some cases, the probe binds to C1131 or C3152 of MYCBP2.

In some instances, the protein is ubiquitin conjugation factor E4A (UBE4A). In some cases, the cysteine residue is C79, wherein the numbering of the amino acid position corresponds to the amino acid position with the UniProt Identifier Q14139. In some cases, the probe binds to C79 of UBE4A.

In some instances, the protein is autophagy-related protein 16-1 (ATG16L1). In some cases, the cysteine residue is C145, wherein the numbering of the amino acid position corresponds to the amino acid position with the UniProt Identifier Q676U5. In some cases, the probe binds to C145 of ATG16L1.

In some instances, the protein is protein arginine N-methyltransferase 5 (PRMT5). In some cases, the cysteine residue is C278, wherein the numbering of the amino acid position corresponds to the amino acid position with the UniProt Identifier 014744. In some cases, the probe binds to C278 of PRMT5.

In some instances, the protein is isocitrate dehydrogenase (IDH2). In some cases, the cysteine residue is C154, wherein the numberings of the amino acid positions correspond to the amino acid positions with the UniProt Identifier P48735. In some cases, the probe binds to C154 of IDH2.

In some instances, the protein is antigen peptide transporter 2 (TAP2). In some cases, the cysteine residue is C641, wherein the numbering of the amino acid position corresponds to the amino acid position with the UniProt Identifier Q03519. In some cases, the probe binds to C641 of TAP2.

In some instances, the protein is tapasin (TAPBP). In some cases, the cysteine residue is C440, wherein the numbering of the amino acid position corresponds to the amino acid position with the UniProt Identifier O15533. In some cases, the probe binds to C440 of TAPBP.

In some instances, the protein is protein unc-93 homolog B1 (UNC93B1). In some cases, the cysteine residue is C583, wherein the numbering of the amino acid position corresponds to the amino acid position with the UniProt Identifier Q9H1C4. In some cases, the probe binds to C583 of UNC93B1.

In some instances, the protein is probable ATP-dependent RNA helicase DDX60 (DDX60). In some cases, the cysteine residue is C1051, wherein the numbering of the amino acid position corresponds to the amino acid position with the UniProt Identifier Q81Y21. In some cases, the probe binds to C1051 of DDX60.

In some embodiments, the protein is B-cell lymphoma 6 protein (BCL6) and the cysteine residue is C121, C175, C232, C254, C296, C339, C348, C354, C414, C548, or C663, wherein the numbering of the amino acid position corresponds to the amino acid position with the UniProt Identifier P41182. In some cases, the probe binds to C121, C175, C232, C254, C296, C339, C348, C354, C414, C548, or C663 of BCL6.

In some instances, the protein is B-cell lymphoma 6 protein. In some cases, the cysteine residue is C121, wherein the numbering of the amino acid position corresponds to the amino acid position with the UniProt Identifier P41182. In some cases, the probe binds to C121 of BCL6.

In some instances, the protein is B-cell lymphoma 6 protein. In some cases, the cysteine residue is C175, wherein the numbering of the amino acid position corresponds to the amino acid position with the UniProt Identifier P41182. In some cases, the probe binds to C175 of BCL6.

In some instances, the protein is B-cell lymphoma 6 protein. In some cases, the cysteine residue is C232, wherein the numbering of the amino acid position corresponds to the amino acid position with the UniProt Identifier P41182. In some cases, the probe binds to C232 of BCL6.

In some instances, the protein is B-cell lymphoma 6 protein. In some cases, the cysteine residue is C254, wherein the numbering of the amino acid position corresponds to the amino acid position with the UniProt Identifier P41182. In some cases, the probe binds to C254 of BCL6.

In some instances, the protein is B-cell lymphoma 6 protein. In some cases, the cysteine residue is C296, wherein the numbering of the amino acid position corresponds to the amino acid position with the UniProt Identifier P41182. In some cases, the probe binds to C296 of BCL6.

In some instances, the protein is B-cell lymphoma 6 protein. In some cases, the cysteine residue is C339, wherein the numbering of the amino acid position corresponds to the amino acid position with the UniProt Identifier P41182. In some cases, the probe binds to C339 of BCL6.

In some instances, the protein is B-cell lymphoma 6 protein. In some cases, the cysteine residue is C348, wherein the numbering of the amino acid position corresponds to the amino acid position with the UniProt Identifier P41182. In some cases, the probe binds to C348 of BCL6.

In some instances, the protein is B-cell lymphoma 6 protein. In some cases, the cysteine residue is C354, wherein the numbering of the amino acid position corresponds to the amino acid position with the UniProt Identifier P41182. In some cases, the probe binds to C354 of BCL6.

In some instances, the protein is B-cell lymphoma 6 protein. In some cases, the cysteine residue is C414, wherein the numbering of the amino acid position corresponds to the amino acid position with the UniProt Identifier P41182. In some cases, the probe binds to C414 of BCL6.

In some instances, the protein is B-cell lymphoma 6 protein. In some cases, the cysteine residue is C548, wherein the numbering of the amino acid position corresponds to the amino acid position with the UniProt Identifier P41182. In some cases, the probe binds to C548 of BCL6.

In some instances, the protein is B-cell lymphoma 6 protein. In some cases, the cysteine residue is C663, wherein the numbering of the amino acid position corresponds to the amino acid position with the UniProt Identifier P41182. In some cases, the probe binds to C663 of BCL6.

In some embodiments, the protein is Polycomb group RING finger protein 6 (PCGF6) and the cysteine residue is C56, C137, or C155, wherein the numbering of the amino acid position corresponds to the amino acid position with the UniProt Identifier Q9BYE7. In some cases, the probe binds to C56, C137, or C155 of PCGF6.

In some instances, the protein is Polycomb group RING finger protein 6. In some cases, the cysteine residue is C56, wherein the numbering of the amino acid position corresponds to the amino acid position with the UniProt Identifier Q9BYE7. In some cases, the probe binds to C56 of PCGF6.

In some instances, the protein is Polycomb group RING finger protein 6. In some cases, the cysteine residue is C137, wherein the numbering of the amino acid position corresponds to the amino acid position with the UniProt Identifier Q9BYE7. In some cases, the probe binds to C137 of PCGF6.

In some instances, the protein is Polycomb group RING finger protein 6. In some cases, the cysteine residue is C155, wherein the numbering of the amino acid position corresponds to the amino acid position with the UniProt Identifier Q9BYE7. In some cases, the probe binds to C155 of PCGF6.

In some embodiments, the protein is E3 ubiquitin-protein ligase CBL-B (CBLB) and the cysteine residue is C60, C345, C376, C435, C436, C470, C523, C535, C594, C607, C686, C741, or C895, wherein the numbering of the amino acid position corresponds to the amino acid position with the UniProt Identifier Q13191. In some cases, the probe binds to C60, C345, C376, C435, C436, C470, C523, C535, C594, C607, C686, C741, or C895 of CBLB.

In some instances, the protein is E3 ubiquitin-protein ligase CBL-B. In some cases, the cysteine residue is C60, wherein the numbering of the amino acid position corresponds to the amino acid position with the UniProt Identifier Q13191. In some cases, the probe binds to C60 of CBLB.

In some instances, the protein is E3 ubiquitin-protein ligase CBL-B. In some cases, the cysteine residue is C345, wherein the numbering of the amino acid position corresponds to the amino acid position with the UniProt Identifier Q13191. In some cases, the probe binds to C345 of CBLB.

In some instances, the protein is E3 ubiquitin-protein ligase CBL-B. In some cases, the cysteine residue is C376, wherein the numbering of the amino acid position corresponds to the amino acid position with the UniProt Identifier Q13191. In some cases, the probe binds to C376 of CBLB.

In some instances, the protein is E3 ubiquitin-protein ligase CBL-B. In some cases, the cysteine residue is C435, wherein the numbering of the amino acid position corresponds to the amino acid position with the UniProt Identifier Q13191. In some cases, the probe binds to C435 of CBLB.

In some instances, the protein is E3 ubiquitin-protein ligase CBL-B. In some cases, the cysteine residue is C436, wherein the numbering of the amino acid position corresponds to the amino acid position with the UniProt Identifier Q13191. In some cases, the probe binds to C436 of CBLB.

In some instances, the protein is E3 ubiquitin-protein ligase CBL-B. In some cases, the cysteine residue is C470, wherein the numbering of the amino acid position corresponds to the amino acid position with the UniProt Identifier Q13191. In some cases, the probe binds to C470 of CBLB.

In some instances, the protein is E3 ubiquitin-protein ligase CBL-B. In some cases, the cysteine residue is C523, wherein the numbering of the amino acid position corresponds to the amino acid position with the UniProt Identifier Q13191. In some cases, the probe binds to C523 of CBLB.

In some instances, the protein is E3 ubiquitin-protein ligase CBL-B. In some cases, the cysteine residue is C535, wherein the numbering of the amino acid position corresponds to the amino acid position with the UniProt Identifier Q13191. In some cases, the probe binds to C535 of CBLB.

In some instances, the protein is E3 ubiquitin-protein ligase CBL-B. In some cases, the cysteine residue is C594, wherein the numbering of the amino acid position corresponds to the amino acid position with the UniProt Identifier Q13191. In some cases, the probe binds to C594 of CBLB.

In some instances, the protein is E3 ubiquitin-protein ligase CBL-B. In some cases, the cysteine residue is C607, wherein the numbering of the amino acid position corresponds to the amino acid position with the UniProt Identifier Q13191. In some cases, the probe binds to C607 of CBLB.

In some instances, the protein is E3 ubiquitin-protein ligase CBL-B. In some cases, the cysteine residue is C686, wherein the numbering of the amino acid position corresponds to the amino acid position with the UniProt Identifier Q13191. In some cases, the probe binds to C686 of CBLB.

In some instances, the protein is E3 ubiquitin-protein ligase CBL-B. In some cases, the cysteine residue is C741, wherein the numbering of the amino acid position corresponds to the amino acid position with the UniProt Identifier Q13191. In some cases, the probe binds to C741 of CBLB.

In some instances, the protein is E3 ubiquitin-protein ligase CBL-B. In some cases, the cysteine residue is C895, wherein the numbering of the amino acid position corresponds to the amino acid position with the UniProt Identifier Q13191. In some cases, the probe binds to C895 of CBLB.

In some embodiments, the protein is Elongin-B (ELOB) and the cysteine residue is C60 or C89, wherein the numbering of the amino acid position corresponds to the amino acid position with the UniProt Identifier Q15370. In some cases, the probe binds to C60 or C89 of ELOB.

In some instances, the protein is Elongin-B. In some cases, the cysteine residue is C60, wherein the numbering of the amino acid position corresponds to the amino acid position with the UniProt Identifier Q15370. In some cases, the probe binds to C60 of ELOB.

In some instances, the protein is Elongin-B. In some cases, the cysteine residue is C89, wherein the numbering of the amino acid position corresponds to the amino acid position with the UniProt Identifier Q15370. In some cases, the probe binds to 89C of ELOB.

In some embodiments, the protein is Elongin-C (ELOC) and the cysteine residue is C11 or C74, wherein the numbering of the amino acid position corresponds to the amino acid position with the UniProt Identifier Q15369. In some cases, the probe binds to C11 or C74 of ELOC.

In some instances, the protein is Elongin-C. In some cases, the cysteine residue is C11, wherein the numbering of the amino acid position corresponds to the amino acid position with the UniProt Identifier Q15369. In some cases, the probe binds to C11 of ELOC.

In some instances, the protein is Elongin-C. In some cases, the cysteine residue is C74, wherein the numbering of the amino acid position corresponds to the amino acid position with the UniProt Identifier Q15369. In some cases, the probe binds to C74 of ELOC.

In some embodiments, the protein is F-box only protein 22 (FBXO22) and the cysteine residue is C47, C117, C227, C228, or C378, wherein the numbering of the amino acid position corresponds to the amino acid position with the UniProt Identifier Q8NEZ5. In some cases, the probe binds to C47, C117, C227, C228, or C378 of FBXO22.

In some instances, the protein is F-box only protein 22. In some cases, the cysteine residue is C47, wherein the numbering of the amino acid position corresponds to the amino acid position with the UniProt Identifier Q8NEZ5. In some cases, the probe binds to C47 of FBXO22.

In some instances, the protein is F-box only protein 22. In some cases, the cysteine residue is C117, wherein the numbering of the amino acid position corresponds to the amino acid position with the UniProt Identifier Q8NEZ5. In some cases, the probe binds to C117 of FBXO22.

In some instances, the protein is F-box only protein 22. In some cases, the cysteine residue is C227, wherein the numbering of the amino acid position corresponds to the amino acid position with the UniProt Identifier Q8NEZ5. In some cases, the probe binds to C227 of FBXO22.

In some instances, the protein is F-box only protein 22. In some cases, the cysteine residue is C228, wherein the numbering of the amino acid position corresponds to the amino acid position with the UniProt Identifier Q8NEZ5. In some cases, the probe binds to C228 of FBXO22.

In some instances, the protein is F-box only protein 22. In some cases, the cysteine residue is C378, wherein the numbering of the amino acid position corresponds to the amino acid position with the UniProt Identifier Q8NEZ5. In some cases, the probe binds to C378 of FBXO22.

In some embodiments, the protein is Kelch repeat and BTB domain-containing protein 4 (KBTBD4) and the cysteine residue is C68, C201, C274, C301, C455, or C472, wherein the numbering of the amino acid position corresponds to the amino acid position with the UniProt Identifier Q9NVX7. In some cases, the probe binds to C68, C201, C274, C301, C455, or C472 of KBTBD4.

In some instances, the protein is Kelch repeat and BTB domain-containing protein 4. In some cases, the cysteine residue is C68, wherein the numbering of the amino acid position corresponds to the amino acid position with the UniProt Identifier Q9NVX7. In some cases, the probe binds to C68 of KBTBD4.

In some instances, the protein is Kelch repeat and BTB domain-containing protein 4. In some cases, the cysteine residue is C201, wherein the numbering of the amino acid position corresponds to the amino acid position with the UniProt Identifier Q9NVX7. In some cases, the probe binds to C201 of KBTBD4.

In some instances, the protein is Kelch repeat and BTB domain-containing protein 4. In some cases, the cysteine residue is C274, wherein the numbering of the amino acid position corresponds to the amino acid position with the UniProt Identifier Q9NVX7. In some cases, the probe binds to C274 of KBTBD4.

In some instances, the protein is Kelch repeat and BTB domain-containing protein 4. In some cases, the cysteine residue is C301, wherein the numbering of the amino acid position corresponds to the amino acid position with the UniProt Identifier Q9NVX7. In some cases, the probe binds to C301 of KBTBD4.

In some instances, the protein is Kelch repeat and BTB domain-containing protein 4. In some cases, the cysteine residue is C455, wherein the numbering of the amino acid position corresponds to the amino acid position with the UniProt Identifier Q9NVX7. In some cases, the probe binds to C455 of KBTBD4.

In some instances, the protein is Kelch repeat and BTB domain-containing protein 4. In some cases, the cysteine residue is C472, wherein the numbering of the amino acid position corresponds to the amino acid position with the UniProt Identifier Q9NVX7. In some cases, the probe binds to C472 of KBTBD4.

In some embodiments, the protein is Kelch-like ECH-associated protein 1 (KEAP1) and the cysteine residue is C23, C38, C151, C226, C241, C257, C288, C297, C319, C434, C613, C622, or C624 wherein the numbering of the amino acid position corresponds to the amino acid position with the UniProt Identifier Q14145. In some cases, the probe binds to C23, C38, C151, C226, C241, C257, C288, C297, C319, C434, C613, C622, or C624 of KEAP1.

In some instances, the protein is Kelch-like ECH-associated protein 1. In some cases, the cysteine residue is C23, wherein the numbering of the amino acid position corresponds to the amino acid position with the UniProt Identifier Q14145. In some cases, the probe binds to C23 of KEAP1.

In some instances, the protein is Kelch-like ECH-associated protein 1. In some cases, the cysteine residue is C38, wherein the numbering of the amino acid position corresponds to the amino acid position with the UniProt Identifier Q14145. In some cases, the probe binds to C38 of KEAP1.

In some instances, the protein is Kelch-like ECH-associated protein 1. In some cases, the cysteine residue is C151, wherein the numbering of the amino acid position corresponds to the amino acid position with the UniProt Identifier Q14145. In some cases, the probe binds to C151 of KEAP1.

In some instances, the protein is Kelch-like ECH-associated protein 1. In some cases, the cysteine residue is C226, wherein the numbering of the amino acid position corresponds to the amino acid position with the UniProt Identifier Q14145. In some cases, the probe binds to C226 of KEAP1.

In some instances, the protein is Kelch-like ECH-associated protein 1. In some cases, the cysteine residue is C241, wherein the numbering of the amino acid position corresponds to the amino acid position with the UniProt Identifier Q14145. In some cases, the probe binds to C241 of KEAP1.

In some instances, the protein is Kelch-like ECH-associated protein 1. In some cases, the cysteine residue is C257, wherein the numbering of the amino acid position corresponds to the amino acid position with the UniProt Identifier Q14145. In some cases, the probe binds to C257 of KEAP1.

In some instances, the protein is Kelch-like ECH-associated protein 1. In some cases, the cysteine residue is C288, wherein the numbering of the amino acid position corresponds to the amino acid position with the UniProt Identifier Q14145. In some cases, the probe binds to C288 of KEAP1.

In some instances, the protein is Kelch-like ECH-associated protein 1. In some cases, the cysteine residue is C297, wherein the numbering of the amino acid position corresponds to the amino acid position with the UniProt Identifier Q14145. In some cases, the probe binds to C297 of KEAP1.

In some instances, the protein is Kelch-like ECH-associated protein 1. In some cases, the cysteine residue is C319, wherein the numbering of the amino acid position corresponds to the amino acid position with the UniProt Identifier Q14145. In some cases, the probe binds to C319 of KEAP1.

In some instances, the protein is Kelch-like ECH-associated protein 1. In some cases, the cysteine residue is C434, wherein the numbering of the amino acid position corresponds to the amino acid position with the UniProt Identifier Q14145. In some cases, the probe binds to C434 of KEAP1.

In some instances, the protein is Kelch-like ECH-associated protein 1. In some cases, the cysteine residue is C613, wherein the numbering of the amino acid position corresponds to the amino acid position with the UniProt Identifier Q14145. In some cases, the probe binds to C613 of KEAP1.

In some instances, the protein is Kelch-like ECH-associated protein 1. In some cases, the cysteine residue is C624 wherein the numbering of the amino acid position corresponds to the amino acid position with the UniProt Identifier Q14145. In some cases, the probe binds to C622 of KEAP1.

In some instances, the protein is Kelch-like ECH-associated protein 1. In some cases, the cysteine residue is C624 wherein the numbering of the amino acid position corresponds to the amino acid position with the UniProt Identifier Q14145. In some cases, the probe binds to C624 of KEAP1.

In some embodiments, the protein is E3 ubiquitin-protein ligase pellino homolog 11 (PELI1) and the cysteine residue is C61, C212, or C282 wherein the numbering of the amino acid position corresponds to the amino acid position with the UniProt Identifier Q96FA3. In some cases, the probe binds to C61, C212, or C282 of PELI1.

In some instances, the protein is E3 ubiquitin-protein ligase pellino homolog 11. In some cases, the cysteine residue is C61, wherein the numbering of the amino acid position corresponds to the amino acid position with the UniProt Identifier Q96FA3. In some cases, the probe binds to C61 of PELI1.

In some instances, the protein is E3 ubiquitin-protein ligase pellino homolog 11. In some cases, the cysteine residue is C212 wherein the numbering of the amino acid position corresponds to the amino acid position with the UniProt Identifier Q96FA3. In some cases, the probe binds to C212 of PELI1.

In some instances, the protein is E3 ubiquitin-protein ligase pellino homolog 11. In some cases, the cysteine residue is C282 wherein the numbering of the amino acid position corresponds to the amino acid position with the UniProt Identifier Q96FA3. In some cases, the probe binds to C282 of PELI1.

In some embodiments, the protein is E3 ubiquitin-protein ligase RNF128 (RNF128) and the cysteine residue is C295, C303, C314, or C317 wherein the numbering of the amino acid position corresponds to the amino acid position with the UniProt Identifier Q8TEB7. In some cases, the probe binds to C295, C303, C314, or C317 of RNF128.

In some instances, the protein is E3 ubiquitin-protein ligase RNF128. In some cases, the cysteine residue is C295, wherein the numbering of the amino acid position corresponds to the amino acid position with the UniProt Identifier Q8TEB7. In some cases, the probe binds to C295 of RNF128.

In some instances, the protein is E3 ubiquitin-protein ligase RNF128. In some cases, the cysteine residue is C303, wherein the numbering of the amino acid position corresponds to the amino acid position with the UniProt Identifier Q8TEB7. In some cases, the probe binds to C303 of RNF128.

In some instances, the protein is E3 ubiquitin-protein ligase RNF128. In some cases, the cysteine residue is C314 wherein the numbering of the amino acid position corresponds to the amino acid position with the UniProt Identifier Q8TEB7. In some cases, the probe binds to C314 of RNF128.

In some instances, the protein is E3 ubiquitin-protein ligase RNF128. In some cases, the cysteine residue is C317 wherein the numbering of the amino acid position corresponds to the amino acid position with the UniProt Identifier Q8TEB7. In some cases, the probe binds to C317 of RNF128.

In some embodiments, the protein is TNF receptor-associated factor 6 (TRAF6) and the cysteine residue is C85, C105, C134, C139, C182, C235, C349, or C366, wherein the numbering of the amino acid position corresponds to the amino acid position with the UniProt Identifier Q9Y4K3. In some cases, the probe binds to C85, C105, C134, C139, C182, C235, C349, or C366 of TRAF6.

In some instances, the protein is TNF receptor-associated factor 6. In some cases, the cysteine residue is C85, wherein the numbering of the amino acid position corresponds to the amino acid position with the UniProt Identifier Q9Y4K3. In some cases, the probe binds to C85 of TRAF6.

In some instances, the protein is TNF receptor-associated factor 6. In some cases, the cysteine residue is C105, wherein the numbering of the amino acid position corresponds to the amino acid position with the UniProt Identifier Q9Y4K3. In some cases, the probe binds to C105 of TRAF6.

In some instances, the protein is TNF receptor-associated factor 6. In some cases, the cysteine residue is C134, wherein the numbering of the amino acid position corresponds to the amino acid position with the UniProt Identifier Q9Y4K3. In some cases, the probe binds to C134 of TRAF6.

In some instances, the protein is TNF receptor-associated factor 6. In some cases, the cysteine residue is C139, wherein the numbering of the amino acid position corresponds to the amino acid position with the UniProt Identifier Q9Y4K3. In some cases, the probe binds to C139 of TRAF6.

In some instances, the protein is TNF receptor-associated factor 6. In some cases, the cysteine residue is C182, wherein the numbering of the amino acid position corresponds to the amino acid position with the UniProt Identifier Q9Y4K3. In some cases, the probe binds to C182 of TRAF6.

In some instances, the protein is TNF receptor-associated factor 6. In some cases, the cysteine residue is C235, wherein the numbering of the amino acid position corresponds to the amino acid position with the UniProt Identifier Q9Y4K3. In some cases, the probe binds to C235 of TRAF6.

In some instances, the protein is TNF receptor-associated factor 6. In some cases, the cysteine residue is C349, wherein the numbering of the amino acid position corresponds to the amino acid position with the UniProt Identifier Q9Y4K3. In some cases, the probe binds to C349 of TRAF6.

In some instances, the protein is TNF receptor-associated factor 6. In some cases, the cysteine residue is C366, wherein the numbering of the amino acid position corresponds to the amino acid position with the UniProt Identifier Q9Y4K3. In some cases, the probe binds to C366 of TRAF6.

In some embodiments, the protein is (E3-independent) E2 ubiquitin-conjugating enzyme (UBE20) and the cysteine residue is C101, C182, C208, C230, C244, C314, C341, C370, C375, C400, C406, C585, C598, C910, C913, C1040, C1099, or C1288, wherein the numbering of the amino acid position corresponds to the amino acid position with the UniProt Identifier Q9C0C9. In some cases, the probe binds to C101, C182, C208, C230, C244, C314, C341, C370, C375, C400, C406, C585, C598, C910, C913, C1040, C1099, or C1288 of UBE20.

In some instances, the protein is (E3-independent) E2 ubiquitin-conjugating enzyme and the cysteine residue is C101, wherein the numbering of the amino acid position corresponds to the amino acid position with the UniProt Identifier Q9C0C9. In some cases, the probe binds to C101 of UBE20.

In some instances, the protein is (E3-independent) E2 ubiquitin-conjugating enzyme and the cysteine residue is C182, wherein the numbering of the amino acid position corresponds to the amino acid position with the UniProt Identifier Q9C0C9. In some cases, the probe binds to C182 of UBE20.

In some instances, the protein is (E3-independent) E2 ubiquitin-conjugating enzyme and the cysteine residue is C208, wherein the numbering of the amino acid position corresponds to the amino acid position with the UniProt Identifier Q9C0C9. In some cases, the probe binds to C208 of UBE20.

In some instances, the protein is (E3-independent) E2 ubiquitin-conjugating enzyme and the cysteine residue is C230, wherein the numbering of the amino acid position corresponds to the amino acid position with the UniProt Identifier Q9C0C9. In some cases, the probe binds to C230 of UBE20.

In some instances, the protein is (E3-independent) E2 ubiquitin-conjugating enzyme and the cysteine residue is C244, wherein the numbering of the amino acid position corresponds to the amino acid position with the UniProt Identifier Q9C0C9. In some cases, the probe binds to C244 of UBE20.

In some instances, the protein is (E3-independent) E2 ubiquitin-conjugating enzyme and the cysteine residue is C314, wherein the numbering of the amino acid position corresponds to the amino acid position with the UniProt Identifier Q9C0C9. In some cases, the probe binds to C314 of UBE20.

In some instances, the protein is (E3-independent) E2 ubiquitin-conjugating enzyme and the cysteine residue is C341, wherein the numbering of the amino acid position corresponds to the amino acid position with the UniProt Identifier Q9C0C9. In some cases, the probe binds to C341 of UBE20.

In some instances, the protein is (E3-independent) E2 ubiquitin-conjugating enzyme and the cysteine residue is C370, wherein the numbering of the amino acid position corresponds to the amino acid position with the UniProt Identifier Q9C0C9. In some cases, the probe binds to C370 of UBE20.

In some instances, the protein is (E3-independent) E2 ubiquitin-conjugating enzyme and the cysteine residue is C375, wherein the numbering of the amino acid position corresponds to the amino acid position with the UniProt Identifier Q9C0C9. In some cases, the probe binds to C375 of UBE20.

In some instances, the protein is (E3-independent) E2 ubiquitin-conjugating enzyme and the cysteine residue is C400, wherein the numbering of the amino acid position corresponds to the amino acid position with the UniProt Identifier Q9C0C9. In some cases, the probe binds to C400 of UBE20.

In some instances, the protein is (E3-independent) E2 ubiquitin-conjugating enzyme and the cysteine residue is C406, wherein the numbering of the amino acid position corresponds to the amino acid position with the UniProt Identifier Q9C0C9. In some cases, the probe binds to C406 of UBE20.

In some instances, the protein is (E3-independent) E2 ubiquitin-conjugating enzyme and the cysteine residue is C585, wherein the numbering of the amino acid position corresponds to the amino acid position with the UniProt Identifier Q9C0C9. In some cases, the probe binds to C585 of UBE20.

In some instances, the protein is (E3-independent) E2 ubiquitin-conjugating enzyme and the cysteine residue is C598, wherein the numbering of the amino acid position corresponds to the amino acid position with the UniProt Identifier Q9C0C9. In some cases, the probe binds to C598 of UBE20.

In some instances, the protein is (E3-independent) E2 ubiquitin-conjugating enzyme and the cysteine residue is C910, wherein the numbering of the amino acid position corresponds to the amino acid position with the UniProt Identifier Q9C0C9. In some cases, the probe binds to C910 of UBE20.

In some instances, the protein is (E3-independent) E2 ubiquitin-conjugating enzyme and the cysteine residue is C913, wherein the numbering of the amino acid position corresponds to the amino acid position with the UniProt Identifier Q9C0C9. In some cases, the probe binds to C913 of UBE20.

In some instances, the protein is (E3-independent) E2 ubiquitin-conjugating enzyme and the cysteine residue is C1040, wherein the numbering of the amino acid position corresponds to the amino acid position with the UniProt Identifier Q9C0C9. In some cases, the probe binds to C1040 of UBE20.

In some instances, the protein is (E3-independent) E2 ubiquitin-conjugating enzyme and the cysteine residue is C1099, wherein the numbering of the amino acid position corresponds to the amino acid position with the UniProt Identifier Q9C0C9. In some cases, the probe binds to C1099 of UBE20.

In some instances, the protein is (E3-independent) E2 ubiquitin-conjugating enzyme and the cysteine residue is C1288, wherein the numbering of the amino acid position corresponds to the amino acid position with the UniProt Identifier Q9C0C9. In some cases, the probe binds to C1288 of UBE20.

In some embodiments, also described herein include a synthetic ligand that inhibits a covalent interaction between a protein and a probe, wherein in the absence of the synthetic ligand, the probe binds to a cysteine residue illustrated in Table 1A or Table 2A; and wherein the probe has a structure represented by Formula (I):

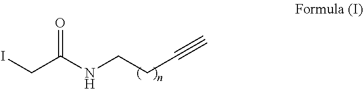

wherein,
n is 0-8.

In some instances, n is 0, 1, 2, 3, 4, 5, 6, 7, or 8. In some instances, n is 1. In some instances, n is 2. In some instances, n is 3. In some instances, n is 4. In some instances, n is 5. In some instances, n is 6. In some instances, n is 7. In some instances, n is 8.

In some instances, the synthetic ligand comprises a structure represented by Formula II:

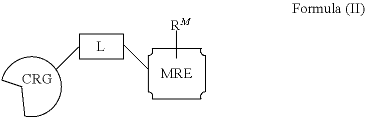

wherein,
CRG-L is optional, and when present is a covalent reactive group comprising a Michael acceptor moiety, a leaving group moiety, or a moiety capable of forming a covalent bond to the thiol group of a cysteine residue, and L is a linker;

MRE is a molecular recognition element that is capable of interacting with the protein; and R^{M} is optional, and when present comprises a binding element that binds to a second protein or another compound.

In some cases, the Michael acceptor moiety comprises an alkene or an alkyne moiety. In some cases, the Michael acceptor moiety comprises an alkene moiety. In some cases, the Michael acceptor moiety comprises an alkyne moiety.

In some instances, L is a cleavable linker. In other instances, L is a non-cleavable linker.

In some cases, MRE comprises a small molecule compound, a polynucleotide, a polypeptide or fragments thereof, or a peptidomimetic. In some embodiments, MRE is a small molecule compound. In some embodiments, MRE is a polynucleotide. In some embodiments, MRE is a polypeptide or fragments thereof. In some embodiments, MRE is a peptidomimetic.

In some embodiments, the synthetic ligand has a structure represented by Formula (IIA) or Formula (IIB):

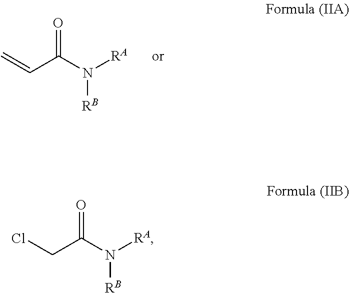

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl; or
R^{A} and R^{B} together with the nitrogen to which they are attached form a substituted or unsubstituted 5, 6, 7 or 8-membered heterocyclic ring A, optionally having one additional heteroatom moiety independently selected from NR¹, O, or S; and
R¹ is H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

In some embodiments, R^{A} is H or D.

In some embodiments, R^{B} is substituted or unsubstituted aryl or substituted or unsubstituted heteroaryl. In some embodiments, R^{B} is substituted or unsubstituted aryl. In some embodiments, R^{B} is substituted or unsubstituted heteroaryl.

In some embodiments, R^{B} is substituted aryl. In some embodiments, R^{B} is aryl, substituted with one or more substituents selected from the group consisting of halogen, C₁-C₄fluoroalkyl, —CN, and —NO₂.

In some embodiments, R^{A} and R^{B} together with the nitrogen to which they are attached form a substituted or unsubstituted 6 or 7-membered heterocyclic ring A. In some embodiments, ring A is a 6-membered heterocyclic ring. In some embodiments, In some embodiments, ring A is 6-membered heterocyclic ring substituted with substituted or unsubstituted aryl or substituted or unsubstituted heteroaryl. In some embodiments, ring A is 6-membered heterocyclic ring fused with substituted or unsubstituted aryl or substituted or unsubstituted heteroaryl.

In some cases, the synthetic ligand is

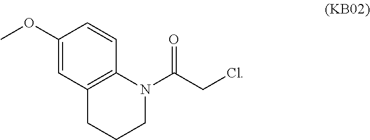 [SMILES: COc1ccc2c(c1)CCCN2C(=O)CCl]

In some cases, the synthetic ligand is:

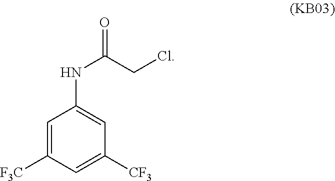 [SMILES: O=C(CCl)Nc1cc(C(F)(F)F)cc(C(F)(F)F)c1]

In some cases, the synthetic ligand is:

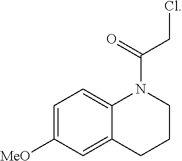 [SMILES: COc1ccc2c(c1)CCCN2C(=O)CCl]

In some cases, the synthetic ligand is:

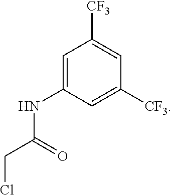 [SMILES: O=C(CCl)Nc1cc(C(F)(F)F)cc(C(F)(F)F)c1]

In some cases, the synthetic ligand is:

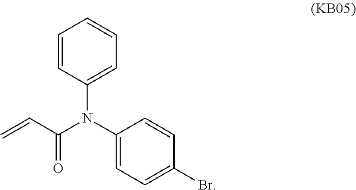 [SMILES: C=CC(=O)N(c1ccccc1)c1ccc(Br)cc1]

In some instances, the protein is Anaphase-promoting complex subunit 16, Anaphase-promoting complex subunit 7, Apoptosis-resistant E3 ubiquitin protein ligase 1, Transcription regulator protein BACH1, Transcription regulator protein BACH2, Baculoviral IAP repeat-containing protein 2, Baculoviral IAP repeat-containing protein 3, DDB1- and CUL4-associated factor 17, Denticleless protein homolog, F-box only protein 11, F-box only protein 30, E3 ubiquitin-protein ligase HECTD1, Probable E3 ubiquitin-protein ligase HERC1, Probable E3 ubiquitin-protein ligase HERC4, E3 ISG15-protein ligase HERC5, E3 ubiquitin-protein ligase HUWE1, Kelch repeat and BTB domain-containing protein 8, Kelch-like ECH-associated protein 1, MYCBP2 Probable E3 ubiquitin-protein ligase MYCBP2, E3 ubiquitin-protein ligase MYCBP2, Polycomb group RING finger protein 2, E3 SUMO-protein ligase PIAS4, Protein PML, E3 ubiquitin-protein ligase RING1, E3 ubiquitin-protein ligase RING2, E3 ubiquitin-protein ligase BRE1A, E3 ubiquitin-protein ligase RNF213, RING finger protein 214, E3 ubiquitin-protein ligase RNF25, E3 ubiquitin-protein ligase BRE1B, RING-box protein 2, Tumor necrosis factor alpha-induced protein 3, E3 ubiquitin-protein ligase TRIM33, E3 ubiquitin-protein ligase TRIM56, Tripartite motif-containing protein 65, E3 ubiquitin-protein ligase TRIM71, E3 ubiquitin-protein ligase TRIP12, (E3-independent) E2 ubiquitin-conjugating enzyme, UBE4A Ubiquitin conjugation factor E4 A, Ubiquitin conjugation factor E4 A, E3 ubiquitin-protein ligase UBR2, E3 ubiquitin-protein ligase UBR4, E3 ubiquitin-protein ligase UBR5, Protein VPRBP, Vacuolar protein sorting-associated protein 18 homolog, or Nuclear-interacting partner of ALK.

In some embodiments, the cysteine-containing protein is a member of the E3-RING family. In some instances, the probe binds to a cysteine residue of a member of the E3-RING family. In some instances, the members comprise Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, and TNF receptor-associated factor 6. In some cases, the probe binds to a cysteine residue of Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, or TNF receptor-associated factor 6.

In some instances, the cysteine-containing protein is a member of the Cullin RING ligase (CRL) family. In some cases, the members comprise Elongin-B and Elongin-C. In some cases, the probe binds to a cysteine residue of Elongin-B or Elongin-C.

In some instances, the cysteine-containing protein is B-cell lymphoma 6 protein. In some cases, the probe binds to a cysteine residue of B-cell lymphoma 6 protein.

In some instances, the cysteine-containing protein is (E3-independent) E2 ubiquitin-conjugating enzyme. In some cases, the probe binds to a cysteine residue of (E3-independent) E2 ubiquitin-conjugating enzyme.

In some embodiments, the protein is B-cell lymphoma 6 protein (BCL6) and the cysteine residue is C121, C175, C232, C254, C296, C339, C348, C354, C414, C548, or C663, wherein the numbering of the amino acid position corresponds to the amino acid position with the UniProt Identifier P41182. In some cases, the synthetic ligand inhibits a covalent interaction between C121, C175, C232, C254, C296, C339, C348, C354, C414, C548, or C663 of BCL6 and the probe.

In some embodiments, the protein is Polycomb group RING finger protein 6 (PCGF6) and the cysteine residue is C56, C137, or C155, wherein the numbering of the amino acid position corresponds to the amino acid position with the UniProt Identifier Q9BYE7. In some cases, the synthetic ligand inhibits a covalent interaction between C56, C137, or C155 of PCGF6 and the probe.

In some embodiments, the protein is E3 ubiquitin-protein ligase CBL-B (CBLB) and the cysteine residue is C60, C345, C376, C435, C436, C470, C523, C535, C594, C607, C686, C741, or C895, wherein the numbering of the amino acid position corresponds to the amino acid position with the UniProt Identifier Q13191. In some cases, the synthetic ligand inhibits a covalent interaction between C60, C345, C376, C435, C436, C470, C523, C535, C594, C607, C686, C741, or C895 of CBLB and the probe.

In some embodiments, the protein is E3 ubiquitin-protein ligase CBL-B (CBLB) and the cysteine residue is C607, wherein the numbering of the amino acid position corresponds to the amino acid position with the UniProt Identifier Q13191. In some cases, the synthetic ligand inhibits a covalent interaction between C607 of CBLB and the probe.

In some embodiments, the protein is Elongin-B (ELOB) and the cysteine residue is C60 or C89, wherein the numbering of the amino acid position corresponds to the amino acid position with the UniProt Identifier Q15370. In some cases, the synthetic ligand inhibits a covalent interaction between C60 or C89 of ELOB and the probe.

In some embodiments, the protein is Elongin-C (ELOC) and the cysteine residue is C11 or C74, wherein the numbering of the amino acid position corresponds to the amino acid position with the UniProt Identifier Q15369. In some cases, the synthetic ligand inhibits a covalent interaction between C11 or C74 of ELOC and the probe.

In some embodiments, the protein is F-box only protein 22 (FBXO22) and the cysteine residue is C47, C117, C227, C228, or C378, wherein the numbering of the amino acid position corresponds to the amino acid position with the UniProt Identifier Q8NEZ5. In some cases, the synthetic ligand inhibits a covalent interaction between C47, C117, C227, C228, or C378 of FBXO22 and the probe.

In some embodiments, the protein is Kelch repeat and BTB domain-containing protein 4 (KBTBD4) and the cysteine residue is C68, C201, C274, C301, C455, or C472, wherein the numbering of the amino acid position corresponds to the amino acid position with the UniProt Identifier Q9NVX7.

In some cases, the synthetic ligand inhibits a covalent interaction between C68, C201, C274, C301, C455, or C472 of KBTBD4 and the probe.

In some embodiments, the protein is Kelch repeat and BTB domain-containing protein 4 and the cysteine residue is C68, wherein the numbering of the amino acid position corresponds to the amino acid position with the UniProt Identifier Q9NVX7. In some cases, the synthetic ligand inhibits a covalent interaction between C68 of KBTBD4 and the probe.

In some embodiments, the protein is Kelch-like ECH-associated protein 1 (KEAP1) and the cysteine residue is C23, C38, C151, C226, C241, C257, C288, C297, C319, C434, C613, C622, or C624 wherein the numbering of the amino acid position corresponds to the amino acid position with the UniProt Identifier Q14145. In some cases, the synthetic ligand inhibits a covalent interaction between C23, C38, C151, C226, C241, C257, C288, C297, C319, C434, C613, C622, or C624 of KEAP1 and the probe.

In some embodiments, the protein is Kelch-like ECH-associated protein 1 and the cysteine residue is C23, C38, C151, C241, C257, C288, C297, C319, C613, or C624, wherein the numbering of the amino acid position corresponds to the amino acid position with the UniProt Identifier Q14145. In some cases, the synthetic ligand inhibits a covalent interaction between C23, C38, C151, C241, C257, C288, C297, C319, C613, or C624 of KEAP1 and the probe.

In some embodiments, the protein is E3 ubiquitin-protein ligase pellino homolog 11 (PELI1) and the cysteine residue is C61, C212, or C282 wherein the numbering of the amino acid position corresponds to the amino acid position with the UniProt Identifier Q96FA3. In some cases, the synthetic ligand inhibits a covalent interaction between C61, C212, or C282 of PELI1 and the probe.

In some embodiments, the protein is E3 ubiquitin-protein ligase pellino homolog 1 and the cysteine residue is C282, wherein the numbering of the amino acid position corresponds to the amino acid position with the UniProt Identifier Q96FA3. In some cases, the synthetic ligand inhibits a covalent interaction between C282 of PELI1 and the probe.

In some embodiments, the protein is E3 ubiquitin-protein ligase RNF128 (RNF128) and the cysteine residue is C295, C303, C314, or C317 wherein the numbering of the amino acid position corresponds to the amino acid position with the UniProt Identifier Q8TEB7. In some cases, the synthetic ligand inhibits a covalent interaction between C295, C303, C314, or C317 of RNF128 and the probe.

In some embodiments, the protein is E3 ubiquitin-protein ligase RNF128 and the cysteine residue is C317, wherein the numbering of the amino acid position corresponds to the amino acid position with the UniProt Identifier Q8TEB7. In some cases, the synthetic ligand inhibits a covalent interaction between C317 of RNF128 and the probe.

In some embodiments, the protein is TNF receptor-associated factor 6 (TRAF6) and the cysteine residue is C85, C105, C134, C139, C182, C235, C349, or C366, wherein the numbering of the amino acid position corresponds to the amino acid position with the UniProt Identifier Q9Y4K3. In some cases, the synthetic ligand inhibits a covalent interaction between C85, C105, C134, C139, C182, C235, C349, or C366 of TRAF6 and the probe.

In some embodiments, the protein is (E3-independent) E2 ubiquitin-conjugating enzyme (UBE20) and the cysteine residue is C101, C182, C208, C230, C244, C314, C341, C370, C375, C400, C406, C585, C598, C910, C913, C1040, C1099, or C1288, wherein the numbering of the amino acid position corresponds to the amino acid position with the UniProt Identifier Q9C0C9. In some cases, the synthetic ligand inhibits a covalent interaction between C101, C182, C208, C230, C244, C314, C341, C370, C375, C400, C406, C585, C598, C910, C913, C1040, C1099, or C1288 of UBE20 and the probe.

In some embodiments, the protein is (E3-independent) E2 ubiquitin-conjugating enzyme and the cysteine residue is C370, C400, C910, or C913, wherein the numbering of the amino acid position corresponds to the amino acid position with the UniProt Identifier Q9C0C9. In some cases, the synthetic ligand inhibits a covalent interaction between C370, C400, C910, or C913 of UBE20 and the probe.

In some instances, the protein is E3 ubiquitin-protein ligase TRIP12 (TRIP12) and the cysteine residue is C1959, wherein the numbering of the amino acid position corresponds to the amino acid position with the UniProt Identifier Q14669. In some cases, the synthetic ligand inhibits a covalent interaction between C1959 of TRIP12 and the probe.

In some instances, the protein is anaphase-promoting complex subunit 16 (ANAPC16) and the cysteine residue is C55, wherein the numbering of the amino acid position corresponds to the amino acid position with the UniProt Identifier Q96DE5. In some cases, the synthetic ligand inhibits a covalent interaction between C55 of ANAPC16 and the probe.

In some instances, the protein is probable E3 ubiquitin-protein ligase MYCBP2 (MYCBP2) and the cysteine residue is C3152, wherein the numberings of the amino acid positions correspond to the amino acid positions with the UniProt Identifier 075592. In some cases, the synthetic ligand inhibits a covalent interaction between C3152 of MYCBP2 and the probe.

In some instances, the protein is ubiquitin conjugation factor E4 A (UBE4A) and the cysteine residue is C79, wherein the numbering of the amino acid position corresponds to the amino acid position with the UniProt Identifier Q14139. In some cases, the synthetic ligand inhibits a covalent interaction between C79 of UBE4A and the probe.

In some instances, the protein is autophagy-related protein 16-1 (ATG16L1) and the cysteine residue is C145, wherein the numbering of the amino acid position corresponds to the amino acid position with the UniProt Identifier Q676U5. In some cases, the synthetic ligand inhibits a covalent interaction between C145 of ATG16L1 and the probe.

In some instances, the protein is protein arginine N-methyltransferase 5 (PRMT5) and the cysteine residue is C278, wherein the numbering of the amino acid position corresponds to the amino acid position with the UniProt Identifier 014744. In some cases, the synthetic ligand inhibits a covalent interaction between C278 of PRMT5 and the probe.

In some instances, the protein is isocitrate dehydrogenase (IDH2) and the cysteine residue is C154, wherein the numberings of the amino acid positions correspond to the amino acid positions with the UniProt Identifier P48735. In some cases, the synthetic ligand inhibits a covalent interaction between C154 of IDH2 and the probe.

In some instances, the protein is antigen peptide transporter 2 (TAP2) and the cysteine residue is C641, wherein the numbering of the amino acid position corresponds to the amino acid position with the UniProt Identifier Q03519. In some cases, the synthetic ligand inhibits a covalent interaction between C641 of TAP2 and the probe.

In some instances, the protein is tapasin (TAPBP) and the cysteine residue is C440, wherein the numbering of the amino acid position corresponds to the amino acid position with the UniProt Identifier O15533. In some cases, the synthetic ligand inhibits a covalent interaction between C440 of TAPBP and the probe.

In some instances, the protein is protein unc-93 homolog B1 (UNC93B1) and the cysteine residue is C583, wherein the numbering of the amino acid position corresponds to the amino acid position with the UniProt Identifier Q9H1C4. In some cases, the synthetic ligand inhibits a covalent interaction between C583 of UNC93B1 and the probe.

In some instances, the protein is probable ATP-dependent RNA helicase DDX60 (DDX60) and the cysteine residue is C1051, wherein the numbering of the amino acid position corresponds to the amino acid position with the UniProt Identifier Q81Y21. In some cases, the synthetic ligand inhibits a covalent interaction between C1051 of DDX60 and the probe.

In some instances, the synthetic ligand further comprises a second moiety that interacts with a second protein. In some cases, the second protein is not a protein illustrated in Table 1A or Table 2A.

In some embodiments, additionally described herein include a protein binding domain wherein said protein binding domain comprises a cysteine residue illustrated in Table 1A or Table 2A, wherein said cysteine forms an adduct with a compound of Formula I,

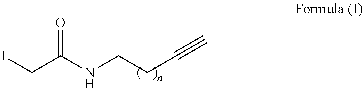

and wherein a compound of Formula IIA or Formula IIB interferes with the formation of the cysteine adduct by the compound of Formula I, wherein Formula (IIA) or Formula (IIB) have the structure:

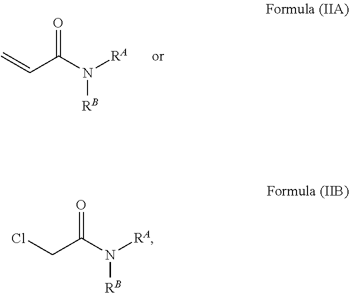

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl; or
or R^{A} and R^{B} together with the nitrogen to which they are attached form a 5, 6, 7 or 8-membered heterocyclic ring A, optionally having one additional heteroatom moiety independently selected from NR¹, O, or S; wherein A is optionally substituted; and
R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl

In some instances, n is 0, 1, 2, 3, 4, 5, 6, 7, or 8. In some instances, n is 1. In some instances, n is 2. In some instances, n is 3. In some instances, n is 4. In some instances, n is 5. In some instances, n is 6. In some instances, n is 7. In some instances, n is 8.

In some instances, the protein is Anaphase-promoting complex subunit 16, Anaphase-promoting complex subunit 7, Apoptosis-resistant E3 ubiquitin protein ligase 1, Transcription regulator protein BACH1, Transcription regulator protein BACH2, Baculoviral IAP repeat-containing protein 2, Baculoviral IAP repeat-containing protein 3, DDB1- and CUL4-associated factor 17, Denticleless protein homolog, F-box only protein 11, F-box only protein 30, E3 ubiquitin-protein ligase HECTD1, Probable E3 ubiquitin-protein ligase HERC1, Probable E3 ubiquitin-protein ligase HERC4, E3 ISG15-protein ligase HERC5, E3 ubiquitin-protein ligase HUWE1, Kelch repeat and BTB domain-containing protein 8, Kelch-like ECH-associated protein 1, MYCBP2 Probable E3 ubiquitin-protein ligase MYCBP2, E3 ubiquitin-protein ligase MYCBP2, Polycomb group RING finger protein 2, E3 SUMO-protein ligase PIAS4, Protein PML, E3 ubiquitin-protein ligase RING1, E3 ubiquitin-protein ligase RING2, E3 ubiquitin-protein ligase BRE1A, E3 ubiquitin-protein ligase RNF213, RING finger protein 214, E3 ubiquitin-protein ligase RNF25, E3 ubiquitin-protein ligase BRE1B, RING-box protein 2, Tumor necrosis factor alpha-induced protein 3, E3 ubiquitin-protein ligase TRIM33, E3 ubiquitin-protein ligase TRIM56, Tripartite motif-containing protein 65, E3 ubiquitin-protein ligase TRIM71, E3 ubiquitin-protein ligase TRIP12, (E3-independent) E2 ubiquitin-conjugating enzyme, UBE4A Ubiquitin conjugation factor E4 A, Ubiquitin conjugation factor E4 A, E3 ubiquitin-protein ligase UBR2, E3 ubiquitin-protein ligase UBR4, E3 ubiquitin-protein ligase UBR5, Protein VPRBP, Vacuolar protein sorting-associated protein 18 homolog, or Nuclear-interacting partner of ALK.

In some embodiments, the cysteine-containing protein is a member of the E3-RING family. In some instances, the probe binds to a cysteine residue of a member of the E3-RING family. In some instances, the members comprise Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, and TNF receptor-associated factor 6. In some cases, the probe binds to a cysteine residue of Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, or TNF receptor-associated factor 6.

In some instances, the cysteine-containing protein is a member of the Cullin RING ligase (CRL) family. In some cases, the members comprise Elongin-B and Elongin-C. In some cases, the probe binds to a cysteine residue of Elongin-B or Elongin-C.

In some instances, the cysteine-containing protein is B-cell lymphoma 6 protein. In some cases, the probe binds to a cysteine residue of B-cell lymphoma 6 protein.

In some instances, the cysteine-containing protein is (E3-independent) E2 ubiquitin-conjugating enzyme. In some cases, the probe binds to a cysteine residue of (E3-independent) E2 ubiquitin-conjugating enzyme.

In some embodiments, the protein is B-cell lymphoma 6 protein (BCL6) and the cysteine residue is C121, C175, C232, C254, C296, C339, C348, C354, C414, C548, or C663, wherein the numbering of the amino acid position corresponds to the amino acid position with the UniProt Identifier P41182. In some cases, the protein binding domain comprises C121, C175, C232, C254, C296, C339, C348, C354, C414, C548, or C663.

In some embodiments, the protein is Polycomb group RING finger protein 6 (PCGF6) and the cysteine residue is C56, C137, or C155, wherein the numbering of the amino acid position corresponds to the amino acid position with the UniProt Identifier Q9BYE7. In some cases, the protein binding domain comprises C56, C137, or C155.

In some embodiments, the protein is E3 ubiquitin-protein ligase CBL-B (CBLB) and the cysteine residue is C60, C345, C376, C435, C436, C470, C523, C535, C594, C607, C686, C741, or C895, wherein the numbering of the amino acid position corresponds to the amino acid position with the UniProt Identifier Q13191. In some cases, the protein binding domain comprises C60, C345, C376, C435, C436, C470, C523, C535, C594, C607, C686, C741, or C895.

In some embodiments, the protein is E3 ubiquitin-protein ligase CBL-B (CBLB) and the cysteine residue is C607, wherein the numbering of the amino acid position corresponds to the amino acid position with the UniProt Identifier Q13191. In some cases, the protein binding domain comprises C607.

In some embodiments, the protein is Elongin-B (ELOB) and the cysteine residue is C60 or C89, wherein the numbering of the amino acid position corresponds to the amino acid position with the UniProt Identifier Q15370. In some cases, the protein binding domain comprises C60 or C89.

In some embodiments, the protein is Elongin-C (ELOC) and the cysteine residue is C11 or C74, wherein the numbering of the amino acid position corresponds to the amino acid position with the UniProt Identifier Q15369. In some cases, the protein binding domain comprises C11 or C74.

In some embodiments, the protein is F-box only protein 22 (FBXO22) and the cysteine residue is C47, C117, C227, C228, or C378, wherein the numbering of the amino acid position corresponds to the amino acid position with the UniProt Identifier Q8NEZ5. In some cases, the protein binding domain comprises C47, C117, C227, C228, or C378.

In some embodiments, the protein is Kelch repeat and BTB domain-containing protein 4 (KBTBD4) and the cysteine residue is C68, C201, C274, C301, C455, or C472, wherein the numbering of the amino acid position corresponds to the amino acid position with the UniProt Identifier Q9NVX7. In some cases, the protein binding domain comprises C68, C201, C274, C301, C455, or C472.

In some embodiments, the protein is Kelch repeat and BTB domain-containing protein 4 and the cysteine residue is C68, wherein the numbering of the amino acid position corresponds to the amino acid position with the UniProt Identifier Q9NVX7. In some cases, the protein binding domain comprises C68.

In some embodiments, the protein is Kelch-like ECH-associated protein 1 (KEAP1) and the cysteine residue is C23, C38, C151, C226, C241, C257, C288, C297, C319, C434, C613, C622, or C624 wherein the numbering of the amino acid position corresponds to the amino acid position with the UniProt Identifier Q14145. In some cases, the protein binding domain comprises C23, C38, C151, C226, C241, C257, C288, C297, C319, C434, C613, C622, or C624.

In some embodiments, the protein is Kelch-like ECH-associated protein 1 and the cysteine residue is C23, C38, C151, C241, C257, C288, C297, C319, C613, or C624, wherein the numbering of the amino acid position corresponds to the amino acid position with the UniProt Identifier Q14145. In some cases, the protein binding domain comprises C23, C38, C151, C241, C257, C288, C297, C319, C613, or C624.

In some embodiments, the protein is E3 ubiquitin-protein ligase pellino homolog 11 (PELI1) and the cysteine residue is C61, C212, or C282 wherein the numbering of the amino acid position corresponds to the amino acid position with the UniProt Identifier Q96FA3. In some cases, the protein binding domain comprises C61, C212, or C282.

In some embodiments, the protein is E3 ubiquitin-protein ligase pellino homolog 1 and the cysteine residue is C282, wherein the numbering of the amino acid position corresponds to the amino acid position with the UniProt Identifier Q96FA3. In some cases, the protein binding domain comprises C282.

In some embodiments, the protein is E3 ubiquitin-protein ligase RNF128 (RNF128) and the cysteine residue is C295, C303, C314, or C317 wherein the numbering of the amino acid position corresponds to the amino acid position with the UniProt Identifier Q8TEB7. In some cases, the protein binding domain comprises C295, C303, C314, or C317.

In some embodiments, the protein is E3 ubiquitin-protein ligase RNF128 and the cysteine residue is C317, wherein the numbering of the amino acid position corresponds to the amino acid position with the UniProt Identifier Q8TEB7. In some cases, the protein binding domain comprises C317.

In some embodiments, the protein is TNF receptor-associated factor 6 (TRAF6) and the cysteine residue is C85, C105, C134, C139, C182, C235, C349, or C366, wherein the numbering of the amino acid position corresponds to the amino acid position with the UniProt Identifier Q9Y4K3. In some cases, the protein binding domain comprises C85, C105, C134, C139, C182, C235, C349, or C366.

In some embodiments, the protein is (E3-independent) E2 ubiquitin-conjugating enzyme (UBE20) and the cysteine residue is C101, C182, C208, C230, C244, C314, C341, C370, C375, C400, C406, C585, C598, C910, C913, C1040, C1099, or C1288, wherein the numbering of the amino acid position corresponds to the amino acid position with the UniProt Identifier Q9C0C9. In some cases, the protein binding domain comprises C101, C182, C208, C230, C244, C314, C341, C370, C375, C400, C406, C585, C598, C910, C913, C1040, C1099, or C1288.

In some embodiments, the protein is (E3-independent) E2 ubiquitin-conjugating enzyme and the cysteine residue is C370, C400, C910, or C913, wherein the numbering of the amino acid position corresponds to the amino acid position with the UniProt Identifier Q9C0C9. In some cases, the protein binding domain comprises C370, C400, C910, or C913.

In some instances, the protein is E3 ubiquitin-protein ligase TRIP12 (TRIP12) and the cysteine residue is C1959, wherein the numberings of the amino acid positions correspond to the amino acid positions with the UniProt Identifier Q14669. In some cases, the protein binding domain comprises C1959.

In some instances, the protein is anaphase-promoting complex subunit 16 (ANAPC16) and the cysteine residue is C55, wherein the numbering of the amino acid position corresponds to the amino acid position with the UniProt Identifier Q96DE5. In some cases, the protein binding domain comprises C55.

In some instances, the protein is probable E3 ubiquitin-protein ligase MYCBP2 (MYCBP2) and the cysteine residue is C3152, wherein the numberings of the amino acid positions correspond to the amino acid positions with the UniProt Identifier 075592. In some cases, the protein binding domain comprises C3152.

In some instances, the protein is ubiquitin conjugation factor E4 A (UBE4A) and the cysteine residue is C79, wherein the numbering of the amino acid position corresponds to the amino acid position with the UniProt Identifier Q14139. In some cases, the protein binding domain comprises C79.

In some instances, the protein is autophagy-related protein 16-1 (ATG16L1) and the cysteine residue is C145, wherein the numbering of the amino acid position corresponds to the amino acid position with the UniProt Identifier Q676U5. In some cases, the protein binding domain comprises C145.

In some instances, the protein is protein arginine N-methyltransferase 5 (PRMT5) and the cysteine residue is C278, wherein the numbering of the amino acid position corresponds to the amino acid position with the UniProt Identifier 014744. In some cases, the protein binding domain comprises C278.

In some instances, the protein is isocitrate dehydrogenase (IDH2) and the cysteine residue is C154, wherein the numberings of the amino acid positions correspond to the amino acid positions with the UniProt Identifier P48735. In some cases, the protein binding domain comprises C154.

In some instances, the protein is antigen peptide transporter 2 (TAP2) and the cysteine residue is C641, wherein the numbering of the amino acid position corresponds to the amino acid position with the UniProt Identifier Q03519. In some cases, the protein binding domain comprises C641.

In some instances, the protein is tapasin (TAPBP) and the cysteine residue is C440, wherein the numbering of the amino acid position corresponds to the amino acid position with the UniProt Identifier O15533. In some cases, the protein binding domain comprises C440.

In some instances, the protein is protein unc-93 homolog B1 (UNC93B1) and the cysteine residue is C583, wherein the numbering of the amino acid position corresponds to the amino acid position with the UniProt Identifier Q9H1C4. In some cases, the protein binding domain comprises C583.

In some instances, the protein is probable ATP-dependent RNA helicase DDX60 (DDX60) and the cysteine residue is C1051, wherein the numbering of the amino acid position corresponds to the amino acid position with the UniProt Identifier Q81Y21. In some cases, the protein binding domain comprises C1051.

In some embodiments, further disclosed herein is a method for identifying a synthetic ligand that interacts with a protein comprising a cysteine residue illustrated in Table 1A or Table 2A, comprising exposing, in a reaction vessel, the protein to the synthetic ligand and a probe that has a structure represented by Formula (I):

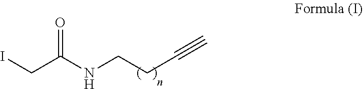

wherein
n is 0-8; and
measuring the amount of the probe that has covalently bound to the cysteine residue relative to the amount of probe that has covalently bound to the same cysteine residue in the absence of the synthetic ligand.

In some instances, the protein is Anaphase-promoting complex subunit 16, Anaphase-promoting complex subunit 7, Apoptosis-resistant E3 ubiquitin protein ligase 1, Transcription regulator protein BACH1, Transcription regulator protein BACH2, Baculoviral IAP repeat-containing protein 2, Baculoviral IAP repeat-containing protein 3, DDB1- and CUL4-associated factor 17, Denticleless protein homolog, F-box only protein 11, F-box only protein 30, E3 ubiquitin-protein ligase HECTD1, Probable E3 ubiquitin-protein ligase HERC1, Probable E3 ubiquitin-protein ligase HERC4, E3 ISG15-protein ligase HERC5, E3 ubiquitin-protein ligase HUWE1, Kelch repeat and BTB domain-containing protein 8, Kelch-like ECH-associated protein 1, MYCBP2 Probable E3 ubiquitin-protein ligase MYCBP2, E3 ubiquitin-protein ligase MYCBP2, Polycomb group RING finger protein 2, E3 SUMO-protein ligase PIAS4, Protein PML, E3 ubiquitin-protein ligase RING1, E3 ubiquitin-protein ligase RING2, E3 ubiquitin-protein ligase BRE1A, E3 ubiquitin-protein ligase RNF213, RING finger protein 214, E3 ubiquitin-protein ligase RNF25, E3 ubiquitin-protein ligase BRE1B, RING-box protein 2, Tumor necrosis factor alpha-induced protein 3, E3 ubiquitin-protein ligase TRIM33, E3 ubiquitin-protein ligase TRIM56, Tripartite motif-containing protein 65, E3 ubiquitin-protein ligase TRIM71, E3 ubiquitin-protein ligase TRIP12, (E3-independent) E2 ubiquitin-conjugating enzyme, UBE4A Ubiquitin conjugation factor E4 A, Ubiquitin conjugation factor E4 A, E3 ubiquitin-protein ligase UBR2, E3 ubiquitin-protein ligase UBR4, E3 ubiquitin-protein ligase UBR5, Protein VPRBP, Vacuolar protein sorting-associated protein 18 homolog, or Nuclear-interacting partner of ALK.

In some embodiments, the cysteine-containing protein is a member of the E3-RING family. In some instances, the probe binds to a cysteine residue of a member of the E3-RING family. In some instances, the members comprise Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, and TNF receptor-associated factor 6. In some cases, the probe binds to a cysteine residue of Polycomb group RING finger protein 6, E3 ubiquitin-protein ligase CBL-B, F-box only protein 22, Elongin-B, Elongin-C, Kelch repeat and BTB domain domain-containing protein 4, Kelch-like ECH-associated protein 1, E3 ubiquitin-protein ligase pellino homolog 1, E3 ubiquitin-protein ligase RNF128, or TNF receptor-associated factor 6.

In some instances, the cysteine-containing protein is a member of the Cullin RING ligase (CRL) family. In some cases, the members comprise Elongin-B and Elongin-C. In some cases, the probe binds to a cysteine residue of Elongin-B or Elongin-C.

In some instances, the cysteine-containing protein is B-cell lymphoma 6 protein. In some cases, the probe binds to a cysteine residue of B-cell lymphoma 6 protein.

In some instances, the cysteine-containing protein is (E3-independent) E2 ubiquitin-conjugating enzyme. In some cases, the probe binds to a cysteine residue of (E3-independent) E2 ubiquitin-conjugating enzyme.

In some embodiments, the protein is B-cell lymphoma 6 protein (BCL6) and the cysteine residue is C121, C175, C232, C254, C296, C339, C348, C354, C414, C548, or C663, wherein the numbering of the amino acid position corresponds to the amino acid position with the UniProt Identifier P41182.

In some embodiments, the protein is Polycomb group RING finger protein 6 (PCGF6) and the cysteine residue is C56, C137, or C155, wherein the numbering of the amino acid position corresponds to the amino acid position with the UniProt Identifier Q9BYE7.

In some embodiments, the protein is E3 ubiquitin-protein ligase CBL-B (CBLB) and the cysteine residue is C60, C345, C376, C435, C436, C470, C523, C535, C594, C607, C686, C741, or C895, wherein the numbering of the amino acid position corresponds to the amino acid position with the UniProt Identifier Q13191.

In some embodiments, the protein is E3 ubiquitin-protein ligase CBL-B (CBLB) and the cysteine residue is C607, wherein the numbering of the amino acid position corresponds to the amino acid position with the UniProt Identifier Q13191.

In some embodiments, the protein is Elongin-B (ELOB) and the cysteine residue is C60 or C89, wherein the numbering of the amino acid position corresponds to the amino acid position with the UniProt Identifier Q15370.

In some embodiments, the protein is Elongin-C (ELOC) and the cysteine residue is C11 or C74, wherein the numbering of the amino acid position corresponds to the amino acid position with the UniProt Identifier Q15369.

In some embodiments, the protein is F-box only protein 22 (FBXO22) and the cysteine residue is C47, C117, C227, C228, or C378, wherein the numbering of the amino acid position corresponds to the amino acid position with the UniProt Identifier Q8NEZ5.

In some embodiments, the protein is Kelch repeat and BTB domain-containing protein 4 (KBTBD4) and the cysteine residue is C68, C201, C274, C301, C455, or C472, wherein the numbering of the amino acid position corresponds to the amino acid position with the UniProt Identifier Q9NVX7.

In some embodiments, the protein is Kelch repeat and BTB domain-containing protein 4 and the cysteine residue is C68, wherein the numbering of the amino acid position corresponds to the amino acid position with the UniProt Identifier Q9NVX7.

In some embodiments, the protein is Kelch-like ECH-associated protein 1 (KEAP1) and the cysteine residue is C23, C38, C151, C226, C241, C257, C288, C297, C319, C434, C613, C622, or C624 wherein the numbering of the amino acid position corresponds to the amino acid position with the UniProt Identifier Q14145.

In some embodiments, the protein is Kelch-like ECH-associated protein 1 and the cysteine residue is C23, C38, C151, C241, C257, C288, C297, C319, C613, or C624, wherein the numbering of the amino acid position corresponds to the amino acid position with the UniProt Identifier Q14145.

In some embodiments, the protein is E3 ubiquitin-protein ligase pellino homolog 11 (PELI1) and the cysteine residue is C61, C212, or C282 wherein the numbering of the amino acid position corresponds to the amino acid position with the UniProt Identifier Q96FA3.

In some embodiments, the protein is E3 ubiquitin-protein ligase pellino homolog 1 and the cysteine residue is C282, wherein the numbering of the amino acid position corresponds to the amino acid position with the UniProt Identifier Q96FA3.

In some embodiments, the protein is E3 ubiquitin-protein ligase RNF128 (RNF128) and the cysteine residue is C295, C303, C314, or C317 wherein the numbering of the amino acid position corresponds to the amino acid position with the UniProt Identifier Q8TEB7.

In some embodiments, the protein is E3 ubiquitin-protein ligase RNF128 and the cysteine residue is C317, wherein the numbering of the amino acid position corresponds to the amino acid position with the UniProt Identifier Q8TEB7.

In some embodiments, the protein is TNF receptor-associated factor 6 (TRAF6) and the cysteine residue is C85, C105, C134, C139, C182, C235, C349, or C366, wherein the numbering of the amino acid position corresponds to the amino acid position with the UniProt Identifier Q9Y4K3.

In some embodiments, the protein is (E3-independent) E2 ubiquitin-conjugating enzyme (UBE20) and the cysteine residue is C101, C182, C208, C230, C244, C314, C341, C370, C375, C400, C406, C585, C598, C910, C913, C1040, C1099, or C1288, wherein the numbering of the amino acid position corresponds to the amino acid position with the UniProt Identifier Q9C0C9.

In some embodiments, the protein is (E3-independent) E2 ubiquitin-conjugating enzyme and the cysteine residue is C370, C400, C910, or C913, wherein the numbering of the amino acid position corresponds to the amino acid position with the UniProt Identifier Q9C0C9.

In some instances, the protein is E3 ubiquitin-protein ligase TRIP12 (TRIP12) and the cysteine residue is C1959, wherein the numbering of the amino acid position corresponds to the amino acid position with the UniProt Identifier Q14669. In some cases, the synthetic ligand inhibits a covalent interaction between C1959 of TRIP12 and the probe.

In some instances, the protein is anaphase-promoting complex subunit 16 (ANAPC16) and the cysteine residue is C55, wherein the numbering of the amino acid position corresponds to the amino acid position with the UniProt Identifier Q96DE5. In some cases, the synthetic ligand inhibits a covalent interaction between C55 of ANAPC16 and the probe.

In some instances, the protein is probable E3 ubiquitin-protein ligase MYCBP2 (MYCBP2) and the cysteine residue is C3152, wherein the numberings of the amino acid positions correspond to the amino acid positions with the UniProt Identifier 075592. In some cases, the synthetic ligand inhibits a covalent interaction between C3152 of MYCBP2 and the probe.

In some instances, the protein is ubiquitin conjugation factor E4 A (UBE4A) and the cysteine residue is C79, wherein the numbering of the amino acid position corresponds to the amino acid position with the UniProt Identifier Q14139. In some cases, the synthetic ligand inhibits a covalent interaction between C79 of UBE4A and the probe.

In some instances, the protein is autophagy-related protein 16-1 (ATG16L1) and the cysteine residue is C145, wherein the numbering of the amino acid position corresponds to the amino acid position with the UniProt Identifier Q676U5. In some cases, the synthetic ligand inhibits a covalent interaction between C145 of ATG16L1 and the probe.

In some instances, the protein is protein arginine N-methyltransferase 5 (PRMT5) and the cysteine residue is C278, wherein the numbering of the amino acid position corresponds to the amino acid position with the UniProt Identifier 014744. In some cases, the synthetic ligand inhibits a covalent interaction between C278 of PRMT5 and the probe.

In some instances, the protein is isocitrate dehydrogenase (IDH2) and the cysteine residue is C154, wherein the numberings of the amino acid positions correspond to the amino acid positions with the UniProt Identifier P48735. In some cases, the synthetic ligand inhibits a covalent interaction between C154 of IDH2 and the probe.

In some instances, the protein is antigen peptide transporter 2 (TAP2) and the cysteine residue is C641, wherein the numbering of the amino acid position corresponds to the amino acid position with the UniProt Identifier Q03519. In some cases, the synthetic ligand inhibits a covalent interaction between C641 of TAP2 and the probe.

In some instances, the protein is tapasin (TAPBP) and the cysteine residue is C440, wherein the numbering of the amino acid position corresponds to the amino acid position with the UniProt Identifier O15533. In some cases, the synthetic ligand inhibits a covalent interaction between C440 of TAPBP and the probe.

In some instances, the protein is protein unc-93 homolog B1 (UNC93B1) and the cysteine residue is C583, wherein the numbering of the amino acid position corresponds to the amino acid position with the UniProt Identifier Q9H1C4. In some cases, the synthetic ligand inhibits a covalent interaction between C583 of UNC93B1 and the probe.

In some instances, the protein is probable ATP-dependent RNA helicase DDX60 (DDX60) and the cysteine residue is C1051, wherein the numbering of the amino acid position corresponds to the amino acid position with the UniProt Identifier Q81Y21. In some cases, the synthetic ligand inhibits a covalent interaction between C1051 of DDX60 and the probe.

### Further Forms of Compounds

In one aspect, the compound of Formula (II), Formula (IIA), or Formula (IIB) possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

Compounds described herein may be formed as, and/or used as, acceptable salts. The type of acceptable salts, include, but are not limited to: (1) acid addition salts, formed by reacting the free base form of the compound with an acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

In some embodiments, the compounds of Formula (I), Formula (II), Formula (IIA), and Formula (IIB) are purchased from a variety of vendors, including Sigma Aldrich, Acros, Fisher, Fluka, Santa Cruz, CombiBlocks, BioBlocks, and Matrix Scientific.

### Cells, Analytical Techniques, and Instrumentation

In certain embodiments, also described herein are methods for profiling a protein described above to determine a reactive or ligandable cysteine residue. In some instances, the methods comprising profiling a cell sample or a cell lysate sample. In some embodiments, the cell sample or cell lysate sample is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the cell sample or cell lysate sample is obtained from a mammalian cell. In some instances, the mammalian cell is an epithelial cell, connective tissue cell, hormone secreting cell, a nerve cell, a skeletal muscle cell, a blood cell, or an immune system cell.

Exemplary mammalian cells include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, and PC12 cell line.

In some instances, the cell sample or cell lysate sample is obtained from cells of a tumor cell line. In some instances, the cell sample or cell lysate sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the cell sample or cell lysate sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the cell sample or cell lysate sample is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the cell sample or cell lysate sample is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract.

In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the cell sample or cell lysate sample is obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

Sample Preparation and Analysis

In some embodiments, a sample solution comprises a cell sample, a cell lysate sample, or a sample comprising isolated proteins. In some instances, the sample solution comprises a solution such as a buffer (e.g. phosphate buffered saline) or a media. In some embodiments, the media is an isotopically labeled media. In some instances, the sample solution is a cell solution.

In some embodiments, the solution sample (e.g., cell sample, cell lysate sample, or comprising isolated proteins) is incubated with a compound of Formula (I) for analysis of protein-probe interactions. In some instances, the solution sample (e.g., cell sample, cell lysate sample, or comprising isolated proteins) is further incubated in the presence of an additional compound probe prior to addition of the compound of Formula (I). In other instances, the solution sample (e.g., cell sample, cell lysate sample, or comprising isolated proteins) is further incubated with a ligand, in which the ligand does not contain a photoreactive moiety and/or an alkyne group. In such instances, the solution sample is incubated with a probe and a ligand for competitive protein profiling analysis.

In some cases, the cell sample or the cell lysate sample is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a solution sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a solution sample (e.g. cell sample, cell lysate sample, or comprising isolated proteins) with a labeling group (e.g., an isotopically labeled labeling group) to tag one or more proteins of interest for further analysis. In such cases, the labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag as described in Weerapana, et al., “Quantitative reactivity profiling predicts functional cysteines in proteomes,” Nature 468(7325): 790-795.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical means. In some instances, the protein from the probe-protein complexes is fragmented by a chemical means. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage K1F endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electrospray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by method as described in Weerapana et al., “Quantitative reactivity profiling predicts functional cysteines in proteomes,” Nature, 468:790-795 (2010).

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In some embodiments, a value is assigned to each of the protein from the probe-protein complex. In some embodiments, the value assigned to each of the protein from the probe-protein complex is obtained from the mass spectroscopy analysis. In some instances, the value is the area-under-the curve from a plot of signal intensity as a function of mass-to-charge ratio. In some instances, the value correlates with the reactivity of a Cys residue within a protein. In some instances, the value correlates with the reactivity of a Lys residue within a protein.

In some instances, a ratio between a first value obtained from a first protein sample and a second value obtained from a second protein sample is calculated. In some instances, the ratio is greater than 2.5, 3, 3.5, 4, 4.5, 5, 6, 7, 8, 9, 10, 11, 12, 13, 14, 15, 16, 17, 18, 19, or 20. In some cases, the ratio is at most 20.

In some instances, the ratio is calculated based on averaged values. In some instances, the averaged value is an average of at least two, three, or four values of the protein from each cell solution, or that the protein is observed at least two, three, or four times in each cell solution and a value is assigned to each observed time. In some instances, the ratio further has a standard deviation of less than 12, 10, or 8.

In some instances, a value is not an averaged value. In some instances, the ratio is calculated based on value of a protein observed only once in a cell population. In some instances, the ratio is assigned with a value of 20.

### Kits/Article of Manufacture

Disclosed herein, in certain embodiments, are kits and articles of manufacture for use to generate a protein-probe adduct or with one or more methods described herein. In some embodiments, described herein is a kit for detecting protein ligand interaction. In some embodiments, such kit includes small molecule ligands described herein, small molecule fragments or libraries, compound probes described herein, and/or controls, and reagents suitable for carrying out one or more of the methods described herein. In some instances, the kit further comprises samples, such as a cell sample, and suitable solutions such as buffers or media. In some embodiments, the kit further comprises recombinant cereblon protein for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

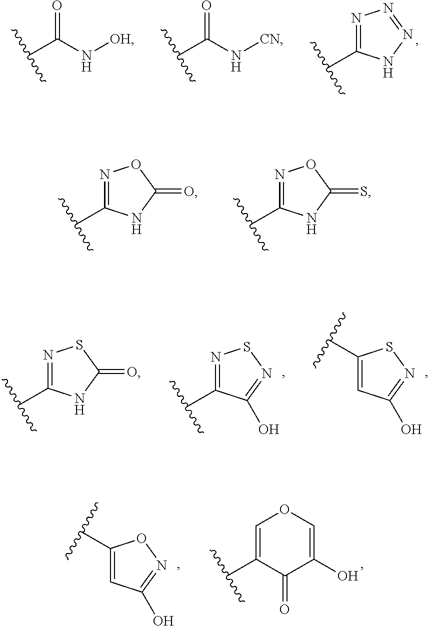

and the like.

“Cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the monocyclic cyclcoalkyl is cyclopentyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g. —NH₂, —NHR, —N(R)₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

The section headings used herein are for organizational purposes only and are not to be construed as limiting the subject matter described.

### EXAMPLES

These examples are provided for illustrative purposes only and not to limit the scope of the claims provided herein.

### Example 1

Table 1A and Table 1B illustrate exemplary proteins and cysteine site residues described herein.

TABLE 1A

| | | | | | KB02 | KBO3 |
|---|---|---|---|---|---|---|
| uniprot | Res# | Symbol | Description | E3? | Liganded | Liganded |
| Q96DE5 | 55 | ANAPC16 | ANAPC16 Anaphase-promoting | yes | YES | |
| | | | complex subunit 16 | | | |
| Q96DE5 | 55 | ANAPC16 | Anaphase-promoting complex subunit 16 | Yes | YES | |
| Q9UJX3 | 329 | ANAPC7 | Anaphase-promoting complex subunit 7 | Yes | YES | |
| O15033 | 382 | AREL1 | Apoptosis-resistant E3 ubiquitin protein | Yes | YES | |
| | | | ligase 1 | | | |
| O14867 | 224 | BACH1 | Transcription regulator protein BACH1 | Yes | YES | YES |
| Q9BYV9 | 370 | BACH2 | Transcription regulator protein BACH2 | Yes | YES | |
| Q13490 | 45 | BIRC2 | Baculoviral IAP repeat-containing | Yes | YES | YES |
| | | | protein 2 | | | |
| Q13489 | 28 | BIRC3 | Baculoviral IAP repeat-containing | Yes | YES | |
| | | | protein 3 | | | |
| Q5H9S7 | 20 | DCAF17 | DDB1- and CUL4-associated factor 17 | Yes | YES | |
| Q9NZJ0 | 141 | DTL | Denticleless protein homolog | Yes | YES | |
| Q9NZJ0 | 136 | DTL | Denticleless protein homolog | Yes | YES | |
| Q86XK2 | 581 | FBXO11 | F-box only protein 11 | Yes | YES | |
| Q8TB52 | 725 | FBXO30 | F-box only protein 30 | Yes | YES | |
| Q9ULT8 | 1855 | HECTD1 | E3 ubiquitin-protein ligase HECTD1 | Yes | YES | |
| Q15751 | 56 | HERC1 | Probable E3 ubiquitin-protein ligase | Yes | YES | |
| | | | HERC1 | | | |
| Q15751 | 1941 | HERC1 | Probable E3 ubiquitin-protein ligase | Yes | YES | |
| | | | HERC1 | | | |
| Q5GLZ8 | 60 | HERC4 | Probable E3 ubiquitin-protein ligase | Yes | YES | |
| | | | HERC4 | | | |
| Q9UII4 | 994 | HERC5 | E3 ISG15--protein ligase HERC5 | Yes | YES | YES |
| Q7Z6Z7 | 2181 | HUWE1 | E3 ubiquitin-protein ligase HUWE1 | Yes | YES | |
| Q8NFY9 | 181 | KBTBD8 | Kelch repeat and BTB domain- | Yes | YES | |
| | | | containing protein 8 | | | |
| Q14145 | 288 | KEAP1 | Kelch-like ECH-associated protein 1 | Yes | YES | |
| O75592 | 3152 | MYCBP2 | MYCBP2 Probable E3 ubiquitin-protein | yes | YES | |
| | | | ligase MYCBP2 | | | |
| O75592 | 1131 | MYCBP2 | E3 ubiquitin-protein ligase MYCBP2 | Yes | YES | |
| O75592 | 3152 | MYCBP2 | E3 ubiquitin-protein ligase MYCBP2 | Yes | YES | |
| P35227 | 233 | PCGF2 | Polycomb group RING finger protein 2 | Yes | YES | |
| Q8N2W9 | 326 | PIAS4 | E3 SUMO-protein ligase PIAS4 | Yes | YES | YES |
| P29590 | 484 | PML | Protein PML | Yes | YES | |
| P29590 | 479 | PML | Protein PML | Yes | YES | |
| Q06587 | 72 | RING1 | E3 ubiquitin-protein ligase RING1 | Yes | YES | YES |
| Q06587 | 51 | RING1 | E3 ubiquitin-protein ligase RING1 | Yes | | YES |
| Q99496 | 54 | RNF2 | E3 ubiquitin-protein ligase RING2 | Yes | | YES |
| Q5VTR2 | 383 | RNF20 | E3 ubiquitin-protein ligase BRE1A | Yes | YES | |
| Q63HN8 | 1510 | RNF213 | E3 ubiquitin-protein ligase RNF213 | Yes | YES | |
| Q63HN8 | 4407 | RNF213 | E3 ubiquitin-protein ligase RNF213 | Yes | YES | |
| Q63HN8 | 4258 | RNF213 | E3 ubiquitin-protein ligase RNF213 | Yes | YES | |
| Q8ND24 | 655 | RNF214 | RING finger protein 214 | Yes | YES | YES |
| Q96BH1 | 417 | RNF25 | E3 ubiquitin-protein ligase RNF25 | Yes | YES | |
| O75150 | 890 | RNF40 | E3 ubiquitin-protein ligase BRE1B | Yes | YES | |
| Q9UBF6 | 88 | RNF7 | RING-box protein 2 | Yes | YES | |
| P21580 | 579 | TNFAIP3 | Tumor necrosis factor alpha-induced | Yes | YES | |
| | | | protein 3 | | | |
| P21580 | 54 | TNFAIP3 | Tumor necrosis factor alpha-induced | Yes | YES | YES |
| | | | protein 3 | | | |
| P21580 | 536 | TNFAIP3 | Tumor necrosis factor alpha-induced | Yes | YES | |
| | | | protein 3 | | | |
| Q9UPN9 | 153 | TRIM33 | E3 ubiquitin-protein ligase TRIM33 | Yes | | YES |
| Q9UPN9 | 582 | TRIM33 | E3 ubiquitin-protein ligase TRIM33 | Yes | YES | |
| Q9BRZ2 | 338 | TRIM56 | E3 ubiquitin-protein ligase TRIM56 | Yes | YES | |
| Q9BRZ2 | 36 | TRIM56 | E3 ubiquitin-protein ligase TRIM56 | Yes | | YES |
| Q6PJ69 | 320 | TRIM65 | Tripartite motif-containing protein 65 | Yes | YES | |
| Q2Q1W2 | 636 | TRIM71 | E3 ubiquitin-protein ligase TRIM71 | Yes | YES | |
| Q14669 | 1959 | TRIP12 | TRIP12 E3 ubiquitin-protein ligase | yes | YES | |
| | | | TRIP12 | | | |
| Q14669 | 1959 | TRIP12 | E3 ubiquitin-protein ligase TRIP12 | Yes | YES | |
| Q9C0C9 | 370 | UBE2O | (E3-independent) E2 ubiquitin- | Yes | YES | YES |
| | | | conjugating enzyme | | | |
| Q9C0C9 | 910 | UBE2O | (E3-independent) E2 ubiquitin- | Yes | YES | |
| | | | conjugating enzyme | | | |
| Q9C0C9 | 913 | UBE2O | (E3-independent) E2 ubiquitin- | Yes | YES | |
| | | | conjugating enzyme | | | |
| Q9C0C9 | 400 | UBE2O | (E3-independent) E2 ubiquitin- | Yes | YES | |
| | | | conjugating enzyme | | | |
| Q14139 | 79 | UBE4A | UBE4A Ubiquitin conjugation factor E4 A | yes | YES | |
| Q14139 | 79 | UBE4A | Ubiquitin conjugation factor E4 A | Yes | YES | |
| Q8IWV8 | 1717 | UBR2 | E3 ubiquitin-protein ligase UBR2 | Yes | YES | |
| Q5T4S7 | 4049 | UBR4 | E3 ubiquitin-protein ligase UBR4 | Yes | YES | |
| O95071 | 2314 | UBR5 | E3 ubiquitin-protein ligase UBR5 | Yes | YES | |
| Q9Y4B6 | 69 | VPRBP | Protein VPRBP | Yes | YES | |
| Q9Y4B6 | 967 | VPRBP | Protein VPRBP | Yes | YES | |
| Q9P253 | 806 | VPS18 | Vacuolar protein sorting-associated | Yes | YES | |
| | | | protein 18 homolog | | | |
| Q86WB0 | 406 | ZC3HC1 | Nuclear-interacting partner of ALK | Yes | YES | |

| | | | | | |
|---|---|---|---|---|---|
| P28288 | 472 | ABCD3 | ABCD3 ATP-binding cassette sub-family D | YES | |
| | | | member 3 | | |
| P28288 | 477 | ABCD3 | ABCD3 ATP-binding cassette sub-family D | YES | |
| | | | member 3 | | |
| P42765 | 382 | ACAA2 | ACAA2 3-ketoacyl-CoA thiolase, mitochondrial | YES | |
| Q86UL3 | 325 | AGPAT6 | AGPAT6 Glycerol-3-phosphate acyltransferase 4 | YES | YES |
| Q92667 | 376 | AKAP1 | AKAP1 A-kinase anchor protein 1, mitochondrial | YES | |
| P54886 | 612 | ALDH18A1 | ALDH18A1 Delta-1-pyrroline-5-carboxylate | YES | |
| | | | synthase | | |
| Q3SY69 | 707 | ALDH1L2 | ALDH1L2 Mitochondrial 10- | | YES |
| | | | formyltetrahydrofolate dehydrogen | | |
| Q6DCA0 | 153 | AMMECR1L | AMMECR1L AMMECR1-like protein | | YES |
| Q6NXT1 | 230 | ANKRD54 | ANKRD54 Ankyrin repeat domain-containing | YES | |
| | | | protein 54 | | |
| Q676U5 | 145 | ATG16L1 | ATG16L1 Autophagy-related protein 16-1 | YES | |
| P38606 | 277 | ATP6V1A | ATP6V1A V-type proton ATPase catalytic | YES | YES |
| | | | subunit A | | |
| Q00536 | 206 | CDK16 | CDK16 Cyclin-dependent kinase 16 | YES | YES |
| Q00537 | 233 | CDK17 | CDK17 Cyclin-dependent kinase 17 | YES | YES |
| Q07002 | 183 | CDK18 | CDK18 Cyclin-dependent kinase 18 | YES | YES |
| Q9C0F1 | 28 | CEP44 | CEP44 Centrosomal protein of 44 kDa | YES | |
| P12277 | 283 | CKB | CKB Creatine kinase B-type | YES | |
| P12532 | 316 | CKMT1B | CKMT1B Creatine kinase U-type, mitochondrial | YES | YES |
| P53634 | 258 | CTSC | CTSC Dipeptidyl peptidase 1 | | YES |
| Q9UHI6 | 577 | DDX20 | DDX20 Probable ATP-dependent RNA helicase | YES | |
| | | | DDX20 | | |
| Q9BQ39 | 603 | DDX50 | DDX50 ATP-dependent RNA helicase DDX50 | YES | |
| Q8IY21 | 1051 | DDX60 | DDX60 Probable ATP-dependent RNA helicase | YES | |
| | | | DDX60 | | |
| Q9NUI1 | 120 | DECR2 | DECR2 Peroxisomal 2,4-dienoyl-CoA reductase | YES | YES |
| Q14689 | 6 | DIP2A | DIP2A Disco-interacting protein 2 homolog A | YES | YES |
| P68104 | 370 | EEF1A1 | EEF1A1 Elongation factor 1-alpha 1 | YES | YES |
| P68104 | 363 | EEF1A1 | EEF1A1 Elongation factor 1-alpha 1 | YES | YES |
| Q9NZB2 | 919 | FAM120A | FAM120A Constitutive coactivator of PPAR- | YES | |
| | | | gamma-like protei | | |
| P49327 | 161 | FASN | FASN Fatty acid synthase | YES | |
| Q7L8L6 | 670 | FASTKD5 | FASTKD5 FAST kinase domain-containing | YES | |
| | | | protein 5 | | |
| P37268 | 6 | FDFT1 | FDFT1 Squalene synthase | | YES |
| Q96CP2 | 64 | FLYWCH2 | FLYWCH2 FLYWCH family member 2 | YES | |
| Q14353 | 91 | GAMT | GAMT Guanidinoacetate N-methyltransferase | YES | |
| Q92616 | 648 | GCN1L1 | GCN1L1 Translational activator GCN1 | | YES |
| Q96RP9 | 146 | GFM1 | GFM1 Elongation factor G, mitochondrial | YES | |
| Q08379 | 794 | GOLGA2 | GOLGA2 Golgin subfamily A member 2 | YES | |
| P55084 | 458 | HADHB | HADHB Trifunctional enzyme subunit beta, | YES | |
| | | | mitochondrial | | |
| P68431 | 111 | HIST1H3J | HIST1H3J Histone H3.1 | YES | YES |
| P30519 | 265 | HMOX2 | HMOX2 Heme oxygenase 2 | YES | YES |
| Q8WVV9 | 464 | HNRPLL | HNRPLL Heterogeneous nuclear | YES | |
| | | | ribonucleoprotein L-like | | |
| P48735 | 154 | IDH2 | IDH2 Isocitrate dehydrogenase | YES | |
| Q8N201 | 1534 | INTS1 | INTS1 Integrator complex subunit 1 | | YES |
| Q06136 | 121 | KDSR | KDSR 3-ketodihydrosphingosine reductase | YES | |
| Q86YS7 | 867 | KIAA0528 | KIAA0528 Uncharacterized protein KIAA0528 | YES | |
| O75153 | 333 | KIAA0664 | KIAA0664 Clustered mitochondria protein | YES | |
| | | | homolog | | |
| Q9H9P8 | 187 | L2HGDH | L2HGDH L-2-hydroxyglutarate dehydrogenase, | YES | |
| | | | mitochondrial | | |
| Q96JM7 | 233 | L3MBTL3 | L3MBTL3 Lethal(3)malignant brain tumor-like | YES | |
| | | | protein 3 | | |
| Q99538 | 50 | LGMN | LGMN Legumain | YES | |
| P46821 | 382 | MAP1B | MAP1B Microtubule-associated protein 1B | | YES |
| P53779 | 283 | MAPK10 | MAPK10 Mitogen-activated protein kinase 10 | YES | |
| P45983 | 245 | MAPK8 | MAPK8 Mitogen-activated protein kinase 8 | YES | |
| P03915 | 518 | MT-ND5 | MT-ND5 NADH-ubiquinone oxidoreductase | YES | YES |
| | | | chain 5 | | |
| Q9BXJ9 | 817 | NAA15 | NAA15 N-alpha-acetyltransferase 15, NatA | YES | |
| | | | auxiliary subun | | |
| Q8TD19 | 260 | NEK9 | NEK9 Serine/threonine-protein kinase Nek9 | YES | |
| Q9Y3T9 | 585 | NOC2L | NOC2L Nucleolar complex protein 2 homolog | YES | |
| O00567 | 211 | NOP56 | NOP56 Nucleolar protein 56 | YES | |
| Q9H857 | 504 | NT5DC2 | NT5DC2 5-nucleotidase domain-containing | YES | |
| | | | protein 2 | | |
| Q9H857 | 507 | NT5DC2 | NT5DC2 5-nucleotidase domain-containing | YES | |
| | | | protein 2 | | |
| P53384 | 31 | NUBP1 | NUBP1 Cytosolic Fe—S cluster assembly factor | YES | YES |
| | | | NUBP1 | | |
| Q14690 | 361 | PDCD11 | PDCD11 Protein RRP5 homolog | YES | |
| Q7Z3K3 | 547 | POGZ | POGZ Pogo transposable element with ZNF | YES | |
| | | | domain | | |
| Q96CD2 | 173 | PPCDC | PPCDC Phosphopantothenoylcysteine | YES | |
| | | | decarboxylase | | |
| O14744 | 278 | PRMT5 | PRMT5 Protein arginine N-methyltransferase 5 | YES | |
| P04843 | 477 | RPN1 | RPN1 Dolichyl-diphosphooligosaccharide-- | YES | |
| | | | protein glycosy | | |
| Q15418 | 223 | RPS6KA1 | RPS6KA1 Ribosomal protein S6 kinase alpha-1 | YES | |
| P51812 | 229 | RPS6KA3 | RPS6KA3 Ribosomal protein S6 kinase alpha-3 | YES | |
| Q6P087 | 147 | RPUSD3 | RPUSD3 RNA pseudouridylate synthase domain- | YES | |
| | | | containing pro | | |
| Q14684 | 155 | RRP1B | RRP1B Ribosomal RNA processing protein 1 | YES | |
| | | | homolog B | | |
| O43159 | 451 | RRP8 | RRP8 Ribosomal RNA-processing protein 8 | YES | |
| Q9UGP8 | 295 | SEC63 | SEC63 Translocation protein SEC63 homolog | YES | |
| O15047 | 1648 | SETD1A | SETD1A Histone-lysine N-methyltransferase | YES | |
| | | | SETD1A | | |
| Q9Y5X2 | 200 | SNX8 | SNX8 Sorting nexin-8 | YES | |
| Q96R06 | 382 | SPAG5 | SPAG5 Sperm-associated antigen 5 | YES | YES |
| Q13501 | 131 | SQSTM1 | SQSTM1 Sequestosome-1 | YES | |
| Q9UQ35 | 1029 | SRRM2 | SRRM2 Serine/arginine repetitive matrix protein 2 | YES | |
| Q9UQ35 | 1036 | SRRM2 | SRRM2 Serine/arginine repetitive matrix protein 2 | YES | |
| P28290 | 901 | SSFA2 | SSFA2 Sperm-specific antigen 2 | | YES |
| Q03519 | 641 | TAP2 | TAP2 Antigen peptide transporter 2 | YES | YES |
| O15533 | 440 | TAPBP | TAPBP Tapasin | YES | |
| Q8TC07 | 24 | TBC1D15 | TBC1D15 TBC1 domain family member 15 | YES | |
| Q7Z2T5 | 239 | TRMT1L | TRMT1L TRMT1-like protein | YES | |
| Q13748 | 4 | TUBA3D | TUBA3D Tubulin alpha-3C/D chain | YES | YES |
| Q13748 | 20 | TUBA3D | TUBA3D Tubulin alpha-3C/D chain | YES | YES |
| Q9H1C4 | 583 | UNC93B1 | UNC93B1 Protein unc-93 homolog B1 | YES | |
| P45880 | 227 | VDAC2 | VDAC2 Voltage-dependent anion-selective | YES | YES |
| | | | channel protein | | |
| P45880 | 210 | VDAC2 | VDAC2 Voltage-dependent anion-selective | YES | YES |
| | | | channel protein | | |
| Q9Y277 | 229 | VDAC3 | VDAC3 Voltage-dependent anion-selective | YES | |
| | | | channel protein | | |
| Q8IV63 | 191 | VRK3 | VRK3 Inactive serine/threonine-protein kinase | YES | |
| | | | VRK3 | | |
| Q5VUA4 | 1681 | ZNF318 | ZNF318 Zinc finger protein 318 | YES | |
| Q14966 | 1023 | ZNF638 | ZNF638 Zinc finger protein 638 | YES | |
| Q14966 | 1026 | ZNF638 | ZNF638 Zinc finger protein 638 | YES | |
| Q14966 | 1023 | ZNF638 | ZNF638 Zinc finger protein 638 | YES | |
| Q9H582 | 507 | ZNF644 | ZNF644 Zinc finger protein 644 | YES | |

TABLE 1B

| uniprot | KB02¹ | KB02² | KB02³ | KB02⁴ | KB03⁵ | KB03⁶ | KB03⁷ | KB03⁸ |
|---|---|---|---|---|---|---|---|---|
| Q96DE5 | 75.05 | 34.265 | 75.46 | 46.17 | 28.34 | −0.24 | 36.5 | 20.4 |
| Q96DE5 | 75.1 | 34.3 | 75.5 | 46.2 | 28.3 | −0.2 | 36.5 | 20.4 |
| Q9UJX3 | 77.1 | 51.2 | 77.1 | 58.1 | 68.4 | 47.5 | 73.4 | 60.9 |
| O15033 | | 77.2 | | 77.2 | 55.1 | 49.1 | 55.1 | 56.8 |
| O14867 | 87.8 | 61.6 | 87.8 | 61.6 | 85.3 | 53.8 | 85.3 | 53.8 |
| Q9BYV9 | 77.6 | 40.9 | 77.6 | 42.2 | 62.8 | 17.2 | 62.8 | 25.4 |
| Q13490 | 95.7 | 84.9 | 98.1 | 92.3 | 85.9 | 41.4 | 92.0 | 47.9 |
| Q13489 | 76.9 | 25.8 | 76.9 | 27.2 | 18.7 | −2.6 | 18.7 | 1.3 |
| Q5H9S7 | 91.7 | 40.3 | 91.7 | 60.8 | | −21.2 | | −21.2 |
| Q9NZJ0 | 93.6 | 85.0 | 93.6 | 85.0 | 70.5 | 36.7 | 70.5 | 36.7 |
| Q9NZJ0 | 77.3 | 33.7 | 77.3 | 33.7 | 69.3 | 29.9 | 69.3 | 29.9 |
| Q86XK2 | 83.2 | 43.1 | 98.3 | 63.5 | 20.3 | −20.1 | 28.6 | −13.9 |
| Q8TB52 | 54.1 | 35.6 | 80.1 | 56.3 | 25.0 | 4.3 | 39.5 | 10.4 |
| Q9ULT8 | 87.1 | 29.5 | 87.1 | 43.3 | 72.6 | 0.1 | 72.6 | 1.2 |
| Q15751 | 90.7 | 45.6 | 91.8 | 62.8 | 58.0 | 8.9 | 68.4 | 20.5 |
| Q15751 | | 63.2 | | 77.7 | | 2.0 | | 2.9 |
| Q5GLZ8 | 79.8 | 32.6 | 82.2 | 40.3 | 58.8 | 15.4 | 62.3 | 42.0 |
| Q9UII4 | 85.3 | 18.9 | 85.3 | 18.9 | 96.8 | 83.4 | 96.8 | 83.4 |
| Q7Z6Z7 | | 76.4 | | 76.4 | | 9.7 | | 9.7 |
| Q8NFY9 | 79.2 | 36.7 | 79.2 | 38.4 | 21.5 | −7.4 | 21.5 | 5.3 |
| Q14145 | 82.3 | 37.7 | 82.3 | 37.7 | | | | |
| O75592 | 71.85 | 20.98 | 76.84 | 28.88 | 25.92 | −7.46 | 28.63 | 4.67 |
| O75592 | 82.8 | 42.5 | 82.8 | 55.0 | 30.7 | −12.9 | 30.7 | 0.0 |
| O75592 | 71.9 | 21.0 | 76.8 | 28.9 | 25.9 | −7.5 | 28.6 | 4.7 |
| P35227 | 79.2 | 35.2 | 80.1 | 41.7 | 35.9 | −4.4 | 49.9 | 5.9 |
| Q8N2W9 | 96.5 | 91.8 | 96.5 | 96.0 | 75.4 | 30.1 | 87.0 | 35.5 |
| P29590 | 75.5 | 26.1 | 84.7 | 44.1 | 65.9 | 0.4 | 65.9 | 10.2 |
| P29590 | 72.4 | 33.3 | 79.4 | 58.9 | 44.4 | −3.1 | 60.8 | 15.3 |
| Q06587 | 83.1 | 50.3 | 83.1 | 50.3 | 83.4 | 32.4 | 83.4 | 32.4 |
| Q06587 | 24.0 | −2.4 | 24.0 | 4.8 | 76.8 | 0.5 | 76.8 | 18.1 |
| Q99496 | 24.0 | −2.4 | 24.0 | 4.8 | 76.8 | 0.5 | 76.8 | 18.1 |
| Q5VTR2 | 86.1 | 57.7 | 94.1 | 62.1 | 32.6 | 0.6 | 37.3 | 11.0 |
| Q63HN8 | 93.0 | 79.9 | 96.5 | 88.1 | 58.4 | 5.6 | 66.6 | 15.3 |
| Q63HN8 | 87.1 | 56.7 | 87.1 | 68.4 | 39.2 | −21.4 | 39.2 | −15.8 |
| Q63HN8 | | 70.3 | | 74.7 | 26.0 | −9.8 | 26.0 | −1.2 |
| Q8ND24 | 74.8 | 45.8 | 83.2 | 50.3 | 65.9 | 24.8 | 96.4 | 35.5 |
| Q96BH1 | 77.7 | 33.5 | 77.7 | 33.5 | 62.0 | 14.3 | 62.0 | 14.3 |
| O75150 | 91.8 | 56.5 | 93.4 | 79.4 | 24.5 | −9.9 | 29.2 | −5.2 |
| Q9UBF6 | | 75.2 | | 75.2 | | 20.4 | | 20.4 |
| P21580 | 99.8 | 99.2 | 99.8 | 99.8 | 56.8 | 28.1 | 56.8 | 52.6 |
| P21580 | 98.9 | 83.8 | 99.6 | 90.4 | 99.3 | 97.1 | 99.3 | 98.2 |
| P21580 | 86.6 | 47.0 | 86.6 | 53.4 | 46.1 | 28.5 | 46.1 | 31.2 |
| Q9UPN9 | 33.1 | 4.4 | 51.7 | 30.1 | 56.8 | 10.3 | 85.7 | 86.6 |
| Q9UPN9 | 81.0 | 24.1 | 84.6 | 43.7 | 57.4 | 1.1 | 70.5 | 22.0 |
| Q9BRZ2 | 87.7 | 40.3 | 88.3 | 54.3 | | 11.8 | | 25.9 |
| Q9BRZ2 | 37.9 | 6.0 | 48.6 | 18.4 | 78.3 | 24.0 | 78.3 | 32.2 |
| Q6PJ69 | 87.6 | 45.8 | 87.7 | 58.0 | 60.2 | 19.4 | 70.9 | 35.1 |
| Q2Q1W2 | 87.2 | 33.5 | 87.2 | 33.5 | 64.2 | 17.2 | 64.2 | 17.2 |
| Q14669 | 75.47 | 20.33 | 75.47 | 24.93 | 48.04 | 0.28 | 48.04 | 6.39 |
| Q14669 | 75.5 | 20.3 | 75.5 | 24.9 | 48.0 | 0.3 | 48.0 | 6.4 |
| Q9C0C9 | 74.3 | 21.4 | 89.5 | 49.4 | 53.0 | 2.0 | 76.5 | 30.4 |
| Q9C0C9 | 83.8 | 34.8 | 83.8 | 34.8 | 34.3 | 35.7 | 34.3 | 35.7 |
| Q9C0C9 | 77.5 | 19.1 | 77.5 | 19.1 | 30.1 | 16.7 | 30.1 | 16.7 |
| Q9C0C9 | 76.2 | 49.9 | 76.2 | 49.9 | 59.4 | 23.1 | 59.4 | 23.1 |
| Q14139 | 90.45 | 9.64 | 90.45 | 16.75 | 19.24 | 2.64 | 24.72 | 10.39 |
| Q14139 | 90.5 | 9.6 | 90.5 | 16.8 | 19.2 | 2.6 | 24.7 | 10.4 |
| Q8IWV8 | 90.9 | 60.0 | 92.7 | 72.1 | 60.0 | 8.3 | 65.9 | 18.5 |
| Q5T4S7 | 71.9 | 18.6 | 82.0 | 24.0 | 29.3 | −4.9 | 49.5 | 13.1 |
| O95071 | 66.6 | 17.3 | 76.2 | 19.3 | 26.8 | −2.4 | 33.0 | 9.2 |
| Q9Y4B6 | 84.1 | 52.4 | 84.1 | 56.8 | 59.5 | 30.2 | 59.5 | 61.2 |
| Q9Y4B6 | 77.3 | 30.3 | 77.3 | 30.3 | 28.2 | −0.9 | 28.2 | −0.9 |
| Q9P253 | 85.0 | 35.2 | 85.0 | 45.1 | 47.0 | 16.2 | 47.0 | 19.6 |
| Q86WB0 | 65.5 | 13.9 | 77.7 | 34.0 | 34.6 | −8.7 | 53.8 | 6.3 |
| P28288 | 84.24 | 10.775 | 84.24 | 30.56 | 34.93 | −5.085 | 34.93 | 3.18 |
| P28288 | 86.86 | 29.54 | 87.59 | 43.9 | 35.18 | −12.80 | 39.85 | −0.23 |
| P42765 | 94.11 | 92.98 | 94.11 | 92.98 | −0.69 | 3.45 | −0.69 | 3.45 |
| Q86UL3 | 93.71 | 72.63 | 93.71 | 88.26 | 85.19 | 65.705 | 85.19 | 66.91 |
| Q92667 | 84.17 | 22.975 | 84.17 | 43.22 | 45.04 | −0.33 | 57.81 | 18.92 |
| P54886 | 80.12 | 34.8575 | 80.76 | 40.24 | 66.23 | 13.91 | 66.31 | 34.21 |
| Q3SY69 | 35.43 | −9.84 | 35.43 | −9.84 | 77.55 | 52.7 | 77.55 | 52.7 |
| Q6DCA0 | | | | | 85.74 | 91.46 | 85.74 | 91.46 |
| Q6NXT1 | 75.08 | 28.77 | 77.15 | 46.44 | 44.21 | −0.72 | 45.29 | 7.02 |
| Q676U5 | 80 | 16.2813 | 80 | 16.28 | 18.73 | −47.09 | 18.73 | −47.09 |
| P38606 | 84.565 | 56.1875 | 86.62 | 67.33 | 69.985 | 18.165 | 76.55 | 27.98 |
| Q00536 | 88.13 | 48.8275 | 90.78 | 69.25 | 79.225 | 28.2075 | 80.44 | 50.67 |
| Q00537 | 88.13 | 48.8275 | 90.78 | 69.25 | 79.225 | 28.2075 | 80.44 | 50.67 |
| Q07002 | 88.13 | 48.8275 | 90.78 | 69.25 | 79.225 | 28.2075 | 80.44 | 50.67 |
| Q9C0F1 | 78.74 | 16.65 | 78.74 | 16.65 | 69.9 | 14.85 | 69.9 | 14.85 |
| P12277 | 95.3 | 84.26 | 95.3 | 85.17 | 71.73 | 15.155 | 71.73 | 23.24 |
| P12532 | 91.53 | 80.325 | 96.43 | 96.46 | 81.71 | 29.59 | 86.19 | 42.35 |
| P53634 | 50.925 | 36.695 | 64.88 | 62.83 | 95.44 | 76.05 | 95.44 | 76.05 |
| Q9UHI6 | 74.765 | 21.5175 | 85.27 | 39.22 | 31.49 | −6.7225 | 37.31 | 0.85 |
| Q9BQ39 | 91.56 | 41.415 | 95.81 | 60.06 | 22.82 | −13.925 | 28.63 | −9.62 |
| Q8IY21 | 98.68 | 88.54 | 98.68 | 93.26 | 73.07 | 19.7 | 73.07 | 29.56 |
| Q9NUI1 | 55.92 | 76.8 | 55.92 | 76.8 | 85 | 66.9 | 85 | 66.9 |
| Q14689 | 85.31 | 24.24 | 85.31 | 24.24 | 91.21 | 39.34 | 91.21 | 39.34 |
| P68104 | 79.795 | 31.885 | 86.92 | 48.77 | 74.35 | 24 | 87.08 | 46.21 |
| P68104 | 78.125 | 36.0625 | 87.29 | 50.66 | 86.19 | 21.28333 | 86.19 | 43.3 |
| Q9NZB2 | 82.72 | 2.92 | 82.72 | 8.53 | 55.62 | −8.925 | 55.62 | 13.09 |
| P49327 | | 90.93 | | 90.93 | | −26.25 | | −26.25 |
| Q7L8L6 | 75.66 | 38.15 | 75.66 | 38.15 | 45.06 | 14.55 | 57.23 | 14.75 |
| P37268 | 69.42 | 19.56 | 69.42 | 30.82 | 82.93 | 37.70333 | 82.93 | 59.08 |
| Q96CP2 | 79.89 | 18.1 | 79.89 | 25.4 | 41.635 | 2.25 | 41.66 | 12.97 |
| Q14353 | 87.425 | 41.34 | 88.2 | 46.77 | 6.05 | −25.93 | 16.85 | −9.9 |
| Q92616 | 55.68 | 11.47 | 57.17 | 30.58 | 78.745 | 25.61 | 80.3 | 28.59 |
| Q96RP9 | 80.72 | 34.48 | 80.72 | 34.48 | | | | |
| Q08379 | 85.65 | 30.41 | 85.65 | 30.41 | 54.38 | 1.85 | 54.38 | 1.85 |
| P55084 | 91.7 | 71.865 | 94.32 | 80.88 | 60.9 | 15.245 | 64.33 | 26.24 |
| P68431 | 86.28 | 27.43 | 86.28 | 47.9 | 79.39 | 22.28 | 79.39 | 50.87 |
| P30519 | 93.98 | 74.475 | 98.47 | 89.99 | 88.6 | 81.435 | 98.54 | 93.81 |
| Q8WVV9 | 90.12 | 39.085 | 90.12 | 49.46 | 61.83 | 3.35 | 61.83 | 4.14 |
| P48735 | 37.86 | 20.30 | 75.27 | 54.11 | 70.11 | 43.06 | 73.43 | 63.35 |
| Q8N201 | 24.155 | −2.61 | 26.58 | 3.23 | 70.05 | 36.495 | 77.65 | 51.25 |
| Q06136 | | 79.49 | | 79.49 | | −22.72 | | −22.72 |
| Q86YS7 | 71.91 | 20.55 | 76.77 | 29.09 | 33.415 | −6.4125 | 46.81 | 7.23 |
| O75153 | 86.65 | 15.665 | 86.65 | 29.11 | 51.405 | 9.323333 | 73.65 | 23.11 |
| Q9H9P8 | 73.53 | 34.0075 | 75.71 | 40.09 | 67.435 | 26.9925 | 70.21 | 39.03 |
| Q96JM7 | 77.37 | 24.805 | 77.37 | 32.93 | 57.17 | 10.83 | 57.17 | 16.63 |
| Q99538 | 78.38 | 20.31 | 78.38 | 33.6 | 60.93 | 0.755 | 60.93 | 12.45 |
| P46821 | 71.07 | 27.9 | 71.07 | 27.9 | 75.17 | 37.5 | 75.17 | 37.5 |
| P53779 | 76.89 | 26.11 | 78.63 | 44.42 | 40.875 | 12.33 | 44.06 | 17.22 |
| P45983 | 76.89 | 26.11 | 78.63 | 44.42 | 40.875 | 12.33 | 44.06 | 17.22 |
| P03915 | | 88.34 | | 88.34 | | 83.61 | | 83.61 |
| Q9BXJ9 | 87.75 | 14.80 | 87.75 | 30.16 | 14.66 | −31.6767 | 14.66 | −21.23 |
| Q8TD19 | 79.22 | 30.62 | 79.22 | 30.62 | 56.18 | 9.47 | 56.18 | 9.47 |
| Q9Y3T9 | 93.26 | 39.755 | 95.32 | 48.21 | 56.82 | 7.195 | 59.2 | 29.97 |
| O00567 | 75.79 | 14.04 | 75.79 | 36.67 | 68.12 | 27.36 | 68.12 | 51.21 |
| Q9H857 | 79.98 | 9.89 | 79.98 | 9.89 | 66.1 | 10.83 | 66.1 | 10.83 |
| Q9H857 | 77.1 | 28.22 | 77.1 | 31.68 | 65.33 | 9.86 | 65.33 | 18.84 |
| P53384 | 63.785 | 25.0375 | 76.77 | 44.6 | 55.66 | 7.3775 | 75.8 | 28.64 |
| Q14690 | 85.92 | 39.81 | 85.92 | 50.22 | 46.645 | 3.78 | 49.26 | 19.85 |
| Q7Z3K3 | 88.955 | 43.4525 | 89.05 | 62.36 | 13.995 | −8.9575 | 15.64 | 2.21 |
| Q96CD2 | 87.25 | 35.095 | 87.25 | 46.12 | 71.73 | 13.215 | 71.73 | 23.85 |
| O14744 | 91.94 | 33.17 | 91.94 | 33.17 | 45.47 | 16.62 | 45.47 | 16.62 |
| P04843 | 89.175 | 36.5375 | 92.37 | 47.76 | 23.85 | −5.035 | 28.71 | 9.24 |
| Q15418 | 75.6 | 39.63 | 75.6 | 39.63 | 30.61 | 1.03 | 30.61 | 1.03 |
| P51812 | 75.6 | 21.44 | 75.6 | 39.63 | 30.61 | −4.76667 | 30.61 | 1.03 |
| Q6P087 | 95.86 | 70.79 | 95.86 | 79.96 | 49.47 | 9.07 | 49.47 | 14.63 |
| Q14684 | 76.37 | 29.87 | 80.26 | 42.52 | 66.05 | 18.31333 | 70.79 | 27.08 |
| O43159 | 84.6 | 27.285 | 85.51 | 39.05 | 48.93 | −3.99 | 57.64 | 14.62 |
| Q9UGP8 | 86.655 | 37.09 | 91.28 | 44.36 | 48.96 | 4.99 | 55.53 | 17.92 |
| O15047 | 72.83 | 36.6275 | 76.52 | 54.8 | 38.365 | −0.4575 | 40.15 | 15.12 |
| Q9Y5X2 | | 75.58 | | 75.58 | | 36.21 | | 36.21 |
| Q96R06 | 84.21 | 58.18 | 84.21 | 58.18 | 86.56 | 53.77 | 86.56 | 53.77 |
| Q13501 | 80.385 | 22.825 | 80.81 | 41.08 | 54.29 | −14.15 | 56.86 | 11.89 |
| Q9UQ35 | 72.45 | 31.6 | 88.59 | 39.96 | 33.105 | 0.7 | 55.3 | 1.76 |
| Q9UQ35 | 87.74 | 18.93 | 87.74 | 39.43 | 53.28 | −5.29333 | 53.28 | 5.78 |
| P28290 | 65.99 | 22.48 | 65.99 | 22.48 | 76.66 | 34.29 | 76.66 | 34.29 |
| Q03519 | 99.1948 | 85.26225 | 99.1948 | 88.9828 | 94.0932 | 70.56835 | 94.0932 | 71.9622 |
| O15533 | 99.1316 | 98.8859 | 99.1316 | 99.1295 | 68.7451 | 28.53795 | 68.7451 | 31.4264 |
| Q8TC07 | 86.57 | 22.025 | 86.57 | 30.32 | 47.51 | 0.88 | 47.51 | 7.83 |
| Q7Z2T5 | 65.645 | 5.516667 | 75.12 | 12.37 | 36.38 | −17.83 | 36.38 | −4.62 |
| Q13748 | 84.97 | 9.925 | 84.97 | 25.78 | 77.83 | 15.975 | 77.83 | 49.24 |
| Q13748 | 90.9 | 9.25 | 90.9 | 30.23 | 91.98 | 14.425 | 91.98 | 42.36 |
| Q9H1C4 | 89.88683 | 35.09511 | 92.03 | 59.48 | 70.38475 | 13.00236 | 72.3195 | 29.9385 |
| P45880 | 84.085 | 53.2675 | 93.19 | 72.22 | 79.44 | 73.7325 | 94.31 | 85.47 |
| P45880 | 92.73 | 54.92 | 92.73 | 54.92 | 95.08 | 92.99 | 95.08 | 92.99 |
| Q9Y277 | 74.03 | 23.8025 | 75.01 | 33.34 | 44.31 | 4.9525 | 51.97 | 21.56 |
| Q8IV63 | 82.09 | 19.46 | 82.09 | 25.34 | 14.41 | −10.03 | 14.41 | −8.38 |
| Q5VUA4 | 71.6 | 32.505 | 77.86 | 38.01 | 40.555 | −8.115 | 55.07 | −1.63 |
| Q14966 | 62.76 | 10.52 | 91.48 | 18.82 | 58.34 | −4.38 | 58.34 | −4.38 |
| Q14966 | 75.07 | 33.24 | 75.07 | 33.24 | 63.04 | 17.48 | 63.04 | 17.48 |
| Q14966 | 62.76 | 10.52 | 91.48 | 18.82 | 58.34 | −4.38 | 58.34 | −4.38 |
| Q9H582 | 79.04 | 37.4675 | 82.24 | 63 | 67.685 | 11.2825 | 72.77 | 24.53 |

¹KB02_500 ave;
²KB02_100 ave;
³KB02_500 Max;
⁴KB02_100 Max;
⁵KB03_500 ave;
⁶KB03_100 ave;
⁷KB03_500 Max:
⁸KB03_100 Max;

Table 2A and illustrate additional exemplary proteins and cysteine site residues described herein.

TABLE 2A

| UniProt | Description | Symbol | Residue |
|---|---|---|---|
| Q969K4 | Ankyrin repeat and BTB/POZ domain-containing protein 1 | ABTB1 | 62 |
| Q969K4 | Ankyrin repeat and BTB/POZ domain-containing protein 1 | ABTB1 | 72 |
| Q969K4 | Ankyrin repeat and BTB/POZ domain-containing protein 1 | ABTB1 | 95 |
| Q969K4 | Ankyrin repeat and BTB/POZ domain-containing protein 1 | ABTB1 | 222 |
| Q969K4 | Ankyrin repeat and BTB/POZ domain-containing protein 1 | ABTB1 | 285 |
| Q8N961 | Ankyrin repeat and BTB/POZ domain-containing protein 2 | ABTB2 | 27 |
| Q8N961 | Ankyrin repeat and BTB/POZ domain-containing protein 2 | ABTB2 | 361 |
| Q8N961 | Ankyrin repeat and BTB/POZ domain-containing protein 2 | ABTB2 | 393 |
| Q8N961 | Ankyrin repeat and BTB/POZ domain-containing protein 2 | ABTB2 | 460 |
| Q8N961 | Ankyrin repeat and BTB/POZ domain-containing protein 2 | ABTB2 | 470 |
| Q8N961 | Ankyrin repeat and BTB/POZ domain-containing protein 2 | ABTB2 | 497 |
| Q9C0C7 | Activating molecule in BECN1-regulated autophagy protein 1 | AMBRA1 | 87 |
| Q9C0C7 | Activating molecule in BECN1-regulated autophagy protein l | AMBRA1 | 301 |
| Q9C0C7 | Activating molecule in BECN1-regulated autophagy protein 1 | AMBRA1 | 302 |
| Q9C0C7 | Activating molecule in BECN1-regulated autophagy protein 1 | AMBRA1 | 501 |
| Q9C0C7 | Activating molecule in BECN1-regulated autophagy protein 1 | AMBRA1 | 571 |
| Q9C0C7 | Activating molecule in BECN1-regulated autophagy protein 1 | AMBRA1 | 1270 |
| Q9UKV5 | E3 ubiquitin-protein ligase AMFR | AMFR | 364 |
| Q9H1A4 | Anaphase-promoting complex subunit 1 | ANAPC1 | 28 |
| Q9H1A4 | Anaphase-promoting complex subunit 1 | ANAPC1 | 437 |
| Q9H1A4 | Anaphase-promoting complex subunit 1 | ANAPC1 | 575 |
| Q9H1A4 | Anaphase-promoting complex subunit 1 | ANAPC1 | 962 |
| Q9H1A4 | Anaphase-promoting complex subunit 1 | ANAPC1 | 988 |
| Q9H1A4 | Anaphase-promoting complex subunit 1 | ANAPC1 | 1074 |
| Q9UM13 | Anaphase-promoting complex subunit 10 | ANAPC10 | 173 |
| Q9NYG5 | Anaphase-promoting complex subunit 11 | ANAPC11 | 7 |
| Q9NYG5 | Anaphase-promoting complex subunit 11 | ANAPC11 | 34 |
| Q9NYG5 | Anaphase-promoting complex subunit 11 | ANAPC11 | 73 |
| Q9NYG5 | Anaphase-promoting complex subunit 11 | ANAPC11 | 76 |
| P60006 | Anaphase-promoting complex subunit 15 | ANAPC15 | 24 |
| Q96DE5 | Anaphase-promoting complex subunit 16 | ANAPC16 | 55 |
| Q9UJX6 | Anaphase-promoting complex subunit 2 | ANAPC2 | 179 |
| Q9UJX6 | Anaphase-promoting complex subunit 2 | ANAPC2 | 221 |
| Q9UJX6 | Anaphase-promoting complex subunit 2 | ANAPC2 | 224 |
| Q9UJX6 | Anaphase-promoting complex subunit 2 | ANAPC2 | 231 |
| Q9UJX6 | Anaphase-promoting complex subunit 2 | ANAPC2 | 233 |
| Q9UJX6 | Anaphase-promoting complex subunit 2 | ANAPC2 | 394 |
| Q9UJX6 | Anaphase-promoting complex subunit 2 | ANAPC2 | 557 |
| Q9UJX5 | Anaphase-promoting complex subunit 4 | ANAPC4 | 7 |
| Q9UJX4 | Anaphase-promoting complex subunit 5 | ANAPC5 | 86 |
| Q9UJX4 | Anaphase-promoting complex subunit 5 | ANAPC5 | 203 |
| Q9UJX4 | Anaphase-promoting complex subunit 5 | ANAPC5 | 312 |
| Q9UJX4 | Anaphase-promoting complex subunit 5 | ANAPC5 | 339 |
| Q9UJX4 | Anaphase-promoting complex subunit 5 | ANAPC5 | 571 |
| Q9UJX4 | Anaphase-promoting complex subunit 5 | ANAPC5 | 670 |
| Q9UJX4 | Anaphase-promoting complex subunit 5 | ANAPC5 | 733 |
| Q9UJX3 | Anaphase-promoting complex subunit 7 | ANAPC7 | 131 |
| Q9UJX3 | Anaphase-promoting complex subunit 7 | ANAPC7 | 145 |
| Q9UJX3 | Anaphase-promoting complex subunit 7 | ANAPC7 | 199 |
| Q9UJX3 | Anaphase-promoting complex subunit 7 | ANAPC7 | 259 |
| Q9UJX3 | Anaphase-promoting complex subunit 7 | ANAPC7 | 329 |
| Q9UJX3 | Anaphase-promoting complex subunit 7 | ANAPC7 | 347 |
| Q9UJX3 | Anaphase-promoting complex subunit 7 | ANAPC7 | 405 |
| Q9UJX3 | Anaphase-promoting complex subunit 7 | ANAPC7 | 409 |
| Q9UJX3 | Anaphase-promoting complex subunit 7 | ANAPC7 | 451 |
| Q9UJX3 | Anaphase-promoting complex subunit 7 | ANAPC7 | 509 |
| Q9P2R3 | Rabankyrin-5 | ANKFY1 | 34 |
| Q9P2R3 | Rabankyrin-5 | ANKFY1 | 389 |
| Q9P2R3 | Rabankyrin-5 | ANKFY1 | 460 |
| Q9P2R3 | Rabankyrin-5 | ANKFY1 | 499 |
| Q9P2R3 | Rabankyrin-5 | ANKFY1 | 675 |
| Q9P2R3 | Rabankyrin-5 | ANKFY1 | 712 |
| Q9P2R3 | Rabankyrin-5 | ANKFY1 | 716 |
| Q9P2R3 | Rabankyrin-5 | ANKFY1 | 724 |
| Q9P2R3 | Rabankyrin-5 | ANKFY1 | 742 |
| Q9P2R3 | Rabankyrin-5 | ANKFY1 | 749 |
| Q9P2R3 | Rabankyrin-5 | ANKFY1 | 843 |
| Q9P2R3 | Rabankyrin-5 | ANKFY1 | 995 |
| Q9P2R3 | Rabankyrin-5 | ANKFY1 | 1060 |
| Q9P2R3 | Rabankyrin-5 | ANKFY1 | 1105 |
| Q9P2R3 | Rabankyrin-5 | ANKFY1 | 1110 |
| Q9P2R3 | Rabankyrin-5 | ANKFY1 | 1113 |
| Q9P2R3 | Rabankyrin-5 | ANKFY1 | 1156 |
| Q9P2R3 | Rabankyrin-5 | ANKFY1 | 1159 |
| Q9P2G1 | Ankyrin repeat and IBR domain-containing protein 1 | ANKIB1 | 116 |
| Q9P2G1 | Ankyrin repeat and IBR domain-containing protein 1 | ANKIB1 | 160 |
| Q9P2G1 | Ankyrin repeat and IBR domain-containing protein 1 | ANKIB1 | 184 |
| Q9P2G1 | Ankyrin repeat and IBR domain-containing protein 1 | ANKIB1 | 280 |
| Q9P2G1 | Ankyrin repeat and IBR domain-containing protein 1 | ANKIB1 | 359 |
| Q9P2G1 | Ankyrin repeat and IBR domain-containing protein 1 | ANKIB1 | 468 |
| Q9P2G1 | Ankyrin repeat and IBR domain-containing protein 1 | ANKIB1 | 519 |
| Q9P2G1 | Ankyrin repeat and IBR domain-containing protein 1 | ANKIB1 | 532 |
| Q9P2G1 | Ankyrin repeat and IBR domain-containing protein 1 | ANKIB1 | 640 |
| Q9P2G1 | Ankyrin repeat and IBR domain-containing protein 1 | ANKIB1 | 715 |
| Q92624 | Amyloid protein-binding protein 2 | APPBP2 | 54 |
| Q92624 | Amyloid protein-binding protein 2 | APPBP2 | 393 |
| O15033 | Apoptosis-resistant E3 ubiquitin protein ligase 1 | AREL1 | 382 |
| Q9Y4X5 | E3 ubiquitin-protein ligase ARIH1 | ARIH1 | 161 |
| Q9Y4X5 | E3 ubiquitin-protein ligase ARIH1 | ARIH1 | 186 |
| Q9Y4X5 | E3 ubiquitin-protein ligase ARIH1 | ARIH1 | 189 |
| Q9Y4X5 | E3 ubiquitin-protein ligase ARIH1 | ARIH1 | 203 |
| Q9Y4X5 | E3 ubiquitin-protein ligase ARIH1 | ARIH1 | 208 |
| Q9Y4X5 | E3 ubiquitin-protein ligase ARIH1 | ARIH1 | 211 |
| Q9Y4X5 | E3 ubiquitin-protein ligase ARIH1 | ARIH1 | 269 |
| Q9Y4X5 | E3 ubiquitin-protein ligase ARIH1 | ARIH1 | 276 |
| Q9Y4X5 | E3 ubiquitin-protein ligase ARIH1 | ARIH1 | 281 |
| Q9Y4X5 | E3 ubiquitin-protein ligase ARIH1 | ARIH1 | 304 |
| Q9Y4X5 | E3 ubiquitin-protein ligase ARIH1 | ARIH1 | 307 |
| Q9Y4X5 | E3 ubiquitin-protein ligase ARIH1 | ARIH1 | 317 |
| Q9Y4X5 | E3 ubiquitin-protein ligase ARIH1 | ARIH1 | 327 |
| Q9Y4X5 | E3 ubiquitin-protein ligase ARIH1 | ARIH1 | 357 |
| Q9Y4X5 | E3 ubiquitin-protein ligase ARIH1 | ARIH1 | 372 |
| Q9Y4X5 | E3 ubiquitin-protein ligase ARIH1 | ARIH1 | 375 |
| Q9Y4X5 | E3 ubiquitin-protein ligase ARIH1 | ARIH1 | 389 |
| Q9Y4X5 | E3 ubiquitin-protein ligase ARIH1 | ARIH1 | 418 |
| O95376 | E3 ubiquitin-protein ligase ARIH2 | ARIH2 | 142 |
| O95376 | E3 ubiquitin-protein ligase ARIH2 | ARIH2 | 156 |
| O95376 | E3 ubiquitin-protein ligase ARIH2 | ARIH2 | 161 |
| O95376 | E3 ubiquitin-protein ligase ARIH2 | ARIH2 | 164 |
| O95376 | E3 ubiquitin-protein ligase ARIH2 | ARIH2 | 169 |
| O95376 | E3 ubiquitin-protein ligase ARIH2 | ARIH2 | 183 |
| O95376 | E3 ubiquitin-protein ligase ARIH2 | ARIH2 | 188 |
| O95376 | E3 ubiquitin-protein ligase ARIH2 | ARIH2 | 228 |
| O95376 | E3 ubiquitin-protein ligase ARIH2 | ARIH2 | 233 |
| O95376 | E3 ubiquitin-protein ligase ARIH2 | ARIH2 | 249 |
| O95376 | E3 ubiquitin-protein ligase ARIH2 | ARIH2 | 252 |
| O95376 | E3 ubiquitin-protein ligase ARIH2 | ARIH2 | 257 |
| O95376 | E3 ubiquitin-protein ligase ARIH2 | ARIH2 | 270 |
| O95376 | E3 ubiquitin-protein ligase ARIH2 | ARIH2 | 280 |
| O95376 | E3 ubiquitin-protein ligase ARIH2 | ARIH2 | 300 |
| O95376 | E3 ubiquitin-protein ligase ARIH2 | ARIH2 | 303 |
| O95376 | E3 ubiquitin-protein ligase ARIH2 | ARIH2 | 310 |
| O95376 | E3 ubiquitin-protein ligase ARIH2 | ARIH2 | 318 |
| O95376 | E3 ubiquitin-protein ligase ARIH2 | ARIH2 | 323 |
| O95376 | E3 ubiquitin-protein ligase ARIH2 | ARIH2 | 326 |
| O95376 | E3 ubiquitin-protein ligase ARIH2 | ARIH2 | 340 |
| Q96C12 | Armadillo repeat-containing protein 5 | ARMC5 | 273 |
| Q96C12 | Armadillo repeat-containing protein 5 | ARMC5 | 579 |
| Q96C12 | Armadillo repeat-containing protein 5 | ARMC5 | 665 |
| Q96C12 | Armadillo repeat-containing protein 5 | ARMC5 | 884 |
| Q96C12 | Armadillo repeat-containing protein 5 | ARMC5 | 894 |
| Q9Y576 | Ankyrin repeat and SOCS box protein 1 | ASB1 | 29 |
| Q9Y576 | Ankyrin repeat and SOCS box protein 1 | ASB1 | 36 |
| Q9Y576 | Ankyrin repeat and SOCS box protein 1 | ASB1 | 73 |
| Q9Y576 | Ankyrin repeat and SOCS box protein 1 | ASB1 | 79 |
| Q9Y576 | Ankyrin repeat and SOCS box protein 1 | ASB1 | 93 |
| Q9Y576 | Ankyrin repeat and SOCS box protein 1 | ASB1 | 301 |
| Q8WXK3 | Ankyrin repeat and SOCS box protein 13 | ASB13 | 9 |
| Q8WXK3 | Ankyrin repeat and SOCS box protein 13 | ASB13 | 90 |
| Q8WXK3 | Ankyrin repeat and SOCS box protein 13 | ASB13 | 148 |
| Q8WXK3 | Ankyrin repeat and SOCS box protein 13 | ASB13 | 165 |
| Q8WXK3 | Ankyrin repeat and SOCS box protein 13 | ASB13 | 231 |
| Q96Q27 | Ankyrin repeat and SOCS box protein 2 | ASB2 | 133 |
| Q96Q27 | Ankyrin repeat and SOCS box protein 2 | ASB2 | 440 |
| Q96Q27 | Ankyrin repeat and SOCS box protein 2 | ASB2 | 493 |
| Q96Q27 | Ankyrin repeat and SOCS box protein 2 | ASB2 | 552 |
| Q9NWX5 | Ankyrin repeat and SOCS box protein 6 | ASB6 | 153 |
| Q9NWX5 | Ankyrin repeat and SOCS box protein 6 | ASB6 | 256 |
| Q9NWX5 | Ankyrin repeat and SOCS box protein 6 | ASB6 | 266 |
| Q9NWX5 | Ankyrin repeat and SOCS box protein 6 | ASB6 | 313 |
| Q9H765 | Ankyrin repeat and SOCS box protein 8 | ASB8 | 237 |
| Q96DX5 | Ankyrin repeat and SOCS box protein 9 | ASB9 | 175 |
| Q96DX5 | Ankyrin repeat and SOCS box protein 9 | ASB9 | 216 |
| Q96DX5 | Ankyrin repeat and SOCS box protein 9 | ASB9 | 269 |
| P46100 | Transcriptional regulator ATRX | ATRX | 174 |
| P46100 | Transcriptional regulator ATRX | ATRX | 197 |
| P46100 | Transcriptional regulator ATRX | ATRX | 220 |
| P46100 | Transcriptional regulator ATRX | ATRX | 223 |
| P46100 | Transcriptional regulator ATRX | ATRX | 231 |
| P46100 | Transcriptional regulator ATRX | ATRX | 232 |
| P46100 | Transcriptional regulator ATRX | ATRX | 235 |
| P46100 | Transcriptional regulator ATRX | ATRX | 240 |
| P46100 | Transcriptional regulator ATRX | ATRX | 324 |
| P46100 | Transcriptional regulator ATRX | ATRX | 335 |
| P46100 | Transcriptional regulator ATRX | ATRX | 450 |
| P46100 | Transcriptional regulator ATRX | ATRX | 605 |
| P46100 | Transcriptional regulator ATRX | ATRX | 618 |
| P46100 | Transcriptional regulator ATRX | ATRX | 681 |
| P46100 | Transcriptional regulator ATRX | ATRX | 954 |
| P46100 | Transcriptional regulator ATRX | ATRX | 1026 |
| P46100 | Transcriptional regulator ATRX | ATRX | 1074 |
| P46100 | Transcriptional regulator ATRX | ATRX | 1094 |
| P46100 | Transcriptional regulator ATRX | ATRX | 1107 |
| P46100 | Transcriptional regulator ATRX | ATRX | 1242 |
| P46100 | Transcriptional regulator ATRX | ATRX | 1531 |
| P46100 | Transcriptional regulator ATRX | ATRX | 1574 |
| P46100 | Transcriptional regulator ATRX | ATRX | 1575 |
| P46100 | Transcriptional regulator ATRX | ATRX | 1576 |
| P46100 | Transcriptional regulator ATRX | ATRX | 1590 |
| P46100 | Transcriptional regulator ATRX | ATRX | 1595 |
| P46100 | Transcriptional regulator ATRX | ATRX | 1718 |
| P46100 | Transcriptional regulator ATRX | ATRX | 1760 |
| P46100 | Transcriptional regulator ATRX | ATRX | 1789 |
| P46100 | Transcriptional regulator ATRX | ATRX | 1814 |
| P46100 | Transcriptional regulator ATRX | ATRX | 1888 |
| P46100 | Transcriptional regulator ATRX | ATRX | 2404 |
| Q9NXR7 | BRISC and BRCA1-A complex member 2 | BABAM2 | 34 |
| Q9NXR7 | BRISC and BRCA1-A complex member 2 | BABAM2 | 44 |
| Q9NXR7 | BRISC and BRCA1-A complex member 2 | BABAM2 | 53 |
| Q9NXR7 | BRISC and BRCA1-A complex member 2 | BABAM2 | 129 |
| Q9NXR7 | BRISC and BRCA1-A complex member 2 | BABAM2 | 245 |
| Q9NXR7 | BRISC and BRCA1-A complex member 2 | BABAM2 | 254 |
| O14867 | Transcription regulator protein BACH1 | BACH1 | 140 |
| O14867 | Transcription regulator protein BACH1 | BACH1 | 145 |
| O14867 | Transcription regulator protein BACH1 | BACH1 | 150 |
| O14867 | Transcription regulator protein BACH1 | BACH1 | 184 |
| O14867 | Transcription regulator protein BACH1 | BACH1 | 224 |
| O14867 | Transcription regulator protein BACH1 | BACH1 | 263 |
| O14867 | Transcription regulator protein BACH1 | BACH1 | 270 |
| O14867 | Transcription regulator protein BACH1 | BACH1 | 299 |
| O14867 | Transcription regulator protein BACH1 | BACH1 | 406 |
| O14867 | Transcription regulator protein BACH1 | BACH1 | 412 |
| O14867 | Transcription regulator protein BACH1 | BACH1 | 435 |
| O14867 | Transcription regulator protein BACH1 | BACH1 | 522 |
| O14867 | Transcription regulator protein BACH1 | BACH1 | 557 |
| O14867 | Transcription regulator protein BACH1 | BACH1 | 581 |
| O14867 | Transcription regulator protein BACH1 | BACH1 | 621 |
| O14867 | Transcription regulator protein BACH1 | BACH1 | 646 |
| O14867 | Transcription regulator protein BACH1 | BACH1 | 683 |
| O14867 | Transcription regulator protein BACH1 | BACH1 | 715 |
| Q9BYV9 | Transcription regulator protein BACH2 | BACH2 | 149 |
| Q9BYV9 | Transcription regulator protein BACH2 | BACH2 | 156 |
| Q9BYV9 | Transcription regulator protein BACH2 | BACH2 | 238 |
| Q9BYV9 | Transcription regulator protein BACH2 | BACH2 | 328 |
| Q9BYV9 | Transcription regulator protein BACH2 | BACH2 | 340 |
| Q9BYV9 | Transcription regulator protein BACH2 | BACH2 | 370 |
| Q9BYV9 | Transcription regulator protein BACH2 | BACH2 | 420 |
| Q9BYV9 | Transcription regulator protein BACH2 | BACH2 | 500 |
| Q9BYV9 | Transcription regulator protein BACH2 | BACH2 | 507 |
| Q9BYV9 | Transcription regulator protein BACH2 | BACH2 | 670 |
| Q9BYV9 | Transcription regulator protein BACH2 | BACH2 | 676 |
| Q9BYV9 | Transcription regulator protein BACH2 | BACH2 | 683 |
| Q9BYV9 | Transcription regulator protein BACH2 | BACH2 | 698 |
| Q9BYV9 | Transcription regulator protein BACH2 | BACH2 | 714 |
| Q9BYV9 | Transcription regulator protein BACH2 | BACH2 | 823 |
| Q9BYV9 | Transcription regulator protein BACH2 | BACH2 | 830 |
| Q99728 | BRCA1-associated RING domain protein 1 | BARD1 | 108 |
| Q99728 | BRCA1-associated RING domain protein 1 | BARD1 | 304 |
| Q99728 | BRCA1-associated RING domain protein 1 | BARD1 | 362 |
| Q99728 | BRCA1-associated RING domain protein 1 | BARD1 | 368 |
| Q99728 | BRCA1-associated RING domain protein 1 | BARD1 | 557 |
| Q99728 | BRCA1-associated RING domain protein 1 | BARD1 | 743 |
| P41182 | B-cell lymphoma 6 protein | BCL6 | 121 |
| P41182 | B-cell lymphoma 6 protein | BCL6 | 175 |
| P41182 | B-cell lymphoma 6 protein | BCL6 | 232 |
| P41182 | B-cell lymphoma 6 protein | BCL6 | 254 |
| P41182 | B-cell lymphoma 6 protein | BCL6 | 296 |
| P41182 | B-cell lymphoma 6 protein | BCL6 | 339 |
| P41182 | B-cell lymphoma 6 protein | BCL6 | 348 |
| P41182 | B-cell lymphoma 6 protein | BCL6 | 354 |
| P41182 | B-cell lymphoma 6 protein | BCL6 | 414 |
| P41182 | B-cell lymphoma 6 protein | BCL6 | 548 |
| P41182 | B-cell lymphoma 6 protein | BCL6 | 663 |
| Q13490 | Baculoviral IAP repeat-containing protein 2 | BIRC2 | 45 |
| Q13490 | Baculoviral IAP repeat-containing protein 2 | BIRC2 | 83 |
| Q13490 | Baculoviral IAP repeat-containing protein 2 | BIRC2 | 85 |
| Q13490 | Baculoviral IAP repeat-containing protein 2 | BIRC2 | 220 |
| Q13490 | Baculoviral IAP repeat-containing protein 2 | BIRC2 | 247 |
| Q13490 | Baculoviral IAP repeat-containing protein 2 | BIRC2 | 308 |
| Q13490 | Baculoviral IAP repeat-containing protein 2 | BIRC2 | 315 |
| Q13490 | Baculoviral IAP repeat-containing protein 2 | BIRC2 | 474 |
| Q13490 | Baculoviral IAP repeat-containing protein 2 | BIRC2 | 602 |
| Q13490 | Baculoviral IAP repeat-containing protein 2 | BIRC2 | 605 |
| Q13489 | Baculoviral IAP repeat-containing protein 3 | BIRC3 | 28 |
| Q13489 | Baculoviral IAP repeat-containing protein 3 | BIRC3 | 66 |
| Q13489 | Baculoviral IAP repeat-containing protein 3 | BIRC3 | 68 |
| Q13489 | Baculoviral IAP repeat-containing protein 3 | BIRC3 | 93 |
| Q13489 | Baculoviral IAP repeat-containing protein 3 | BIRC3 | 164 |
| Q13489 | Baculoviral IAP repeat-containing protein 3 | BIRC3 | 205 |
| Q13489 | Baculoviral IAP repeat-containing protein 3 | BIRC3 | 232 |
| Q13489 | Baculoviral IAP repeat-containing protein 3 | BIRC3 | 294 |
| Q13489 | Baculoviral IAP repeat-containing protein 3 | BIRC3 | 301 |
| Q13489 | Baculoviral IAP repeat-containing protein 3 | BIRC3 | 581 |
| Q13489 | Baculoviral IAP repeat-containing protein 3 | BIRC3 | 588 |
| Q13489 | Baculoviral IAP repeat-containing protein 3 | BIRC3 | 591 |
| Q9NR09 | Baculoviral IAP repeat-containing protein 6 | BIRC6 | 62 |
| Q9NR09 | Baculoviral IAP repeat-containing protein 6 | BIRC6 | 65 |
| Q9NR09 | Baculoviral IAP repeat-containing protein 6 | BIRC6 | 136 |
| Q9NR09 | Baculoviral IAP repeat-containing protein 6 | BIRC6 | 355 |
| Q9NR09 | Baculoviral IAP repeat-containing protein 6 | BIRC6 | 381 |
| Q9NR09 | Baculoviral IAP repeat-containing protein 6 | BIRC6 | 390 |
| Q9NR09 | Baculoviral IAP repeat-containing protein 6 | BIRC6 | 396 |
| Q9NR09 | Baculoviral IAP repeat-containing protein 6 | BIRC6 | 410 |
| Q9NR09 | Baculoviral IAP repeat-containing protein 6 | BIRC6 | 463 |
| Q9NR09 | Baculoviral IAP repeat-containing protein 6 | BIRC6 | 543 |
| Q9NR09 | Baculoviral IAP repeat-containing protein 6 | BIRC6 | 613 |
| Q9NR09 | Baculoviral IAP repeat-containing protein 6 | BIRC6 | 752 |
| Q9NR09 | Baculoviral IAP repeat-containing protein 6 | BIRC6 | 777 |
| Q9NR09 | Baculoviral IAP repeat-containing protein 6 | BIRC6 | 946 |
| Q9NR09 | Baculoviral IAP repeat-containing protein 6 | BIRC6 | 970 |
| Q9NR09 | Baculoviral IAP repeat-containing protein 6 | BIRC6 | 1045 |
| Q9NR09 | Baculoviral IAP repeat-containing protein 6 | BIRC6 | 1099 |
| Q9NR09 | Baculoviral IAP repeat-containing protein 6 | BIRC6 | 1236 |
| Q9NR09 | Baculoviral IAP repeat-containing protein 6 | BIRC6 | 1319 |
| Q9NR09 | Baculoviral IAP repeat-containing protein 6 | BIRC6 | 1417 |
| Q9NR09 | Baculoviral IAP repeat-containing protein 6 | BIRC6 | 1420 |
| Q9NR09 | Baculoviral IAP repeat-containing protein 6 | BIRC6 | 1517 |
| Q9NR09 | Baculoviral IAP repeat-containing protein 6 | BIRC6 | 1547 |
| Q9NR09 | Baculoviral IAP repeat-containing protein 6 | BIRC6 | 1926 |
| Q9NR09 | Baculoviral IAP repeat-containing protein 6 | BIRC6 | 1932 |
| Q9NR09 | Baculoviral IAP repeat-containing protein 6 | BIRC6 | 2123 |
| Q9NR09 | Baculoviral IAP repeat-containing protein 6 | BIRC6 | 2892 |
| Q9NR09 | Baculoviral IAP repeat-containing protein 6 | BIRC6 | 2910 |
| Q9NR09 | Baculoviral IAP repeat-containing protein 6 | BIRC6 | 3075 |
| Q9NR09 | Baculoviral IAP repeat-containing protein 6 | BIRC6 | 3404 |
| Q9NR09 | Baculoviral IAP repeat-containing protein 6 | BIRC6 | 3673 |
| Q9NR09 | Baculoviral IAP repeat-containing protein 6 | BIRC6 | 3786 |
| Q9NR09 | Baculoviral IAP repeat-containing protein 6 | BIRC6 | 3830 |
| Q9NR09 | Baculoviral IAP repeat-containing protein 6 | BIRC6 | 4183 |
| Q9NR09 | Baculoviral IAP repeat-containing protein 6 | BIRC6 | 4443 |
| Q9NR09 | Baculoviral IAP repeat-containing protein 6 | BIRC6 | 4750 |
| Q9NR09 | Baculoviral IAP repeat-containing protein 6 | BIRC6 | 4813 |
| Q9NR09 | Baculoviral IAP repeat-containing protein 6 | BIRC6 | 4828 |
| Q96P09 | Baculoviral IAP repeat-containing protein 8 | BIRC8 | 66 |
| Q7Z569 | BRCA1-associated protein | BRAP | 47 |
| Q7Z569 | BRCA1-associated protein | BRAP | 110 |
| Q7Z569 | BRCA1-associated protein | BRAP | 221 |
| Q7Z569 | BRCA1-associated protein | BRAP | 264 |
| Q7Z569 | BRCA1-associated protein | BRAP | 267 |
| Q7Z569 | BRCA1-associated protein | BRAP | 283 |
| Q7Z569 | BRCA1-associated protein | BRAP | 291 |
| Q7Z569 | BRCA1-associated protein | BRAP | 300 |
| Q7Z569 | BRCA1-associated protein | BRAP | 306 |
| Q7Z569 | BRCA1-associated protein | BRAP | 452 |
| Q7Z569 | BRCA1-associated protein | BRAP | 473 |
| Q7Z569 | BRCA1-associated protein | BRAP | 519 |
| P38398 | Breast cancer type 1 susceptibility protein | BRCA1 | 24 |
| P38398 | Breast cancer type 1 susceptibility protein | BRCA1 | 27 |
| P38398 | Breast cancer type 1 susceptibility protein | BRCA1 | 39 |
| P38398 | Breast cancer type 1 susceptibility protein | BRCA1 | 44 |
| P38398 | Breast cancer type 1 susceptibility protein | BRCA1 | 61 |
| P38398 | Breast cancer type 1 susceptibility protein | BRCA1 | 64 |
| P38398 | Breast cancer type 1 susceptibility protein | BRCA1 | 197 |
| P38398 | Breast cancer type 1 susceptibility protein | BRCA1 | 226 |
| P38398 | Breast cancer type 1 susceptibility protein | BRCA1 | 274 |
| P38398 | Breast cancer type 1 susceptibility protein | BRCA1 | 305 |
| P38398 | Breast cancer type 1 susceptibility protein | BRCA1 | 328 |
| P38398 | Breast cancer type 1 susceptibility protein | BRCA1 | 348 |
| P38398 | Breast cancer type 1 susceptibility protein | BRCA1 | 360 |
| P38398 | Breast cancer type 1 susceptibility protein | BRCA1 | 442 |
| P38398 | Breast cancer type 1 susceptibility protein | BRCA1 | 636 |
| P38398 | Breast cancer type 1 susceptibility protein | BRCA1 | 644 |
| P38398 | Breast cancer type 1 susceptibility protein | BRCA1 | 712 |
| P38398 | Breast cancer type 1 susceptibility protein | BRCA1 | 801 |
| P38398 | Breast cancer type 1 susceptibility protein | BRCA1 | 805 |
| P38398 | Breast cancer type 1 susceptibility protein | BRCA1 | 818 |
| P38398 | Breast cancer type 1 susceptibility protein | BRCA1 | 882 |
| P38398 | Breast cancer type 1 susceptibility protein | BRCA1 | 903 |
| P38398 | Breast cancer type 1 susceptibility protein | BRCA1 | 953 |
| P38398 | Breast cancer type 1 susceptibility protein | BRCA1 | 1103 |
| P38398 | Breast cancer type 1 susceptibility protein | BRCA1 | 1225 |
| P38398 | Breast cancer type 1 susceptibility protein | BRCA1 | 1251 |
| P38398 | Breast cancer type 1 susceptibility protein | BRCA1 | 1270 |
| P38398 | Breast cancer type 1 susceptibility protein | BRCA1 | 1291 |
| P38398 | Breast cancer type 1 susceptibility protein | BRCA1 | 1300 |
| P38398 | Breast cancer type 1 susceptibility protein | BRCA1 | 1513 |
| Q9H0C5 | BTB/POZ domain-containing protein 1 | BTBD1 | 167 |
| Q9H0C5 | BTB/POZ domain-containing protein 1 | BTBD1 | 252 |
| Q9H0C5 | BTB/POZ domain-containing protein 1 | BTBD1 | 422 |
| Q9BSF8 | BTB/POZ domain-containing protein 10 | BTBD10 | 86 |
| Q9BSF8 | BTB/POZ domain-containing protein 10 | BTBD10 | 143 |
| Q9BSF8 | BTB/POZ domain-containing protein 10 | BTBD10 | 419 |
| A6QL63 | Ankyrin repeat and BTB/POZ domain-containing protein | BTBD11 | 261 |
| | BTBD11 | | |
| A6QL63 | Ankyrin repeat and BTB/POZ domain-containing protein | BTBD11 | 552 |
| | BTBD11 | | |
| A6QL63 | Ankyrin repeat and BTB/POZ domain-containing protein | BTBD11 | 554 |
| | BTBD11 | | |
| A6QL63 | Ankyrin repeat and BTB/POZ domain-containing protein | BTBD11 | 579 |
| | BTBD11 | | |
| A6QL63 | Ankyrin repeat and BTB/POZ domain-containing protein | BTBD11 | 844 |
| | BTBD11 | | |
| Q9BX70 | BTB/POZ domain-containing protein 2 | BTBD2 | 372 |
| Q9P203 | BTB/POZ domain-containing protein 7 | BTBD7 | 827 |
| Q9P203 | BTB/POZ domain-containing protein 7 | BTBD7 | 953 |
| Q5XKL5 | BTB/POZ domain-containing protein 8 | BTBD8 | 4 |
| Q5XKL5 | BTB/POZ domain-containing protein 8 | BTBD8 | 31 |
| Q96Q07 | BTB/POZ domain-containing protein 9 | BTBD9 | 159 |
| Q96Q07 | BTB/POZ domain-containing protein 9 | BTBD9 | 318 |
| Q9Y297 | F-box/WD repeat-containing protein 1A | BTRC | 83 |
| Q9Y297 | F-box/WD repeat-containing protein 1A | BTRC | 87 |
| Q9Y297 | F-box/WD repeat-containing protein 1A | BTRC | 105 |
| Q9Y297 | F-box/WD repeat-containing protein 1A | BTRC | 335 |
| Q9Y297 | F-box/WD repeat-containing protein 1A | BTRC | 471 |
| Q9Y297 | F-box/WD repeat-containing protein 1A | BTRC | 495 |
| Q9Y297 | F-box/WD repeat-containing protein 1A | BTRC | 498 |
| Q9Y297 | F-box/WD repeat-containing protein 1A | BTRC | 547 |
| Q86VP6 | Cullin-associated NEDD8-dissociated protein 1 | CAND1 | 71 |
| Q86VP6 | Cullin-associated NEDD8-dissociated protein 1 | CAND1 | 92 |
| Q86VP6 | Cullin-associated NEDD8-dissociated protein 1 | CAND1 | 131 |
| Q86VP6 | Cullin-associated NEDD8-dissociated protein 1 | CAND1 | 179 |
| Q86VP6 | Cullin-associated NEDD8-dissociated protein 1 | CAND1 | 237 |
| Q86VP6 | Cullin-associated NEDD8-dissociated protein 1 | CAND1 | 301 |
| Q86VP6 | Cullin-associated NEDD8-dissociated protein 1 | CAND1 | 356 |
| Q86VP6 | Cullin-associated NEDD8-dissociated protein 1 | CAND1 | 413 |
| Q86VP6 | Cullin-associated NEDD8-dissociated protein 1 | CAND1 | 453 |
| Q86VP6 | Cullin-associated NEDD8-dissociated protein 1 | CAND1 | 454 |
| Q86VP6 | Cullin-associated NEDD8-dissociated protein 1 | CAND1 | 571 |
| Q86VP6 | Cullin-associated NEDD8-dissociated protein 1 | CAND1 | 592 |
| Q86VP6 | Cullin-associated NEDD8-dissociated protein 1 | CAND1 | 802 |
| Q86VP6 | Cullin-associated NEDD8-dissociated protein 1 | CAND1 | 940 |
| Q86VP6 | Cullin-associated NEDD8-dissociated protein 1 | CAND1 | 942 |
| Q86VP6 | Cullin-associated NEDD8-dissociated protein 1 | CAND1 | 954 |
| Q86VP6 | Cullin-associated NEDD8-dissociated protein 1 | CAND1 | 1007 |
| Q86VP6 | Cullin-associated NEDD8-dissociated protein 1 | CAND1 | 1134 |
| Q86VP6 | Cullin-associated NEDD8-dissociated protein 1 | CAND1 | 1153 |
| O75155 | Cullin-associated NEDD8-dissociated protein 2 | CAND2 | 92 |
| O75155 | Cullin-associated NEDD8-dissociated protein 2 | CAND2 | 131 |
| O75155 | Cullin-associated NEDD8-dissociated protein 2 | CAND2 | 179 |
| O75155 | Cullin-associated NEDD8-dissociated protein 2 | CAND2 | 206 |
| O75155 | Cullin-associated NEDD8-dissociated protein 2 | CAND2 | 240 |
| O75155 | Cullin-associated NEDD8-dissociated protein 2 | CAND2 | 304 |
| O75155 | Cullin-associated NEDD8-dissociated protein 2 | CAND2 | 358 |
| O75155 | Cullin-associated NEDD8-dissociated protein 2 | CAND2 | 375 |
| O75155 | Cullin-associated NEDD8-dissociated protein 2 | CAND2 | 594 |
| O75155 | Cullin-associated NEDD8-dissociated protein 2 | CAND2 | 807 |
| O75155 | Cullin-associated NEDD8-dissociated protein 2 | CAND2 | 815 |
| O75155 | Cullin-associated NEDD8-dissociated protein 2 | CAND2 | 828 |
| O75155 | Cullin-associated NEDD8-dissociated protein 2 | CAND2 | 945 |
| O75155 | Cullin-associated NEDD8-dissociated protein 2 | CAND2 | 959 |
| O75155 | Cullin-associated NEDD8-dissociated protein 2 | CAND2 | 1139 |
| O75155 | Cullin-associated NEDD8-dissociated protein 2 | CAND2 | 1158 |
| P22681 | E3 ubiquitin-protein ligase CBL | CBL | 70 |
| P22681 | E3 ubiquitin-protein ligase CBL | CBL | 353 |
| P22681 | E3 ubiquitin-protein ligase CBL | CBL | 384 |
| P22681 | E3 ubiquitin-protein ligase CBL | CBL | 421 |
| P22681 | E3 ubiquitin-protein ligase CBL | CBL | 508 |
| P22681 | E3 ubiquitin-protein ligase CBL | CBL | 567 |
| P22681 | E3 ubiquitin-protein ligase CBL | CBL | 572 |
| P22681 | E3 ubiquitin-protein ligase CBL | CBL | 659 |
| P22681 | E3 ubiquitin-protein ligase CBL | CBL | 840 |
| Q13191 | E3 ubiquitin-protein ligase CBL-B | CBLB | 60 |
| Q13191 | E3 ubiquitin-protein ligase CBL-B | CBLB | 345 |
| Q13191 | E3 ubiquitin-protein ligase CBL-B | CBLB | 376 |
| Q13191 | E3 ubiquitin-protein ligase CBL-B | CBLB | 435 |
| Q13191 | E3 ubiquitin-protein ligase CBL-B | CBLB | 436 |
| Q13191 | E3 ubiquitin-protein ligase CBL-B | CBLB | 470 |
| Q13191 | E3 ubiquitin-protein ligase CBL-B | CBLB | 523 |
| Q13191 | E3 ubiquitin-protein ligase CBL-B | CBLB | 535 |
| Q13191 | E3 ubiquitin-protein ligase CBL-B | CBLB | 594 |
| Q13191 | E3 ubiquitin-protein ligase CBL-B | CBLB | 607 |
| Q13191 | E3 ubiquitin-protein ligase CBL-B | CBLB | 686 |
| Q13191 | E3 ubiquitin-protein ligase CBL-B | CBLB | 741 |
| Q13191 | E3 ubiquitin-protein ligase CBL-B | CBLB | 895 |
| Q9ULV8 | E3 ubiquitin-protein ligase CBL-C | CBLC | 168 |
| Q9ULV8 | E3 ubiquitin-protein ligase CBL-C | CBLC | 182 |
| Q9ULV8 | E3 ubiquitin-protein ligase CBL-C | CBLC | 189 |
| Q9ULV8 | E3 ubiquitin-protein ligase CBL-C | CBLC | 267 |
| Q9ULV8 | E3 ubiquitin-protein ligase CBL-C | CBLC | 354 |
| Q75N03 | E3 ubiquitin-protein ligase Hakai | CBLL1 | 73 |
| Q75N03 | E3 ubiquitin-protein ligase Hakai | CBLL1 | 112 |
| Q75N03 | E3 ubiquitin-protein ligase Hakai | CBLL1 | 130 |
| Q75N03 | E3 ubiquitin-protein ligase Hakai | CBLL1 | 133 |
| Q75N03 | E3 ubiquitin-protein ligase Hakai | CBLL1 | 145 |
| Q75N03 | E3 ubiquitin-protein ligase Hakai | CBLL1 | 148 |
| Q75N03 | E3 ubiquitin-protein ligase Hakai | CBLL1 | 158 |
| Q75N03 | E3 ubiquitin-protein ligase Hakai | CBLL1 | 172 |
| O00257 | E3 SUMO-protein ligase CBX4 | CBX4 | 185 |
| O00257 | E3 SUMO-protein ligase CBX4 | CBX4 | 413 |
| O00257 | E3 SUMO-protein ligase CBX4 | CBX4 | 424 |
| O00257 | E3 SUMO-protein ligase CBX4 | CBX4 | 438 |
| O60826 | Coiled-coil domain-containing protein 22 | CCDC22 | 215 |
| O60826 | Coiled-coil domain-containing protein 22 | CCDC22 | 369 |
| O60826 | Coiled-coil domain-containing protein 22 | CCDC22 | 441 |
| P55774 | C-C motif chemokine 18 | CCL18 | 18 |
| P55774 | C-C motif chemokine 18 | CCL18 | 20 |
| P41002 | Cyclin-F | CCNF | 176 |
| P41002 | Cyclin-F | CCNF | 240 |
| P41002 | Cyclin-F | CCNF | 255 |
| Q13042 | Cell division cycle protein 16 homolog | CDC16 | 79 |
| Q13042 | Cell division cycle protein 16 homolog | CDC16 | 133 |
| Q13042 | Cell division cycle protein 16 homolog | CDC16 | 194 |
| Q13042 | Cell division cycle protein 16 homolog | CDC16 | 245 |
| Q13042 | Cell division cycle protein 16 homolog | CDC16 | 367 |
| Q13042 | Cell division cycle protein 16 homolog | CDC16 | 455 |
| Q13042 | Cell division cycle protein 16 homolog | CDC16 | 544 |
| Q12834 | Cell division cycle protein 20 homolog | CDC20 | 313 |
| Q12834 | Cell division cycle protein 20 homolog | CDC20 | 364 |
| Q12834 | Cell division cycle protein 20 homolog | CDC20 | 388 |
| Q12834 | Cell division cycle protein 20 homolog | CDC20 | 472 |
| Q9UJX2 | Cell division cycle protein 23 homolog | CDC23 | 109 |
| Q9UJX2 | Cell division cycle protein 23 homolog | CDC23 | 324 |
| Q9UJX2 | Cell division cycle protein 23 homolog | CDC23 | 442 |
| Q9UJX2 | Cell division cycle protein 23 homolog | CDC23 | 489 |
| Q9UJX2 | Cell division cycle protein 23 homolog | CDC23 | 500 |
| Q9UJX2 | Cell division cycle protein 23 homolog | CDC23 | 523 |
| Q9UJX2 | Cell division cycle protein 23 homolog | CDC23 | 532 |
| Q9UJX2 | Cell division cycle protein 23 homolog | CDC23 | 536 |
| Q9UJX2 | Cell division cycle protein 23 homolog | CDC23 | 537 |
| P30260 | Cell division cycle protein 27 homolog | CDC27 | 66 |
| P30260 | Cell division cycle protein 27 homolog | CDC27 | 71 |
| P30260 | Cell division cycle protein 27 homolog | CDC27 | 78 |
| P30260 | Cell division cycle protein 27 homolog | CDC27 | 115 |
| P30260 | Cell division cycle protein 27 homolog | CDC27 | 137 |
| P30260 | Cell division cycle protein 27 homolog | CDC27 | 156 |
| P30260 | Cell division cycle protein 27 homolog | CDC27 | 179 |
| P30260 | Cell division cycle protein 27 homolog | CDC27 | 184 |
| P30260 | Cell division cycle protein 27 homolog | CDC27 | 477 |
| P30260 | Cell division cycle protein 27 homolog | CDC27 | 481 |
| P30260 | Cell division cycle protein 27 homolog | CDC27 | 576 |
| P30260 | Cell division cycle protein 27 homolog | CDC27 | 623 |
| P30260 | Cell division cycle protein 27 homolog | CDC27 | 673 |
| P30260 | Cell division cycle protein 27 homolog | CDC27 | 705 |
| Q6P1J9 | Parafibromin | CDC73 | 145 |
| Q6P1J9 | Parafibromin | CDC73 | 397 |
| Q99675 | Cell growth regulator with RING finger domain protein 1 | CGRRF1 | 117 |
| Q99675 | Cell growth regulator with RING finger domain protein 1 | CGRRF1 | 277 |
| Q99675 | Cell growth regulator with RING finger domain protein 1 | CGRRF1 | 293 |
| Q99675 | Cell growth regulator with RING finger domain protein 1 | CGRRF1 | 295 |
| Q99675 | Cell growth regulator with RING finger domain protein 1 | CGRRF1 | 298 |
| Q99675 | Cell growth regulator with RING finger domain protein 1 | CGRRF1 | 319 |
| Q96EP1 | E3 ubiquitin-protein ligase CHFR | CHFR | 46 |
| Q96EP1 | E3 ubiquitin-protein ligase CHFR | CHFR | 61 |
| Q96EP1 | E3 ubiquitin-protein ligase CHFR | CHFR | 95 |
| Q96EP1 | E3 ubiquitin-protein ligase CHFR | CHFR | 206 |
| Q96EP1 | E3 ubiquitin-protein ligase CHFR | CHFR | 431 |
| Q96EP1 | E3 ubiquitin-protein ligase CHFR | CHFR | 513 |
| Q96EP1 | E3 ubiquitin-protein ligase CHFR | CHFR | 529 |
| Q96EP1 | E3 ubiquitin-protein ligase CHFR | CHFR | 601 |
| Q96EP1 | E3 ubiquitin-protein ligase CHFR | CHFR | 603 |
| Q96EP1 | E3 ubiquitin-protein ligase CHFR | CHFR | 604 |
| Q96EP1 | E3 ubiquitin-protein ligase CHFR | CHFR | 635 |
| Q96EP1 | E3 ubiquitin-protein ligase CHFR | CHFR | 657 |
| Q9NSE2 | Cytokine-inducible SH2-containing protein | CISH | 69 |
| Q9NSE2 | Cytokine-inducible SH2-containing protein | CISH | 144 |
| Q9NSE2 | Cytokine-inducible SH2-containing protein | CISH | 167 |
| Q9NSE2 | Cytokine-inducible SH2-containing protein | CISH | 227 |
| P10909 | Clusterin | CLU | 102 |
| P10909 | Clusterin | CLU | 285 |
| P10909 | Clusterin | CLU | 295 |
| P10909 | Clusterin | CLU | 302 |
| P10909 | Clusterin | CLU | 313 |
| O95628 | CCR4-NOT transcription complex subunit 4 | CNOT4 | 53 |
| O95628 | CCR4-NOT transcription complex subunit 4 | CNOT4 | 56 |
| O95628 | CCR4-NOT transcription complex subunit 4 | CNOT4 | 175 |
| O95628 | CCR4-NOT transcription complex subunit 4 | CNOT4 | 196 |
| O95628 | CCR4-NOT transcription complex subunit 4 | CNOT4 | 210 |
| Q86X83 | COMM domain-containing protein 2 | COMMD2 | 191 |
| Q9UBI1 | COMM domain-containing protein 3 | COMMD3 | 90 |
| Q9UBI1 | COMM domain-containing protein 3 | COMMD3 | 101 |
| Q9GZQ3 | COMM domain-containing protein 5 | COMMD5 | 64 |
| Q7Z4G1 | COMM domain-containing protein 6 | COMMD6 | 35 |
| Q7Z4G1 | COMM domain-containing protein 6 | COMMD6 | 60 |
| Q9P000 | COMM domain-containing protein 9 | COMMD9 | 147 |
| Q9P000 | COMM domain-containing protein 9 | COMMD9 | 160 |
| Q8NHY2 | E3 ubiquitin-protein ligase COP1 | COP1 | 91 |
| Q8NHY2 | E3 ubiquitin-protein ligase COP1 | COP1 | 120 |
| Q8NHY2 | E3 ubiquitin-protein ligase COP1 | COP1 | 136 |
| Q8NHY2 | E3 ubiquitin-protein ligase COP1 | COP1 | 151 |
| Q8NHY2 | E3 ubiquitin-protein ligase COP1 | COP1 | 156 |
| Q8NHY2 | E3 ubiquitin-protein ligase COP1 | COP1 | 159 |
| Q8NHY2 | E3 ubiquitin-protein ligase COP1 | COP1 | 170 |
| Q8NHY2 | E3 ubiquitin-protein ligase COP1 | COP1 | 370 |
| Q8NHY2 | E3 ubiquitin-protein ligase COP1 | COP1 | 395 |
| Q8NHY2 | E3 ubiquitin-protein ligase COP1 | COP1 | 632 |
| Q13616 | Cullin-1 | CUL1 | 149 |
| Q13616 | Cullin-1 | CUL1 | 170 |
| Q13616 | Cullin-1 | CUL1 | 291 |
| Q13616 | Cullin-1 | CUL1 | 355 |
| Q13616 | Cullin-1 | CUL1 | 426 |
| Q13616 | Cullin-1 | CUL1 | 496 |
| Q13616 | Cullin-1 | CUL1 | 594 |
| Q13616 | Cullin-1 | CUL1 | 752 |
| Q13617 | Cullin-2 | CUL2 | 103 |
| Q13617 | Cullin-2 | CUL2 | 266 |
| Q13617 | Cullin-2 | CUL2 | 282 |
| Q13617 | Cullin-2 | CUL2 | 385 |
| Q13617 | Cullin-2 | CUL2 | 395 |
| Q13617 | Cullin-2 | CUL2 | 465 |
| Q13617 | Cullin-2 | CUL2 | 721 |
| Q13618 | Cullin-3 | CUL3 | 156 |
| Q13618 | Cullin-3 | CUL3 | 187 |
| Q13618 | Cullin-3 | CUL3 | 251 |
| Q13618 | Cullin-3 | CUL3 | 298 |
| Q13618 | Cullin-3 | CUL3 | 314 |
| Q13618 | Cullin-3 | CUL3 | 316 |
| Q13618 | Cullin-3 | CUL3 | 462 |
| Q13618 | Cullin-3 | CUL3 | 464 |
| Q13618 | Cullin-3 | CUL3 | 522 |
| Q13618 | Cullin-3 | CUL3 | 636 |
| Q13619 | Cullin-4A | CUL4A | 94 |
| Q13619 | Cullin-4A | CUL4A | 110 |
| Q13619 | Cullin-4A | CUL4A | 138 |
| Q13619 | Cullin-4A | CUL4A | 241 |
| Q13619 | Cullin-4A | CUL4A | 289 |
| Q13619 | Cullin-4A | CUL4A | 483 |
| Q13619 | Cullin-4A | CUL4A | 633 |
| Q13620 | Cullin-4B | CUL4B | 102 |
| Q13620 | Cullin-4B | CUL4B | 248 |
| Q13620 | Cullin-4B | CUL4B | 264 |
| Q13620 | Cullin-4B | CUL4B | 292 |
| Q13620 | Cullin-4B | CUL4B | 637 |
| Q13620 | Cullin-4B | CUL4B | 787 |
| Q13620 | Cullin-4B | CUL4B | 810 |
| Q93034 | Cullin-5 | CUL5 | 51 |
| Q93034 | Cullin-5 | CUL5 | 104 |
| Q93034 | Cullin-5 | CUL5 | 112 |
| Q93034 | Cullin-5 | CUL5 | 188 |
| Q93034 | Cullin-5 | CUL5 | 404 |
| Q14999 | Cullin-7 | CUL7 | 60 |
| Q14999 | Cullin-7 | CUL7 | 562 |
| Q14999 | Cullin-7 | CUL7 | 952 |
| Q14999 | Cullin-7 | CUL7 | 1106 |
| Q14999 | Cullin-7 | CUL7 | 1173 |
| Q14999 | Cullin-7 | CUL7 | 1201 |
| Q14999 | Cullin-7 | CUL7 | 1405 |
| Q14999 | Cullin-7 | CUL7 | 1413 |
| Q14999 | Cullin-7 | CUL7 | 1569 |
| Q14999 | Cullin-7 | CUL7 | 1601 |
| Q14999 | Cullin-7 | CUL7 | 1616 |
| Q14999 | Cullin-7 | CUL7 | 1688 |
| Q8IWT3 | Cullin-9 | CUL9 | 47 |
| Q8IWT3 | Cullin-9 | CUL9 | 1281 |
| Q8IWT3 | Cullin-9 | CUL9 | 1340 |
| Q8IWT3 | Cullin-9 | CUL9 | 1426 |
| Q8IWT3 | Cullin-9 | CUL9 | 1960 |
| Q8IWT3 | Cullin-9 | CUL9 | 1969 |
| Q8IWT3 | Cullin-9 | CUL9 | 2177 |
| Q8IWT3 | Cullin-9 | CUL9 | 2249 |
| Q8IWT3 | Cullin-9 | CUL9 | 2257 |
| Q8IWT3 | Cullin-9 | CUL9 | 2262 |
| Q8IWT3 | Cullin-9 | CUL9 | 2404 |
| Q9Y4B6 | DDB1- and CUL4-associated factor 1 | DCAF1 | 69 |
| Q9Y4B6 | DDB1- and CUL4-associated factor 1 | DCAF1 | 106 |
| Q9Y4B6 | DDB1- and CUL4-associated factor 1 | DCAF1 | 521 |
| Q9Y4B6 | DDB1- and CUL4-associated factor 1 | DCAF1 | 784 |
| Q9Y4B6 | DDB1- and CUL4-associated factor 1 | DCAF1 | 967 |
| Q9Y4B6 | DDB1- and CUL4-associated factor 1 | DCAF1 | 1019 |
| Q9Y4B6 | DDB1- and CUL4-associated factor 1 | DCAF1 | 1026 |
| Q9Y4B6 | DDB1- and CUL4-associated factor 1 | DCAF1 | 1037 |
| Q9Y4B6 | DDB1- and CUL4-associated factor 1 | DCAF1 | 1070 |
| Q9Y4B6 | DDB1- and CUL4-associated factor 1 | DCAF1 | 1113 |
| Q5QP82 | DDB1- and CUL4-associated factor 10 | DCAF10 | 102 |
| Q5QP82 | DDB1- and CUL4-associated factor 10 | DCAF10 | 213 |
| Q5QP82 | DDB1- and CUL4-associated factor 10 | DCAF10 | 228 |
| Q5QP82 | DDB1- and CUL4-associated factor 10 | DCAF10 | 247 |
| Q5QP82 | DDB1- and CUL4-associated factor 10 | DCAF10 | 290 |
| Q5QP82 | DDB1- and CUL4-associated factor 10 | DCAF10 | 310 |
| Q5QP82 | DDB1- and CUL4-associated factor 10 | DCAF10 | 369 |
| Q5QP82 | DDB1- and CUL4-associated factor 10 | DCAF10 | 412 |
| Q5QP82 | DDB1- and CUL4-associated factor 10 | DCAF10 | 479 |
| Q5QP82 | DDB1- and CUL4-associated factor 10 | DCAF10 | 504 |
| Q5QP82 | DDB1- and CUL4-associated factor 10 | DCAF10 | 510 |
| Q5QP82 | DDB1- and CUL4-associated factor 10 | DCAF10 | 541 |
| Q5QP82 | DDB1- and CUL4-associated factor 10 | DCAF10 | 547 |
| Q8TEB1 | DDB1- and CUL4-associated factor 11 | DCAF11 | 143 |
| Q8TEB1 | DDB1- and CUL4-associated factor 11 | DCAF11 | 177 |
| Q8TEB1 | DDB1- and CUL4-associated factor 11 | DCAF11 | 191 |
| Q8TEB1 | DDB1- and CUL4-associated factor 11 | DCAF11 | 288 |
| Q8TEB1 | DDB1- and CUL4-associated factor 11 | DCAF11 | 460 |
| Q5T6F0 | DDB1- and CUL4-associated factor 12 | DCAF12 | 109 |
| Q5T6F0 | DDB1- and CUL4-associated factor 12 | DCAF12 | 141 |
| Q5T6F0 | DDB1- and CUL4-associated factor 12 | DCAF12 | 296 |
| Q5T6F0 | DDB1- and CUL4-associated factor 12 | DCAF12 | 380 |
| Q5T6F0 | DDB1- and CUL4-associated factor 12 | DCAF12 | 425 |
| Q5VU92 | DDB1- and CUL4-associated factor 12-like protein 1 | DCAF12L1 | 270 |
| Q5VU92 | DDB1- and CUL4-associated factor 12-like protein 1 | DCAF12L1 | 345 |
| Q9NV06 | DDB1- and CUL4-associated factor 13 | DCAF13 | 71 |
| Q9NV06 | DDB1- and CUL4-associated factor 13 | DCAF13 | 87 |
| Q9NV06 | DDB1- and CUL4-associated factor 13 | DCAF13 | 120 |
| Q9NV06 | DDB1- and CUL4-associated factor 13 | DCAF13 | 174 |
| Q9NV06 | DDB1- and CUL4-associated factor 13 | DCAF13 | 190 |
| Q9NV06 | DDB1- and CUL4-associated factor 13 | DCAF13 | 215 |
| Q9NV06 | DDB1- and CUL4-associated factor 13 | DCAF13 | 330 |
| Q9NV06 | DDB1- and CUL4-associated factor 13 | DCAF13 | 342 |
| Q66K64 | DDB1- and CUL4-associated factor 15 | DCAF15 | 62 |
| Q66K64 | DDB1- and CUL4-associated factor 15 | DCAF15 | 193 |
| Q66K64 | DDB1- and CUL4-associated factor 15 | DCAF15 | 211 |
| Q66K64 | DDB1- and CUL4-associated factor 15 | DCAF15 | 347 |
| Q66K64 | DDB1- and CUL4-associated factor 15 | DCAF15 | 365 |
| Q9NXF7 | DDB1- and CUL4-associated factor 16 | DCAF16 | 100 |
| Q9NXF7 | DDB1- and CUL4-associated factor 16 | DCAF16 | 103 |
| Q9NXF7 | DDB1- and CUL4-associated factor 16 | DCAF16 | 119 |
| Q9NXF7 | DDB1- and CUL4-associated factor 16 | DCAF16 | 173 |
| Q9NXF7 | DDB1- and CUL4-associated factor 16 | DCAF16 | 178 |
| Q9NXF7 | DDB1- and CUL4-associated factor 16 | DCAF16 | 179 |
| Q5H9S7 | DDB1- and CUL4-associated factor 17 | DCAF17 | 20 |
| Q5H9S7 | DDB1- and CUL4-associated factor 17 | DCAF17 | 74 |
| Q5H9S7 | DDB1- and CUL4-associated factor 17 | DCAF17 | 456 |
| Q8WV16 | DDB1- and CUL4-associated factor 4 | DCAF4 | 88 |
| Q8WV16 | DDB1- and CUL4-associated factor 4 | DCAF4 | 355 |
| Q96JK2 | DDB1- and CUL4-associated factor 5 | DCAF5 | 182 |
| Q96JK2 | DDB1- and CUL4-associated factor 5 | DCAF5 | 280 |
| Q96JK2 | DDB1- and CUL4-associated factor 5 | DCAF5 | 349 |
| Q96JK2 | DDB1- and CUL4-associated factor 5 | DCAF5 | 368 |
| Q96JK2 | DDB1- and CUL4-associated factor 5 | DCAF5 | 503 |
| Q96JK2 | DDB1- and CUL4-associated factor 5 | DCAF5 | 711 |
| Q96JK2 | DDB1- and CUL4-associated factor 5 | DCAF5 | 728 |
| Q96JK2 | DDB1- and CUL4-associated factor 5 | DCAF5 | 812 |
| Q96JK2 | DDB1- and CUL4-associated factor 5 | DCAF5 | 829 |
| Q96JK2 | DDB1- and CUL4-associated factor 5 | DCAF5 | 883 |
| Q58WW2 | DDB1- and CUL4-associated factor 6 | DCAF6 | 105 |
| Q58WW2 | DDB1- and CUL4-associated factor 6 | DCAF6 | 177 |
| Q58WW2 | DDB1- and CUL4-associated factor 6 | DCAF6 | 190 |
| Q58WW2 | DDB1- and CUL4-associated factor 6 | DCAF6 | 211 |
| Q58WW2 | DDB1- and CUL4-associated factor 6 | DCAF6 | 513 |
| Q58WW2 | DDB1- and CUL4-associated factor 6 | DCAF6 | 593 |
| P61962 | DDB1- and CUL4-associated factor 7 | DCAF7 | 61 |
| P61962 | DDB1- and CUL4-associated factor 7 | DCAF7 | 109 |
| P61962 | DDB1- and CUL4-associated factor 7 | DCAF7 | 256 |
| P61962 | DDB1- and CUL4-associated factor 7 | DCAF7 | 268 |
| P61962 | DDB1- and CUL4-associated factor 7 | DCAF7 | 280 |
| A6NGE4 | DDB1- and CUL4-associated factor 8-like protein 1 | DCAF8L1 | 177 |
| P0C7V8 | DDB1- and CUL4-associated factor 8-like protein 2 | DCAF8L2 | 209 |
| Q96GG9 | DCN1-like protein 1 | DCUN1D1 | 29 |
| Q96GG9 | DCN1-like protein 1 | DCUN1D1 | 115 |
| Q16531 | DNA damage-binding protein 1 | DDB1 | 87 |
| Q16531 | DNA damage-binding protein 1 | DDB1 | 128 |
| Q16531 | DNA damage-binding protein 1 | DDB1 | 173 |
| Q16531 | DNA damage-binding protein 1 | DDB1 | 260 |
| Q16531 | DNA damage-binding protein 1 | DDB1 | 378 |
| Q16531 | DNA damage-binding protein 1 | DDB1 | 652 |
| Q16531 | DNA damage-binding protein 1 | DDB1 | 725 |
| Q16531 | DNA damage-binding protein 1 | DDB1 | 732 |
| Q16531 | DNA damage-binding protein 1 | DDB1 | 903 |
| Q92466 | DNA damage-binding protein 2 | DDB2 | 48 |
| Q92466 | DNA damage-binding protein 2 | DDB2 | 52 |
| Q92466 | DNA damage-binding protein 2 | DDB2 | 66 |
| Q92466 | DNA damage-binding protein 2 | DDB2 | 253 |
| Q92466 | DNA damage-binding protein 2 | DDB2 | 254 |
| Q92466 | DNA damage-binding protein 2 | DDB2 | 322 |
| Q92466 | DNA damage-binding protein 2 | DDB2 | 364 |
| Q92782 | Zinc finger protein neuro-d4 | DPF1 | 50 |
| Q92782 | Zinc finger protein neuro-d4 | DPF1 | 122 |
| Q92782 | Zinc finger protein neuro-d4 | DPF1 | 128 |
| Q92782 | Zinc finger protein neuro-d4 | DPF1 | 149 |
| Q92782 | Zinc finger protein neuro-d4 | DPF1 | 168 |
| Q92782 | Zinc finger protein neuro-d4 | DPF1 | 260 |
| Q92782 | Zinc finger protein neuro-d4 | DPF1 | 269 |
| Q92782 | Zinc finger protein neuro-d4 | DPF1 | 305 |
| Q92782 | Zinc finger protein neuro-d4 | DPF1 | 308 |
| Q9NZJ0 | Denticleless protein homolog | DTL | 136 |
| Q9NZJ0 | Denticleless protein homolog | DTL | 141 |
| Q9NZJ0 | Denticleless protein homolog | DTL | 157 |
| Q9NZJ0 | Denticleless protein homolog | DTL | 367 |
| Q9NZJ0 | Denticleless protein homolog | DTL | 369 |
| Q9NZJ0 | Denticleless protein homolog | DTL | 379 |
| Q9NZJ0 | Denticleless protein homolog | DTL | 438 |
| Q9NZJ0 | Denticleless protein homolog | DTL | 450 |
| Q9NZJ0 | Denticleless protein homolog | DTL | 454 |
| Q9NZJ0 | Denticleless protein homolog | DTL | 544 |
| Q9NZJ0 | Denticleless protein homolog | DTL | 559 |
| Q9NZJ0 | Denticleless protein homolog | DTL | 573 |
| Q9NZJ0 | Denticleless protein homolog | DTL | 589 |
| Q9NZJ0 | Denticleless protein homolog | DTL | 590 |
| Q9NZJ0 | Denticleless protein homolog | DTL | 633 |
| Q9NZJ0 | Denticleless protein homolog | DTL | 643 |
| Q9NZJ0 | Denticleless protein homolog | DTL | 710 |
| Q9NZJ0 | Denticleless protein homolog | DTL | 722 |
| Q86Y01 | E3 ubiquitin-protein ligase DTX1 | DTX1 | 349 |
| Q86Y01 | E3 ubiquitin-protein ligase DTX1 | DTX1 | 378 |
| Q86Y01 | E3 ubiquitin-protein ligase DTX1 | DTX1 | 468 |
| Q86Y01 | E3 ubiquitin-protein ligase DTX1 | DTX1 | 471 |
| Q86Y01 | E3 ubiquitin-protein ligase DTX1 | DTX1 | 536 |
| Q86UW9 | Probable E3 ubiquitin-protein ligase DTX2 | DTX2 | 169 |
| Q86UW9 | Probable E3 ubiquitin-protein ligase DTX2 | DTX2 | 196 |
| Q86UW9 | Probable E3 ubiquitin-protein ligase DTX2 | DTX2 | 198 |
| Q86UW9 | Probable E3 ubiquitin-protein ligase DTX2 | DTX2 | 201 |
| Q86UW9 | Probable E3 ubiquitin-protein ligase DTX2 | DTX2 | 351 |
| Q86UW9 | Probable E3 ubiquitin-protein ligase DTX2 | DTX2 | 537 |
| Q8N9I9 | Probable E3 ubiquitin-protein ligase DTX3 | DTX3 | 15 |
| Q8N9I9 | Probable E3 ubiquitin-protein ligase DTX3 | DTX3 | 105 |
| Q8N9I9 | Probable E3 ubiquitin-protein ligase DTX3 | DTX3 | 164 |
| Q8N9I9 | Probable E3 ubiquitin-protein ligase DTX3 | DTX3 | 167 |
| Q8N9I9 | Probable E3 ubiquitin-protein ligase DTX3 | DTX3 | 185 |
| Q8N9I9 | Probable E3 ubiquitin-protein ligase DTX3 | DTX3 | 188 |
| Q8N9I9 | Probable E3 ubiquitin-protein ligase DTX3 | DTX3 | 199 |
| Q8N9I9 | Probable E3 ubiquitin-protein ligase DTX3 | DTX3 | 202 |
| Q8N9I9 | Probable E3 ubiquitin-protein ligase DTX3 | DTX3 | 272 |
| Q8N9I9 | Probable E3 ubiquitin-protein ligase DTX3 | DTX3 | 317 |
| Q8TDB6 | E3 ubiquitin-protein ligase DTX3L | DTX3L | 130 |
| Q8TDB6 | E3 ubiquitin-protein ligase DTX3L | DTX3L | 144 |
| Q8TDB6 | E3 ubiquitin-protein ligase DTX3L | DTX3L | 159 |
| Q8TDB6 | E3 ubiquitin-protein ligase DTX3L | DTX3L | 175 |
| Q8TDB6 | E3 ubiquitin-protein ligase DTX3L | DTX3L | 219 |
| Q8TDB6 | E3 ubiquitin-protein ligase DTX3L | DTX3L | 250 |
| Q8TDB6 | E3 ubiquitin-protein ligase DTX3L | DTX3L | 309 |
| Q8TDB6 | E3 ubiquitin-protein ligase DTX3L | DTX3L | 406 |
| Q8TDB6 | E3 ubiquitin-protein ligase DTX3L | DTX3L | 417 |
| Q8TDB6 | E3 ubiquitin-protein ligase DTX3L | DTX3L | 561 |
| Q8TDB6 | E3 ubiquitin-protein ligase DTX3L | DTX3L | 564 |
| Q8TDB6 | E3 ubiquitin-protein ligase DTX3L | DTX3L | 577 |
| Q8TDB6 | E3 ubiquitin-protein ligase DTX3L | DTX3L | 582 |
| Q8TDB6 | E3 ubiquitin-protein ligase DTX3L | DTX3L | 585 |
| Q8TDB6 | E3 ubiquitin-protein ligase DTX3L | DTX3L | 596 |
| Q8TDB6 | E3 ubiquitin-protein ligase DTX3L | DTX3L | 599 |
| Q86Y13 | E3 ubiquitin-protein ligase DZIP3 | DZIP3 | 261 |
| Q86Y13 | E3 ubiquitin-protein ligase DZIP3 | DZIP3 | 468 |
| Q86Y13 | E3 ubiquitin-protein ligase DZIP3 | DZIP3 | 1078 |
| Q66K89 | Transcription factor E4F1 | E4F1 | 273 |
| Q66K89 | Transcription factor E4F1 | E4F1 | 361 |
| Q66K89 | Transcription factor E4F1 | E4F1 | 496 |
| P11161 | E3 SUMO-protein ligase EGR2 | EGR2 | 251 |
| P11161 | E3 SUMO-protein ligase EGR2 | EGR2 | 443 |
| P11161 | E3 SUMO-protein ligase EGR2 | EGR2 | 469 |
| Q14241 | Elongin-A | ELOA | 209 |
| Q14241 | Elongin-A | ELOA | 339 |
| Q14241 | Elongin-A | ELOA | 568 |
| Q14241 | Elongin-A | ELOA | 581 |
| Q14241 | Elongin-A | ELOA | 610 |
| Q15370 | Elongin-B | ELOB | 60 |
| Q15370 | Elongin-B | ELOB | 89 |
| Q15369 | Elongin-C | ELOC | 11 |
| Q15369 | Elongin-C | ELOC | 74 |
| O14682 | Ectoderm-neural cortex protein 1 | ENC1 | 59 |
| O14682 | Ectoderm-neural cortex protein 1 | ENC1 | 227 |
| O14682 | Ectoderm-neural cortex protein 1 | ENC1 | 287 |
| O14682 | Ectoderm-neural cortex protein 1 | ENC1 | 307 |
| O14682 | Ectoderm-neural cortex protein 1 | ENC1 | 340 |
| O14682 | Ectoderm-neural cortex protein 1 | ENC1 | 464 |
| O14682 | Ectoderm-neural cortex protein 1 | ENC1 | 468 |
| O14682 | Ectoderm-neural cortex protein 1 | ENC1 | 478 |
| O14682 | Ectoderm-neural cortex protein 1 | ENC1 | 506 |
| O14682 | Ectoderm-neural cortex protein 1 | ENC1 | 531 |
| Q13216 | DNA excision repair protein ERCC-8 | ERCC8 | 84 |
| Q13216 | DNA excision repair protein ERCC-8 | ERCC8 | 88 |
| Q13216 | DNA excision repair protein ERCC-8 | ERCC8 | 157 |
| Q13216 | DNA excision repair protein ERCC-8 | ERCC8 | 171 |
| Q13216 | DNA excision repair protein ERCC-8 | ERCC8 | 178 |
| Q13216 | DNA excision repair protein ERCC-8 | ERCC8 | 222 |
| Q13216 | DNA excision repair protein ERCC-8 | ERCC8 | 252 |
| Q13216 | DNA excision repair protein ERCC-8 | ERCC8 | 288 |
| Q13216 | DNA excision repair protein ERCC-8 | ERCC8 | 340 |
| Q13216 | DNA excision repair protein ERCC-8 | ERCC8 | 356 |
| Q9NW38 | E3 ubiquitin-protein ligase FANCL | FANCL | 12 |
| Q9NW38 | E3 ubiquitin-protein ligase FANCL | FANCL | 251 |
| Q8NFZ0 | F-box DNA helicase 1 | FBH1 | 156 |
| Q8NFZ0 | F-box DNA helicase 1 | FBH1 | 289 |
| Q8NFZ0 | F-box DNA helicase 1 | FBH1 | 302 |
| Q8NFZ0 | F-box DNA helicase 1 | FBH1 | 508 |
| Q8NFZ0 | F-box DNA helicase 1 | FBH1 | 615 |
| Q8NFZ0 | F-box DNA helicase 1 | FBH1 | 894 |
| Q8NFZ0 | F-box DNA helicase 1 | FBH1 | 999 |
| Q8NFZ0 | F-box DNA helicase 1 | FBH1 | 1002 |
| Q9NXK8 | F-box/LRR-repeat protein 12 | FBXL12 | 101 |
| Q9NXK8 | F-box/LRR-repeat protein 12 | FBXL12 | 108 |
| Q9NXK8 | F-box/LRR-repeat protein 12 | FBXL12 | 134 |
| Q9NXK8 | F-box/LRR-repeat protein 12 | FBXL12 | 212 |
| Q9NXK8 | F-box/LRR-repeat protein 12 | FBXL12 | 304 |
| Q9NXK8 | F-box/LRR-repeat protein 12 | FBXL12 | 310 |
| Q8N1E6 | F-box/LRR-repeat protein 14 | FBXL14 | 313 |
| Q8N1E6 | F-box/LRR-repeat protein 14 | FBXL14 | 382 |
| Q9H469 | F-box/LRR-repeat protein 15 | FBXL15 | 124 |
| Q9H469 | F-box/LRR-repeat protein 15 | FBXL15 | 139 |
| Q9H469 | F-box/LRR-repeat protein 15 | FBXL15 | 150 |
| Q9H469 | F-box/LRR-repeat protein 15 | FBXL15 | 218 |
| Q9H469 | F-box/LRR-repeat protein 15 | FBXL15 | 244 |
| Q8N461 | F-box/LRR-repeat protein 16 | FBXL16 | 74 |
| Q9UF56 | F-box/LRR-repeat protein 17 | FBXL17 | 207 |
| Q9UF56 | F-box/LRR-repeat protein 17 | FBXL17 | 210 |
| Q9UF56 | F-box/LRR-repeat protein 17 | FBXL17 | 295 |
| Q9UF56 | F-box/LRR-repeat protein 17 | FBXL17 | 437 |
| Q96ME1 | F-box/LRR-repeat protein 18 | FBXL18 | 63 |
| Q96ME1 | F-box/LRR-repeat protein 18 | FBXL18 | 104 |
| Q96ME1 | F-box/LRR-repeat protein 18 | FBXL18 | 129 |
| Q96ME1 | F-box/LRR-repeat protein 18 | FBXL18 | 166 |
| Q96ME1 | F-box/LRR-repeat protein 18 | FBXL18 | 190 |
| Q96ME1 | F-box/LRR-repeat protein 18 | FBXL18 | 324 |
| Q96ME1 | F-box/LRR-repeat protein 18 | FBXL18 | 353 |
| Q96ME1 | F-box/LRR-repeat protein 18 | FBXL18 | 356 |
| Q96ME1 | F-box/LRR-repeat protein 18 | FBXL18 | 382 |
| Q96ME1 | F-box/LRR-repeat protein 18 | FBXL18 | 383 |
| Q96ME1 | F-box/LRR-repeat protein 18 | FBXL18 | 406 |
| Q96ME1 | F-box/LRR-repeat protein 18 | FBXL18 | 423 |
| Q96ME1 | F-box/LRR-repeat protein 18 | FBXL18 | 459 |
| Q96ME1 | F-box/LRR-repeat protein 18 | FBXL18 | 468 |
| Q96ME1 | F-box/LRR-repeat protein 18 | FBXL18 | 513 |
| Q96ME1 | F-box/LRR-repeat protein 18 | FBXL18 | 557 |
| Q96ME1 | F-box/LRR-repeat protein 18 | FBXL18 | 620 |
| Q6PCT2 | F-box/LRR-repeat protein 19 | FBXL19 | 56 |
| Q6PCT2 | F-box/LRR-repeat protein 19 | FBXL19 | 72 |
| Q6PCT2 | F-box/LRR-repeat protein 19 | FBXL19 | 142 |
| Q6PCT2 | F-box/LRR-repeat protein 19 | FBXL19 | 145 |
| Q6PCT2 | F-box/LRR-repeat protein 19 | FBXL19 | 246 |
| Q6PCT2 | F-box/LRR-repeat protein 19 | FBXL19 | 273 |
| Q6PCT2 | F-box/LRR-repeat protein 19 | FBXL19 | 314 |
| Q6PCT2 | F-box/LRR-repeat protein 19 | FBXL19 | 637 |
| Q6PCT2 | F-box/LRR-repeat protein 19 | FBXL19 | 644 |
| Q6PCT2 | F-box/LRR-repeat protein 19 | FBXL19 | 670 |
| Q9UKC9 | F-box/LRR-repeat protein 2 | FBXL2 | 192 |
| Q96IG2 | F-box/LRR-repeat protein 20 | FBXL20 | 101 |
| Q96IG2 | F-box/LRR-repeat protein 20 | FBXL20 | 127 |
| Q96IG2 | F-box/LRR-repeat protein 20 | FBXL20 | 135 |
| Q96IG2 | F-box/LRR-repeat protein 20 | FBXL20 | 205 |
| Q96IG2 | F-box/LRR-repeat protein 20 | FBXL20 | 389 |
| Q9UKT7 | F-box/LRR-repeat protein 3 | FBXL3 | 63 |
| Q9UKT7 | F-box/LRR-repeat protein 3 | FBXL3 | 78 |
| Q9UKT7 | F-box/LRR-repeat protein 3 | FBXL3 | 137 |
| Q9UKT7 | F-box/LRR-repeat protein 3 | FBXL3 | 208 |
| Q9UKT7 | F-box/LRR-repeat protein 3 | FBXL3 | 331 |
| Q9UKT7 | F-box/LRR-repeat protein 3 | FBXL3 | 340 |
| Q9UKA2 | F-box/LRR-repeat protein 4 | FBXL4 | 107 |
| Q9UKA2 | F-box/LRR-repeat protein 4 | FBXL4 | 194 |
| Q9UKA2 | F-box/LRR-repeat protein 4 | FBXL4 | 307 |
| Q9UKA1 | F-box/LRR-repeat protein 5 | FBXL5 | 159 |
| Q8N531 | F-box/LRR-repeat protein 6 | FBXL6 | 219 |
| Q8N531 | F-box/LRR-repeat protein 6 | FBXL6 | 327 |
| Q8N531 | F-box/LRR-repeat protein 6 | FBXL6 | 368 |
| Q8N531 | F-box/LRR-repeat protein 6 | FBXL6 | 407 |
| Q8N531 | F-box/LRR-repeat protein 6 | FBXL6 | 439 |
| Q96CD0 | F-box/LRR-repeat protein 8 | FBXL8 | 41 |
| Q96CD0 | F-box/LRR-repeat protein 8 | FBXL8 | 190 |
| Q96CD0 | F-box/LRR-repeat protein 8 | FBXL8 | 228 |
| Q9UK96 | F-box only protein 10 | FBXO10 | 52 |
| Q9UK96 | F-box only protein 10 | FBXO10 | 97 |
| Q9UK96 | F-box only protein 10 | FBXO10 | 309 |
| Q9UK96 | F-box only protein 10 | FBXO10 | 447 |
| Q9UK96 | F-box only protein 10 | FBXO10 | 778 |
| Q86XK2 | F-box only protein 11 | FBXO11 | 113 |
| Q86XK2 | F-box only protein 11 | FBXO11 | 581 |
| Q96EF6 | F-box only protein 17 | FBXO17 | 94 |
| Q96EF6 | F-box only protein 17 | FBXO17 | 114 |
| Q96EF6 | F-box only protein 17 | FBXO17 | 193 |
| Q9UK22 | F-box only protein 2 | FBXO2 | 71 |
| Q9UK22 | F-box only protein 2 | FBXO2 | 92 |
| Q9UK22 | F-box only protein 2 | FBXO2 | 137 |
| Q9UK22 | F-box only protein 2 | FBXO2 | 176 |
| Q9UK22 | F-box only protein 2 | FBXO2 | 215 |
| O94952 | F-box only protein 21 | FBXO21 | 66 |
| O94952 | F-box only protein 21 | FBXO21 | 560 |
| Q8NEZ5 | F-box only protein 22 | FBXO22 | 47 |
| Q8NEZ5 | F-box only protein 22 | FBXO22 | 117 |
| Q8NEZ5 | F-box only protein 22 | FBXO22 | 227 |
| Q8NEZ5 | F-box only protein 22 | FBXO22 | 228 |
| Q8NEZ5 | F-box only protein 22 | FBXO22 | 378 |
| Q8NI29 | F-box only protein 27 | FBXO27 | 99 |
| Q8NI29 | F-box only protein 27 | FBXO27 | 162 |
| Q9UK99 | F-box only protein 3 | FBXO3 | 37 |
| Q9UK99 | F-box only protein 3 | FBXO3 | 74 |
| Q8TB52 | F-box only protein 30 | FBXO30 | 18 |
| Q8TB52 | F-box only protein 30 | FBXO30 | 28 |
| Q8TB52 | F-box only protein 30 | FBXO30 | 44 |
| Q8TB52 | F-box only protein 30 | FBXO30 | 53 |
| Q8TB52 | F-box only protein 30 | FBXO30 | 67 |
| Q8TB52 | F-box only protein 30 | FBXO30 | 317 |
| Q8TB52 | F-box only protein 30 | FBXO30 | 366 |
| Q8TB52 | F-box only protein 30 | FBXO30 | 396 |
| Q8TB52 | F-box only protein 30 | FBXO30 | 523 |
| Q8TB52 | F-box only protein 30 | FBXO30 | 555 |
| Q8TB52 | F-box only protein 30 | FBXO30 | 562 |
| Q8TB52 | F-box only protein 30 | FBXO30 | 570 |
| Q8TB52 | F-box only protein 30 | FBXO30 | 592 |
| Q8TB52 | F-box only protein 30 | FBXO30 | 687 |
| Q8TB52 | F-box only protein 30 | FBXO30 | 707 |
| Q8TB52 | F-box only protein 30 | FBXO30 | 723 |
| Q8TB52 | F-box only protein 30 | FBXO30 | 725 |
| Q5XUX0 | F-box only protein 31 | FBXO31 | 8 |
| Q5XUX0 | F-box only protein 31 | FBXO31 | 118 |
| Q5XUX0 | F-box only protein 31 | FBXO31 | 131 |
| Q5XUX0 | F-box only protein 31 | FBXO31 | 230 |
| Q5XUX0 | F-box only protein 31 | FBXO31 | 440 |
| Q969P5 | F-box only protein 32 | FBXO32 | 231 |
| Q7Z6M2 | F-box only protein 33 | FBXO33 | 95 |
| Q9NWN3 | F-box only protein 34 | FBXO34 | 128 |
| Q9NWN3 | F-box only protein 34 | FBXO34 | 225 |
| Q9NWN3 | F-box only protein 34 | FBXO34 | 229 |
| Q9NWN3 | F-box only protein 34 | FBXO34 | 647 |
| Q9NWN3 | F-box only protein 34 | FBXO34 | 691 |
| Q6PIJ6 | F-box only protein 38 | FBXO38 | 11 |
| Q6PIJ6 | F-box only protein 38 | FBXO38 | 206 |
| Q6PIJ6 | F-box only protein 38 | FBXO38 | 242 |
| Q6PIJ6 | F-box only protein 38 | FBXO38 | 303 |
| Q6PIJ6 | F-box only protein 38 | FBXO38 | 401 |
| Q6PIJ6 | F-box only protein 38 | FBXO38 | 410 |
| Q6PIJ6 | F-box only protein 38 | FBXO38 | 670 |
| Q6PIJ6 | F-box only protein 38 | FBXO38 | 674 |
| Q6PIJ6 | F-box only protein 38 | FBXO38 | 704 |
| Q6PIJ6 | F-box only protein 38 | FBXO38 | 776 |
| Q6PIJ6 | F-box only protein 38 | FBXO38 | 777 |
| Q6PIJ6 | F-box only protein 38 | FBXO38 | 778 |
| Q6PIJ6 | F-box only protein 38 | FBXO38 | 791 |
| Q6PIJ6 | F-box only protein 38 | FBXO38 | 803 |
| Q6PIJ6 | F-box only protein 38 | FBXO38 | 855 |
| Q6PIJ6 | F-box only protein 38 | FBXO38 | 978 |
| Q9UKT5 | F-box only protein 4 | FBXO4 | 146 |
| Q9UKT5 | F-box only protein 4 | FBXO4 | 147 |
| Q9UKT5 | F-box only protein 4 | FBXO4 | 283 |
| Q6P3S6 | F-box only protein 42 | FBXO42 | 89 |
| Q6P3S6 | F-box only protein 42 | FBXO42 | 302 |
| Q6P3S6 | F-box only protein 42 | FBXO42 | 403 |
| Q9H4M3 | F-box only protein 44 | FBXO44 | 30 |
| Q9H4M3 | F-box only protein 44 | FBXO44 | 170 |
| P0C2W1 | F-box/SPRY domain-containing protein 1 | FBXO45 | 69 |
| P0C2W1 | F-box/SPRY domain-containing protein 1 | FBXO45 | 98 |
| P0C2W1 | F-box/SPRY domain-containing protein 1 | FBXO45 | 119 |
| P0C2W1 | F-box/SPRY domain-containing protein 1 | FBXO45 | 218 |
| Q6PJ61 | F-box only protein 46 | FBXO46 | 109 |
| Q6PJ61 | F-box only protein 46 | FBXO46 | 224 |
| Q6PJ61 | F-box only protein 46 | FBXO46 | 288 |
| Q6PJ61 | F-box only protein 46 | FBXO46 | 306 |
| Q6PJ61 | F-box only protein 46 | FBXO46 | 394 |
| Q6PJ61 | F-box only protein 46 | FBXO46 | 545 |
| Q6PJ61 | F-box only protein 46 | FBXO46 | 576 |
| Q6PJ61 | F-box only protein 46 | FBXO46 | 589 |
| Q9UKT4 | F-box only protein 5 | FBXO5 | 6 |
| Q9UKT4 | F-box only protein 5 | FBXO5 | 8 |
| Q9UKT4 | F-box only protein 5 | FBXO5 | 15 |
| Q9UKT4 | F-box only protein 5 | FBXO5 | 17 |
| Q9UKT4 | F-box only protein 5 | FBXO5 | 36 |
| Q9UKT4 | F-box only protein 5 | FBXO5 | 49 |
| Q9UKT4 | F-box only protein 5 | FBXO5 | 53 |
| Q9UKT4 | F-box only protein 5 | FBXO5 | 83 |
| Q9UKT4 | F-box only protein 5 | FBXO5 | 86 |
| Q9UKT4 | F-box only protein 5 | FBXO5 | 95 |
| Q9UKT4 | F-box only protein 5 | FBXO5 | 202 |
| Q9UKT4 | F-box only protein 5 | FBXO5 | 381 |
| Q9UKT4 | F-box only protein 5 | FBXO5 | 389 |
| Q9UKT4 | F-box only protein 5 | FBXO5 | 396 |
| Q9UKT4 | F-box only protein 5 | FBXO5 | 401 |
| Q9UKT4 | F-box only protein 5 | FBXO5 | 406 |
| Q9UKT4 | F-box only protein 5 | FBXO5 | 409 |
| Q9UKT4 | F-box only protein 5 | FBXO5 | 411 |
| Q9UKT4 | F-box only protein 5 | FBXO5 | 419 |
| Q9NRD1 | F-box only protein 6 | FBXO6 | 37 |
| Q9Y3I1 | F-box only protein 7 | FBXO7 | 36 |
| Q9Y3I1 | F-box only protein 7 | FBXO7 | 286 |
| Q9UK97 | F-box only protein 9 | FBXO9 | 68 |
| Q9UKB1 | F-box/WD repeat-containing protein 11 | FBXW11 | 76 |
| Q9UKB1 | F-box/WD repeat-containing protein 11 | FBXW11 | 145 |
| Q9UKB1 | F-box/WD repeat-containing protein 11 | FBXW11 | 245 |
| Q9UKB1 | F-box/WD repeat-containing protein 11 | FBXW11 | 272 |
| Q9UKB1 | F-box/WD repeat-containing protein 11 | FBXW11 | 408 |
| Q9UKB1 | F-box/WD repeat-containing protein 11 | FBXW11 | 432 |
| Q9UKB1 | F-box/WD repeat-containing protein 11 | FBXW11 | 435 |
| Q9UKB1 | F-box/WD repeat-containing protein 11 | FBXW11 | 484 |
| Q9UKT8 | F-box/WD repeat-containing protein 2 | FBXW2 | 327 |
| P57775 | F-box/WD repeat-containing protein 4 | FBXW4 | 64 |
| P57775 | F-box/WD repeat-containing protein 4 | FBXW4 | 108 |
| Q969U6 | F-box/WD repeat-containing protein 5 | FBXW5 | 277 |
| Q969U6 | F-box/WD repeat-containing protein 5 | FBXW5 | 503 |
| Q969H0 | F-box/WD repeat-containing protein 7 | FBXW7 | 202 |
| Q969H0 | F-box/WD repeat-containing protein 7 | FBXW7 | 390 |
| Q8N3Y1 | F-box/WD repeat-containing protein 8 | FBXW8 | 139 |
| Q8N3Y1 | F-box/WD repeat-containing protein 8 | FBXW8 | 176 |
| Q8N3Y1 | F-box/WD repeat-containing protein 8 | FBXW8 | 575 |
| Q8N3Y1 | F-box/WD repeat-containing protein 8 | FBXW8 | 579 |
| Q5XUX1 | F-box/WD repeat-containing protein 9 | FBXW9 | 107 |
| Q5XUX1 | F-box/WD repeat-containing protein 9 | FBXW9 | 146 |
| Q5XUX1 | F-box/WD repeat-containing protein 9 | FBXW9 | 240 |
| Q5XUX1 | F-box/WD repeat-containing protein 9 | FBXW9 | 271 |
| Q5XUX1 | F-box/WD repeat-containing protein 9 | FBXW9 | 407 |
| Q9BSK4 | Protein fem-1 homolog A | FEM1A | 641 |
| Q9UK73 | Protein fem-1 homolog B | FEM1B | 186 |
| Q9UK73 | Protein fem-1 homolog B | FEM1B | 244 |
| Q9UK73 | Protein fem-1 homolog B | FEM1B | 363 |
| Q7L622 | G2/M phase-specific E3 ubiquitin-protein ligase | G2E3 | 106 |
| Q7L622 | G2/M phase-specific E3 ubiquitin-protein ligase | G2E3 | 285 |
| Q7L622 | G2/M phase-specific E3 ubiquitin-protein ligase | G2E3 | 580 |
| Q9H2C0 | Gigaxonin | GAN | 30 |
| Q9H2C0 | Gigaxonin | GAN | 248 |
| Q9H2C0 | Gigaxonin | GAN | 294 |
| Q9H2C0 | Gigaxonin | GAN | 296 |
| Q96IK5 | Germ cell-less protein-like 1 | GMCL1 | 44 |
| Q96IK5 | Germ cell-less protein-like 1 | GMCL1 | 46 |
| Q13888 | General transcription factor IIH subunit 2 | GTF2H2 | 83 |
| Q13888 | General transcription factor IIH subunit 2 | GTF2H2 | 139 |
| Q13888 | General transcription factor IIH subunit 2 | GTF2H2 | 208 |
| Q13888 | General transcription factor IIH subunit 2 | GTF2H2 | 247 |
| Q13888 | General transcription factor IIH subunit 2 | GTF2H2 | 299 |
| Q13888 | General transcription factor IIH subunit 2 | GTF2H2 | 305 |
| Q13888 | General transcription factor IIH subunit 2 | GTF2H2 | 308 |
| Q13888 | General transcription factor IIH subunit 2 | GTF2H2 | 348 |
| Q9H116 | GDNF-inducible zinc finger protein 1 | GZF1 | 319 |
| Q9H116 | GDNF-inducible zinc finger protein 1 | GZF1 | 322 |
| Q9H116 | GDNF-inducible zinc finger protein 1 | GZF1 | 379 |
| Q9H116 | GDNF-inducible zinc finger protein 1 | GZF1 | 382 |
| Q9H116 | GDNF-inducible zinc finger protein 1 | GZF1 | 548 |
| Q8IYU2 | E3 ubiquitin-protein ligase HACE1 | HACE1 | 99 |
| Q8IYU2 | E3 ubiquitin-protein ligase HACE1 | HACE1 | 481 |
| Q8IYU2 | E3 ubiquitin-protein ligase HACE1 | HACE1 | 489 |
| Q8IYU2 | E3 ubiquitin-protein ligase HACE1 | HACE1 | 508 |
| Q8IYU2 | E3 ubiquitin-protein ligase HACE1 | HACE1 | 567 |
| Q9ULT8 | E3 ubiquitin-protein ligase HECTD1 | HECTD1 | 44 |
| Q9ULT8 | E3 ubiquitin-protein ligase HECTD1 | HECTD1 | 48 |
| Q9ULT8 | E3 ubiquitin-protein ligase HECTD1 | HECTD1 | 58 |
| Q9ULT8 | E3 ubiquitin-protein ligase HECTD1 | HECTD1 | 88 |
| Q9ULT8 | E3 ubiquitin-protein ligase HECTD1 | HECTD1 | 209 |
| Q9ULT8 | E3 ubiquitin-protein ligase HECTD1 | HECTD1 | 254 |
| Q9ULT8 | E3 ubiquitin-protein ligase HECTD1 | HECTD1 | 316 |
| Q9ULT8 | E3 ubiquitin-protein ligase HECTD1 | HECTD1 | 369 |
| Q9ULT8 | E3 ubiquitin-protein ligase HECTD1 | HECTD1 | 436 |
| Q9ULT8 | E3 ubiquitin-protein ligase HECTD1 | HECTD1 | 487 |
| Q9ULT8 | E3 ubiquitin-protein ligase HECTD1 | HECTD1 | 507 |
| Q9ULT8 | E3 ubiquitin-protein ligase HECTD1 | HECTD1 | 878 |
| Q9ULT8 | E3 ubiquitin-protein ligase HECTD1 | HECTD1 | 936 |
| Q9ULT8 | E3 ubiquitin-protein ligase HECTD1 | HECTD1 | 1368 |
| Q9ULT8 | E3 ubiquitin-protein ligase HECTD1 | HECTD1 | 1389 |
| Q9ULT8 | E3 ubiquitin-protein ligase HECTD1 | HECTD1 | 1811 |
| Q9ULT8 | E3 ubiquitin-protein ligase HECTD1 | HECTD1 | 1855 |
| Q9ULT8 | E3 ubiquitin-protein ligase HECTD1 | HECTD1 | 1893 |
| Q9ULT8 | E3 ubiquitin-protein ligase HECTD1 | HECTD1 | 1944 |
| Q9ULT8 | E3 ubiquitin-protein ligase HECTD1 | HECTD1 | 1995 |
| Q9ULT8 | E3 ubiquitin-protein ligase HECTD1 | HECTD1 | 2071 |
| Q9ULT8 | E3 ubiquitin-protein ligase HECTD1 | HECTD1 | 2086 |
| Q9ULT8 | E3 ubiquitin-protein ligase HECTD1 | HECTD1 | 2224 |
| Q9ULT8 | E3 ubiquitin-protein ligase HECTD1 | HECTD1 | 2415 |
| Q9ULT8 | E3 ubiquitin-protein ligase HECTD1 | HECTD1 | 2545 |
| Q9ULT8 | E3 ubiquitin-protein ligase HECTD1 | HECTD1 | 2579 |
| Q5T447 | E3 ubiquitin-protein ligase HECTD3 | HECTD3 | 112 |
| Q5T447 | E3 ubiquitin-protein ligase HECTD3 | HECTD3 | 133 |
| Q5T447 | E3 ubiquitin-protein ligase HECTD3 | HECTD3 | 143 |
| Q5T447 | E3 ubiquitin-protein ligase HECTD3 | HECTD3 | 459 |
| Q5T447 | E3 ubiquitin-protein ligase HECTD3 | HECTD3 | 487 |
| Q5T447 | E3 ubiquitin-protein ligase HECTD3 | HECTD3 | 494 |
| Q5T447 | E3 ubiquitin-protein ligase HECTD3 | HECTD3 | 823 |
| Q5T447 | E3 ubiquitin-protein ligase HECTD3 | HECTD3 | 838 |
| Q9Y4D8 | Probable E3 ubiquitin-protein ligase HECTD4 | HECTD4 | 330 |
| Q9Y4D8 | Probable E3 ubiquitin-protein ligase HECTD4 | HECTD4 | 586 |
| Q9Y4D8 | Probable E3 ubiquitin-protein ligase HECTD4 | HECTD4 | 1084 |
| Q9Y4D8 | Probable E3 ubiquitin-protein ligase HECTD4 | HECTD4 | 1488 |
| Q9Y4D8 | Probable E3 ubiquitin-protein ligase HECTD4 | HECTD4 | 1580 |
| Q9Y4D8 | Probable E3 ubiquitin-protein ligase HECTD4 | HECTD4 | 1636 |
| Q9Y4D8 | Probable E3 ubiquitin-protein ligase HECTD4 | HECTD4 | 1783 |
| Q9Y4D8 | Probable E3 ubiquitin-protein ligase HECTD4 | HECTD4 | 2036 |
| Q9Y4D8 | Probable E3 ubiquitin-protein ligase HECTD4 | HECTD4 | 2786 |
| Q9Y4D8 | Probable E3 ubiquitin-protein ligase HECTD4 | HECTD4 | 3504 |
| Q9Y4D8 | Probable E3 ubiquitin-protein ligase HECTD4 | HECTD4 | 3607 |
| Q9Y4D8 | Probable E3 ubiquitin-protein ligase HECTD4 | HECTD4 | 3659 |
| Q9Y4D8 | Probable E3 ubiquitin-protein ligase HECTD4 | HECTD4 | 3787 |
| Q9Y4D8 | Probable E3 ubiquitin-protein ligase HECTD4 | HECTD4 | 3964 |
| Q9Y4D8 | Probable E3 ubiquitin-protein ligase HECTD4 | HECTD4 | 3985 |
| Q76N89 | E3 ubiquitin-protein ligase HECW1 | HECW1 | 1200 |
| Q76N89 | E3 ubiquitin-protein ligase HECW1 | HECW1 | 1574 |
| Q9P2P5 | E3 ubiquitin-protein ligase HECW2 | HECW2 | 224 |
| Q9P2P5 | E3 ubiquitin-protein ligase HECW2 | HECW2 | 436 |
| Q9P2P5 | E3 ubiquitin-protein ligase HECW2 | HECW2 | 660 |
| Q9P2P5 | E3 ubiquitin-protein ligase HECW2 | HECW2 | 949 |
| Q9P2P5 | E3 ubiquitin-protein ligase HECW2 | HECW2 | 1540 |
| Q15751 | Probable E3 ubiquitin-protein ligase HERC1 | HERC1 | 56 |
| Q15751 | Probable E3 ubiquitin-protein ligase HERC1 | HERC1 | 750 |
| Q15751 | Probable E3 ubiquitin-protein ligase HERC1 | HERC1 | 1192 |
| Q15751 | Probable E3 ubiquitin-protein ligase HERC1 | HERC1 | 1195 |
| Q15751 | Probable E3 ubiquitin-protein ligase HERC1 | HERC1 | 1228 |
| Q15751 | Probable E3 ubiquitin-protein ligase HERC1 | HERC1 | 1282 |
| Q15751 | Probable E3 ubiquitin-protein ligase HERC1 | HERC1 | 1388 |
| Q15751 | Probable E3 ubiquitin-protein ligase HERC1 | HERC1 | 1570 |
| Q15751 | Probable E3 ubiquitin-protein ligase HERC1 | HERC1 | 1941 |
| Q15751 | Probable E3 ubiquitin-protein ligase HERC1 | HERC1 | 1968 |
| Q15751 | Probable E3 ubiquitin-protein ligase HERC1 | HERC1 | 2525 |
| Q15751 | Probable E3 ubiquitin-protein ligase HERC1 | HERC1 | 2547 |
| Q15751 | Probable E3 ubiquitin-protein ligase HERC1 | HERC1 | 3002 |
| Q15751 | Probable E3 ubiquitin-protein ligase HERC1 | HERC1 | 3262 |
| Q15751 | Probable E3 ubiquitin-protein ligase HERC1 | HERC1 | 3272 |
| Q15751 | Probable E3 ubiquitin-protein ligase HERC1 | HERC1 | 3849 |
| Q15751 | Probable E3 ubiquitin-protein ligase HERC1 | HERC1 | 4486 |
| Q15751 | Probable E3 ubiquitin-protein ligase HERC1 | HERC1 | 4728 |
| Q15751 | Probable E3 ubiquitin-protein ligase HERC1 | HERC1 | 4811 |
| Q15751 | Probable E3 ubiquitin-protein ligase HERC1 | HERC1 | 4837 |
| O95714 | E3 ubiquitin-protein ligase HERC2 | HERC2 | 33 |
| O95714 | E3 ubiquitin-protein ligase HERC2 | HERC2 | 349 |
| O95714 | E3 ubiquitin-protein ligase HERC2 | HERC2 | 412 |
| O95714 | E3 ubiquitin-protein ligase HERC2 | HERC2 | 521 |
| O95714 | E3 ubiquitin-protein ligase HERC2 | HERC2 | 914 |
| O95714 | E3 ubiquitin-protein ligase HERC2 | HERC2 | 998 |
| O95714 | E3 ubiquitin-protein ligase HERC2 | HERC2 | 1005 |
| O95714 | E3 ubiquitin-protein ligase HERC2 | HERC2 | 1037 |
| O95714 | E3 ubiquitin-protein ligase HERC2 | HERC2 | 1201 |
| O95714 | E3 ubiquitin-protein ligase HERC2 | HERC2 | 1378 |
| O95714 | E3 ubiquitin-protein ligase HERC2 | HERC2 | 1410 |
| O95714 | E3 ubiquitin-protein ligase HERC2 | HERC2 | 1419 |
| O95714 | E3 ubiquitin-protein ligase HERC2 | HERC2 | 1525 |
| O95714 | E3 ubiquitin-protein ligase HERC2 | HERC2 | 1825 |
| O95714 | E3 ubiquitin-protein ligase HERC2 | HERC2 | 2025 |
| O95714 | E3 ubiquitin-protein ligase HERC2 | HERC2 | 2095 |
| O95714 | E3 ubiquitin-protein ligase HERC2 | HERC2 | 2111 |
| O95714 | E3 ubiquitin-protein ligase HERC2 | HERC2 | 2726 |
| O95714 | E3 ubiquitin-protein ligase HERC2 | HERC2 | 2760 |
| O95714 | E3 ubiquitin-protein ligase HERC2 | HERC2 | 2770 |
| O95714 | E3 ubiquitin-protein ligase HERC2 | HERC2 | 2808 |
| O95714 | E3 ubiquitin-protein ligase HERC2 | HERC2 | 2902 |
| O95714 | E3 ubiquitin-protein ligase HERC2 | HERC2 | 3558 |
| O95714 | E3 ubiquitin-protein ligase HERC2 | HERC2 | 3723 |
| O95714 | E3 ubiquitin-protein ligase HERC2 | HERC2 | 3864 |
| O95714 | E3 ubiquitin-protein ligase HERC2 | HERC2 | 3865 |
| O95714 | E3 ubiquitin-protein ligase HERC2 | HERC2 | 4762 |
| O95714 | E3 ubiquitin-protein ligase HERC2 | HERC2 | 4788 |
| Q15034 | Probable E3 ubiquitin-protein ligase HERC3 | HERC3 | 321 |
| Q15034 | Probable E3 ubiquitin-protein ligase HERC3 | HERC3 | 333 |
| Q15034 | Probable E3 ubiquitin-protein ligase HERC3 | HERC3 | 552 |
| Q5GLZ8 | Probable E3 ubiquitin-protein ligase HERC4 | HERC4 | 41 |
| Q5GLZ8 | Probable E3 ubiquitin-protein ligase HERC4 | HERC4 | 58 |
| Q5GLZ8 | Probable E3 ubiquitin-protein ligase HERC4 | HERC4 | 60 |
| Q5GLZ8 | Probable E3 ubiquitin-protein ligase HERC4 | HERC4 | 160 |
| Q5GLZ8 | Probable E3 ubiquitin-protein ligase HERC4 | HERC4 | 175 |
| Q5GLZ8 | Probable E3 ubiquitin-protein ligase HERC4 | HERC4 | 248 |
| Q5GLZ8 | Probable E3 ubiquitin-protein ligase HERC4 | HERC4 | 249 |
| Q5GLZ8 | Probable E3 ubiquitin-protein ligase HERC4 | HERC4 | 301 |
| Q5GLZ8 | Probable E3 ubiquitin-protein ligase HERC4 | HERC4 | 352 |
| Q5GLZ8 | Probable E3 ubiquitin-protein ligase HERC4 | HERC4 | 384 |
| Q5GLZ8 | Probable E3 ubiquitin-protein ligase HERC4 | HERC4 | 392 |
| Q5GLZ8 | Probable E3 ubiquitin-protein ligase HERC4 | HERC4 | 513 |
| Q5GLZ8 | Probable E3 ubiquitin-protein ligase HERC4 | HERC4 | 708 |
| Q5GLZ8 | Probable E3 ubiquitin-protein ligase HERC4 | HERC4 | 1025 |
| Q9UII4 | E3 ISG15--protein ligase HERC5 | HERC5 | 58 |
| Q9UII4 | E3 ISG15--protein ligase HERC5 | HERC5 | 90 |
| Q9UII4 | E3 ISG15--protein ligase HERC5 | HERC5 | 424 |
| Q9UII4 | E3 ISG15--protein ligase HERC5 | HERC5 | 979 |
| Q9UII4 | E3 ISG15--protein ligase HERC5 | HERC5 | 994 |
| Q8IVU3 | Probable E3 ubiquitin-protein ligase HERC6 | HERC6 | 91 |
| Q8IVU3 | Probable E3 ubiquitin-protein ligase HERC6 | HERC6 | 135 |
| Q8IVU3 | Probable E3 ubiquitin-protein ligase HERC6 | HERC6 | 557 |
| Q8IVU3 | Probable E3 ubiquitin-protein ligase HERC6 | HERC6 | 985 |
| P49773 | Histidine triad nucleotide-binding protein 1 | HINT1 | 38 |
| P49773 | Histidine triad nucleotide-binding protein 1 | HINT1 | 84 |
| Q14527 | Helicase-like transcription factor | HLTF | 360 |
| Q14527 | Helicase-like transcription factor | HLTF | 442 |
| Q14527 | Helicase-like transcription factor | HLTF | 461 |
| Q14527 | Helicase-like transcription factor | HLTF | 763 |
| Q14527 | Helicase-like transcription factor | HLTF | 775 |
| Q14527 | Helicase-like transcription factor | HLTF | 780 |
| Q14527 | Helicase-like transcription factor | HLTF | 812 |
| Q14527 | Helicase-like transcription factor | HLTF | 896 |
| Q14527 | Helicase-like transcription factor | HLTF | 942 |
| Q7Z6Z7 | E3 ubiquitin-protein ligase HUWE1 | HUWE1 | 19 |
| Q7Z6Z7 | E3 ubiquitin-protein ligase HUWE1 | HUWE1 | 29 |
| Q7Z6Z7 | E3 ubiquitin-protein ligase HUWE1 | HUWE1 | 182 |
| Q7Z6Z7 | E3 ubiquitin-protein ligase HUWE1 | HUWE1 | 183 |
| Q7Z6Z7 | E3 ubiquitin-protein ligase HUWE1 | HUWE1 | 349 |
| Q7Z6Z7 | E3 ubiquitin-protein ligase HUWE1 | HUWE1 | 471 |
| Q7Z6Z7 | E3 ubiquitin-protein ligase HUWE1 | HUWE1 | 612 |
| Q7Z6Z7 | E3 ubiquitin-protein ligase HUWE1 | HUWE1 | 624 |
| Q7Z6Z7 | E3 ubiquitin-protein ligase HUWE1 | HUWE1 | 689 |
| Q7Z6Z7 | E3 ubiquitin-protein ligase HUWE1 | HUWE1 | 699 |
| Q7Z6Z7 | E3 ubiquitin-protein ligase HUWE1 | HUWE1 | 790 |
| Q7Z6Z7 | E3 ubiquitin-protein ligase HUWE1 | HUWE1 | 821 |
| Q7Z6Z7 | E3 ubiquitin-protein ligase HUWE1 | HUWE1 | 1074 |
| Q7Z6Z7 | E3 ubiquitin-protein ligase HUWE1 | HUWE1 | 1133 |
| Q7Z6Z7 | E3 ubiquitin-protein ligase HUWE1 | HUWE1 | 1159 |
| Q7Z6Z7 | E3 ubiquitin-protein ligase HUWE1 | HUWE1 | 1252 |
| Q7Z6Z7 | E3 ubiquitin-protein ligase HUWE1 | HUWE1 | 1278 |
| Q7Z6Z7 | E3 ubiquitin-protein ligase HUWE1 | HUWE1 | 1401 |
| Q7Z6Z7 | E3 ubiquitin-protein ligase HUWE1 | HUWE1 | 1421 |
| Q7Z6Z7 | E3 ubiquitin-protein ligase HUWE1 | HUWE1 | 1462 |
| Q7Z6Z7 | E3 ubiquitin-protein ligase HUWE1 | HUWE1 | 1628 |
| Q7Z6Z7 | E3 ubiquitin-protein ligase HUWE1 | HUWE1 | 1832 |
| Q7Z6Z7 | E3 ubiquitin-protein ligase HUWE1 | HUWE1 | 1879 |
| Q7Z6Z7 | E3 ubiquitin-protein ligase HUWE1 | HUWE1 | 1891 |
| Q7Z6Z7 | E3 ubiquitin-protein ligase HUWE1 | HUWE1 | 1892 |
| Q7Z6Z7 | E3 ubiquitin-protein ligase HUWE1 | HUWE1 | 2181 |
| Q7Z6Z7 | E3 ubiquitin-protein ligase HUWE1 | HUWE1 | 2190 |
| Q7Z6Z7 | E3 ubiquitin-protein ligase HUWE1 | HUWE1 | 2655 |
| Q7Z6Z7 | E3 ubiquitin-protein ligase HUWE1 | HUWE1 | 2666 |
| Q7Z6Z7 | E3 ubiquitin-protein ligase HUWE1 | HUWE1 | 2721 |
| Q7Z6Z7 | E3 ubiquitin-protein ligase HUWE1 | HUWE1 | 3213 |
| Q7Z6Z7 | E3 ubiquitin-protein ligase HUWE1 | HUWE1 | 3239 |
| Q7Z6Z7 | E3 ubiquitin-protein ligase HUWE1 | HUWE1 | 3259 |
| Q7Z6Z7 | E3 ubiquitin-protein ligase HUWE1 | HUWE1 | 3296 |
| Q7Z6Z7 | E3 ubiquitin-protein ligase HUWE1 | HUWE1 | 3333 |
| Q7Z6Z7 | E3 ubiquitin-protein ligase HUWE1 | HUWE1 | 3361 |
| Q7Z6Z7 | E3 ubiquitin-protein ligase HUWE1 | HUWE1 | 3372 |
| Q7Z6Z7 | E3 ubiquitin-protein ligase HUWE1 | HUWE1 | 3375 |
| Q7Z6Z7 | E3 ubiquitin-protein ligase HUWE1 | HUWE1 | 3385 |
| Q7Z6Z7 | E3 ubiquitin-protein ligase HUWE1 | HUWE1 | 3635 |
| Q7Z6Z7 | E3 ubiquitin-protein ligase HUWE1 | HUWE1 | 3658 |
| Q7Z6Z7 | E3 ubiquitin-protein ligase HUWE1 | HUWE1 | 4099 |
| Q7Z6Z7 | E3 ubiquitin-protein ligase HUWE1 | HUWE1 | 4126 |
| Q7Z6Z7 | E3 ubiquitin-protein ligase HUWE1 | HUWE1 | 4211 |
| Q7Z6Z7 | E3 ubiquitin-protein ligase HUWE1 | HUWE1 | 4341 |
| Q7Z6Z7 | E3 ubiquitin-protein ligase HUWE1 | HUWE1 | 4367 |
| Q9P2D0 | Inhibitor of Bruton tyrosine kinase | IBTK | 41 |
| Q9P2D0 | Inhibitor of Bruton tyrosine kinase | IBTK | 62 |
| Q9P2D0 | Inhibitor of Bruton tyrosine kinase | IBTK | 201 |
| Q9P2D0 | Inhibitor of Bruton tyrosine kinase | IBTK | 217 |
| Q9P2D0 | Inhibitor of Bruton tyrosine kinase | IBTK | 231 |
| Q9P2D0 | Inhibitor of Bruton tyrosine kinase | IBTK | 325 |
| Q9P2D0 | Inhibitor of Bruton tyrosine kinase | IBTK | 522 |
| Q9P2D0 | Inhibitor of Bruton tyrosine kinase | IBTK | 764 |
| Q9P2D0 | Inhibitor of Bruton tyrosine kinase | IBTK | 1135 |
| Q9Y573 | Actin-binding protein IPP | IPP | 260 |
| Q9Y573 | Actin-binding protein IPP | IPP | 272 |
| Q9Y573 | Actin-binding protein IPP | IPP | 312 |
| Q8IU81 | Interferon regulatory factor 2-binding protein 1 | IRF2BP1 | 12 |
| Q8IU81 | Interferon regulatory factor 2-binding protein 1 | IRF2BP1 | 15 |
| Q8IU81 | Interferon regulatory factor 2-binding protein 1 | IRF2BP1 | 36 |
| Q8IU81 | Interferon regulatory factor 2-binding protein 1 | IRF2BP1 | 207 |
| Q8IU81 | Interferon regulatory factor 2-binding protein 1 | IRF2BP1 | 239 |
| Q8IU81 | Interferon regulatory factor 2-binding protein 1 | IRF2BP1 | 280 |
| Q8IU81 | Interferon regulatory factor 2-binding protein 1 | IRF2BP1 | 363 |
| Q8IU81 | Interferon regulatory factor 2-binding protein 1 | IRF2BP1 | 503 |
| Q8IU81 | Interferon regulatory factor 2-binding protein 1 | IRF2BP1 | 518 |
| Q8IU81 | Interferon regulatory factor 2-binding protein 1 | IRF2BP1 | 527 |
| Q8IU81 | Interferon regulatory factor 2-binding protein 1 | IRF2BP1 | 530 |
| Q7Z5L9 | Interferon regulatory factor 2-binding protein 2 | IRF2BP2 | 16 |
| Q7Z5L9 | Interferon regulatory factor 2-binding protein 2 | IRF2BP2 | 19 |
| Q7Z5L9 | Interferon regulatory factor 2-binding protein 2 | IRF2BP2 | 37 |
| Q7Z5L9 | Interferon regulatory factor 2-binding protein 2 | IRF2BP2 | 40 |
| Q7Z5L9 | Interferon regulatory factor 2-binding protein 2 | IRF2BP2 | 65 |
| Q7Z5L9 | Interferon regulatory factor 2-binding protein 2 | IRF2BP2 | 509 |
| Q7Z5L9 | Interferon regulatory factor 2-binding protein 2 | IRF2BP2 | 521 |
| Q7Z5L9 | Interferon regulatory factor 2-binding protein 2 | IRF2BP2 | 530 |
| Q7Z5L9 | Interferon regulatory factor 2-binding protein 2 | IRF2BP2 | 533 |
| Q7Z5L9 | Interferon regulatory factor 2-binding protein 2 | IRF2BP2 | 555 |
| Q9H1B7 | Interferon regulatory factor 2-binding protein-like | IRF2BPL | 14 |
| Q9H1B7 | Interferon regulatory factor 2-binding protein-like | IRF2BPL | 17 |
| Q9H1B7 | Interferon regulatory factor 2-binding protein-like | IRF2BPL | 35 |
| Q9H1B7 | Interferon regulatory factor 2-binding protein-like | IRF2BPL | 38 |
| Q9H1B7 | Interferon regulatory factor 2-binding protein-like | IRF2BPL | 63 |
| Q9H1B7 | Interferon regulatory factor 2-binding protein-like | IRF2BPL | 298 |
| Q9H1B7 | Interferon regulatory factor 2-binding protein-like | IRF2BPL | 445 |
| Q9H1B7 | Interferon regulatory factor 2-binding protein-like | IRF2BPL | 504 |
| Q9H1B7 | Interferon regulatory factor 2-binding protein-like | IRF2BPL | 718 |
| Q9H1B7 | Interferon regulatory factor 2-binding protein-like | IRF2BPL | 730 |
| Q9H1B7 | Interferon regulatory factor 2-binding protein-like | IRF2BPL | 739 |
| Q9H1B7 | Interferon regulatory factor 2-binding protein-like | IRF2BPL | 742 |
| Q9H1B7 | Interferon regulatory factor 2-binding protein-like | IRF2BPL | 758 |
| Q9H1B7 | Interferon regulatory factor 2-binding protein-like | IRF2BPL | 764 |
| Q96J02 | E3 ubiquitin-protein ligase Itchy homolog | ITCH | 57 |
| Q96J02 | E3 ubiquitin-protein ligase Itchy homolog | ITCH | 835 |
| Q96J02 | E3 ubiquitin-protein ligase Itchy homolog | ITCH | 871 |
| Q9Y6Y0 | Influenza virus NS1A-binding protein | IVNS1ABP | 32 |
| Q9Y6Y0 | Influenza virus NS1A-binding protein | IVNS1ABP | 39 |
| Q9Y6Y0 | Influenza virus NS1A-binding protein | IVNS1ABP | 52 |
| Q9Y6Y0 | Influenza virus NS1A-binding protein | IVNS1ABP | 53 |
| Q9Y6Y0 | Influenza virus NS1A-binding protein | IVNS1ABP | 121 |
| Q9Y6Y0 | Influenza virus NS1A-binding protein | IVNS1ABP | 134 |
| Q9Y6Y0 | Influenza virus NS1A-binding protein | IVNS1ABP | 143 |
| Q9Y6Y0 | Influenza virus NS1A-binding protein | IVNS1ABP | 187 |
| Q9Y6Y0 | Influenza virus NS1A-binding protein | IVNS1ABP | 274 |
| Q9Y6Y0 | Influenza virus NS1A-binding protein | IVNS1ABP | 381 |
| Q9Y6Y0 | Influenza virus NS1A-binding protein | IVNS1ABP | 454 |
| Q9Y6Y0 | Influenza virus NS1A-binding protein | IVNS1ABP | 564 |
| Q9Y6Y0 | Influenza virus NS1A-binding protein | IVNS1ABP | 575 |
| O94819 | Kelch repeat and BTB domain-containing protein 11 | KBTBD11 | 99 |
| O94819 | Kelch repeat and BTB domain-containing protein 11 | KBTBD11 | 257 |
| O94819 | Kelch repeat and BTB domain-containing protein 11 | KBTBD11 | 421 |
| O94819 | Kelch repeat and BTB domain-containing protein 11 | KBTBD11 | 485 |
| O94819 | Kelch repeat and BTB domain-containing protein 11 | KBTBD11 | 529 |
| O94819 | Kelch repeat and BTB domain-containing protein 11 | KBTBD11 | 537 |
| O94819 | Kelch repeat and BTB domain-containing protein 11 | KBTBD11 | 555 |
| Q8IY47 | Kelch repeat and BTB domain-containing protein 2 | KBTBD2 | 145 |
| Q8IY47 | Kelch repeat and BTB domain-containing protein 2 | KBTBD2 | 299 |
| Q8IY47 | Kelch repeat and BTB domain-containing protein 2 | KBTBD2 | 349 |
| Q8IY47 | Kelch repeat and BTB domain-containing protein 2 | KBTBD2 | 524 |
| Q8NAB2 | Kelch repeat and BTB domain-containing protein 3 | KBTBD3 | 137 |
| Q9NVX7 | Kelch repeat and BTB domain-containing protein 4 | KBTBD4 | 68 |
| Q9NVX7 | Kelch repeat and BTB domain-containing protein 4 | KBTBD4 | 201 |
| Q9NVX7 | Kelch repeat and BTB domain-containing protein 4 | KBTBD4 | 274 |
| Q9NVX7 | Kelch repeat and BTB domain-containing protein 4 | KBTBD4 | 301 |
| Q9NVX7 | Kelch repeat and BTB domain-containing protein 4 | KBTBD4 | 455 |
| Q9NVX7 | Kelch repeat and BTB domain-containing protein 4 | KBTBD4 | 472 |
| Q86V97 | Kelch repeat and BTB domain-containing protein 6 | KBTBD6 | 84 |
| Q86V97 | Kelch repeat and BTB domain-containing protein 6 | KBTBD6 | 93 |
| Q86V97 | Kelch repeat and BTB domain-containing protein 6 | KBTBD6 | 341 |
| Q86V97 | Kelch repeat and BTB domain-containing protein 6 | KBTBD6 | 375 |
| Q86V97 | Kelch repeat and BTB domain-containing protein 6 | KBTBD6 | 456 |
| Q8WVZ9 | Kelch repeat and BTB domain-containing protein 7 | KBTBD7 | 84 |
| Q8WVZ9 | Kelch repeat and BTB domain-containing protein 7 | KBTBD7 | 93 |
| Q8WVZ9 | Kelch repeat and BTB domain-containing protein 7 | KBTBD7 | 341 |
| Q8NFY9 | Kelch repeat and BTB domain-containing protein 8 | KBTBD8 | 181 |
| Q8NFY9 | Kelch repeat and BTB domain-containing protein 8 | KBTBD8 | 265 |
| Q8NFY9 | Kelch repeat and BTB domain-containing protein 8 | KBTBD8 | 276 |
| Q8NFY9 | Kelch repeat and BTB domain-containing protein 8 | KBTBD8 | 306 |
| Q8NFY9 | Kelch repeat and BTB domain-containing protein 8 | KBTBD8 | 318 |
| Q8NFY9 | Kelch repeat and BTB domain-containing protein 8 | KBTBD8 | 389 |
| Q8NFY9 | Kelch repeat and BTB domain-containing protein 8 | KBTBD8 | 413 |
| Q8NFY9 | Kelch repeat and BTB domain-containing protein 8 | KBTBD8 | 490 |
| Q8NFY9 | Kelch repeat and BTB domain-containing protein 8 | KBTBD8 | 587 |
| Q8NFY9 | Kelch repeat and BTB domain-containing protein 8 | KBTBD8 | 596 |
| Q9P0J7 | E3 ubiquitin-protein ligase KCMF1 | KCMF1 | 12 |
| Q9P0J7 | E3 ubiquitin-protein ligase KCMF1 | KCMF1 | 33 |
| Q9P0J7 | E3 ubiquitin-protein ligase KCMF1 | KCMF1 | 54 |
| Q719H9 | BTB/POZ domain-containing protein KCTD1 | KCTD1 | 140 |
| Q719H9 | BTB/POZ domain-containing protein KCTD1 | KCTD1 | 142 |
| Q9H3F6 | BTB/POZ domain-containing adapter for CUL3-mediated | KCTD10 | 80 |
| | RhoA degradation protein 3 | | |
| Q9H3F6 | BTB/POZ domain-containing adapter for CUL3-mediated | KCTD10 | 138 |
| | RhoA degradation protein 3 | | |
| Q9H3F6 | BTB/POZ domain-containing adapter for CUL3-mediated | KCTD10 | 227 |
| | RhoA degradation protein 3 | | |
| Q9H3F6 | BTB/POZ domain-containing adapter for CUL3-mediated | KCTD10 | 228 |
| | RhoA degradation protein 3 | | |
| Q96CX2 | BTB/POZ domain-containing protein KCTD12 | KCTD12 | 50 |
| Q96CX2 | BTB/POZ domain-containing protein KCTD12 | KCTD12 | 239 |
| Q8WZ19 | BTB/POZ domain-containing adapter for CUL3-mediated | KCTD13 | 237 |
| | RhoA degradation protein 1 | | |
| Q8WZ19 | BTB/POZ domain-containing adapter for CUL3-mediated | KCTD13 | 238 |
| | RhoA degradation protein 1 | | |
| Q96SI1 | BTB/POZ domain-containing protein KCTD15 | KCTD15 | 166 |
| Q96SI1 | BTB/POZ domain-containing protein KCTD15 | KCTD15 | 168 |
| Q96SI1 | BTB/POZ domain-containing protein KCTD15 | KCTD15 | 202 |
| Q7Z5Y7 | BTB/POZ domain-containing protein KCTD20 | KCTD20 | 19 |
| Q7Z5Y7 | BTB/POZ domain-containing protein KCTD20 | KCTD20 | 82 |
| Q7Z5Y7 | BTB/POZ domain-containing protein KCTD20 | KCTD20 | 327 |
| Q7Z5Y7 | BTB/POZ domain-containing protein KCTD20 | KCTD20 | 369 |
| Q4G0X4 | BTB/POZ domain-containing protein KCTD21 | KCTD21 | 237 |
| Q9Y597 | BTB/POZ domain-containing protein KCTD3 | KCTD3 | 112 |
| Q9Y597 | BTB/POZ domain-containing protein KCTD3 | KCTD3 | 530 |
| Q9Y597 | BTB/POZ domain-containing protein KCTD3 | KCTD3 | 541 |
| Q9Y597 | BTB/POZ domain-containing protein KCTD3 | KCTD3 | 599 |
| Q9Y597 | BTB/POZ domain-containing protein KCTD3 | KCTD3 | 606 |
| Q9Y597 | BTB/POZ domain-containing protein KCTD3 | KCTD3 | 704 |
| Q8WVF5 | BTB/POZ domain-containing protein KCTD4 | KCTD4 | 30 |
| Q9NXV2 | BTB/POZ domain-containing protein KCTD5 | KCTD5 | 25 |
| Q9NXV2 | BTB/POZ domain-containing protein KCTD5 | KCTD5 | 28 |
| Q9NXV2 | BTB/POZ domain-containing protein KCTD5 | KCTD5 | 63 |
| Q8NC69 | BTB/POZ domain-containing protein KCTD6 | KCTD6 | 182 |
| Q96MP8 | BTB/POZ domain-containing protein KCTD7 | KCTD7 | 204 |
| Q6ZWB6 | BTB/POZ domain-containing protein KCTD8 | KCTD8 | 238 |
| Q6ZWB6 | BTB/POZ domain-containing protein KCTD8 | KCTD8 | 431 |
| Q7L273 | BTB/POZ domain-containing protein KCTD9 | KCTD9 | 220 |
| Q7L273 | BTB/POZ domain-containing protein KCTD9 | KCTD9 | 256 |
| Q7L273 | BTB/POZ domain-containing protein KCTD9 | KCTD9 | 257 |
| Q7L273 | BTB/POZ domain-containing protein KCTD9 | KCTD9 | 272 |
| Q7L273 | BTB/POZ domain-containing protein KCTD9 | KCTD9 | 282 |
| Q7L273 | BTB/POZ domain-containing protein KCTD9 | KCTD9 | 293 |
| Q9Y2K7 | Lysine-specific demethylase 2A | KDM2A | 200 |
| Q9Y2K7 | Lysine-specific demethylase 2A | KDM2A | 429 |
| Q9Y2K7 | Lysine-specific demethylase 2A | KDM2A | 443 |
| Q9Y2K7 | Lysine-specific demethylase 2A | KDM2A | 463 |
| Q9Y2K7 | Lysine-specific demethylase 2A | KDM2A | 477 |
| Q9Y2K7 | Lysine-specific demethylase 2A | KDM2A | 577 |
| Q9Y2K7 | Lysine-specific demethylase 2A | KDM2A | 582 |
| Q9Y2K7 | Lysine-specific demethylase 2A | KDM2A | 585 |
| Q9Y2K7 | Lysine-specific demethylase 2A | KDM2A | 588 |
| Q9Y2K7 | Lysine-specific demethylase 2A | KDM2A | 604 |
| Q9Y2K7 | Lysine-specific demethylase 2A | KDM2A | 609 |
| Q9Y2K7 | Lysine-specific demethylase 2A | KDM2A | 620 |
| Q9Y2K7 | Lysine-specific demethylase 2A | KDM2A | 623 |
| Q9Y2K7 | Lysine-specific demethylase 2A | KDM2A | 672 |
| Q9Y2K7 | Lysine-specific demethylase 2A | KDM2A | 675 |
| Q9Y2K7 | Lysine-specific demethylase 2A | KDM2A | 708 |
| Q9Y2K7 | Lysine-specific demethylase 2A | KDM2A | 840 |
| Q8NHM5 | Lysine-specific demethylase 2B | KDM2B | 533 |
| Q8NHM5 | Lysine-specific demethylase 2B | KDM2B | 630 |
| Q8NHM5 | Lysine-specific demethylase 2B | KDM2B | 646 |
| Q8NHM5 | Lysine-specific demethylase 2B | KDM2B | 719 |
| Q8NHM5 | Lysine-specific demethylase 2B | KDM2B | 722 |
| Q8NHM5 | Lysine-specific demethylase 2B | KDM2B | 770 |
| Q8NHM5 | Lysine-specific demethylase 2B | KDM2B | 1035 |
| Q8NHM5 | Lysine-specific demethylase 2B | KDM2B | 1110 |
| Q8NHM5 | Lysine-specific demethylase 2B | KDM2B | 1292 |
| Q8NHM5 | Lysine-specific demethylase 2B | KDM2B | 1296 |
| Q14145 | Kelch-like ECH-associated protein 1 | KEAP1 | 23 |
| Q14145 | Kelch-like ECH-associated protein 1 | KEAP1 | 38 |
| Q14145 | Kelch-like ECH-associated protein 1 | KEAP1 | 151 |
| Q14145 | Kelch-like ECH-associated protein 1 | KEAP1 | 226 |
| Q14145 | Kelch-like ECH-associated protein 1 | KEAP1 | 241 |
| Q14145 | Kelch-like ECH-associated protein 1 | KEAP1 | 257 |
| Q14145 | Kelch-like ECH-associated protein 1 | KEAP1 | 288 |
| Q14145 | Kelch-like ECH-associated protein 1 | KEAP1 | 297 |
| Q14145 | Kelch-like ECH-associated protein 1 | KEAP1 | 319 |
| Q14145 | Kelch-like ECH-associated protein 1 | KEAP1 | 434 |
| Q14145 | Kelch-like ECH-associated protein 1 | KEAP1 | 613 |
| Q14145 | Kelch-like ECH-associated protein 1 | KEAP1 | 622 |
| Q14145 | Kelch-like ECH-associated protein 1 | KEAP1 | 624 |
| Q9HCI6 | E3 SUMO-protein ligase KIAA1586 | KIAA1586 | 98 |
| Q9HCI6 | E3 SUMO-protein ligase KIAA1586 | KIAA1586 | 281 |
| Q9HCI6 | E3 SUMO-protein ligase KIAA1586 | KIAA1586 | 758 |
| Q6PID8 | Kelch domain-containing protein 10 | KLHDC10 | 73 |
| Q6PID8 | Kelch domain-containing protein 10 | KLHDC10 | 254 |
| Q6PID8 | Kelch domain-containing protein 10 | KLHDC10 | 318 |
| Q9Y2U9 | Kelch domain-containing protein 2 | KLHDC2 | 137 |
| Q9Y2U9 | Kelch domain-containing protein 2 | KLHDC2 | 223 |
| Q9Y2U9 | Kelch domain-containing protein 2 | KLHDC2 | 374 |
| Q9Y2U9 | Kelch domain-containing protein 2 | KLHDC2 | 385 |
| Q9BQ90 | Kelch domain-containing protein 3 | KLHDC3 | 33 |
| Q9BQ90 | Kelch domain-containing protein 3 | KLHDC3 | 102 |
| Q9BQ90 | Kelch domain-containing protein 3 | KLHDC3 | 134 |
| Q9BQ90 | Kelch domain-containing protein 3 | KLHDC3 | 213 |
| Q9BQ90 | Kelch domain-containing protein 3 | KLHDC3 | 229 |
| Q9BQ90 | Kelch domain-containing protein 3 | KLHDC3 | 289 |
| Q9NR64 | Kelch-like protein 1 | KLHL1 | 525 |
| Q9NR64 | Kelch-like protein 1 | KLHL1 | 634 |
| Q9NVR0 | Kelch-like protein 11 | KLHL11 | 99 |
| Q9NVR0 | Kelch-like protein 11 | KLHL11 | 318 |
| Q9NVR0 | Kelch-like protein 11 | KLHL11 | 341 |
| Q9NVR0 | Kelch-like protein 11 | KLHL11 | 570 |
| Q9NVR0 | Kelch-like protein 11 | KLHL11 | 635 |
| Q9NVR0 | Kelch-like protein 11 | KLHL11 | 657 |
| Q9NVR0 | Kelch-like protein 11 | KLHL11 | 708 |
| Q53G59 | Kelch-like protein 12 | KLHL12 | 244 |
| Q53G59 | Kelch-like protein 12 | KLHL12 | 248 |
| Q53G59 | Kelch-like protein 12 | KLHL12 | 511 |
| Q9P2N7 | Kelch-like protein 13 | KLHL13 | 134 |
| Q9P2N7 | Kelch-like protein 13 | KLHL13 | 601 |
| Q9P2G3 | Kelch-like protein 14 | KLHL14 | 33 |
| Q9P2G3 | Kelch-like protein 14 | KLHL14 | 46 |
| Q9P2G3 | Kelch-like protein 14 | KLHL14 | 593 |
| Q9P2G3 | Kelch-like protein 14 | KLHL14 | 623 |
| O94889 | Kelch-like protein 18 | KLHL18 | 38 |
| O94889 | Kelch-like protein 18 | KLHL18 | 523 |
| Q9Y2M5 | Kelch-like protein 20 | KLHL20 | 25 |
| Q9Y2M5 | Kelch-like protein 20 | KLHL20 | 356 |
| Q53GT1 | Kelch-like protein 22 | KLHL22 | 22 |
| Q8NBE8 | Kelch-like protein 23 | KLHL23 | 245 |
| Q8NBE8 | Kelch-like protein 23 | KLHL23 | 431 |
| Q6TFL4 | Kelch-like protein 24 | KLHL24 | 281 |
| Q6TFL4 | Kelch-like protein 24 | KLHL24 | 321 |
| Q6TFL4 | Kelch-like protein 24 | KLHL24 | 468 |
| Q6TFL4 | Kelch-like protein 24 | KLHL24 | 542 |
| Q9H0H3 | Kelch-like protein 25 | KLHL25 | 31 |
| Q9H0H3 | Kelch-like protein 25 | KLHL25 | 287 |
| Q9H0H3 | Kelch-like protein 25 | KLHL25 | 307 |
| Q9H0H3 | Kelch-like protein 25 | KLHL25 | 340 |
| Q9H0H3 | Kelch-like protein 25 | KLHL25 | 531 |
| Q53HC5 | Kelch-like protein 26 | KLHL26 | 221 |
| Q53HC5 | Kelch-like protein 26 | KLHL26 | 249 |
| Q53HC5 | Kelch-like protein 26 | KLHL26 | 480 |
| Q53HC5 | Kelch-like protein 26 | KLHL26 | 554 |
| Q53HC5 | Kelch-like protein 26 | KLHL26 | 555 |
| Q53HC5 | Kelch-like protein 26 | KLHL26 | 603 |
| Q0D2K2 | Kelch-like protein 30 | KLHL30 | 432 |
| Q8N4N3 | Kelch-like protein 36 | KLHL36 | 254 |
| Q8N4N3 | Kelch-like protein 36 | KLHL36 | 263 |
| Q8N4N3 | Kelch-like protein 36 | KLHL36 | 308 |
| Q8N4N3 | Kelch-like protein 36 | KLHL36 | 523 |
| Q9C0H6 | Kelch-like protein 4 | KLHL4 | 35 |
| Q9C0H6 | Kelch-like protein 4 | KLHL4 | 182 |
| Q9C0H6 | Kelch-like protein 4 | KLHL4 | 284 |
| Q9C0H6 | Kelch-like protein 4 | KLHL4 | 296 |
| Q9C0H6 | Kelch-like protein 4 | KLHL4 | 633 |
| Q9C0H6 | Kelch-like protein 4 | KLHL4 | 639 |
| Q9P2K6 | Kelch-like protein 42 | KLHL42 | 15 |
| Q96PQ7 | Kelch-like protein 5 | KLHL5 | 322 |
| Q96PQ7 | Kelch-like protein 5 | KLHL5 | 334 |
| Q96PQ7 | Kelch-like protein 5 | KLHL5 | 533 |
| Q96PQ7 | Kelch-like protein 5 | KLHL5 | 677 |
| Q8WZ60 | Kelch-like protein 6 | KLHL6 | 252 |
| Q8WZ60 | Kelch-like protein 6 | KLHL6 | 284 |
| Q8WZ60 | Kelch-like protein 6 | KLHL6 | 339 |
| Q8WZ60 | Kelch-like protein 6 | KLHL6 | 488 |
| Q8WZ60 | Kelch-like protein 6 | KLHL6 | 508 |
| Q8WZ60 | Kelch-like protein 6 | KLHL6 | 539 |
| Q8WZ60 | Kelch-like protein 6 | KLHL6 | 551 |
| Q8WZ60 | Kelch-like protein 6 | KLHL6 | 556 |
| Q8IXQ5 | Kelch-like protein 7 | KLHL7 | 158 |
| Q8IXQ5 | Kelch-like protein 7 | KLHL7 | 259 |
| Q9P2G9 | Kelch-like protein 8 | KLHL8 | 282 |
| Q9P2G9 | Kelch-like protein 8 | KLHL8 | 590 |
| Q9P2J3 | Kelch-like protein 9 | KLHL9 | 19 |
| Q9P2J3 | Kelch-like protein 9 | KLHL9 | 92 |
| Q9P2J3 | Kelch-like protein 9 | KLHL9 | 559 |
| Q08380 | Galectin-3-binding protein | LGALS3BP | 93 |
| Q08380 | Galectin-3-binding protein | LGALS3BP | 279 |
| Q08380 | Galectin-3-binding protein | LGALS3BP | 526 |
| Q08380 | Galectin-3-binding protein | LGALS3BP | 561 |
| Q8TBB1 | E3 ubiquitin-protein ligase LNX | LNX1 | 632 |
| Q8TBB1 | E3 ubiquitin-protein ligase LNX | LNX1 | 704 |
| Q8N448 | Ligand of Numb protein X 2 | LNX2 | 120 |
| Q8N448 | Ligand of Numb protein X 2 | LNX2 | 127 |
| Q8N448 | Ligand of Numb protein X 2 | LNX2 | 137 |
| Q8N448 | Ligand of Numb protein X 2 | LNX2 | 307 |
| Q8N448 | Ligand of Numb protein X 2 | LNX2 | 506 |
| Q1L5Z9 | LON peptidase N-terminal domain and RING finger protein 2 | LONRF2 | 143 |
| Q1L5Z9 | LON peptidase N-terminal domain and RING finger protein 2 | LONRF2 | 158 |
| Q1L5Z9 | LON peptidase N-terminal domain and RING finger protein 2 | LONRF2 | 163 |
| Q1L5Z9 | LON peptidase N-terminal domain and RING finger protein 2 | LONRF2 | 166 |
| Q1L5Z9 | LON peptidase N-terminal domain and RING finger protein 2 | LONRF2 | 190 |
| Q1L5Z9 | LON peptidase N-terminal domain and RING finger protein 2 | LONRF2 | 195 |
| Q1L5Z9 | LON peptidase N-terminal domain and RING finger protein 2 | LONRF2 | 298 |
| Q1L5Z9 | LON peptidase N-terminal domain and RING finger protein 2 | LONRF2 | 464 |
| Q1L5Z9 | LON peptidase N-terminal domain and RING finger protein 2 | LONRF2 | 469 |
| Q1L5Z9 | LON peptidase N-terminal domain and RING finger protein 2 | LONRF2 | 543 |
| Q1L5Z9 | LON peptidase N-terminal domain and RING finger protein 2 | LONRF2 | 568 |
| Q496Y0 | LON peptidase N-terminal domain and RING finger protein 3 | LONRF3 | 178 |
| Q496Y0 | LON peptidase N-terminal domain and RING finger protein 3 | LONRF3 | 181 |
| Q496Y0 | LON peptidase N-terminal domain and RING finger protein 3 | LONRF3 | 424 |
| Q96L50 | Leucine-rich repeat protein 1 | LRR1 | 33 |
| Q96L50 | Leucine-rich repeat protein 1 | LRR1 | 91 |
| Q96L50 | Leucine-rich repeat protein 1 | LRR1 | 114 |
| Q96L50 | Leucine-rich repeat protein 1 | LRR1 | 246 |
| Q96L50 | Leucine-rich repeat protein 1 | LRR1 | 266 |
| Q96L50 | Leucine-rich repeat protein 1 | LRR1 | 359 |
| Q15345 | Leucine-rich repeat-containing protein 41 | LRRC41 | 123 |
| Q15345 | Leucine-rich repeat-containing protein 41 | LRRC41 | 170 |
| Q15345 | Leucine-rich repeat-containing protein 41 | LRRC41 | 266 |
| Q15345 | Leucine-rich repeat-containing protein 41 | LRRC41 | 297 |
| Q15345 | Leucine-rich repeat-containing protein 41 | LRRC41 | 481 |
| Q15345 | Leucine-rich repeat-containing protein 41 | LRRC41 | 520 |
| Q15345 | Leucine-rich repeat-containing protein 41 | LRRC41 | 607 |
| Q15345 | Leucine-rich repeat-containing protein 41 | LRRC41 | 735 |
| Q15345 | Leucine-rich repeat-containing protein 41 | LRRC41 | 743 |
| Q6UWE0 | E3 ubiquitin-protein ligase LRSAM1 | LRSAM1 | 23 |
| Q6UWE0 | E3 ubiquitin-protein ligase LRSAM1 | LRSAM1 | 39 |
| Q6UWE0 | E3 ubiquitin-protein ligase LRSAM1 | LRSAM1 | 75 |
| Q6UWE0 | E3 ubiquitin-protein ligase LRSAM1 | LRSAM1 | 193 |
| Q6UWE0 | E3 ubiquitin-protein ligase LRSAM1 | LRSAM1 | 205 |
| Q6UWE0 | E3 ubiquitin-protein ligase LRSAM1 | LRSAM1 | 397 |
| Q6UWE0 | E3 ubiquitin-protein ligase LRSAM1 | LRSAM1 | 418 |
| Q6UWE0 | E3 ubiquitin-protein ligase LRSAM1 | LRSAM1 | 699 |
| Q6UWE0 | E3 ubiquitin-protein ligase LRSAM1 | LRSAM1 | 700 |
| Q6UWE0 | E3 ubiquitin-protein ligase LRSAM1 | LRSAM1 | 706 |
| Q6UWE0 | E3 ubiquitin-protein ligase LRSAM1 | LRSAM1 | 709 |
| O94822 | E3 ubiquitin-protein ligase listerin | LTN1 | 91 |
| O94822 | E3 ubiquitin-protein ligase listerin | LTN1 | 186 |
| O94822 | E3 ubiquitin-protein ligase listerin | LTN1 | 227 |
| O94822 | E3 ubiquitin-protein ligase listerin | LTN1 | 237 |
| O94822 | E3 ubiquitin-protein ligase listerin | LTN1 | 498 |
| O94822 | E3 ubiquitin-protein ligase listerin | LTN1 | 555 |
| O94822 | E3 ubiquitin-protein ligase listerin | LTN1 | 595 |
| O94822 | E3 ubiquitin-protein ligase listerin | LTN1 | 723 |
| O94822 | E3 ubiquitin-protein ligase listerin | LTN1 | 751 |
| O94822 | E3 ubiquitin-protein ligase listerin | LTN1 | 753 |
| O94822 | E3 ubiquitin-protein ligase listerin | LTN1 | 869 |
| O94822 | E3 ubiquitin-protein ligase listerin | LTN1 | 981 |
| O94822 | E3 ubiquitin-protein ligase listerin | LTN1 | 999 |
| O94822 | E3 ubiquitin-protein ligase listerin | LTN1 | 1131 |
| O94822 | E3 ubiquitin-protein ligase listerin | LTN1 | 1146 |
| O94822 | E3 ubiquitin-protein ligase listerin | LTN1 | 1159 |
| O94822 | E3 ubiquitin-protein ligase listerin | LTN1 | 1174 |
| O94822 | E3 ubiquitin-protein ligase listerin | LTN1 | 1565 |
| O94822 | E3 ubiquitin-protein ligase listerin | LTN1 | 1745 |
| O94822 | E3 ubiquitin-protein ligase listerin | LTN1 | 1758 |
| O94822 | E3 ubiquitin-protein ligase listerin | LTN1 | 1761 |
| Q8N653 | Leucine-zipper-like transcriptional regulator 1 | LZTR1 | 34 |
| Q8N653 | Leucine-zipper-like transcriptional regulator 1 | LZTR1 | 106 |
| Q8N653 | Leucine-zipper-like transcriptional regulator 1 | LZTR1 | 228 |
| Q8N653 | Leucine-zipper-like transcriptional regulator 1 | LZTR1 | 229 |
| Q8N653 | Leucine-zipper-like transcriptional regulator 1 | LZTR1 | 236 |
| Q8N653 | Leucine-zipper-like transcriptional regulator 1 | LZTR1 | 342 |
| Q8N653 | Leucine-zipper-like transcriptional regulator 1 | LZTR1 | 423 |
| Q8N653 | Leucine-zipper-like transcriptional regulator 1 | LZTR1 | 427 |
| Q8N653 | Leucine-zipper-like transcriptional regulator 1 | LZTR1 | 760 |
| Q13233 | Mitogen-activated protein kinase kinase kinase 1 | MAP3K1 | 193 |
| Q13233 | Mitogen-activated protein kinase kinase kinase 1 | MAP3K1 | 491 |
| Q13233 | Mitogen-activated protein kinase kinase kinase 1 | MAP3K1 | 691 |
| Q13233 | Mitogen-activated protein kinase kinase kinase 1 | MAP3K1 | 916 |
| Q13233 | Mitogen-activated protein kinase kinase kinase 1 | MAP3K1 | 1012 |
| Q13233 | Mitogen-activated protein kinase kinase kinase 1 | MAP3K1 | 1033 |
| Q13233 | Mitogen-activated protein kinase kinase kinase 1 | MAP3K1 | 1257 |
| Q13233 | Mitogen-activated protein kinase kinase kinase 1 | MAP3K1 | 1314 |
| Q13233 | Mitogen-activated protein kinase kinase kinase 1 | MAP3K1 | 1488 |
| Q8TCQ1 | E3 ubiquitin-protein ligase MARCH1 | MARCH1 | 73 |
| Q8TCQ1 | E3 ubiquitin-protein ligase MARCH1 | MARCH1 | 85 |
| Q8TCQ1 | E3 ubiquitin-protein ligase MARCH1 | MARCH1 | 97 |
| Q8TCQ1 | E3 ubiquitin-protein ligase MARCH1 | MARCH1 | 99 |
| Q8TCQ1 | E3 ubiquitin-protein ligase MARCH1 | MARCH1 | 241 |
| Q8TCQ1 | E3 ubiquitin-protein ligase MARCH1 | MARCH1 | 254 |
| Q86UD3 | E3 ubiquitin-protein ligase MARCH3 | MARCH3 | 6 |
| Q9NX47 | E3 ubiquitin-protein ligase MARCH5 | MARCH5 | 17 |
| Q9NX47 | E3 ubiquitin-protein ligase MARCH5 | MARCH5 | 33 |
| Q9NX47 | E3 ubiquitin-protein ligase MARCH5 | MARCH5 | 46 |
| Q9NX47 | E3 ubiquitin-protein ligase MARCH5 | MARCH5 | 65 |
| Q9NX47 | E3 ubiquitin-protein ligase MARCH5 | MARCH5 | 68 |
| Q9NX47 | E3 ubiquitin-protein ligase MARCH5 | MARCH5 | 188 |
| O60337 | E3 ubiquitin-protein ligase MARCH6 | MARCH6 | 26 |
| O60337 | E3 ubiquitin-protein ligase MARCH6 | MARCH6 | 28 |
| O60337 | E3 ubiquitin-protein ligase MARCH6 | MARCH6 | 39 |
| O60337 | E3 ubiquitin-protein ligase MARCH6 | MARCH6 | 52 |
| Q9H992 | E3 ubiquitin-protein ligase MARCH7 | MARCH7 | 96 |
| Q9H992 | E3 ubiquitin-protein ligase MARCH7 | MARCH7 | 99 |
| Q9H992 | E3 ubiquitin-protein ligase MARCH7 | MARCH7 | 394 |
| Q9H992 | E3 ubiquitin-protein ligase MARCH7 | MARCH7 | 552 |
| Q9H992 | E3 ubiquitin-protein ligase MARCH7 | MARCH7 | 555 |
| Q9H992 | E3 ubiquitin-protein ligase MARCH7 | MARCH7 | 570 |
| Q9H992 | E3 ubiquitin-protein ligase MARCH7 | MARCH7 | 572 |
| Q9H992 | E3 ubiquitin-protein ligase MARCH7 | MARCH7 | 583 |
| Q9H992 | E3 ubiquitin-protein ligase MARCH7 | MARCH7 | 604 |
| Q9H992 | E3 ubiquitin-protein ligase MARCH7 | MARCH7 | 607 |
| Q00987 | E3 ubiquitin-protein ligase Mdm2 | MDM2 | 77 |
| Q00987 | E3 ubiquitin-protein ligase Mdm2 | MDM2 | 206 |
| Q00987 | E3 ubiquitin-protein ligase Mdm2 | MDM2 | 207 |
| Q00987 | E3 ubiquitin-protein ligase Mdm2 | MDM2 | 362 |
| Q00987 | E3 ubiquitin-protein ligase Mdm2 | MDM2 | 449 |
| Q00987 | E3 ubiquitin-protein ligase Mdm2 | MDM2 | 464 |
| Q00987 | E3 ubiquitin-protein ligase Mdm2 | MDM2 | 475 |
| Q00987 | E3 ubiquitin-protein ligase Mdm2 | MDM2 | 478 |
| O15151 | Protein Mdm4 | MDM4 | 165 |
| O15151 | Protein Mdm4 | MDM4 | 362 |
| O15151 | Protein Mdm4 | MDM4 | 463 |
| Q96G25 | Mediator of RNA polymerase II transcription subunit 8 | MED8 | 31 |
| Q96G25 | Mediator of RNA polymerase II transcription subunit 8 | MED8 | 147 |
| A1L020 | RNA-binding protein MEX3A | MEX3A | 21 |
| A1L020 | RNA-binding protein MEX3A | MEX3A | 116 |
| A1L020 | RNA-binding protein MEX3A | MEX3A | 135 |
| A1L020 | RNA-binding protein MEX3A | MEX3A | 328 |
| A1L020 | RNA-binding protein MEX3A | MEX3A | 341 |
| A1L020 | RNA-binding protein MEX3A | MEX3A | 505 |
| A1L020 | RNA-binding protein MEX3A | MEX3A | 508 |
| Q6ZN04 | RNA-binding protein MEX3B | MEX3B | 438 |
| Q5U5Q3 | RNA-binding E3 ubiquitin-protein ligase MEX3C | MEX3C | 235 |
| Q5U5Q3 | RNA-binding E3 ubiquitin-protein ligase MEX3C | MEX3C | 319 |
| Q5U5Q3 | RNA-binding E3 ubiquitin-protein ligase MEX3C | MEX3C | 647 |
| Q86XN8 | RNA-binding protein MEX3D | MEX3D | 362 |
| Q86XN8 | RNA-binding protein MEX3D | MEX3D | 488 |
| Q86XN8 | RNA-binding protein MEX3D | MEX3D | 636 |
| Q86XN8 | RNA-binding protein MEX3D | MEX3D | 639 |
| O60291 | E3 ubiquitin-protein ligase MGRN1 | MGRN1 | 293 |
| O60291 | E3 ubiquitin-protein ligase MGRN1 | MGRN1 | 299 |
| O60291 | E3 ubiquitin-protein ligase MGRN1 | MGRN1 | 302 |
| O60291 | E3 ubiquitin-protein ligase MGRN1 | MGRN1 | 313 |
| O60291 | E3 ubiquitin-protein ligase MGRN1 | MGRN1 | 316 |
| O60291 | E3 ubiquitin-protein ligase MGRN1 | MGRN1 | 356 |
| O60291 | E3 ubiquitin-protein ligase MGRN1 | MGRN1 | 428 |
| O60291 | E3 ubiquitin-protein ligase MGRN1 | MGRN1 | 528 |
| Q86YT6 | E3 ubiquitin-protein ligase MIB1 | MIB1 | 85 |
| Q86YT6 | E3 ubiquitin-protein ligase MIB1 | MIB1 | 88 |
| Q86YT6 | E3 ubiquitin-protein ligase MIB1 | MIB1 | 112 |
| Q86YT6 | E3 ubiquitin-protein ligase MIB1 | MIB1 | 217 |
| Q86YT6 | E3 ubiquitin-protein ligase MIB1 | MIB1 | 342 |
| Q86YT6 | E3 ubiquitin-protein ligase MIB1 | MIB1 | 757 |
| Q86YT6 | E3 ubiquitin-protein ligase MIB1 | MIB1 | 786 |
| Q86YT6 | E3 ubiquitin-protein ligase MIB1 | MIB1 | 843 |
| Q86YT6 | E3 ubiquitin-protein ligase MIB1 | MIB1 | 869 |
| Q86YT6 | E3 ubiquitin-protein ligase MIB1 | MIB1 | 913 |
| Q86YT6 | E3 ubiquitin-protein ligase MIB1 | MIB1 | 963 |
| Q86YT6 | E3 ubiquitin-protein ligase MIB1 | MIB1 | 966 |
| Q86YT6 | E3 ubiquitin-protein ligase MIB1 | MIB1 | 985 |
| Q86YT6 | E3 ubiquitin-protein ligase MIB1 | MIB1 | 995 |
| Q96AX9 | E3 ubiquitin-protein ligase MIB2 | MIB2 | 152 |
| Q96AX9 | E3 ubiquitin-protein ligase MIB2 | MIB2 | 323 |
| Q96AX9 | E3 ubiquitin-protein ligase MIB2 | MIB2 | 905 |
| Q96AX9 | E3 ubiquitin-protein ligase MIB2 | MIB2 | 911 |
| Q96AX9 | E3 ubiquitin-protein ligase MIB2 | MIB2 | 924 |
| Q96AX9 | E3 ubiquitin-protein ligase MIB2 | MIB2 | 969 |
| Q96AX9 | E3 ubiquitin-protein ligase MIB2 | MIB2 | 972 |
| Q96AX9 | E3 ubiquitin-protein ligase MIB2 | MIB2 | 988 |
| Q96AX9 | E3 ubiquitin-protein ligase MIB2 | MIB2 | 991 |
| O15344 | E3 ubiquitin-protein ligase Midline-1 | MID1 | 42 |
| O15344 | E3 ubiquitin-protein ligase Midline-1 | MID1 | 119 |
| O15344 | E3 ubiquitin-protein ligase Midline-1 | MID1 | 122 |
| O15344 | E3 ubiquitin-protein ligase Midline-1 | MID1 | 134 |
| O15344 | E3 ubiquitin-protein ligase Midline-1 | MID1 | 137 |
| O15344 | E3 ubiquitin-protein ligase Midline-1 | MID1 | 142 |
| O15344 | E3 ubiquitin-protein ligase Midline-1 | MID1 | 145 |
| O15344 | E3 ubiquitin-protein ligase Midline-1 | MID1 | 248 |
| O15344 | E3 ubiquitin-protein ligase Midline-1 | MID1 | 302 |
| O15344 | E3 ubiquitin-protein ligase Midline-1 | MID1 | 578 |
| O15344 | E3 ubiquitin-protein ligase Midline-1 | MID1 | 580 |
| O15344 | E3 ubiquitin-protein ligase Midline-1 | MID1 | 647 |
| O15344 | E3 ubiquitin-protein ligase Midline-1 | MID1 | 662 |
| Q9UJV3 | Probable E3 ubiquitin-protein ligase MID2 | MID2 | 630 |
| Q9UJV3 | Probable E3 ubiquitin-protein ligase MID2 | MID2 | 718 |
| Q9UHC7 | E3 ubiquitin-protein ligase makorin-1 | MKRN1 | 69 |
| Q9UHC7 | E3 ubiquitin-protein ligase makorin-1 | MKRN1 | 75 |
| Q9UHC7 | E3 ubiquitin-protein ligase makorin-1 | MKRN1 | 98 |
| Q9UHC7 | E3 ubiquitin-protein ligase makorin-1 | MKRN1 | 186 |
| Q9UHC7 | E3 ubiquitin-protein ligase makorin-1 | MKRN1 | 214 |
| Q9UHC7 | E3 ubiquitin-protein ligase makorin-1 | MKRN1 | 222 |
| Q9UHC7 | E3 ubiquitin-protein ligase makorin-1 | MKRN1 | 259 |
| Q9UHC7 | E3 ubiquitin-protein ligase makorin-1 | MKRN1 | 281 |
| Q9UHC7 | E3 ubiquitin-protein ligase makorin-1 | MKRN1 | 284 |
| Q9UHC7 | E3 ubiquitin-protein ligase makorin-1 | MKRN1 | 305 |
| Q9UHC7 | E3 ubiquitin-protein ligase makorin-1 | MKRN1 | 310 |
| Q9UHC7 | E3 ubiquitin-protein ligase makorin-1 | MKRN1 | 331 |
| Q9UHC7 | E3 ubiquitin-protein ligase makorin-1 | MKRN1 | 334 |
| Q9UHC7 | E3 ubiquitin-protein ligase makorin-1 | MKRN1 | 380 |
| Q9H000 | Probable E3 ubiquitin-protein ligase makorin-2 | MKRN2 | 16 |
| Q9H000 | Probable E3 ubiquitin-protein ligase makorin-2 | MKRN2 | 45 |
| Q9H000 | Probable E3 ubiquitin-protein ligase makorin-2 | MKRN2 | 133 |
| Q9H000 | Probable E3 ubiquitin-protein ligase makorin-2 | MKRN2 | 171 |
| Q9H000 | Probable E3 ubiquitin-protein ligase makorin-2 | MKRN2 | 185 |
| Q9H000 | Probable E3 ubiquitin-protein ligase makorin-2 | MKRN2 | 193 |
| Q9H000 | Probable E3 ubiquitin-protein ligase makorin-2 | MKRN2 | 216 |
| Q9H000 | Probable E3 ubiquitin-protein ligase makorin-2 | MKRN2 | 241 |
| Q9H000 | Probable E3 ubiquitin-protein ligase makorin-2 | MKRN2 | 262 |
| Q9H000 | Probable E3 ubiquitin-protein ligase makorin-2 | MKRN2 | 267 |
| Q9H000 | Probable E3 ubiquitin-protein ligase makorin-2 | MKRN2 | 270 |
| Q9H000 | Probable E3 ubiquitin-protein ligase makorin-2 | MKRN2 | 288 |
| Q9H000 | Probable E3 ubiquitin-protein ligase makorin-2 | MKRN2 | 291 |
| Q9H000 | Probable E3 ubiquitin-protein ligase makorin-2 | MKRN2 | 337 |
| Q13064 | Probable E3 ubiquitin-protein ligase makorin-3 | MKRN3 | 35 |
| Q13064 | Probable E3 ubiquitin-protein ligase makorin-3 | MKRN3 | 244 |
| P51948 | CDK-activating kinase assembly factor MAT1 | MNAT1 | 46 |
| P51948 | CDK-activating kinase assembly factor MAT1 | MNAT1 | 49 |
| P51948 | CDK-activating kinase assembly factor MAT1 | MNAT1 | 293 |
| Q68DK7 | Male-specific lethal 1 homolog | MSL1 | 74 |
| Q68DK7 | Male-specific lethal 1 homolog | MSL1 | 104 |
| Q68DK7 | Male-specific lethal 1 homolog | MSL1 | 125 |
| Q68DK7 | Male-specific lethal 1 homolog | MSL1 | 221 |
| Q68DK7 | Male-specific lethal 1 homolog | MSL1 | 322 |
| Q68DK7 | Male-specific lethal 1 homolog | MSL1 | 369 |
| Q68DK7 | Male-specific lethal 1 homolog | MSL1 | 377 |
| Q68DK7 | Male-specific lethal 1 homolog | MSL1 | 434 |
| Q68DK7 | Male-specific lethal 1 homolog | MSL1 | 460 |
| Q68DK7 | Male-specific lethal 1 homolog | MSL1 | 598 |
| Q9HCI7 | E3 ubiquitin-protein ligase MSL2 | MSL2 | 62 |
| Q9HCI7 | E3 ubiquitin-protein ligase MSL2 | MSL2 | 67 |
| Q9HCI7 | E3 ubiquitin-protein ligase MSL2 | MSL2 | 107 |
| Q9HCI7 | E3 ubiquitin-protein ligase MSL2 | MSL2 | 476 |
| Q9HCI7 | E3 ubiquitin-protein ligase MSL2 | MSL2 | 483 |
| Q9HCI7 | E3 ubiquitin-protein ligase MSL2 | MSL2 | 498 |
| Q969V5 | Mitochondrial ubiquitin ligase activator of NFKB 1 | MUL1 | 113 |
| Q969V5 | Mitochondrial ubiquitin ligase activator of NFKB 1 | MUL1 | 302 |
| Q969V5 | Mitochondrial ubiquitin ligase activator of NFKB 1 | MUL1 | 305 |
| Q969V5 | Mitochondrial ubiquitin ligase activator of NFKB 1 | MUL1 | 326 |
| Q969V5 | Mitochondrial ubiquitin ligase activator of NFKB 1 | MUL1 | 336 |
| Q969V5 | Mitochondrial ubiquitin ligase activator of NFKB 1 | MUL1 | 339 |
| O75592 | E3 ubiquitin-protein ligase MYCBP2 | MYCBP2 | 114 |
| O75592 | E3 ubiquitin-protein ligase MYCBP2 | MYCBP2 | 119 |
| O75592 | E3 ubiquitin-protein ligase MYCBP2 | MYCBP2 | 167 |
| O75592 | E3 ubiquitin-protein ligase MYCBP2 | MYCBP2 | 184 |
| O75592 | E3 ubiquitin-protein ligase MYCBP2 | MYCBP2 | 607 |
| O75592 | E3 ubiquitin-protein ligase MYCBP2 | MYCBP2 | 760 |
| O75592 | E3 ubiquitin-protein ligase MYCBP2 | MYCBP2 | 767 |
| O75592 | E3 ubiquitin-protein ligase MYCBP2 | MYCBP2 | 772 |
| O75592 | E3 ubiquitin-protein ligase MYCBP2 | MYCBP2 | 773 |
| O75592 | E3 ubiquitin-protein ligase MYCBP2 | MYCBP2 | 900 |
| O75592 | E3 ubiquitin-protein ligase MYCBP2 | MYCBP2 | 1064 |
| O75592 | E3 ubiquitin-protein ligase MYCBP2 | MYCBP2 | 1074 |
| O75592 | E3 ubiquitin-protein ligase MYCBP2 | MYCBP2 | 1111 |
| O75592 | E3 ubiquitin-protein ligase MYCBP2 | MYCBP2 | 1131 |
| O75592 | E3 ubiquitin-protein ligase MYCBP2 | MYCBP2 | 1427 |
| O75592 | E3 ubiquitin-protein ligase MYCBP2 | MYCBP2 | 1437 |
| O75592 | E3 ubiquitin-protein ligase MYCBP2 | MYCBP2 | 1459 |
| O75592 | E3 ubiquitin-protein ligase MYCBP2 | MYCBP2 | 1478 |
| O75592 | E3 ubiquitin-protein ligase MYCBP2 | MYCBP2 | 1868 |
| O75592 | E3 ubiquitin-protein ligase MYCBP2 | MYCBP2 | 1976 |
| O75592 | E3 ubiquitin-protein ligase MYCBP2 | MYCBP2 | 1995 |
| O75592 | E3 ubiquitin-protein ligase MYCBP2 | MYCBP2 | 2006 |
| O75592 | E3 ubiquitin-protein ligase MYCBP2 | MYCBP2 | 2131 |
| O75592 | E3 ubiquitin-protein ligase MYCBP2 | MYCBP2 | 2243 |
| O75592 | E3 ubiquitin-protein ligase MYCBP2 | MYCBP2 | 2531 |
| O75592 | E3 ubiquitin-protein ligase MYCBP2 | MYCBP2 | 2532 |
| O75592 | E3 ubiquitin-protein ligase MYCBP2 | MYCBP2 | 2568 |
| O75592 | E3 ubiquitin-protein ligase MYCBP2 | MYCBP2 | 2662 |
| O75592 | E3 ubiquitin-protein ligase MYCBP2 | MYCBP2 | 2898 |
| O75592 | E3 ubiquitin-protein ligase MYCBP2 | MYCBP2 | 2913 |
| O75592 | E3 ubiquitin-protein ligase MYCBP2 | MYCBP2 | 3152 |
| O75592 | E3 ubiquitin-protein ligase MYCBP2 | MYCBP2 | 3160 |
| O75592 | E3 ubiquitin-protein ligase MYCBP2 | MYCBP2 | 3225 |
| O75592 | E3 ubiquitin-protein ligase MYCBP2 | MYCBP2 | 3326 |
| O75592 | E3 ubiquitin-protein ligase MYCBP2 | MYCBP2 | 3354 |
| O75592 | E3 ubiquitin-protein ligase MYCBP2 | MYCBP2 | 3594 |
| O75592 | E3 ubiquitin-protein ligase MYCBP2 | MYCBP2 | 3786 |
| O75592 | E3 ubiquitin-protein ligase MYCBP2 | MYCBP2 | 3856 |
| O75592 | E3 ubiquitin-protein ligase MYCBP2 | MYCBP2 | 3932 |
| O75592 | E3 ubiquitin-protein ligase MYCBP2 | MYCBP2 | 4074 |
| O75592 | E3 ubiquitin-protein ligase MYCBP2 | MYCBP2 | 4181 |
| O75592 | E3 ubiquitin-protein ligase MYCBP2 | MYCBP2 | 4318 |
| O75592 | E3 ubiquitin-protein ligase MYCBP2 | MYCBP2 | 4355 |
| O75592 | E3 ubiquitin-protein ligase MYCBP2 | MYCBP2 | 4375 |
| O75592 | E3 ubiquitin-protein ligase MYCBP2 | MYCBP2 | 4390 |
| O75592 | E3 ubiquitin-protein ligase MYCBP2 | MYCBP2 | 4408 |
| O75592 | E3 ubiquitin-protein ligase MYCBP2 | MYCBP2 | 4437 |
| O75592 | E3 ubiquitin-protein ligase MYCBP2 | MYCBP2 | 4440 |
| O75592 | E3 ubiquitin-protein ligase MYCBP2 | MYCBP2 | 4506 |
| O75592 | E3 ubiquitin-protein ligase MYCBP2 | MYCBP2 | 4520 |
| O75592 | E3 ubiquitin-protein ligase MYCBP2 | MYCBP2 | 4537 |
| O75592 | E3 ubiquitin-protein ligase MYCBP2 | MYCBP2 | 4540 |
| O75592 | E3 ubiquitin-protein ligase MYCBP2 | MYCBP2 | 4549 |
| O75592 | E3 ubiquitin-protein ligase MYCBP2 | MYCBP2 | 4600 |
| O75592 | E3 ubiquitin-protein ligase MYCBP2 | MYCBP2 | 4614 |
| O75592 | E3 ubiquitin-protein ligase MYCBP2 | MYCBP2 | 4631 |
| O75592 | E3 ubiquitin-protein ligase MYCBP2 | MYCBP2 | 4634 |
| Q9NPC7 | Myoneurin | MYNN | 267 |
| Q9NPC7 | Myoneurin | MYNN | 342 |
| Q9NPC7 | Myoneurin | MYNN | 371 |
| Q96RE7 | Nucleus accumbens-associated protein 1 | NACC1 | 30 |
| Q96RE7 | Nucleus accumbens-associated protein 1 | NACC1 | 128 |
| Q96RE7 | Nucleus accumbens-associated protein 1 | NACC1 | 178 |
| Q96RE7 | Nucleus accumbens-associated protein 1 | NACC1 | 301 |
| Q96RE7 | Nucleus accumbens-associated protein 1 | NACC1 | 387 |
| Q96RE7 | Nucleus accumbens-associated protein 1 | NACC1 | 416 |
| Q96RE7 | Nucleus accumbens-associated protein 1 | NACC1 | 444 |
| Q96RE7 | Nucleus accumbens-associated protein 1 | NACC1 | 464 |
| Q96BF6 | Nucleus accumbens-associated protein 2 | NACC2 | 310 |
| Q96BF6 | Nucleus accumbens-associated protein 2 | NACC2 | 367 |
| Q96BF6 | Nucleus accumbens-associated protein 2 | NACC2 | 393 |
| P46934 | E3 ubiquitin-protein ligase NEDD4 | NEDD4 | 601 |
| P46934 | E3 ubiquitin-protein ligase NEDD4 | NEDD4 | 1286 |
| Q96PU5 | E3 ubiquitin-protein ligase NEDD4-like | NEDD4L | 341 |
| Q96PU5 | E3 ubiquitin-protein ligase NEDD4-like | NEDD4L | 874 |
| Q96PU5 | E3 ubiquitin-protein ligase NEDD4-like | NEDD4L | 942 |
| A8MQ27 | E3 ubiquitin-protein ligase NEURL1B | NEURL1B | 119 |
| A8MQ27 | E3 ubiquitin-protein ligase NEURL1B | NEURL1B | 213 |
| A8MQ27 | E3 ubiquitin-protein ligase NEURL1B | NEURL1B | 300 |
| A8MQ27 | E3 ubiquitin-protein ligase NEURL1B | NEURL1B | 538 |
| Q9BR09 | Neuralized-like protein 2 | NEURL2 | 78 |
| Q96EH8 | E3 ubiquitin-protein ligase NEURL3 | NEURL3 | 141 |
| Q96EH8 | E3 ubiquitin-protein ligase NEURL3 | NEURL3 | 202 |
| Q96EH8 | E3 ubiquitin-protein ligase NEURL3 | NEURL3 | 223 |
| Q12986 | Transcriptional repressor NF-X1 | NFX1 | 32 |
| Q12986 | Transcriptional repressor NF-X1 | NFX1 | 54 |
| Q12986 | Transcriptional repressor NF-X1 | NFX1 | 92 |
| Q12986 | Transcriptional repressor NF-X1 | NFX1 | 190 |
| Q12986 | Transcriptional repressor NF-X1 | NFX1 | 295 |
| Q12986 | Transcriptional repressor NF-X1 | NFX1 | 315 |
| Q12986 | Transcriptional repressor NF-X1 | NFX1 | 444 |
| Q12986 | Transcriptional repressor NF-X1 | NFX1 | 477 |
| Q12986 | Transcriptional repressor NF-X1 | NFX1 | 479 |
| Q12986 | Transcriptional repressor NF-X1 | NFX1 | 542 |
| Q12986 | Transcriptional repressor NF-X1 | NFX1 | 567 |
| Q12986 | Transcriptional repressor NF-X1 | NFX1 | 572 |
| Q12986 | Transcriptional repressor NF-X1 | NFX1 | 576 |
| Q12986 | Transcriptional repressor NF-X1 | NFX1 | 581 |
| Q12986 | Transcriptional repressor NF-X1 | NFX1 | 593 |
| Q12986 | Transcriptional repressor NF-X1 | NFX1 | 596 |
| Q12986 | Transcriptional repressor NF-X1 | NFX1 | 615 |
| Q12986 | Transcriptional repressor NF-X1 | NFX1 | 622 |
| Q12986 | Transcriptional repressor NF-X1 | NFX1 | 626 |
| Q12986 | Transcriptional repressor NF-X1 | NFX1 | 632 |
| Q12986 | Transcriptional repressor NF-X1 | NFX1 | 641 |
| Q12986 | Transcriptional repressor NF-X1 | NFX1 | 645 |
| Q12986 | Transcriptional repressor NF-X1 | NFX1 | 653 |
| Q12986 | Transcriptional repressor NF-X1 | NFX1 | 661 |
| Q12986 | Transcriptional repressor NF-X1 | NFX1 | 672 |
| Q12986 | Transcriptional repressor NF-X1 | NFX1 | 684 |
| Q12986 | Transcriptional repressor NF-X1 | NFX1 | 699 |
| Q12986 | Transcriptional repressor NF-X1 | NFX1 | 703 |
| Q12986 | Transcriptional repressor NF-X1 | NFX1 | 704 |
| Q12986 | Transcriptional repressor NF-X1 | NFX1 | 711 |
| Q12986 | Transcriptional repressor NF-X1 | NFX1 | 715 |
| Q12986 | Transcriptional repressor NF-X1 | NFX1 | 730 |
| Q12986 | Transcriptional repressor NF-X1 | NFX1 | 778 |
| Q12986 | Transcriptional repressor NF-X1 | NFX1 | 795 |
| Q12986 | Transcriptional repressor NF-X1 | NFX1 | 803 |
| Q12986 | Transcriptional repressor NF-X1 | NFX1 | 815 |
| Q12986 | Transcriptional repressor NF-X1 | NFX1 | 822 |
| Q12986 | Transcriptional repressor NF-X1 | NFX1 | 826 |
| Q12986 | Transcriptional repressor NF-X1 | NFX1 | 832 |
| Q12986 | Transcriptional repressor NF-X1 | NFX1 | 841 |
| Q12986 | Transcriptional repressor NF-X1 | NFX1 | 846 |
| Q12986 | Transcriptional repressor NF-X1 | NFX1 | 852 |
| Q12986 | Transcriptional repressor NF-X1 | NFX1 | 856 |
| Q12986 | Transcriptional repressor NF-X1 | NFX1 | 867 |
| Q12986 | Transcriptional repressor NF-X1 | NFX1 | 871 |
| Q12986 | Transcriptional repressor NF-X1 | NFX1 | 877 |
| Q12986 | Transcriptional repressor NF-X1 | NFX1 | 882 |
| Q12986 | Transcriptional repressor NF-X1 | NFX1 | 890 |
| Q12986 | Transcriptional repressor NF-X1 | NFX1 | 892 |
| Q12986 | Transcriptional repressor NF-X1 | NFX1 | 901 |
| Q12986 | Transcriptional repressor NF-X1 | NFX1 | 947 |
| Q12986 | Transcriptional repressor NF-X1 | NFX1 | 1067 |
| Q6ZNB6 | NF-X1-type zinc finger protein NFXL1 | NFXL1 | 163 |
| Q6ZNB6 | NF-X1-type zinc finger protein NFXL1 | NFXL1 | 176 |
| Q6ZNB6 | NF-X1-type zinc finger protein NFXL1 | NFXL1 | 187 |
| Q6ZNB6 | NF-X1-type zinc finger protein NFXL1 | NFXL1 | 212 |
| Q6ZNB6 | NF-X1-type zinc finger protein NFXL1 | NFXL1 | 219 |
| Q6ZNB6 | NF-X1-type zinc finger protein NFXL1 | NFXL1 | 236 |
| Q6ZNB6 | NF-X1-type zinc finger protein NFXL1 | NFXL1 | 265 |
| Q6ZNB6 | NF-X1-type zinc finger protein NFXL1 | NFXL1 | 269 |
| Q6ZNB6 | NF-X1-type zinc finger protein NFXL1 | NFXL1 | 273 |
| Q6ZNB6 | NF-X1-type zinc finger protein NFXL1 | NFXL1 | 278 |
| Q6ZNB6 | NF-X1-type zinc finger protein NFXL1 | NFXL1 | 281 |
| Q6ZNB6 | NF-X1-type zinc finger protein NFXL1 | NFXL1 | 289 |
| Q6ZNB6 | NF-X1-type zinc finger protein NFXL1 | NFXL1 | 291 |
| Q6ZNB6 | NF-X1-type zinc finger protein NFXL1 | NFXL1 | 308 |
| Q6ZNB6 | NF-X1-type zinc finger protein NFXL1 | NFXL1 | 312 |
| Q6ZNB6 | NF-X1-type zinc finger protein NFXL1 | NFXL1 | 318 |
| Q6ZNB6 | NF-X1-type zinc finger protein NFXL1 | NFXL1 | 327 |
| Q6ZNB6 | NF-X1-type zinc finger protein NFXL1 | NFXL1 | 335 |
| Q6ZNB6 | NF-X1-type zinc finger protein NFXL1 | NFXL1 | 343 |
| Q6ZNB6 | NF-X1-type zinc finger protein NFXL1 | NFXL1 | 345 |
| Q6ZNB6 | NF-X1-type zinc finger protein NFXL1 | NFXL1 | 354 |
| Q6ZNB6 | NF-X1-type zinc finger protein NFXL1 | NFXL1 | 361 |
| Q6ZNB6 | NF-X1-type zinc finger protein NFXL1 | NFXL1 | 365 |
| Q6ZNB6 | NF-X1-type zinc finger protein NFXL1 | NFXL1 | 371 |
| Q6ZNB6 | NF-X1-type zinc finger protein NFXL1 | NFXL1 | 376 |
| Q6ZNB6 | NF-X1-type zinc finger protein NFXL1 | NFXL1 | 388 |
| Q6ZNB6 | NF-X1-type zinc finger protein NFXL1 | NFXL1 | 396 |
| Q6ZNB6 | NF-X1-type zinc finger protein NFXL1 | NFXL1 | 398 |
| Q6ZNB6 | NF-X1-type zinc finger protein NFXL1 | NFXL1 | 407 |
| Q6ZNB6 | NF-X1-type zinc finger protein NFXL1 | NFXL1 | 414 |
| Q6ZNB6 | NF-X1-type zinc finger protein NFXL1 | NFXL1 | 418 |
| Q6ZNB6 | NF-X1-type zinc finger protein NFXL1 | NFXL1 | 424 |
| Q6ZNB6 | NF-X1-type zinc finger protein NFXL1 | NFXL1 | 438 |
| Q6ZNB6 | NF-X1-type zinc finger protein NFXL1 | NFXL1 | 441 |
| Q6ZNB6 | NF-X1-type zinc finger protein NFXL1 | NFXL1 | 449 |
| Q6ZNB6 | NF-X1-type zinc finger protein NFXL1 | NFXL1 | 460 |
| Q6ZNB6 | NF-X1-type zinc finger protein NFXL1 | NFXL1 | 476 |
| Q6ZNB6 | NF-X1-type zinc finger protein NFXL1 | NFXL1 | 493 |
| Q6ZNB6 | NF-X1-type zinc finger protein NFXL1 | NFXL1 | 508 |
| Q6ZNB6 | NF-X1-type zinc finger protein NFXL1 | NFXL1 | 512 |
| Q6ZNB6 | NF-X1-type zinc finger protein NFXL1 | NFXL1 | 517 |
| Q6ZNB6 | NF-X1-type zinc finger protein NFXL1 | NFXL1 | 520 |
| Q6ZNB6 | NF-X1-type zinc finger protein NFXL1 | NFXL1 | 528 |
| Q6ZNB6 | NF-X1-type zinc finger protein NFXL1 | NFXL1 | 530 |
| Q6ZNB6 | NF-X1-type zinc finger protein NFXL1 | NFXL1 | 539 |
| Q6ZNB6 | NF-X1-type zinc finger protein NFXL1 | NFXL1 | 550 |
| Q6ZNB6 | NF-X1-type zinc finger protein NFXL1 | NFXL1 | 554 |
| Q6ZNB6 | NF-X1-type zinc finger protein NFXL1 | NFXL1 | 560 |
| Q6ZNB6 | NF-X1-type zinc finger protein NFXL1 | NFXL1 | 570 |
| Q6ZNB6 | NF-X1-type zinc finger protein NFXL1 | NFXL1 | 578 |
| Q6ZNB6 | NF-X1-type zinc finger protein NFXL1 | NFXL1 | 582 |
| Q6ZNB6 | NF-X1-type zinc finger protein NFXL1 | NFXL1 | 589 |
| Q6ZNB6 | NF-X1-type zinc finger protein NFXL1 | NFXL1 | 593 |
| Q6ZNB6 | NF-X1-type zinc finger protein NFXL1 | NFXL1 | 597 |
| Q6ZNB6 | NF-X1-type zinc finger protein NFXL1 | NFXL1 | 652 |
| Q6ZNB6 | NF-X1-type zinc finger protein NFXL1 | NFXL1 | 660 |
| Q6ZNB6 | NF-X1-type zinc finger protein NFXL1 | NFXL1 | 670 |
| Q6ZNB6 | NF-X1-type zinc finger protein NFXL1 | NFXL1 | 675 |
| Q6ZNB6 | NF-X1-type zinc finger protein NFXL1 | NFXL1 | 679 |
| Q6ZNB6 | NF-X1-type zinc finger protein NFXL1 | NFXL1 | 688 |
| Q6ZNB6 | NF-X1-type zinc finger protein NFXL1 | NFXL1 | 698 |
| Q6ZNB6 | NF-X1-type zinc finger protein NFXL1 | NFXL1 | 701 |
| Q6ZNB6 | NF-X1-type zinc finger protein NFXL1 | NFXL1 | 705 |
| Q6ZNB6 | NF-X1-type zinc finger protein NFXL1 | NFXL1 | 713 |
| Q6ZNB6 | NF-X1-type zinc finger protein NFXL1 | NFXL1 | 717 |
| Q6ZNB6 | NF-X1-type zinc finger protein NFXL1 | NFXL1 | 721 |
| Q6ZNB6 | NF-X1-type zinc finger protein NFXL1 | NFXL1 | 729 |
| Q6ZNB6 | NF-X1-type zinc finger protein NFXL1 | NFXL1 | 748 |
| Q6ZNB6 | NF-X1-type zinc finger protein NFXL1 | NFXL1 | 764 |
| Q6ZNB6 | NF-X1-type zinc finger protein NFXL1 | NFXL1 | 765 |
| Q6ZNB6 | NF-X1-type zinc finger protein NFXL1 | NFXL1 | 775 |
| Q6ZNB6 | NF-X1-type zinc finger protein NFXL1 | NFXL1 | 779 |
| Q6ZNB6 | NF-X1-type zinc finger protein NFXL1 | NFXL1 | 783 |
| Q6ZNB6 | NF-X1-type zinc finger protein NFXL1 | NFXL1 | 788 |
| Q6ZNB6 | NF-X1-type zinc finger protein NFXL1 | NFXL1 | 792 |
| Q6ZNB6 | NF-X1-type zinc finger protein NFXL1 | NFXL1 | 800 |
| Q6ZNB6 | NF-X1-type zinc finger protein NFXL1 | NFXL1 | 802 |
| Q6ZNB6 | NF-X1-type zinc finger protein NFXL1 | NFXL1 | 811 |
| Q6ZNB6 | NF-X1-type zinc finger protein NFXL1 | NFXL1 | 823 |
| Q6ZNB6 | NF-X1-type zinc finger protein NFXL1 | NFXL1 | 827 |
| Q6VVB1 | E3 ubiquitin-protein ligase NHLRC1 | NHLRC1 | 262 |
| Q9Y314 | Nitric oxide synthase-interacting protein | NOSIP | 8 |
| Q9Y314 | Nitric oxide synthase-interacting protein | NOSIP | 45 |
| Q9Y314 | Nitric oxide synthase-interacting protein | NOSIP | 46 |
| Q9Y314 | Nitric oxide synthase-interacting protein | NOSIP | 47 |
| Q9Y314 | Nitric oxide synthase-interacting protein | NOSIP | 185 |
| Q9Y314 | Nitric oxide synthase-interacting protein | NOSIP | 223 |
| Q9Y314 | Nitric oxide synthase-interacting protein | NOSIP | 236 |
| Q9Y314 | Nitric oxide synthase-interacting protein | NOSIP | 250 |
| O96028 | Histone-lysine N-methyltransferase NSD2 | NSD2 | 38 |
| O96028 | Histone-lysine N-methyltransferase NSD2 | NSD2 | 44 |
| O96028 | Histone-lysine N-methyltransferase NSD2 | NSD2 | 144 |
| O96028 | Histone-lysine N-methyltransferase NSD2 | NSD2 | 207 |
| O96028 | Histone-lysine N-methyltransferase NSD2 | NSD2 | 238 |
| O96028 | Histone-lysine N-methyltransferase NSD2 | NSD2 | 294 |
| O96028 | Histone-lysine N-methyltransferase NSD2 | NSD2 | 394 |
| O96028 | Histone-lysine N-methyltransferase NSD2 | NSD2 | 406 |
| O96028 | Histone-lysine N-methyltransferase NSD2 | NSD2 | 464 |
| O96028 | Histone-lysine N-methyltransferase NSD2 | NSD2 | 594 |
| O96028 | Histone-lysine N-methyltransferase NSD2 | NSD2 | 682 |
| O96028 | Histone-lysine N-methyltransferase NSD2 | NSD2 | 686 |
| O96028 | Histone-lysine N-methyltransferase NSD2 | NSD2 | 687 |
| O96028 | Histone-lysine N-methyltransferase NSD2 | NSD2 | 707 |
| O96028 | Histone-lysine N-methyltransferase NSD2 | NSD2 | 710 |
| O96028 | Histone-lysine N-methyltransferase NSD2 | NSD2 | 717 |
| O96028 | Histone-lysine N-methyltransferase NSD2 | NSD2 | 720 |
| O96028 | Histone-lysine N-methyltransferase NSD2 | NSD2 | 730 |
| O96028 | Histone-lysine N-methyltransferase NSD2 | NSD2 | 735 |
| O96028 | Histone-lysine N-methyltransferase NSD2 | NSD2 | 743 |
| O96028 | Histone-lysine N-methyltransferase NSD2 | NSD2 | 759 |
| O96028 | Histone-lysine N-methyltransferase NSD2 | NSD2 | 764 |
| O96028 | Histone-lysine N-methyltransferase NSD2 | NSD2 | 788 |
| O96028 | Histone-lysine N-methyltransferase NSD2 | NSD2 | 813 |
| O96028 | Histone-lysine N-methyltransferase NSD2 | NSD2 | 846 |
| O96028 | Histone-lysine N-methyltransferase NSD2 | NSD2 | 849 |
| O96028 | Histone-lysine N-methyltransferase NSD2 | NSD2 | 899 |
| O96028 | Histone-lysine N-methyltransferase NSD2 | NSD2 | 1016 |
| O96028 | Histone-lysine N-methyltransferase NSD2 | NSD2 | 1018 |
| O96028 | Histone-lysine N-methyltransferase NSD2 | NSD2 | 1026 |
| O96028 | Histone-lysine N-methyltransferase NSD2 | NSD2 | 1032 |
| O96028 | Histone-lysine N-methyltransferase NSD2 | NSD2 | 1046 |
| O96028 | Histone-lysine N-methyltransferase NSD2 | NSD2 | 1052 |
| O96028 | Histone-lysine N-methyltransferase NSD2 | NSD2 | 1056 |
| O96028 | Histone-lysine N-methyltransferase NSD2 | NSD2 | 1102 |
| O96028 | Histone-lysine N-methyltransferase NSD2 | NSD2 | 1144 |
| O96028 | Histone-lysine N-methyltransferase NSD2 | NSD2 | 1148 |
| O96028 | Histone-lysine N-methyltransferase NSD2 | NSD2 | 1167 |
| O96028 | Histone-lysine N-methyltransferase NSD2 | NSD2 | 1183 |
| O96028 | Histone-lysine N-methyltransferase NSD2 | NSD2 | 1193 |
| O96028 | Histone-lysine N-methyltransferase NSD2 | NSD2 | 1198 |
| O96028 | Histone-lysine N-methyltransferase NSD2 | NSD2 | 1242 |
| O96028 | Histone-lysine N-methyltransferase NSD2 | NSD2 | 1245 |
| O96028 | Histone-lysine N-methyltransferase NSD2 | NSD2 | 1254 |
| O96028 | Histone-lysine N-methyltransferase NSD2 | NSD2 | 1267 |
| O96028 | Histone-lysine N-methyltransferase NSD2 | NSD2 | 1296 |
| O96028 | Histone-lysine N-methyltransferase NSD2 | NSD2 | 1315 |
| O96028 | Histone-lysine N-methyltransferase NSD2 | NSD2 | 1323 |
| O96028 | Histone-lysine N-methyltransferase NSD2 | NSD2 | 1324 |
| Q8WV22 | Non-structural maintenance of chromosomes element 1 | NSMCE1 | 40 |
| | homolog | | |
| Q8WV22 | Non-structural maintenance of chromosomes element 1 | NSMCE1 | 215 |
| | homolog | | |
| Q8WV22 | Non-structural maintenance of chromosomes element 1 | NSMCE1 | 228 |
| | homolog | | |
| Q96MF7 | E3 SUMO-protein ligase NSE2 | NSMCE2 | 140 |
| Q96MF7 | E3 SUMO-protein ligase NSE2 | NSMCE2 | 169 |
| Q96MF7 | E3 SUMO-protein ligase NSE2 | NSMCE2 | 185 |
| Q96MF7 | E3 SUMO-protein ligase NSE2 | NSMCE2 | 210 |
| Q96MF7 | E3 SUMO-protein ligase NSE2 | NSMCE2 | 215 |
| Q86WC4 | Osteopetrosis-associated transmembrane protein 1 | OSTM1 | 112 |
| Q8TE49 | OTU domain-containing protein 7A | OTUD7A | 210 |
| Q6GQQ9 | OTU domain-containing protein 7B | OTUD7B | 133 |
| Q6GQQ9 | OTU domain-containing protein 7B | OTUD7B | 194 |
| Q6GQQ9 | OTU domain-containing protein 7B | OTUD7B | 345 |
| Q6GQQ9 | OTU domain-containing protein 7B | OTUD7B | 708 |
| Q6GQQ9 | OTU domain-containing protein 7B | OTUD7B | 720 |
| Q6GQQ9 | OTU domain-containing protein 7B | OTUD7B | 734 |
| Q6GQQ9 | OTU domain-containing protein 7B | OTUD7B | 807 |
| Q6GQQ9 | OTU domain-containing protein 7B | OTUD7B | 819 |
| Q6GQQ9 | OTU domain-containing protein 7B | OTUD7B | 821 |
| Q6GQQ9 | OTU domain-containing protein 7B | OTUD7B | 822 |
| Q9HBE1 | POZ-, AT hook-, and zinc finger-containing protein 1 | PATZ1 | 9 |
| Q9HBE1 | POZ-, AT hook-, and zinc finger-containing protein 1 | PATZ1 | 41 |
| Q9HBE1 | POZ-, AT hook-, and zinc finger-containing protein 1 | PATZ1 | 497 |
| Q9HBE1 | POZ-, AT hook-, and zinc finger-containing protein 1 | PATZ1 | 500 |
| Q9HBE1 | POZ-, AT hook-, and zinc finger-containing protein 1 | PATZ1 | 541 |
| Q9HBE1 | POZ-, AT hook-, and zinc finger-containing protein 1 | PATZ1 | 588 |
| Q15366 | Poly(rC)-binding protein 2 | PCBP2 | 54 |
| Q15366 | Poly(rC)-binding protein 2 | PCBP2 | 109 |
| Q15366 | Poly(rC)-binding protein 2 | PCBP2 | 118 |
| Q15366 | Poly(rC)-binding protein 2 | PCBP2 | 158 |
| Q15366 | Poly(rC)-binding protein 2 | PCBP2 | 163 |
| Q15366 | Poly(rC)-binding protein 2 | PCBP2 | 217 |
| Q15366 | Poly(rC)-binding protein 2 | PCBP2 | 301 |
| P35227 | Polycomb group RING finger protein 2 | PCGF2 | 53 |
| P35227 | Polycomb group RING finger protein 2 | PCGF2 | 56 |
| P35227 | Polycomb group RING finger protein 2 | PCGF2 | 168 |
| P35227 | Polycomb group RING finger protein 2 | PCGF2 | 233 |
| Q3KNV8 | Polycomb group RING finger protein 3 | PCGF3 | 20 |
| Q3KNV8 | Polycomb group RING finger protein 3 | PCGF3 | 113 |
| Q86SE9 | Polycomb group RING finger protein 5 | PCGF5 | 21 |
| Q86SE9 | Polycomb group RING finger protein 5 | PCGF5 | 39 |
| Q86SE9 | Polycomb group RING finger protein 5 | PCGF5 | 42 |
| Q86SE9 | Polycomb group RING finger protein 5 | PCGF5 | 53 |
| Q86SE9 | Polycomb group RING finger protein 5 | PCGF5 | 145 |
| Q9BYE7 | Polycomb group RING finger protein 6 | PCGF6 | 56 |
| Q9BYE7 | Polycomb group RING finger protein 6 | PCGF6 | 137 |
| Q9BYE7 | Polycomb group RING finger protein 6 | PCGF6 | 155 |
| Q96JY6 | PDZ and LIM domain protein 2 | PDLIM2 | 160 |
| Q96JY6 | PDZ and LIM domain protein 2 | PDLIM2 | 286 |
| Q96JY6 | PDZ and LIM domain protein 2 | PDLIM2 | 289 |
| Q96JY6 | PDZ and LIM domain protein 2 | PDLIM2 | 310 |
| Q96JY6 | PDZ and LIM domain protein 2 | PDLIM2 | 313 |
| Q96JY6 | PDZ and LIM domain protein 2 | PDLIM2 | 334 |
| Q9UPQ7 | E3 ubiquitin-protein ligase PDZRN3 | PDZRN3 | 105 |
| Q9UPQ7 | E3 ubiquitin-protein ligase PDZRN3 | PDZRN3 | 117 |
| Q9UPQ7 | E3 ubiquitin-protein ligase PDZRN3 | PDZRN3 | 142 |
| Q9UPQ7 | E3 ubiquitin-protein ligase PDZRN3 | PDZRN3 | 161 |
| Q9UPQ7 | E3 ubiquitin-protein ligase PDZRN3 | PDZRN3 | 162 |
| Q9UPQ7 | E3 ubiquitin-protein ligase PDZRN3 | PDZRN3 | 233 |
| Q6ZMN7 | PDZ domain-containing RING finger protein 4 | PDZRN4 | 802 |
| Q96FA3 | E3 ubiquitin-protein ligase pellino homolog 1 | PELI1 | 61 |
| Q96FA3 | E3 ubiquitin-protein ligase pellino homolog 1 | PELI1 | 212 |
| Q96FA3 | E3 ubiquitin-protein ligase pellino homolog 1 | PELI1 | 282 |
| Q9HAT8 | E3 ubiquitin-protein ligase pellino homolog 2 | PELI2 | 284 |
| Q9HAT8 | E3 ubiquitin-protein ligase pellino homolog 2 | PELI2 | 351 |
| Q9HAT8 | E3 ubiquitin-protein ligase pellino homolog 2 | PELI2 | 373 |
| O60683 | Peroxisome biogenesis factor 10 | PEX10 | 138 |
| O60683 | Peroxisome biogenesis factor 10 | PEX10 | 276 |
| O60683 | Peroxisome biogenesis factor 10 | PEX10 | 307 |
| O00623 | Peroxisome assembly protein 12 | PEX12 | 307 |
| O00623 | Peroxisome assembly protein 12 | PEX12 | 339 |
| Q9BUL5 | PHD finger protein 23 | PHF23 | 359 |
| Q9BUL5 | PHD finger protein 23 | PHF23 | 367 |
| Q9BUL5 | PHD finger protein 23 | PHF23 | 381 |
| Q9BUL5 | PHD finger protein 23 | PHF23 | 384 |
| Q9P1Y6 | PHD and RING finger domain-containing protein 1 | PHRF1 | 126 |
| Q9P1Y6 | PHD and RING finger domain-containing protein 1 | PHRF1 | 131 |
| Q9P1Y6 | PHD and RING finger domain-containing protein 1 | PHRF1 | 145 |
| Q9P1Y6 | PHD and RING finger domain-containing protein 1 | PHRF1 | 407 |
| Q9P1Y6 | PHD and RING finger domain-containing protein 1 | PHRF1 | 558 |
| Q9P1Y6 | PHD and RING finger domain-containing protein 1 | PHRF1 | 585 |
| Q9P1Y6 | PHD and RING finger domain-containing protein 1 | PHRF1 | 658 |
| Q9P1Y6 | PHD and RING finger domain-containing protein 1 | PHRF1 | 710 |
| Q9P1Y6 | PHD and RING finger domain-containing protein 1 | PHRF1 | 797 |
| Q9P1Y6 | PHD and RING finger domain-containing protein 1 | PHRF1 | 878 |
| Q9P1Y6 | PHD and RING finger domain-containing protein 1 | PHRF1 | 949 |
| Q9P1Y6 | PHD and RING finger domain-containing protein 1 | PHRF1 | 962 |
| Q9P1Y6 | PHD and RING finger domain-containing protein 1 | PHRF1 | 1123 |
| O75925 | E3 SUMO-protein ligase PIAS1 | PIAS1 | 49 |
| O75925 | E3 SUMO-protein ligase PIAS1 | PIAS1 | 220 |
| O75925 | E3 SUMO-protein ligase PIAS1 | PIAS1 | 228 |
| O75925 | E3 SUMO-protein ligase PIAS1 | PIAS1 | 335 |
| O75925 | E3 SUMO-protein ligase PIAS1 | PIAS1 | 346 |
| O75925 | E3 SUMO-protein ligase PIAS1 | PIAS1 | 374 |
| O75925 | E3 SUMO-protein ligase PIAS1 | PIAS1 | 377 |
| O75925 | E3 SUMO-protein ligase PIAS1 | PIAS1 | 399 |
| O75925 | E3 SUMO-protein ligase PIAS1 | PIAS1 | 402 |
| O75925 | E3 SUMO-protein ligase PIAS1 | PIAS1 | 435 |
| O75925 | E3 SUMO-protein ligase PIAS1 | PIAS1 | 481 |
| O75928 | E3 SUMO-protein ligase PIAS2 | PIAS2 | 49 |
| O75928 | E3 SUMO-protein ligase PIAS2 | PIAS2 | 192 |
| O75928 | E3 SUMO-protein ligase PIAS2 | PIAS2 | 220 |
| O75928 | E3 SUMO-protein ligase PIAS2 | PIAS2 | 231 |
| O75928 | E3 SUMO-protein ligase PIAS2 | PIAS2 | 346 |
| O75928 | E3 SUMO-protein ligase PIAS2 | PIAS2 | 357 |
| O75928 | E3 SUMO-protein ligase PIAS2 | PIAS2 | 362 |
| O75928 | E3 SUMO-protein ligase PIAS2 | PIAS2 | 385 |
| O75928 | E3 SUMO-protein ligase PIAS2 | PIAS2 | 388 |
| O75928 | E3 SUMO-protein ligase PIAS2 | PIAS2 | 441 |
| O75928 | E3 SUMO-protein ligase PIAS2 | PIAS2 | 454 |
| O75928 | E3 SUMO-protein ligase PIAS2 | PIAS2 | 490 |
| Q9Y6X2 | E3 SUMO-protein ligase PIAS3 | PIAS3 | 49 |
| Q9Y6X2 | E3 SUMO-protein ligase PIAS3 | PIAS3 | 184 |
| Q9Y6X2 | E3 SUMO-protein ligase PIAS3 | PIAS3 | 327 |
| Q9Y6X2 | E3 SUMO-protein ligase PIAS3 | PIAS3 | 338 |
| Q9Y6X2 | E3 SUMO-protein ligase PIAS3 | PIAS3 | 418 |
| Q9Y6X2 | E3 SUMO-protein ligase PIAS3 | PIAS3 | 468 |
| Q9Y6X2 | E3 SUMO-protein ligase PIAS3 | PIAS3 | 582 |
| Q9Y6X2 | E3 SUMO-protein ligase PIAS3 | PIAS3 | 620 |
| Q8N2W9 | E3 SUMO-protein ligase PIAS4 | PIAS4 | 50 |
| Q8N2W9 | E3 SUMO-protein ligase PIAS4 | PIAS4 | 164 |
| Q8N2W9 | E3 SUMO-protein ligase PIAS4 | PIAS4 | 221 |
| Q8N2W9 | E3 SUMO-protein ligase PIAS4 | PIAS4 | 299 |
| Q8N2W9 | E3 SUMO-protein ligase PIAS4 | PIAS4 | 326 |
| Q8N2W9 | E3 SUMO-protein ligase PIAS4 | PIAS4 | 337 |
| Q8N2W9 | E3 SUMO-protein ligase PIAS4 | PIAS4 | 365 |
| Q8N2W9 | E3 SUMO-protein ligase PIAS4 | PIAS4 | 368 |
| Q8N2W9 | E3 SUMO-protein ligase PIAS4 | PIAS4 | 500 |
| Q8NG27 | E3 ubiquitin-protein ligase Praja-1 | PJA1 | 241 |
| Q8NG27 | E3 ubiquitin-protein ligase Praja-1 | PJA1 | 268 |
| Q8NG27 | E3 ubiquitin-protein ligase Praja-1 | PJA1 | 361 |
| Q8NG27 | E3 ubiquitin-protein ligase Praja-1 | PJA1 | 632 |
| Q8NG27 | E3 ubiquitin-protein ligase Praja-1 | PJA1 | 635 |
| O43164 | E3 ubiquitin-protein ligase Praja-2 | PJA2 | 48 |
| O43164 | E3 ubiquitin-protein ligase Praja-2 | PJA2 | 224 |
| O43164 | E3 ubiquitin-protein ligase Praja-2 | PJA2 | 288 |
| O43164 | E3 ubiquitin-protein ligase Praja-2 | PJA2 | 294 |
| O43164 | E3 ubiquitin-protein ligase Praja-2 | PJA2 | 671 |
| O43164 | E3 ubiquitin-protein ligase Praja-2 | PJA2 | 674 |
| P29590 | Protein PML | PML | 57 |
| P29590 | Protein PML | PML | 60 |
| P29590 | Protein PML | PML | 72 |
| P29590 | Protein PML | PML | 77 |
| P29590 | Protein PML | PML | 80 |
| P29590 | Protein PML | PML | 129 |
| P29590 | Protein PML | PML | 132 |
| P29590 | Protein PML | PML | 140 |
| P29590 | Protein PML | PML | 143 |
| P29590 | Protein PML | PML | 148 |
| P29590 | Protein PML | PML | 151 |
| P29590 | Protein PML | PML | 189 |
| P29590 | Protein PML | PML | 204 |
| P29590 | Protein PML | PML | 207 |
| P29590 | Protein PML | PML | 212 |
| P29590 | Protein PML | PML | 213 |
| P29590 | Protein PML | PML | 215 |
| P29590 | Protein PML | PML | 227 |
| P29590 | Protein PML | PML | 338 |
| P29590 | Protein PML | PML | 389 |
| P29590 | Protein PML | PML | 479 |
| P29590 | Protein PML | PML | 484 |
| P29590 | Protein PML | PML | 676 |
| Q13356 | RING-type E3 ubiquitin-protein ligase PPIL2 | PPIL2 | 15 |
| Q13356 | RING-type E3 ubiquitin-protein ligase PPIL2 | PPIL2 | 103 |
| Q13356 | RING-type E3 ubiquitin-protein ligase PPIL2 | PPIL2 | 304 |
| Q13356 | RING-type E3 ubiquitin-protein ligase PPIL2 | PPIL2 | 387 |
| O60260 | E3 ubiquitin-protein ligase parkin | PRKN | 293 |
| Q9UMS4 | Pre-mRNA-processing factor 19 | PRPF19 | 114 |
| Q9UMS4 | Pre-mRNA-processing factor 19 | PRPF19 | 230 |
| Q9UMS4 | Pre-mRNA-processing factor 19 | PRPF19 | 298 |
| Q9UMS4 | Pre-mRNA-processing factor 19 | PRPF19 | 351 |
| Q9UMS4 | Pre-mRNA-processing factor 19 | PRPF19 | 355 |
| Q9UJ41 | Rab5 GDP/GTP exchange factor | RABGEF1 | 157 |
| Q9UJ41 | Rab5 GDP/GTP exchange factor | RABGEF1 | 161 |
| Q9UJ41 | Rab5 GDP/GTP exchange factor | RABGEF1 | 173 |
| Q9UJ41 | Rab5 GDP/GTP exchange factor | RABGEF1 | 176 |
| Q9UJ41 | Rab5 GDP/GTP exchange factor | RABGEF1 | 359 |
| Q9UJ41 | Rab5 GDP/GTP exchange factor | RABGEF1 | 442 |
| Q9UJ41 | Rab5 GDP/GTP exchange factor | RABGEF1 | 471 |
| Q9UJ41 | Rab5 GDP/GTP exchange factor | RABGEF1 | 507 |
| Q9UJ41 | Rab5 GDP/GTP exchange factor | RABGEF1 | 559 |
| Q9UJ41 | Rab5 GDP/GTP exchange factor | RABGEF1 | 621 |
| Q9NS91 | E3 ubiquitin-protein ligase RAD18 | RAD18 | 63 |
| Q9NS91 | E3 ubiquitin-protein ligase RAD18 | RAD18 | 64 |
| Q9NS91 | E3 ubiquitin-protein ligase RAD18 | RAD18 | 207 |
| Q9NS91 | E3 ubiquitin-protein ligase RAD18 | RAD18 | 223 |
| Q9NS91 | E3 ubiquitin-protein ligase RAD18 | RAD18 | 289 |
| Q9NS91 | E3 ubiquitin-protein ligase RAD18 | RAD18 | 388 |
| P49792 | E3 SUMO-protein ligase RanBP2 | RANBP2 | 50 |
| P49792 | E3 SUMO-protein ligase RanBP2 | RANBP2 | 82 |
| P49792 | E3 SUMO-protein ligase RanBP2 | RANBP2 | 105 |
| P49792 | E3 SUMO-protein ligase RanBP2 | RANBP2 | 141 |
| P49792 | E3 SUMO-protein ligase RanBP2 | RANBP2 | 188 |
| P49792 | E3 SUMO-protein ligase RanBP2 | RANBP2 | 206 |
| P49792 | E3 SUMO-protein ligase RanBP2 | RANBP2 | 220 |
| P49792 | E3 SUMO-protein ligase RanBP2 | RANBP2 | 348 |
| P49792 | E3 SUMO-protein ligase RanBP2 | RANBP2 | 503 |
| P49792 | E3 SUMO-protein ligase RanBP2 | RANBP2 | 514 |
| P49792 | E3 SUMO-protein ligase RanBP2 | RANBP2 | 520 |
| P49792 | E3 SUMO-protein ligase RanBP2 | RANBP2 | 524 |
| P49792 | E3 SUMO-protein ligase RanBP2 | RANBP2 | 536 |
| P49792 | E3 SUMO-protein ligase RanBP2 | RANBP2 | 581 |
| P49792 | E3 SUMO-protein ligase RanBP2 | RANBP2 | 707 |
| P49792 | E3 SUMO-protein ligase RanBP2 | RANBP2 | 815 |
| P49792 | E3 SUMO-protein ligase RanBP2 | RANBP2 | 1196 |
| P49792 | E3 SUMO-protein ligase RanBP2 | RANBP2 | 1243 |
| P49792 | E3 SUMO-protein ligase RanBP2 | RANBP2 | 1296 |
| P49792 | E3 SUMO-protein ligase RanBP2 | RANBP2 | 1335 |
| P49792 | E3 SUMO-protein ligase RanBP2 | RANBP2 | 1358 |
| P49792 | E3 SUMO-protein ligase RanBP2 | RANBP2 | 1361 |
| P49792 | E3 SUMO-protein ligase RanBP2 | RANBP2 | 1372 |
| P49792 | E3 SUMO-protein ligase RanBP2 | RANBP2 | 1375 |
| P49792 | E3 SUMO-protein ligase RanBP2 | RANBP2 | 1421 |
| P49792 | E3 SUMO-protein ligase RanBP2 | RANBP2 | 1424 |
| P49792 | E3 SUMO-protein ligase RanBP2 | RANBP2 | 1435 |
| P49792 | E3 SUMO-protein ligase RanBP2 | RANBP2 | 1438 |
| P49792 | E3 SUMO-protein ligase RanBP2 | RANBP2 | 1485 |
| P49792 | E3 SUMO-protein ligase RanBP2 | RANBP2 | 1488 |
| P49792 | E3 SUMO-protein ligase RanBP2 | RANBP2 | 1499 |
| P49792 | E3 SUMO-protein ligase RanBP2 | RANBP2 | 1502 |
| P49792 | E3 SUMO-protein ligase RanBP2 | RANBP2 | 1549 |
| P49792 | E3 SUMO-protein ligase RanBP2 | RANBP2 | 1552 |
| P49792 | E3 SUMO-protein ligase RanBP2 | RANBP2 | 1563 |
| P49792 | E3 SUMO-protein ligase RanBP2 | RANBP2 | 1566 |
| P49792 | E3 SUMO-protein ligase RanBP2 | RANBP2 | 1612 |
| P49792 | E3 SUMO-protein ligase RanBP2 | RANBP2 | 1615 |
| P49792 | E3 SUMO-protein ligase RanBP2 | RANBP2 | 1626 |
| P49792 | E3 SUMO-protein ligase RanBP2 | RANBP2 | 1629 |
| P49792 | E3 SUMO-protein ligase RanBP2 | RANBP2 | 1744 |
| P49792 | E3 SUMO-protein ligase RanBP2 | RANBP2 | 1747 |
| P49792 | E3 SUMO-protein ligase RanBP2 | RANBP2 | 1749 |
| P49792 | E3 SUMO-protein ligase RanBP2 | RANBP2 | 1787 |
| P49792 | E3 SUMO-protein ligase RanBP2 | RANBP2 | 1790 |
| P49792 | E3 SUMO-protein ligase RanBP2 | RANBP2 | 1791 |
| P49792 | E3 SUMO-protein ligase RanBP2 | RANBP2 | 1801 |
| P49792 | E3 SUMO-protein ligase RanBP2 | RANBP2 | 1804 |
| P49792 | E3 SUMO-protein ligase RanBP2 | RANBP2 | 2084 |
| P49792 | E3 SUMO-protein ligase RanBP2 | RANBP2 | 2142 |
| P49792 | E3 SUMO-protein ligase RanBP2 | RANBP2 | 2381 |
| P49792 | E3 SUMO-protein ligase RanBP2 | RANBP2 | 2407 |
| P49792 | E3 SUMO-protein ligase RanBP2 | RANBP2 | 2464 |
| P49792 | E3 SUMO-protein ligase RanBP2 | RANBP2 | 2577 |
| P49792 | E3 SUMO-protein ligase RanBP2 | RANBP2 | 2659 |
| P49792 | E3 SUMO-protein ligase RanBP2 | RANBP2 | 2696 |
| P49792 | E3 SUMO-protein ligase RanBP2 | RANBP2 | 2710 |
| P49792 | E3 SUMO-protein ligase RanBP2 | RANBP2 | 2737 |
| P49792 | E3 SUMO-protein ligase RanBP2 | RANBP2 | 2740 |
| P49792 | E3 SUMO-protein ligase RanBP2 | RANBP2 | 2791 |
| P49792 | E3 SUMO-protein ligase RanBP2 | RANBP2 | 2982 |
| P49792 | E3 SUMO-protein ligase RanBP2 | RANBP2 | 3032 |
| P49792 | E3 SUMO-protein ligase RanBP2 | RANBP2 | 3040 |
| P49792 | E3 SUMO-protein ligase RanBP2 | RANBP2 | 3071 |
| P49792 | E3 SUMO-protein ligase RanBP2 | RANBP2 | 3100 |
| P49792 | E3 SUMO-protein ligase RanBP2 | RANBP2 | 3122 |
| P49792 | E3 SUMO-protein ligase RanBP2 | RANBP2 | 3221 |
| Q7Z6E9 | E3 ubiquitin-protein ligase RBBP6 | RBBP6 | 27 |
| Q7Z6E9 | E3 ubiquitin-protein ligase RBBP6 | RBBP6 | 44 |
| Q7Z6E9 | E3 ubiquitin-protein ligase RBBP6 | RBBP6 | 161 |
| Q7Z6E9 | E3 ubiquitin-protein ligase RBBP6 | RBBP6 | 164 |
| Q7Z6E9 | E3 ubiquitin-protein ligase RBBP6 | RBBP6 | 174 |
| Q7Z6E9 | E3 ubiquitin-protein ligase RBBP6 | RBBP6 | 296 |
| Q7Z6E9 | E3 ubiquitin-protein ligase RBBP6 | RBBP6 | 299 |
| Q7Z6E9 | E3 ubiquitin-protein ligase RBBP6 | RBBP6 | 1566 |
| Q9BYM8 | RanBP-type and C3HC4-type zinc finger-containing protein 1 | RBCK1 | 30 |
| Q9BYM8 | RanBP-type and C3HC4-type zinc finger-containing protein 1 | RBCK1 | 199 |
| Q9BYM8 | RanBP-type and C3HC4-type zinc finger-containing protein 1 | RBCK1 | 216 |
| Q9BYM8 | RanBP-type and C3HC4-type zinc finger-containing protein 1 | RBCK1 | 217 |
| Q9BYM8 | RanBP-type and C3HC4-type zinc finger-containing protein 1 | RBCK1 | 285 |
| Q9BYM8 | RanBP-type and C3HC4-type zinc finger-containing protein 1 | RBCK1 | 300 |
| Q9BYM8 | RanBP-type and C3HC4-type zinc finger-containing protein 1 | RBCK1 | 305 |
| Q9BYM8 | RanBP-type and C3HC4-type zinc finger-containing protein 1 | RBCK1 | 308 |
| Q9BYM8 | RanBP-type and C3HC4-type zinc finger-containing protein 1 | RBCK1 | 323 |
| Q9BYM8 | RanBP-type and C3HC4-type zinc finger-containing protein 1 | RBCK1 | 371 |
| Q9BYM8 | RanBP-type and C3HC4-type zinc finger-containing protein 1 | RBCK1 | 394 |
| Q9BYM8 | RanBP-type and C3HC4-type zinc finger-containing protein 1 | RBCK1 | 411 |
| Q9BYM8 | RanBP-type and C3HC4-type zinc finger-containing protein 1 | RBCK1 | 447 |
| Q9BYM8 | RanBP-type and C3HC4-type zinc finger-containing protein 1 | RBCK1 | 450 |
| Q9BYM8 | RanBP-type and C3HC4-type zinc finger-containing protein 1 | RBCK1 | 460 |
| Q9BYM8 | RanBP-type and C3HC4-type zinc finger-containing protein 1 | RBCK1 | 502 |
| P62877 | E3 ubiquitin-protein ligase RBX1 | RBX1 | 42 |
| P62877 | E3 ubiquitin-protein ligase RBX1 | RBX1 | 94 |
| Q5TC82 | Roquin-1 | RC3H1 | 87 |
| Q5TC82 | Roquin-1 | RC3H1 | 102 |
| Q5TC82 | Roquin-1 | RC3H1 | 434 |
| Q5TC82 | Roquin-1 | RC3H1 | 815 |
| Q5TC82 | Roquin-1 | RC3H1 | 1049 |
| Q9HBD1 | Roquin-2 | RC3H2 | 425 |
| Q9HBD1 | Roquin-2 | RC3H2 | 747 |
| Q9HBD1 | Roquin-2 | RC3H2 | 764 |
| Q9HBD1 | Roquin-2 | RC3H2 | 771 |
| Q8NDN9 | RCC1 and BTB domain-containing protein 1 | RCBTB1 | 72 |
| Q8NDN9 | RCC1 and BTB domain-containing protein 1 | RCBTB1 | 177 |
| Q8NDN9 | RCC1 and BTB domain-containing protein 1 | RCBTB1 | 392 |
| O95199 | RCC1 and BTB domain-containing protein 2 | RCBTB2 | 201 |
| O95199 | RCC1 and BTB domain-containing protein 2 | RCBTB2 | 416 |
| O95199 | RCC1 and BTB domain-containing protein 2 | RCBTB2 | 488 |
| Q96PM5 | RING finger and CHY zinc finger domain-containing protein 1 | RCHY1 | 20 |
| Q96PM5 | RING finger and CHY zinc finger domain-containing protein 1 | RCHY1 | 27 |
| Q96PM5 | RING finger and CHY zinc finger domain-containing protein 1 | RCHY1 | 33 |
| Q96PM5 | RING finger and CHY zinc finger domain-containing protein 1 | RCHY1 | 43 |
| Q96PM5 | RING finger and CHY zinc finger domain-containing protein 1 | RCHY1 | 65 |
| Q96PM5 | RING finger and CHY zinc finger domain-containing protein 1 | RCHY1 | 102 |
| Q96PM5 | RING finger and CHY zinc finger domain-containing protein 1 | RCHY1 | 105 |
| Q96PM5 | RING finger and CHY zinc finger domain-containing protein 1 | RCHY1 | 108 |
| Q96PM5 | RING finger and CHY zinc finger domain-containing protein 1 | RCHY1 | 119 |
| Q96PM5 | RING finger and CHY zinc finger domain-containing protein 1 | RCHY1 | 125 |
| Q96PM5 | RING finger and CHY zinc finger domain-containing protein 1 | RCHY1 | 136 |
| Q96PM5 | RING finger and CHY zinc finger domain-containing protein 1 | RCHY1 | 145 |
| Q96PM5 | RING finger and CHY zinc finger domain-containing protein 1 | RCHY1 | 148 |
| Q96PM5 | RING finger and CHY zinc finger domain-containing protein 1 | RCHY1 | 164 |
| Q96PM5 | RING finger and CHY zinc finger domain-containing protein 1 | RCHY1 | 172 |
| Q96PM5 | RING finger and CHY zinc finger domain-containing protein 1 | RCHY1 | 183 |
| Q96PM5 | RING finger and CHY zinc finger domain-containing protein 1 | RCHY1 | 186 |
| Q8WZ73 | E3 ubiquitin-protein ligase rififylin | RFFL | 47 |
| Q8WZ73 | E3 ubiquitin-protein ligase rififylin | RFFL | 50 |
| Q8WZ73 | E3 ubiquitin-protein ligase rififylin | RFFL | 66 |
| Q8WZ73 | E3 ubiquitin-protein ligase rififylin | RFFL | 71 |
| Q8WZ73 | E3 ubiquitin-protein ligase rififylin | RFFL | 74 |
| Q8WZ73 | E3 ubiquitin-protein ligase rififylin | RFFL | 85 |
| Q8WZ73 | E3 ubiquitin-protein ligase rififylin | RFFL | 123 |
| Q8WZ73 | E3 ubiquitin-protein ligase rififylin | RFFL | 272 |
| Q8WZ73 | E3 ubiquitin-protein ligase rififylin | RFFL | 273 |
| Q8WZ73 | E3 ubiquitin-protein ligase rififylin | RFFL | 319 |
| Q8WZ73 | E3 ubiquitin-protein ligase rififylin | RFFL | 337 |
| Q6PCD5 | E3 ubiquitin-protein ligase RFWD3 | RFWD3 | 307 |
| Q6PCD5 | E3 ubiquitin-protein ligase RFWD3 | RFWD3 | 315 |
| Q6PCD5 | E3 ubiquitin-protein ligase RFWD3 | RFWD3 | 327 |
| Q6PCD5 | E3 ubiquitin-protein ligase RFWD3 | RFWD3 | 330 |
| Q6PCD5 | E3 ubiquitin-protein ligase RFWD3 | RFWD3 | 379 |
| Q6PCD5 | E3 ubiquitin-protein ligase RFWD3 | RFWD3 | 424 |
| Q6PCD5 | E3 ubiquitin-protein ligase RFWD3 | RFWD3 | 450 |
| Q6PCD5 | E3 ubiquitin-protein ligase RFWD3 | RFWD3 | 586 |
| Q6PCD5 | E3 ubiquitin-protein ligase RFWD3 | RFWD3 | 696 |
| Q06587 | E3 ubiquitin-protein ligase RING1 | RING1 | 48 |
| Q06587 | E3 ubiquitin-protein ligase RING1 | RING1 | 51 |
| Q06587 | E3 ubiquitin-protein ligase RING1 | RING1 | 69 |
| Q06587 | E3 ubiquitin-protein ligase RING1 | RING1 | 72 |
| Q06587 | E3 ubiquitin-protein ligase RING1 | RING1 | 84 |
| Q06587 | E3 ubiquitin-protein ligase RING1 | RING1 | 87 |
| Q06587 | E3 ubiquitin-protein ligase RING1 | RING1 | 279 |
| Q06587 | E3 ubiquitin-protein ligase RING1 | RING1 | 332 |
| Q06587 | E3 ubiquitin-protein ligase RING1 | RING1 | 398 |
| Q9NVW2 | E3 ubiquitin-protein ligase RLIM | RLIM | 570 |
| Q9NVW2 | E3 ubiquitin-protein ligase RLIM | RLIM | 573 |
| Q9NVW2 | E3 ubiquitin-protein ligase RLIM | RLIM | 588 |
| Q9NVW2 | E3 ubiquitin-protein ligase RLIM | RLIM | 596 |
| Q8N5U6 | RING finger protein 10 | RNF10 | 218 |
| Q8N5U6 | RING finger protein 10 | RNF10 | 225 |
| Q8N5U6 | RING finger protein 10 | RNF10 | 228 |
| Q8N5U6 | RING finger protein 10 | RNF10 | 366 |
| Q8N5U6 | RING finger protein 10 | RNF10 | 483 |
| Q8N5U6 | RING finger protein 10 | RNF10 | 650 |
| Q6ZNA4 | E3 ubiquitin-protein ligase Arkadia | RNF111 | 68 |
| Q6ZNA4 | E3 ubiquitin-protein ligase Arkadia | RNF111 | 108 |
| Q6ZNA4 | E3 ubiquitin-protein ligase Arkadia | RNF111 | 201 |
| Q6ZNA4 | E3 ubiquitin-protein ligase Arkadia | RNF111 | 592 |
| Q6ZNA4 | E3 ubiquitin-protein ligase Arkadia | RNF111 | 905 |
| Q6ZNA4 | E3 ubiquitin-protein ligase Arkadia | RNF111 | 979 |
| Q6ZNA4 | E3 ubiquitin-protein ligase Arkadia | RNF111 | 982 |
| Q9ULX5 | RING finger protein 112 | RNF112 | 77 |
| O15541 | RING finger protein 113A | RNF113A | 15 |
| O15541 | RING finger protein 113A | RNF113A | 36 |
| O15541 | RING finger protein 113A | RNF113A | 51 |
| O15541 | RING finger protein 113A | RNF113A | 202 |
| O15541 | RING finger protein 113A | RNF113A | 282 |
| O15541 | RING finger protein 113A | RNF113A | 285 |
| O15541 | RING finger protein 113A | RNF113A | 296 |
| O15541 | RING finger protein 113A | RNF113A | 299 |
| Q8IZP6 | RING finger protein 113B | RNF113B | 196 |
| Q9Y508 | E3 ubiquitin-protein ligase RNF114 | RNF114 | 8 |
| Q9Y508 | E3 ubiquitin-protein ligase RNF114 | RNF114 | 29 |
| Q9Y508 | E3 ubiquitin-protein ligase RNF114 | RNF114 | 32 |
| Q9Y508 | E3 ubiquitin-protein ligase RNF114 | RNF114 | 44 |
| Q9Y508 | E3 ubiquitin-protein ligase RNF114 | RNF114 | 49 |
| Q9Y508 | E3 ubiquitin-protein ligase RNF114 | RNF114 | 52 |
| Q9Y508 | E3 ubiquitin-protein ligase RNF114 | RNF114 | 56 |
| Q9Y508 | E3 ubiquitin-protein ligase RNF114 | RNF114 | 64 |
| Q9Y508 | E3 ubiquitin-protein ligase RNF114 | RNF114 | 67 |
| Q9Y508 | E3 ubiquitin-protein ligase RNF114 | RNF114 | 91 |
| Q9Y508 | E3 ubiquitin-protein ligase RNF114 | RNF114 | 94 |
| Q9Y508 | E3 ubiquitin-protein ligase RNF114 | RNF114 | 110 |
| Q9Y508 | E3 ubiquitin-protein ligase RNF114 | RNF114 | 143 |
| Q9Y508 | E3 ubiquitin-protein ligase RNF114 | RNF114 | 146 |
| Q9Y508 | E3 ubiquitin-protein ligase RNF114 | RNF114 | 160 |
| Q9Y508 | E3 ubiquitin-protein ligase RNF114 | RNF114 | 173 |
| Q9Y508 | E3 ubiquitin-protein ligase RNF114 | RNF114 | 176 |
| Q9H920 | RING finger protein 121 | RNF121 | 251 |
| Q9H920 | RING finger protein 121 | RNF121 | 264 |
| Q9H920 | RING finger protein 121 | RNF121 | 272 |
| Q9H920 | RING finger protein 121 | RNF121 | 275 |
| Q5XPI4 | E3 ubiquitin-protein ligase RNF123 | RNF123 | 461 |
| Q5XPI4 | E3 ubiquitin-protein ligase RNF123 | RNF123 | 977 |
| Q5XPI4 | E3 ubiquitin-protein ligase RNF123 | RNF123 | 1180 |
| Q5XPI4 | E3 ubiquitin-protein ligase RNF123 | RNF123 | 1277 |
| Q9BV68 | E3 ubiquitin-protein ligase RNF126 | RNF126 | 13 |
| Q9BV68 | E3 ubiquitin-protein ligase RNF126 | RNF126 | 15 |
| Q9BV68 | E3 ubiquitin-protein ligase RNF126 | RNF126 | 16 |
| Q9BV68 | E3 ubiquitin-protein ligase RNF126 | RNF126 | 29 |
| Q9BV68 | E3 ubiquitin-protein ligase RNF126 | RNF126 | 32 |
| Q9BV68 | E3 ubiquitin-protein ligase RNF126 | RNF126 | 229 |
| Q9BV68 | E3 ubiquitin-protein ligase RNF126 | RNF126 | 232 |
| Q9BV68 | E3 ubiquitin-protein ligase RNF126 | RNF126 | 266 |
| Q8TEB7 | E3 ubiquitin-protein ligase RNF128 | RNF128 | 295 |
| Q8TEB7 | E3 ubiquitin-protein ligase RNF128 | RNF128 | 303 |
| Q8TEB7 | E3 ubiquitin-protein ligase RNF128 | RNF128 | 314 |
| Q8TEB7 | E3 ubiquitin-protein ligase RNF128 | RNF128 | 317 |
| O43567 | E3 ubiquitin-protein ligase RNF13 | RNF13 | 258 |
| O43567 | E3 ubiquitin-protein ligase RNF13 | RNF13 | 264 |
| O43567 | E3 ubiquitin-protein ligase RNF13 | RNF13 | 266 |
| O43567 | E3 ubiquitin-protein ligase RNF13 | RNF13 | 278 |
| O43567 | E3 ubiquitin-protein ligase RNF13 | RNF13 | 281 |
| Q86XS8 | E3 ubiquitin-protein ligase RNF130 | RNF130 | 320 |
| Q8IUD6 | E3 ubiquitin-protein ligase RNF135 | RNF135 | 44 |
| Q8IUD6 | E3 ubiquitin-protein ligase RNF135 | RNF135 | 59 |
| Q8IUD6 | E3 ubiquitin-protein ligase RNF135 | RNF135 | 62 |
| Q8IUD6 | E3 ubiquitin-protein ligase RNF135 | RNF135 | 101 |
| Q8IUD6 | E3 ubiquitin-protein ligase RNF135 | RNF135 | 103 |
| Q8IUD6 | E3 ubiquitin-protein ligase RNF135 | RNF135 | 238 |
| Q8IUD6 | E3 ubiquitin-protein ligase RNF135 | RNF135 | 270 |
| Q8WVD3 | E3 ubiquitin-protein ligase RNF138 | RNF138 | 39 |
| Q8WVD3 | E3 ubiquitin-protein ligase RNF138 | RNF138 | 42 |
| Q8WVD3 | E3 ubiquitin-protein ligase RNF138 | RNF138 | 54 |
| Q8WVD3 | E3 ubiquitin-protein ligase RNF138 | RNF138 | 57 |
| Q8WVD3 | E3 ubiquitin-protein ligase RNF138 | RNF138 | 68 |
| Q8WVD3 | E3 ubiquitin-protein ligase RNF138 | RNF138 | 86 |
| Q8WVD3 | E3 ubiquitin-protein ligase RNF138 | RNF138 | 159 |
| Q8WVD3 | E3 ubiquitin-protein ligase RNF138 | RNF138 | 162 |
| Q9UBS8 | E3 ubiquitin-protein ligase RNF14 | RNF14 | 57 |
| Q9UBS8 | E3 ubiquitin-protein ligase RNF14 | RNF14 | 68 |
| Q9UBS8 | E3 ubiquitin-protein ligase RNF14 | RNF14 | 223 |
| Q9UBS8 | E3 ubiquitin-protein ligase RNF14 | RNF14 | 225 |
| Q9UBS8 | E3 ubiquitin-protein ligase RNF14 | RNF14 | 246 |
| Q9UBS8 | E3 ubiquitin-protein ligase RNF14 | RNF14 | 262 |
| Q9UBS8 | E3 ubiquitin-protein ligase RNF14 | RNF14 | 270 |
| Q9UBS8 | E3 ubiquitin-protein ligase RNF14 | RNF14 | 350 |
| Q9UBS8 | E3 ubiquitin-protein ligase RNF14 | RNF14 | 404 |
| Q9UBS8 | E3 ubiquitin-protein ligase RNF14 | RNF14 | 406 |
| Q9UBS8 | E3 ubiquitin-protein ligase RNF14 | RNF14 | 407 |
| Q9UBS8 | E3 ubiquitin-protein ligase RNF14 | RNF14 | 417 |
| Q9UBS8 | E3 ubiquitin-protein ligase RNF14 | RNF14 | 453 |
| Q8WVD5 | RING finger protein 141 | RNF141 | 169 |
| Q8WVD5 | RING finger protein 141 | RNF141 | 174 |
| Q8WVD5 | RING finger protein 141 | RNF141 | 177 |
| Q8WVD5 | RING finger protein 141 | RNF141 | 191 |
| Q7Z419 | E3 ubiquitin-protein ligase RNF144B | RNF144B | 33 |
| Q7Z419 | E3 ubiquitin-protein ligase RNF144B | RNF144B | 35 |
| Q7Z419 | E3 ubiquitin-protein ligase RNF144B | RNF144B | 53 |
| Q7Z419 | E3 ubiquitin-protein ligase RNF144B | RNF144B | 56 |
| Q7Z419 | E3 ubiquitin-protein ligase RNF144B | RNF144B | 153 |
| Q7Z419 | E3 ubiquitin-protein ligase RNF144B | RNF144B | 155 |
| Q7Z419 | E3 ubiquitin-protein ligase RNF144B | RNF144B | 156 |
| Q7Z419 | E3 ubiquitin-protein ligase RNF144B | RNF144B | 166 |
| Q7Z419 | E3 ubiquitin-protein ligase RNF144B | RNF144B | 193 |
| Q7Z419 | E3 ubiquitin-protein ligase RNF144B | RNF144B | 196 |
| Q7Z419 | E3 ubiquitin-protein ligase RNF144B | RNF144B | 206 |
| Q7Z419 | E3 ubiquitin-protein ligase RNF144B | RNF144B | 240 |
| Q9NTX7 | E3 ubiquitin-protein ligase RNF146 | RNF146 | 25 |
| Q9NTX7 | E3 ubiquitin-protein ligase RNF146 | RNF146 | 37 |
| Q9NTX7 | E3 ubiquitin-protein ligase RNF146 | RNF146 | 40 |
| Q9NTX7 | E3 ubiquitin-protein ligase RNF146 | RNF146 | 57 |
| Q9NTX7 | E3 ubiquitin-protein ligase RNF146 | RNF146 | 60 |
| Q9NTX7 | E3 ubiquitin-protein ligase RNF146 | RNF146 | 185 |
| Q9NTX7 | E3 ubiquitin-protein ligase RNF146 | RNF146 | 354 |
| Q8N7C7 | RING finger protein 148 | RNF148 | 295 |
| Q8N7C7 | RING finger protein 148 | RNF148 | 298 |
| Q8NC42 | E3 ubiquitin-protein ligase RNF149 | RNF149 | 90 |
| Q8NC42 | E3 ubiquitin-protein ligase RNF149 | RNF149 | 295 |
| Q8NC42 | E3 ubiquitin-protein ligase RNF149 | RNF149 | 306 |
| Q8NC42 | E3 ubiquitin-protein ligase RNF149 | RNF149 | 309 |
| Q9ULK6 | RING finger protein 150 | RNF150 | 315 |
| Q9ULK6 | RING finger protein 150 | RNF150 | 318 |
| Q96PX1 | E3 ubiquitin ligase RNF157 | RNF157 | 100 |
| Q96PX1 | E3 ubiquitin ligase RNF157 | RNF157 | 292 |
| Q96PX1 | E3 ubiquitin ligase RNF157 | RNF157 | 312 |
| Q96PX1 | E3 ubiquitin ligase RNF157 | RNF157 | 315 |
| Q6ZSG1 | E3 ubiquitin-protein ligase RNF165 | RNF165 | 331 |
| Q6ZSG1 | E3 ubiquitin-protein ligase RNF165 | RNF165 | 334 |
| Q96A37 | RING finger protein 166 | RNF166 | 117 |
| Q96A37 | RING finger protein 166 | RNF166 | 127 |
| Q9H6Y7 | E3 ubiquitin-protein ligase RNF167 | RNF167 | 248 |
| Q9H6Y7 | E3 ubiquitin-protein ligase RNF167 | RNF167 | 256 |
| Q9H6Y7 | E3 ubiquitin-protein ligase RNF167 | RNF167 | 268 |
| Q9H6Y7 | E3 ubiquitin-protein ligase RNF167 | RNF167 | 271 |
| Q8IYW5 | E3 ubiquitin-protein ligase RNF168 | RNF168 | 50 |
| Q8IYW5 | E3 ubiquitin-protein ligase RNF168 | RNF168 | 54 |
| Q8IYW5 | E3 ubiquitin-protein ligase RNF168 | RNF168 | 190 |
| Q8IYW5 | E3 ubiquitin-protein ligase RNF168 | RNF168 | 326 |
| Q8IYW5 | E3 ubiquitin-protein ligase RNF168 | RNF168 | 348 |
| Q8IYW5 | E3 ubiquitin-protein ligase RNF168 | RNF168 | 355 |
| Q8IYW5 | E3 ubiquitin-protein ligase RNF168 | RNF168 | 402 |
| Q8NCN4 | E3 ubiquitin-protein ligase RNF169 | RNF169 | 28 |
| Q8NCN4 | E3 ubiquitin-protein ligase RNF169 | RNF169 | 103 |
| Q8NCN4 | E3 ubiquitin-protein ligase RNF169 | RNF169 | 132 |
| Q8NCN4 | E3 ubiquitin-protein ligase RNF169 | RNF169 | 293 |
| Q8NCN4 | E3 ubiquitin-protein ligase RNF169 | RNF169 | 337 |
| Q8NCN4 | E3 ubiquitin-protein ligase RNF169 | RNF169 | 387 |
| Q8NCN4 | E3 ubiquitin-protein ligase RNF169 | RNF169 | 390 |
| Q8NCN4 | E3 ubiquitin-protein ligase RNF169 | RNF169 | 529 |
| Q8NCN4 | E3 ubiquitin-protein ligase RNF169 | RNF169 | 530 |
| Q8NCN4 | E3 ubiquitin-protein ligase RNF169 | RNF169 | 563 |
| Q9BXT8 | RING finger protein 17 | RNF17 | 1356 |
| Q9BXT8 | RING finger protein 17 | RNF17 | 1530 |
| Q8N4F7 | RING finger protein 175 | RNF175 | 265 |
| Q8N4F7 | RING finger protein 175 | RNF175 | 273 |
| Q8N4F7 | RING finger protein 175 | RNF175 | 276 |
| Q9P0P0 | E3 ubiquitin-protein ligase RNF181 | RNF181 | 94 |
| Q96GF1 | E3 ubiquitin-protein ligase RNF185 | RNF185 | 76 |
| Q96GF1 | E3 ubiquitin-protein ligase RNF185 | RNF185 | 79 |
| Q9NXI6 | RING finger protein 186 | RNF186 | 97 |
| Q5TA31 | E3 ubiquitin-protein ligase RNF187 | RNF187 | 27 |
| Q5TA31 | E3 ubiquitin-protein ligase RNF187 | RNF187 | 97 |
| Q5TA31 | E3 ubiquitin-protein ligase RNF187 | RNF187 | 105 |
| Q5TA31 | E3 ubiquitin-protein ligase RNF187 | RNF187 | 108 |
| Q9NV58 | E3 ubiquitin-protein ligase RNF19A | RNF19A | 179 |
| Q9NV58 | E3 ubiquitin-protein ligase RNF19A | RNF19A | 246 |
| Q9NV58 | E3 ubiquitin-protein ligase RNF19A | RNF19A | 264 |
| Q9NV58 | E3 ubiquitin-protein ligase RNF19A | RNF19A | 301 |
| Q9NV58 | E3 ubiquitin-protein ligase RNF19A | RNF19A | 460 |
| Q9NV58 | E3 ubiquitin-protein ligase RNF19A | RNF19A | 743 |
| Q6ZMZ0 | E3 ubiquitin-protein ligase RNF19B | RNF19B | 137 |
| Q6ZMZ0 | E3 ubiquitin-protein ligase RNF19B | RNF19B | 251 |
| Q6ZMZ0 | E3 ubiquitin-protein ligase RNF19B | RNF19B | 290 |
| Q6ZMZ0 | E3 ubiquitin-protein ligase RNF19B | RNF19B | 446 |
| Q6ZMZ0 | E3 ubiquitin-protein ligase RNF19B | RNF19B | 695 |
| Q99496 | E3 ubiquitin-protein ligase RING2 | RNF2 | 51 |
| Q99496 | E3 ubiquitin-protein ligase RING2 | RNF2 | 54 |
| Q99496 | E3 ubiquitin-protein ligase RING2 | RNF2 | 72 |
| Q99496 | E3 ubiquitin-protein ligase RING2 | RNF2 | 75 |
| Q99496 | E3 ubiquitin-protein ligase RING2 | RNF2 | 87 |
| Q99496 | E3 ubiquitin-protein ligase RING2 | RNF2 | 90 |
| Q99496 | E3 ubiquitin-protein ligase RING2 | RNF2 | 179 |
| Q5VTR2 | E3 ubiquitin-protein ligase BRE1A | RNF20 | 344 |
| Q5VTR2 | E3 ubiquitin-protein ligase BRE1A | RNF20 | 383 |
| Q5VTR2 | E3 ubiquitin-protein ligase BRE1A | RNF20 | 905 |
| Q5VTR2 | E3 ubiquitin-protein ligase BRE1A | RNF20 | 922 |
| Q5VTR2 | E3 ubiquitin-protein ligase BRE1A | RNF20 | 924 |
| Q5VTR2 | E3 ubiquitin-protein ligase BRE1A | RNF20 | 925 |
| Q5VTR2 | E3 ubiquitin-protein ligase BRE1A | RNF20 | 942 |
| Q5VTR2 | E3 ubiquitin-protein ligase BRE1A | RNF20 | 945 |
| Q5VTR2 | E3 ubiquitin-protein ligase BRE1A | RNF20 | 960 |
| Q63HN8 | E3 ubiquitin-protein ligase RNF213 | RNF213 | 18 |
| Q63HN8 | E3 ubiquitin-protein ligase RNF213 | RNF213 | 21 |
| Q63HN8 | E3 ubiquitin-protein ligase RNF213 | RNF213 | 310 |
| Q63HN8 | E3 ubiquitin-protein ligase RNF213 | RNF213 | 418 |
| Q63HN8 | E3 ubiquitin-protein ligase RNF213 | RNF213 | 438 |
| Q63HN8 | E3 ubiquitin-protein ligase RNF213 | RNF213 | 614 |
| Q63HN8 | E3 ubiquitin-protein ligase RNF213 | RNF213 | 680 |
| Q63HN8 | E3 ubiquitin-protein ligase RNF213 | RNF213 | 700 |
| Q63HN8 | E3 ubiquitin-protein ligase RNF213 | RNF213 | 701 |
| Q63HN8 | E3 ubiquitin-protein ligase RNF213 | RNF213 | 856 |
| Q63HN8 | E3 ubiquitin-protein ligase RNF213 | RNF213 | 876 |
| Q63HN8 | E3 ubiquitin-protein ligase RNF213 | RNF213 | 978 |
| Q63HN8 | E3 ubiquitin-protein ligase RNF213 | RNF213 | 981 |
| Q63HN8 | E3 ubiquitin-protein ligase RNF213 | RNF213 | 1064 |
| Q63HN8 | E3 ubiquitin-protein ligase RNF213 | RNF213 | 1134 |
| Q63HN8 | E3 ubiquitin-protein ligase RNF213 | RNF213 | 1156 |
| Q63HN8 | E3 ubiquitin-protein ligase RNF213 | RNF213 | 1164 |
| Q63HN8 | E3 ubiquitin-protein ligase RNF213 | RNF213 | 1325 |
| Q63HN8 | E3 ubiquitin-protein ligase RNF213 | RNF213 | 1429 |
| Q63HN8 | E3 ubiquitin-protein ligase RNF213 | RNF213 | 1510 |
| Q63HN8 | E3 ubiquitin-protein ligase RNF213 | RNF213 | 1614 |
| Q63HN8 | E3 ubiquitin-protein ligase RNF213 | RNF213 | 1736 |
| Q63HN8 | E3 ubiquitin-protein ligase RNF213 | RNF213 | 1748 |
| Q63HN8 | E3 ubiquitin-protein ligase RNF213 | RNF213 | 1916 |
| Q63HN8 | E3 ubiquitin-protein ligase RNF213 | RNF213 | 1985 |
| Q63HN8 | E3 ubiquitin-protein ligase RNF213 | RNF213 | 2092 |
| Q63HN8 | E3 ubiquitin-protein ligase RNF213 | RNF213 | 2132 |
| Q63HN8 | E3 ubiquitin-protein ligase RNF213 | RNF213 | 2378 |
| Q63HN8 | E3 ubiquitin-protein ligase RNF213 | RNF213 | 2412 |
| Q63HN8 | E3 ubiquitin-protein ligase RNF213 | RNF213 | 2424 |
| Q63HN8 | E3 ubiquitin-protein ligase RNF213 | RNF213 | 2524 |
| Q63HN8 | E3 ubiquitin-protein ligase RNF213 | RNF213 | 2536 |
| Q63HN8 | E3 ubiquitin-protein ligase RNF213 | RNF213 | 2620 |
| Q63HN8 | E3 ubiquitin-protein ligase RNF213 | RNF213 | 2633 |
| Q63HN8 | E3 ubiquitin-protein ligase RNF213 | RNF213 | 2809 |
| Q63HN8 | E3 ubiquitin-protein ligase RNF213 | RNF213 | 2868 |
| Q63HN8 | E3 ubiquitin-protein ligase RNF213 | RNF213 | 2918 |
| Q63HN8 | E3 ubiquitin-protein ligase RNF213 | RNF213 | 2943 |
| Q63HN8 | E3 ubiquitin-protein ligase RNF213 | RNF213 | 3008 |
| Q63HN8 | E3 ubiquitin-protein ligase RNF213 | RNF213 | 3084 |
| Q63HN8 | E3 ubiquitin-protein ligase RNF213 | RNF213 | 3261 |
| Q63HN8 | E3 ubiquitin-protein ligase RNF213 | RNF213 | 3330 |
| Q63HN8 | E3 ubiquitin-protein ligase RNF213 | RNF213 | 3365 |
| Q63HN8 | E3 ubiquitin-protein ligase RNF213 | RNF213 | 3539 |
| Q63HN8 | E3 ubiquitin-protein ligase RNF213 | RNF213 | 3554 |
| Q63HN8 | E3 ubiquitin-protein ligase RNF213 | RNF213 | 3574 |
| Q63HN8 | E3 ubiquitin-protein ligase RNF213 | RNF213 | 3609 |
| Q63HN8 | E3 ubiquitin-protein ligase RNF213 | RNF213 | 3794 |
| Q63HN8 | E3 ubiquitin-protein ligase RNF213 | RNF213 | 3854 |
| Q63HN8 | E3 ubiquitin-protein ligase RNF213 | RNF213 | 3958 |
| Q63HN8 | E3 ubiquitin-protein ligase RNF213 | RNF213 | 3979 |
| Q63HN8 | E3 ubiquitin-protein ligase RNF213 | RNF213 | 3981 |
| Q63HN8 | E3 ubiquitin-protein ligase RNF213 | RNF213 | 3997 |
| Q63HN8 | E3 ubiquitin-protein ligase RNF213 | RNF213 | 4000 |
| Q63HN8 | E3 ubiquitin-protein ligase RNF213 | RNF213 | 4009 |
| Q63HN8 | E3 ubiquitin-protein ligase RNF213 | RNF213 | 4012 |
| Q63HN8 | E3 ubiquitin-protein ligase RNF213 | RNF213 | 4017 |
| Q63HN8 | E3 ubiquitin-protein ligase RNF213 | RNF213 | 4032 |
| Q63HN8 | E3 ubiquitin-protein ligase RNF213 | RNF213 | 4035 |
| Q63HN8 | E3 ubiquitin-protein ligase RNF213 | RNF213 | 4111 |
| Q63HN8 | E3 ubiquitin-protein ligase RNF213 | RNF213 | 4177 |
| Q63HN8 | E3 ubiquitin-protein ligase RNF213 | RNF213 | 4235 |
| Q63HN8 | E3 ubiquitin-protein ligase RNF213 | RNF213 | 4258 |
| Q63HN8 | E3 ubiquitin-protein ligase RNF213 | RNF213 | 4348 |
| Q63HN8 | E3 ubiquitin-protein ligase RNF213 | RNF213 | 4397 |
| Q63HN8 | E3 ubiquitin-protein ligase RNF213 | RNF213 | 4407 |
| Q63HN8 | E3 ubiquitin-protein ligase RNF213 | RNF213 | 4456 |
| Q63HN8 | E3 ubiquitin-protein ligase RNF213 | RNF213 | 4570 |
| Q63HN8 | E3 ubiquitin-protein ligase RNF213 | RNF213 | 4737 |
| Q63HN8 | E3 ubiquitin-protein ligase RNF213 | RNF213 | 4856 |
| Q63HN8 | E3 ubiquitin-protein ligase RNF213 | RNF213 | 4879 |
| Q63HN8 | E3 ubiquitin-protein ligase RNF213 | RNF213 | 4937 |
| Q8ND24 | RING finger protein 214 | RNF214 | 99 |
| Q8ND24 | RING finger protein 214 | RNF214 | 139 |
| Q8ND24 | RING finger protein 214 | RNF214 | 149 |
| Q8ND24 | RING finger protein 214 | RNF214 | 315 |
| Q8ND24 | RING finger protein 214 | RNF214 | 569 |
| Q8ND24 | RING finger protein 214 | RNF214 | 655 |
| Q8ND24 | RING finger protein 214 | RNF214 | 661 |
| Q8ND24 | RING finger protein 214 | RNF214 | 675 |
| Q8ND24 | RING finger protein 214 | RNF214 | 683 |
| Q8ND24 | RING finger protein 214 | RNF214 | 696 |
| Q8ND24 | RING finger protein 214 | RNF214 | 699 |
| Q9NWF9 | E3 ubiquitin-protein ligase RNF216 | RNF216 | 370 |
| Q9NWF9 | E3 ubiquitin-protein ligase RNF216 | RNF216 | 518 |
| Q9NWF9 | E3 ubiquitin-protein ligase RNF216 | RNF216 | 605 |
| Q9NWF9 | E3 ubiquitin-protein ligase RNF216 | RNF216 | 623 |
| Q9NWF9 | E3 ubiquitin-protein ligase RNF216 | RNF216 | 628 |
| Q9NWF9 | E3 ubiquitin-protein ligase RNF216 | RNF216 | 636 |
| Q9NWF9 | E3 ubiquitin-protein ligase RNF216 | RNF216 | 648 |
| Q9NWF9 | E3 ubiquitin-protein ligase RNF216 | RNF216 | 678 |
| Q9NWF9 | E3 ubiquitin-protein ligase RNF216 | RNF216 | 688 |
| Q8TC41 | Probable E3 ubiquitin-protein ligase RNF217 | RNF217 | 278 |
| Q5W0B1 | RING finger protein 219 | RNF219 | 52 |
| Q5W0B1 | RING finger protein 219 | RNF219 | 55 |
| Q5W0B1 | RING finger protein 219 | RNF219 | 65 |
| Q5W0B1 | RING finger protein 219 | RNF219 | 105 |
| Q5W0B1 | RING finger protein 219 | RNF219 | 271 |
| Q5W0B1 | RING finger protein 219 | RNF219 | 321 |
| Q5W0B1 | RING finger protein 219 | RNF219 | 338 |
| Q5W0B1 | RING finger protein 219 | RNF219 | 375 |
| Q5W0B1 | RING finger protein 219 | RNF219 | 390 |
| Q5W0B1 | RING finger protein 219 | RNF219 | 397 |
| Q5W0B1 | RING finger protein 219 | RNF219 | 432 |
| Q5W0B1 | RING finger protein 219 | RNF219 | 458 |
| Q5W0B1 | RING finger protein 219 | RNF219 | 506 |
| Q5W0B1 | RING finger protein 219 | RNF219 | 555 |
| Q5VTB9 | E3 ubiquitin-protein ligase RNF220 | RNF220 | 336 |
| Q5VTB9 | E3 ubiquitin-protein ligase RNF220 | RNF220 | 359 |
| Q5VTB9 | E3 ubiquitin-protein ligase RNF220 | RNF220 | 382 |
| Q5VTB9 | E3 ubiquitin-protein ligase RNF220 | RNF220 | 417 |
| Q5VTB9 | E3 ubiquitin-protein ligase RNF220 | RNF220 | 517 |
| Q5VTB9 | E3 ubiquitin-protein ligase RNF220 | RNF220 | 530 |
| Q5VTB9 | E3 ubiquitin-protein ligase RNF220 | RNF220 | 549 |
| Q5VTB9 | E3 ubiquitin-protein ligase RNF220 | RNF220 | 552 |
| E7ERA6 | RING finger protein 223 | RNF223 | 54 |
| E7ERA6 | RING finger protein 223 | RNF223 | 75 |
| E7ERA6 | RING finger protein 223 | RNF223 | 116 |
| P0DH78 | RING finger protein 224 | RNF224 | 124 |
| P0DH78 | RING finger protein 224 | RNF224 | 140 |
| P0DH78 | RING finger protein 224 | RNF224 | 143 |
| Q96BH1 | E3 ubiquitin-protein ligase RNF25 | RNF25 | 138 |
| Q96BH1 | E3 ubiquitin-protein ligase RNF25 | RNF25 | 153 |
| Q96BH1 | E3 ubiquitin-protein ligase RNF25 | RNF25 | 159 |
| Q96BH1 | E3 ubiquitin-protein ligase RNF25 | RNF25 | 161 |
| Q96BH1 | E3 ubiquitin-protein ligase RNF25 | RNF25 | 198 |
| Q96BH1 | E3 ubiquitin-protein ligase RNF25 | RNF25 | 201 |
| Q96BH1 | E3 ubiquitin-protein ligase RNF25 | RNF25 | 316 |
| Q96BH1 | E3 ubiquitin-protein ligase RNF25 | RNF25 | 360 |
| Q96BH1 | E3 ubiquitin-protein ligase RNF25 | RNF25 | 417 |
| Q9BY78 | E3 ubiquitin-protein ligase RNF26 | RNF26 | 421 |
| Q96EP0 | E3 ubiquitin-protein ligase RNF31 | RNF31 | 59 |
| Q96EP0 | E3 ubiquitin-protein ligase RNF31 | RNF31 | 217 |
| Q96EP0 | E3 ubiquitin-protein ligase RNF31 | RNF31 | 220 |
| Q96EP0 | E3 ubiquitin-protein ligase RNF31 | RNF31 | 229 |
| Q96EP0 | E3 ubiquitin-protein ligase RNF31 | RNF31 | 232 |
| Q96EP0 | E3 ubiquitin-protein ligase RNF31 | RNF31 | 237 |
| Q96EP0 | E3 ubiquitin-protein ligase RNF31 | RNF31 | 473 |
| Q96EP0 | E3 ubiquitin-protein ligase RNF31 | RNF31 | 504 |
| Q96EP0 | E3 ubiquitin-protein ligase RNF31 | RNF31 | 551 |
| Q96EP0 | E3 ubiquitin-protein ligase RNF31 | RNF31 | 572 |
| Q96EP0 | E3 ubiquitin-protein ligase RNF31 | RNF31 | 699 |
| Q96EP0 | E3 ubiquitin-protein ligase RNF31 | RNF31 | 702 |
| Q96EP0 | E3 ubiquitin-protein ligase RNF31 | RNF31 | 717 |
| Q96EP0 | E3 ubiquitin-protein ligase RNF31 | RNF31 | 722 |
| Q96EP0 | E3 ubiquitin-protein ligase RNF31 | RNF31 | 725 |
| Q96EP0 | E3 ubiquitin-protein ligase RNF31 | RNF31 | 817 |
| Q96EP0 | E3 ubiquitin-protein ligase RNF31 | RNF31 | 820 |
| Q96EP0 | E3 ubiquitin-protein ligase RNF31 | RNF31 | 825 |
| Q96EP0 | E3 ubiquitin-protein ligase RNF31 | RNF31 | 841 |
| Q96EP0 | E3 ubiquitin-protein ligase RNF31 | RNF31 | 885 |
| Q96EP0 | E3 ubiquitin-protein ligase RNF31 | RNF31 | 890 |
| Q96EP0 | E3 ubiquitin-protein ligase RNF31 | RNF31 | 898 |
| Q96EP0 | E3 ubiquitin-protein ligase RNF31 | RNF31 | 911 |
| Q96EP0 | E3 ubiquitin-protein ligase RNF31 | RNF31 | 916 |
| Q96EP0 | E3 ubiquitin-protein ligase RNF31 | RNF31 | 930 |
| Q96EP0 | E3 ubiquitin-protein ligase RNF31 | RNF31 | 969 |
| Q9H0A6 | RING finger protein 32 | RNF32 | 165 |
| Q9H0A6 | RING finger protein 32 | RNF32 | 168 |
| Q969K3 | E3 ubiquitin-protein ligase RNF34 | RNF34 | 62 |
| Q969K3 | E3 ubiquitin-protein ligase RNF34 | RNF34 | 65 |
| Q969K3 | E3 ubiquitin-protein ligase RNF34 | RNF34 | 79 |
| Q969K3 | E3 ubiquitin-protein ligase RNF34 | RNF34 | 86 |
| Q969K3 | E3 ubiquitin-protein ligase RNF34 | RNF34 | 102 |
| Q969K3 | E3 ubiquitin-protein ligase RNF34 | RNF34 | 137 |
| Q969K3 | E3 ubiquitin-protein ligase RNF34 | RNF34 | 149 |
| Q969K3 | E3 ubiquitin-protein ligase RNF34 | RNF34 | 286 |
| Q969K3 | E3 ubiquitin-protein ligase RNF34 | RNF34 | 287 |
| Q969K3 | E3 ubiquitin-protein ligase RNF34 | RNF34 | 359 |
| Q9H0F5 | E3 ubiquitin-protein ligase RNF38 | RNF38 | 110 |
| Q9H0F5 | E3 ubiquitin-protein ligase RNF38 | RNF38 | 500 |
| Q9H0F5 | E3 ubiquitin-protein ligase RNF38 | RNF38 | 503 |
| P78317 | E3 ubiquitin-protein ligase RNF4 | RNF4 | 91 |
| P78317 | E3 ubiquitin-protein ligase RNF4 | RNF4 | 154 |
| P78317 | E3 ubiquitin-protein ligase RNF4 | RNF4 | 159 |
| P78317 | E3 ubiquitin-protein ligase RNF4 | RNF4 | 162 |
| O75150 | E3 ubiquitin-protein ligase BRE1B | RNF40 | 69 |
| O75150 | E3 ubiquitin-protein ligase BRE1B | RNF40 | 210 |
| O75150 | E3 ubiquitin-protein ligase BRE1B | RNF40 | 890 |
| O75150 | E3 ubiquitin-protein ligase BRE1B | RNF40 | 948 |
| O75150 | E3 ubiquitin-protein ligase BRE1B | RNF40 | 950 |
| O75150 | E3 ubiquitin-protein ligase BRE1B | RNF40 | 951 |
| O75150 | E3 ubiquitin-protein ligase BRE1B | RNF40 | 971 |
| O75150 | E3 ubiquitin-protein ligase BRE1B | RNF40 | 986 |
| Q9H4P4 | E3 ubiquitin-protein ligase NRDP1 | RNF41 | 90 |
| Q9H4P4 | E3 ubiquitin-protein ligase NRDP1 | RNF41 | 106 |
| Q9H4P4 | E3 ubiquitin-protein ligase NRDP1 | RNF41 | 120 |
| Q68DV7 | E3 ubiquitin-protein ligase RNF43 | RNF43 | 252 |
| Q68DV7 | E3 ubiquitin-protein ligase RNF43 | RNF43 | 451 |
| Q68DV7 | E3 ubiquitin-protein ligase RNF43 | RNF43 | 615 |
| Q68DV7 | E3 ubiquitin-protein ligase RNF43 | RNF43 | 633 |
| Q7L0R7 | RING finger protein 44 | RNF44 | 398 |
| Q7L0R7 | RING finger protein 44 | RNF44 | 417 |
| Q7L0R7 | RING finger protein 44 | RNF44 | 420 |
| Q99942 | E3 ubiquitin-protein ligase RNF5 | RNF5 | 30 |
| Q99942 | E3 ubiquitin-protein ligase RNF5 | RNF5 | 42 |
| Q99942 | E3 ubiquitin-protein ligase RNF5 | RNF5 | 47 |
| Q99942 | E3 ubiquitin-protein ligase RNF5 | RNF5 | 50 |
| Q99942 | E3 ubiquitin-protein ligase RNF5 | RNF5 | 64 |
| Q99942 | E3 ubiquitin-protein ligase RNF5 | RNF5 | 67 |
| Q9Y252 | E3 ubiquitin-protein ligase RNF6 | RNF6 | 667 |
| Q9UBF6 | RING-box protein 2 | RNF7 | 47 |
| Q9UBF6 | RING-box protein 2 | RNF7 | 61 |
| Q9UBF6 | RING-box protein 2 | RNF7 | 64 |
| Q9UBF6 | RING-box protein 2 | RNF7 | 73 |
| Q9UBF6 | RING-box protein 2 | RNF7 | 80 |
| Q9UBF6 | RING-box protein 2 | RNF7 | 87 |
| Q9UBF6 | RING-box protein 2 | RNF7 | 88 |
| Q9UBF6 | RING-box protein 2 | RNF7 | 99 |
| Q9UBF6 | RING-box protein 2 | RNF7 | 102 |
| O76064 | E3 ubiquitin-protein ligase RNF8 | RNF8 | 55 |
| O76064 | E3 ubiquitin-protein ligase RNF8 | RNF8 | 181 |
| O76064 | E3 ubiquitin-protein ligase RNF8 | RNF8 | 300 |
| O76064 | E3 ubiquitin-protein ligase RNF8 | RNF8 | 456 |
| Q5M7Z0 | E3 ubiquitin-protein ligase RNFT1 | RNFT1 | 62 |
| Q5M7Z0 | E3 ubiquitin-protein ligase RNFT1 | RNFT1 | 73 |
| Q5M7Z0 | E3 ubiquitin-protein ligase RNFT1 | RNFT1 | 90 |
| Q5M7Z0 | E3 ubiquitin-protein ligase RNFT1 | RNFT1 | 106 |
| Q5M7Z0 | E3 ubiquitin-protein ligase RNFT1 | RNFT1 | 110 |
| Q5M7Z0 | E3 ubiquitin-protein ligase RNFT1 | RNFT1 | 409 |
| Q5M7Z0 | E3 ubiquitin-protein ligase RNFT1 | RNFT1 | 412 |
| Q96EX2 | RING finger and transmembrane domain-containing protein 2 | RNFT2 | 418 |
| Q96EX2 | RING finger and transmembrane domain-containing protein 2 | RNFT2 | 421 |
| Q96DX4 | RING finger and SPRY domain-containing protein 1 | RSPRY1 | 190 |
| Q96DX4 | RING finger and SPRY domain-containing protein 1 | RSPRY1 | 208 |
| Q96T51 | RUN and FYVE domain-containing protein 1 | RUFY1 | 8 |
| Q96T51 | RUN and FYVE domain-containing protein 1 | RUFY1 | 81 |
| Q96T51 | RUN and FYVE domain-containing protein 1 | RUFY1 | 107 |
| Q96T51 | RUN and FYVE domain-containing protein 1 | RUFY1 | 155 |
| Q96T51 | RUN and FYVE domain-containing protein 1 | RUFY1 | 184 |
| Q96T51 | RUN and FYVE domain-containing protein 1 | RUFY1 | 320 |
| Q96T51 | RUN and FYVE domain-containing protein 1 | RUFY1 | 351 |
| Q96T51 | RUN and FYVE domain-containing protein 1 | RUFY1 | 544 |
| Q96T51 | RUN and FYVE domain-containing protein 1 | RUFY1 | 648 |
| Q96T51 | RUN and FYVE domain-containing protein 1 | RUFY1 | 651 |
| Q96T51 | RUN and FYVE domain-containing protein 1 | RUFY1 | 664 |
| Q96T51 | RUN and FYVE domain-containing protein 1 | RUFY1 | 667 |
| Q96T51 | RUN and FYVE domain-containing protein 1 | RUFY1 | 672 |
| Q96T51 | RUN and FYVE domain-containing protein 1 | RUFY1 | 675 |
| Q96T51 | RUN and FYVE domain-containing protein 1 | RUFY1 | 692 |
| Q96T51 | RUN and FYVE domain-containing protein 1 | RUFY1 | 703 |
| Q7Z6J0 | E3 ubiquitin-protein ligase SH3RF1 | SH3RF1 | 33 |
| Q7Z6J0 | E3 ubiquitin-protein ligase SH3RF1 | SH3RF1 | 36 |
| Q7Z6J0 | E3 ubiquitin-protein ligase SH3RF1 | SH3RF1 | 93 |
| Q7Z6J0 | E3 ubiquitin-protein ligase SH3RF1 | SH3RF1 | 107 |
| Q7Z6J0 | E3 ubiquitin-protein ligase SH3RF1 | SH3RF1 | 279 |
| Q7Z6J0 | E3 ubiquitin-protein ligase SH3RF1 | SH3RF1 | 478 |
| Q7Z6J0 | E3 ubiquitin-protein ligase SH3RF1 | SH3RF1 | 658 |
| Q7Z6J0 | E3 ubiquitin-protein ligase SH3RF1 | SH3RF1 | 700 |
| Q7Z6J0 | E3 ubiquitin-protein ligase SH3RF1 | SH3RF1 | 773 |
| Q7Z6J0 | E3 ubiquitin-protein ligase SH3RF1 | SH3RF1 | 816 |
| Q7Z6J0 | E3 ubiquitin-protein ligase SH3RF1 | SH3RF1 | 831 |
| Q8TEC5 | E3 ubiquitin-protein ligase SH3RF2 | SH3RF2 | 28 |
| Q8TEC5 | E3 ubiquitin-protein ligase SH3RF2 | SH3RF2 | 36 |
| Q8TEC5 | E3 ubiquitin-protein ligase SH3RF2 | SH3RF2 | 134 |
| Q8TEC5 | E3 ubiquitin-protein ligase SH3RF2 | SH3RF2 | 413 |
| Q8TEC5 | E3 ubiquitin-protein ligase SH3RF2 | SH3RF2 | 695 |
| Q8TEJ3 | E3 ubiquitin-protein ligase SH3RF3 | SH3RF3 | 78 |
| Q8TEJ3 | E3 ubiquitin-protein ligase SH3RF3 | SH3RF3 | 194 |
| Q8TEJ3 | E3 ubiquitin-protein ligase SH3RF3 | SH3RF3 | 428 |
| Q8TEJ3 | E3 ubiquitin-protein ligase SH3RF3 | SH3RF3 | 497 |
| Q8TEJ3 | E3 ubiquitin-protein ligase SH3RF3 | SH3RF3 | 776 |
| Q9H0F6 | Sharpin | SHARPIN | 140 |
| Q9H0F6 | Sharpin | SHARPIN | 213 |
| Q9H0F6 | Sharpin | SHARPIN | 248 |
| Q9H0F6 | Sharpin | SHARPIN | 275 |
| Q9H0F6 | Sharpin | SHARPIN | 277 |
| Q8TBC3 | SH3KBP1-binding protein 1 | SHKBP1 | 121 |
| Q8TBC3 | SH3KBP1-binding protein 1 | SHKBP1 | 447 |
| Q8TBC3 | SH3KBP1-binding protein 1 | SHKBP1 | 491 |
| Q8TBC3 | SH3KBP1-binding protein 1 | SHKBP1 | 550 |
| Q8TBC3 | SH3KBP1-binding protein 1 | SHKBP1 | 667 |
| Q149N8 | E3 ubiquitin-protein ligase SHPRH | SHPRH | 171 |
| Q149N8 | E3 ubiquitin-protein ligase SHPRH | SHPRH | 320 |
| Q149N8 | E3 ubiquitin-protein ligase SHPRH | SHPRH | 354 |
| Q149N8 | E3 ubiquitin-protein ligase SHPRH | SHPRH | 439 |
| Q149N8 | E3 ubiquitin-protein ligase SHPRH | SHPRH | 489 |
| Q149N8 | E3 ubiquitin-protein ligase SHPRH | SHPRH | 601 |
| Q149N8 | E3 ubiquitin-protein ligase SHPRH | SHPRH | 680 |
| Q149N8 | E3 ubiquitin-protein ligase SHPRH | SHPRH | 688 |
| Q149N8 | E3 ubiquitin-protein ligase SHPRH | SHPRH | 703 |
| Q149N8 | E3 ubiquitin-protein ligase SHPRH | SHPRH | 706 |
| Q149N8 | E3 ubiquitin-protein ligase SHPRH | SHPRH | 826 |
| Q149N8 | E3 ubiquitin-protein ligase SHPRH | SHPRH | 845 |
| Q149N8 | E3 ubiquitin-protein ligase SHPRH | SHPRH | 885 |
| Q149N8 | E3 ubiquitin-protein ligase SHPRH | SHPRH | 942 |
| Q149N8 | E3 ubiquitin-protein ligase SHPRH | SHPRH | 981 |
| Q149N8 | E3 ubiquitin-protein ligase SHPRH | SHPRH | 982 |
| Q149N8 | E3 ubiquitin-protein ligase SHPRH | SHPRH | 1025 |
| Q149N8 | E3 ubiquitin-protein ligase SHPRH | SHPRH | 1240 |
| Q149N8 | E3 ubiquitin-protein ligase SHPRH | SHPRH | 1435 |
| Q8IUQ4 | E3 ubiquitin-protein ligase SIAH1 | SIAH1 | 16 |
| Q8IUQ4 | E3 ubiquitin-protein ligase SIAH1 | SIAH1 | 71 |
| Q8IUQ4 | E3 ubiquitin-protein ligase SIAH1 | SIAH1 | 72 |
| O43255 | E3 ubiquitin-protein ligase SIAH2 | SIAH2 | 110 |
| O43255 | E3 ubiquitin-protein ligase SIAH2 | SIAH2 | 111 |
| O43255 | E3 ubiquitin-protein ligase SIAH2 | SIAH2 | 114 |
| O43255 | E3 ubiquitin-protein ligase SIAH2 | SIAH2 | 138 |
| P63208 | S-phase kinase-associated protein 1 | SKP1 | 62 |
| P63208 | S-phase kinase-associated protein 1 | SKP1 | 120 |
| P63208 | S-phase kinase-associated protein 1 | SKP1 | 160 |
| Q13309 | S-phase kinase-associated protein 2 | SKP2 | 111 |
| Q13309 | S-phase kinase-associated protein 2 | SKP2 | 113 |
| Q13309 | S-phase kinase-associated protein 2 | SKP2 | 124 |
| Q13309 | S-phase kinase-associated protein 2 | SKP2 | 345 |
| Q13309 | S-phase kinase-associated protein 2 | SKP2 | 390 |
| Q13309 | S-phase kinase-associated protein 2 | SKP2 | 423 |
| Q8IY92 | Structure-specific endonuclease subunit SLX4 | SLX4 | 192 |
| Q8IY92 | Structure-specific endonuclease subunit SLX4 | SLX4 | 296 |
| Q8IY92 | Structure-specific endonuclease subunit SLX4 | SLX4 | 318 |
| Q8IY92 | Structure-specific endonuclease subunit SLX4 | SLX4 | 519 |
| Q8IY92 | Structure-specific endonuclease subunit SLX4 | SLX4 | 926 |
| Q8IY92 | Structure-specific endonuclease subunit SLX4 | SLX4 | 1033 |
| Q8IY92 | Structure-specific endonuclease subunit SLX4 | SLX4 | 1109 |
| Q8IY92 | Structure-specific endonuclease subunit SLX4 | SLX4 | 1239 |
| Q8IY92 | Structure-specific endonuclease subunit SLX4 | SLX4 | 1269 |
| Q8IY92 | Structure-specific endonuclease subunit SLX4 | SLX4 | 1324 |
| Q8IY92 | Structure-specific endonuclease subunit SLX4 | SLX4 | 1482 |
| Q9HCE7 | E3 ubiquitin-protein ligase SMURF1 | SMURF1 | 21 |
| Q9HCE7 | E3 ubiquitin-protein ligase SMURF1 | SMURF1 | 48 |
| Q9HCE7 | E3 ubiquitin-protein ligase SMURF1 | SMURF1 | 152 |
| Q9HCE7 | E3 ubiquitin-protein ligase SMURF1 | SMURF1 | 652 |
| Q9HCE7 | E3 ubiquitin-protein ligase SMURF1 | SMURF1 | 725 |
| Q9HAU4 | E3 ubiquitin-protein ligase SMURF2 | SMURF2 | 20 |
| Q9HAU4 | E3 ubiquitin-protein ligase SMURF2 | SMURF2 | 151 |
| Q9HAU4 | E3 ubiquitin-protein ligase SMURF2 | SMURF2 | 293 |
| Q9HAU4 | E3 ubiquitin-protein ligase SMURF2 | SMURF2 | 356 |
| Q9HAU4 | E3 ubiquitin-protein ligase SMURF2 | SMURF2 | 362 |
| Q9HAU4 | E3 ubiquitin-protein ligase SMURF2 | SMURF2 | 706 |
| Q9HAU4 | E3 ubiquitin-protein ligase SMURF2 | SMURF2 | 716 |
| Q9HAU4 | E3 ubiquitin-protein ligase SMURF2 | SMURF2 | 743 |
| O15524 | Suppressor of cytokine signaling 1 | SOCS1 | 178 |
| O14508 | Suppressor of cytokine signaling 2 | SOCS2 | 5 |
| O14508 | Suppressor of cytokine signaling 2 | SOCS2 | 111 |
| O14508 | Suppressor of cytokine signaling 2 | SOCS2 | 133 |
| O14508 | Suppressor of cytokine signaling 2 | SOCS2 | 174 |
| O14543 | Suppressor of cytokine signaling 3 | SOCS3 | 97 |
| O14543 | Suppressor of cytokine signaling 3 | SOCS3 | 119 |
| O14543 | Suppressor of cytokine signaling 3 | SOCS3 | 193 |
| O14544 | Suppressor of cytokine signaling 6 | SOCS6 | 184 |
| O14544 | Suppressor of cytokine signaling 6 | SOCS6 | 241 |
| O14544 | Suppressor of cytokine signaling 6 | SOCS6 | 342 |
| O14512 | Suppressor of cytokine signaling 7 | SOCS7 | 378 |
| O14512 | Suppressor of cytokine signaling 7 | SOCS7 | 522 |
| O43791 | Speckle-type POZ protein | SPOP | 68 |
| O43791 | Speckle-type POZ protein | SPOP | 93 |
| O43791 | Speckle-type POZ protein | SPOP | 205 |
| O43791 | Speckle-type POZ protein | SPOP | 361 |
| Q6IQ16 | Speckle-type POZ protein-like | SPOPL | 202 |
| Q6IQ16 | Speckle-type POZ protein-like | SPOPL | 379 |
| Q6PJ21 | SPRY domain-containing SOCS box protein 3 | SPSB3 | 321 |
| Q6PJ21 | SPRY domain-containing SOCS box protein 3 | SPSB3 | 347 |
| Q9UNE7 | E3 ubiquitin-protein ligase CHIP | STUB1 | 48 |
| Q9UNE7 | E3 ubiquitin-protein ligase CHIP | STUB1 | 69 |
| Q9UNE7 | E3 ubiquitin-protein ligase CHIP | STUB1 | 83 |
| Q9UNE7 | E3 ubiquitin-protein ligase CHIP | STUB1 | 180 |
| Q9UNE7 | E3 ubiquitin-protein ligase CHIP | STUB1 | 199 |
| Q9UNE7 | E3 ubiquitin-protein ligase CHIP | STUB1 | 232 |
| Q9UNE7 | E3 ubiquitin-protein ligase CHIP | STUB1 | 244 |
| Q9Y2Z0 | Protein SGT1 homolog | SUGT1 | 49 |
| Q9Y2Z0 | Protein SGT1 homolog | SUGT1 | 54 |
| Q9Y2Z0 | Protein SGT1 homolog | SUGT1 | 62 |
| Q9Y2Z0 | Protein SGT1 homolog | SUGT1 | 88 |
| Q9Y2Z0 | Protein SGT1 homolog | SUGT1 | 154 |
| Q86TM6 | E3 ubiquitin-protein ligase synoviolin | SYVN1 | 307 |
| Q86TM6 | E3 ubiquitin-protein ligase synoviolin | SYVN1 | 315 |
| Q86TM6 | E3 ubiquitin-protein ligase synoviolin | SYVN1 | 326 |
| Q86TM6 | E3 ubiquitin-protein ligase synoviolin | SYVN1 | 329 |
| A0AVI4 | E3 ubiquitin-protein ligase TM129 | TMEM129 | 354 |
| Q13829 | BTB/POZ domain-containing adapter for CUL3-mediated | TNFAIP1 | 76 |
| | RhoA degradation protein 2 | | |
| Q13829 | BTB/POZ domain-containing adapter for CUL3-mediated | TNFAIP1 | 224 |
| | RhoA degradation protein 2 | | |
| Q13829 | BTB/POZ domain-containing adapter for CUL3-mediated | TNFAIP1 | 225 |
| | RhoA degradation protein 2 | | |
| P21580 | Tumor necrosis factor alpha-induced protein 3 | TNFAIP3 | 54 |
| P21580 | Tumor necrosis factor alpha-induced protein 3 | TNFAIP3 | 57 |
| P21580 | Tumor necrosis factor alpha-induced protein 3 | TNFAIP3 | 103 |
| P21580 | Tumor necrosis factor alpha-induced protein 3 | TNFAIP3 | 158 |
| P21580 | Tumor necrosis factor alpha-induced protein 3 | TNFAIP3 | 243 |
| P21580 | Tumor necrosis factor alpha-induced protein 3 | TNFAIP3 | 392 |
| P21580 | Tumor necrosis factor alpha-induced protein 3 | TNFAIP3 | 404 |
| P21580 | Tumor necrosis factor alpha-induced protein 3 | TNFAIP3 | 407 |
| P21580 | Tumor necrosis factor alpha-induced protein 3 | TNFAIP3 | 478 |
| P21580 | Tumor necrosis factor alpha-induced protein 3 | TNFAIP3 | 483 |
| P21580 | Tumor necrosis factor alpha-induced protein 3 | TNFAIP3 | 521 |
| P21580 | Tumor necrosis factor alpha-induced protein 3 | TNFAIP3 | 524 |
| P21580 | Tumor necrosis factor alpha-induced protein 3 | TNFAIP3 | 536 |
| P21580 | Tumor necrosis factor alpha-induced protein 3 | TNFAIP3 | 539 |
| P21580 | Tumor necrosis factor alpha-induced protein 3 | TNFAIP3 | 559 |
| P21580 | Tumor necrosis factor alpha-induced protein 3 | TNFAIP3 | 579 |
| P21580 | Tumor necrosis factor alpha-induced protein 3 | TNFAIP3 | 590 |
| P21580 | Tumor necrosis factor alpha-induced protein 3 | TNFAIP3 | 612 |
| P21580 | Tumor necrosis factor alpha-induced protein 3 | TNFAIP3 | 624 |
| P21580 | Tumor necrosis factor alpha-induced protein 3 | TNFAIP3 | 627 |
| P21580 | Tumor necrosis factor alpha-induced protein 3 | TNFAIP3 | 657 |
| P21580 | Tumor necrosis factor alpha-induced protein 3 | TNFAIP3 | 662 |
| P21580 | Tumor necrosis factor alpha-induced protein 3 | TNFAIP3 | 674 |
| P21580 | Tumor necrosis factor alpha-induced protein 3 | TNFAIP3 | 677 |
| P21580 | Tumor necrosis factor alpha-induced protein 3 | TNFAIP3 | 727 |
| P21580 | Tumor necrosis factor alpha-induced protein 3 | TNFAIP3 | 733 |
| P21580 | Tumor necrosis factor alpha-induced protein 3 | TNFAIP3 | 736 |
| P21580 | Tumor necrosis factor alpha-induced protein 3 | TNFAIP3 | 762 |
| P21580 | Tumor necrosis factor alpha-induced protein 3 | TNFAIP3 | 767 |
| P21580 | Tumor necrosis factor alpha-induced protein 3 | TNFAIP3 | 775 |
| P21580 | Tumor necrosis factor alpha-induced protein 3 | TNFAIP3 | 779 |
| P21580 | Tumor necrosis factor alpha-induced protein 3 | TNFAIP3 | 782 |
| Q15025 | TNFAIP3-interacting protein 1 | TNIP1 | 171 |
| Q15025 | TNFAIP3-interacting protein 1 | TNIP1 | 212 |
| Q15025 | TNFAIP3-interacting protein 1 | TNIP1 | 602 |
| Q9NS56 | E3 ubiquitin-protein ligase Topors | TOPORS | 49 |
| Q9NS56 | E3 ubiquitin-protein ligase Topors | TOPORS | 127 |
| Q9NS56 | E3 ubiquitin-protein ligase Topors | TOPORS | 138 |
| Q9NS56 | E3 ubiquitin-protein ligase Topors | TOPORS | 292 |
| Q9NS56 | E3 ubiquitin-protein ligase Topors | TOPORS | 550 |
| Q9NS56 | E3 ubiquitin-protein ligase Topors | TOPORS | 1037 |
| Q13077 | TNF receptor-associated factor 1 | TRAF1 | 37 |
| Q13077 | TNF receptor-associated factor 1 | TRAF1 | 38 |
| Q13077 | TNF receptor-associated factor 1 | TRAF1 | 41 |
| Q13077 | TNF receptor-associated factor 1 | TRAF1 | 54 |
| Q13077 | TNF receptor-associated factor 1 | TRAF1 | 57 |
| Q13077 | TNF receptor-associated factor 1 | TRAF1 | 88 |
| Q13077 | TNF receptor-associated factor 1 | TRAF1 | 95 |
| Q13077 | TNF receptor-associated factor 1 | TRAF1 | 169 |
| Q13077 | TNF receptor-associated factor 1 | TRAF1 | 280 |
| Q13077 | TNF receptor-associated factor 1 | TRAF1 | 285 |
| Q13077 | TNF receptor-associated factor 1 | TRAF1 | 384 |
| Q12933 | TNF receptor-associated factor 2 | TRAF2 | 34 |
| Q12933 | TNF receptor-associated factor 2 | TRAF2 | 37 |
| Q12933 | TNF receptor-associated factor 2 | TRAF2 | 49 |
| Q12933 | TNF receptor-associated factor 2 | TRAF2 | 112 |
| Q12933 | TNF receptor-associated factor 2 | TRAF2 | 124 |
| Q12933 | TNF receptor-associated factor 2 | TRAF2 | 129 |
| Q12933 | TNF receptor-associated factor 2 | TRAF2 | 136 |
| Q12933 | TNF receptor-associated factor 2 | TRAF2 | 156 |
| Q12933 | TNF receptor-associated factor 2 | TRAF2 | 170 |
| Q12933 | TNF receptor-associated factor 2 | TRAF2 | 171 |
| Q12933 | TNF receptor-associated factor 2 | TRAF2 | 189 |
| Q12933 | TNF receptor-associated factor 2 | TRAF2 | 192 |
| Q12933 | TNF receptor-associated factor 2 | TRAF2 | 212 |
| Q12933 | TNF receptor-associated factor 2 | TRAF2 | 216 |
| Q12933 | TNF receptor-associated factor 2 | TRAF2 | 223 |
| Q12933 | TNF receptor-associated factor 2 | TRAF2 | 287 |
| Q12933 | TNF receptor-associated factor 2 | TRAF2 | 303 |
| Q12933 | TNF receptor-associated factor 2 | TRAF2 | 469 |
| Q13114 | TNF receptor-associated factor 3 | TRAF3 | 61 |
| Q13114 | TNF receptor-associated factor 3 | TRAF3 | 68 |
| Q13114 | TNF receptor-associated factor 3 | TRAF3 | 105 |
| Q13114 | TNF receptor-associated factor 3 | TRAF3 | 106 |
| Q13114 | TNF receptor-associated factor 3 | TRAF3 | 180 |
| Q13114 | TNF receptor-associated factor 3 | TRAF3 | 208 |
| Q9BUZ4 | TNF receptor-associated factor 4 | TRAF4 | 90 |
| Q9BUZ4 | TNF receptor-associated factor 4 | TRAF4 | 134 |
| Q9BUZ4 | TNF receptor-associated factor 4 | TRAF4 | 188 |
| Q9BUZ4 | TNF receptor-associated factor 4 | TRAF4 | 195 |
| Q9BUZ4 | TNF receptor-associated factor 4 | TRAF4 | 198 |
| Q9BUZ4 | TNF receptor-associated factor 4 | TRAF4 | 214 |
| Q9BUZ4 | TNF receptor-associated factor 4 | TRAF4 | 221 |
| Q9BUZ4 | TNF receptor-associated factor 4 | TRAF4 | 250 |
| Q9BUZ4 | TNF receptor-associated factor 4 | TRAF4 | 282 |
| Q9BUZ4 | TNF receptor-associated factor 4 | TRAF4 | 332 |
| O00463 | TNF receptor-associated factor 5 | TRAF5 | 12 |
| O00463 | TNF receptor-associated factor 5 | TRAF5 | 65 |
| O00463 | TNF receptor-associated factor 5 | TRAF5 | 68 |
| O00463 | TNF receptor-associated factor 5 | TRAF5 | 81 |
| O00463 | TNF receptor-associated factor 5 | TRAF5 | 98 |
| O00463 | TNF receptor-associated factor 5 | TRAF5 | 116 |
| O00463 | TNF receptor-associated factor 5 | TRAF5 | 132 |
| O00463 | TNF receptor-associated factor 5 | TRAF5 | 139 |
| O00463 | TNF receptor-associated factor 5 | TRAF5 | 144 |
| O00463 | TNF receptor-associated factor 5 | TRAF5 | 161 |
| O00463 | TNF receptor-associated factor 5 | TRAF5 | 188 |
| O00463 | TNF receptor-associated factor 5 | TRAF5 | 195 |
| O00463 | TNF receptor-associated factor 5 | TRAF5 | 401 |
| O00463 | TNF receptor-associated factor 5 | TRAF5 | 521 |
| Q9Y4K3 | TNF receptor-associated factor 6 | TRAF6 | 85 |
| Q9Y4K3 | TNF receptor-associated factor 6 | TRAF6 | 105 |
| Q9Y4K3 | TNF receptor-associated factor 6 | TRAF6 | 134 |
| Q9Y4K3 | TNF receptor-associated factor 6 | TRAF6 | 139 |
| Q9Y4K3 | TNF receptor-associated factor 6 | TRAF6 | 182 |
| Q9Y4K3 | TNF receptor-associated factor 6 | TRAF6 | 235 |
| Q9Y4K3 | TNF receptor-associated factor 6 | TRAF6 | 349 |
| Q9Y4K3 | TNF receptor-associated factor 6 | TRAF6 | 366 |
| Q6Q0C0 | E3 ubiquitin-protein ligase TRAF7 | TRAF7 | 57 |
| Q6Q0C0 | E3 ubiquitin-protein ligase TRAF7 | TRAF7 | 134 |
| Q6Q0C0 | E3 ubiquitin-protein ligase TRAF7 | TRAF7 | 151 |
| Q6Q0C0 | E3 ubiquitin-protein ligase TRAF7 | TRAF7 | 161 |
| Q6Q0C0 | E3 ubiquitin-protein ligase TRAF7 | TRAF7 | 210 |
| Q6Q0C0 | E3 ubiquitin-protein ligase TRAF7 | TRAF7 | 226 |
| Q6Q0C0 | E3 ubiquitin-protein ligase TRAF7 | TRAF7 | 233 |
| Q6Q0C0 | E3 ubiquitin-protein ligase TRAF7 | TRAF7 | 254 |
| Q6Q0C0 | E3 ubiquitin-protein ligase TRAF7 | TRAF7 | 259 |
| Q6Q0C0 | E3 ubiquitin-protein ligase TRAF7 | TRAF7 | 266 |
| Q6Q0C0 | E3 ubiquitin-protein ligase TRAF7 | TRAF7 | 426 |
| Q6Q0C0 | E3 ubiquitin-protein ligase TRAF7 | TRAF7 | 450 |
| Q9BWF2 | E3 ubiquitin-protein ligase TRAIP | TRAIP | 373 |
| Q9BWF2 | E3 ubiquitin-protein ligase TRAIP | TRAIP | 408 |
| Q96F44 | E3 ubiquitin-protein ligase TRIM11 | TRIM11 | 92 |
| Q96F44 | E3 ubiquitin-protein ligase TRIM11 | TRIM11 | 103 |
| Q96F44 | E3 ubiquitin-protein ligase TRIM11 | TRIM11 | 111 |
| Q96F44 | E3 ubiquitin-protein ligase TRIM11 | TRIM11 | 114 |
| Q96F44 | E3 ubiquitin-protein ligase TRIM11 | TRIM11 | 164 |
| Q96F44 | E3 ubiquitin-protein ligase TRIM11 | TRIM11 | 236 |
| Q96F44 | E3 ubiquitin-protein ligase TRIM11 | TRIM11 | 330 |
| Q96F44 | E3 ubiquitin-protein ligase TRIM11 | TRIM11 | 359 |
| O60858 | E3 ubiquitin-protein ligase TRIM13 | TRIM13 | 25 |
| O60858 | E3 ubiquitin-protein ligase TRIM13 | TRIM13 | 30 |
| O60858 | E3 ubiquitin-protein ligase TRIM13 | TRIM13 | 33 |
| Q9C019 | Tripartite motif-containing protein 15 | TRIM15 | 36 |
| Q9C019 | Tripartite motif-containing protein 15 | TRIM15 | 39 |
| Q9C019 | Tripartite motif-containing protein 15 | TRIM15 | 185 |
| Q9C019 | Tripartite motif-containing protein 15 | TRIM15 | 227 |
| Q9C019 | Tripartite motif-containing protein 15 | TRIM15 | 460 |
| Q9C040 | Tripartite motif-containing protein 2 | TRIM2 | 26 |
| Q9C040 | Tripartite motif-containing protein 2 | TRIM2 | 38 |
| Q9C040 | Tripartite motif-containing protein 2 | TRIM2 | 43 |
| Q9C040 | Tripartite motif-containing protein 2 | TRIM2 | 46 |
| Q9C040 | Tripartite motif-containing protein 2 | TRIM2 | 254 |
| P19474 | E3 ubiquitin-protein ligase TRIM21 | TRIM21 | 51 |
| P19474 | E3 ubiquitin-protein ligase TRIM21 | TRIM21 | 54 |
| P19474 | E3 ubiquitin-protein ligase TRIM21 | TRIM21 | 92 |
| P19474 | E3 ubiquitin-protein ligase TRIM21 | TRIM21 | 103 |
| P19474 | E3 ubiquitin-protein ligase TRIM21 | TRIM21 | 111 |
| P19474 | E3 ubiquitin-protein ligase TRIM21 | TRIM21 | 114 |
| P19474 | E3 ubiquitin-protein ligase TRIM21 | TRIM21 | 273 |
| P19474 | E3 ubiquitin-protein ligase TRIM21 | TRIM21 | 359 |
| P19474 | E3 ubiquitin-protein ligase TRIM21 | TRIM21 | 463 |
| Q8IYM9 | E3 ubiquitin-protein ligase TRIM22 | TRIM22 | 15 |
| Q8IYM9 | E3 ubiquitin-protein ligase TRIM22 | TRIM22 | 18 |
| Q8IYM9 | E3 ubiquitin-protein ligase TRIM22 | TRIM22 | 35 |
| Q8IYM9 | E3 ubiquitin-protein ligase TRIM22 | TRIM22 | 56 |
| Q8IYM9 | E3 ubiquitin-protein ligase TRIM22 | TRIM22 | 59 |
| Q8IYM9 | E3 ubiquitin-protein ligase TRIM22 | TRIM22 | 97 |
| Q8IYM9 | E3 ubiquitin-protein ligase TRIM22 | TRIM22 | 108 |
| Q8IYM9 | E3 ubiquitin-protein ligase TRIM22 | TRIM22 | 140 |
| Q8IYM9 | E3 ubiquitin-protein ligase TRIM22 | TRIM22 | 416 |
| Q8IYM9 | E3 ubiquitin-protein ligase TRIM22 | TRIM22 | 441 |
| P36406 | E3 ubiquitin-protein ligase TRIM23 | TRIM23 | 56 |
| P36406 | E3 ubiquitin-protein ligase TRIM23 | TRIM23 | 236 |
| O15164 | Transcription intermediary factor 1-alpha | TRIM24 | 56 |
| O15164 | Transcription intermediary factor 1-alpha | TRIM24 | 59 |
| O15164 | Transcription intermediary factor 1-alpha | TRIM24 | 73 |
| O15164 | Transcription intermediary factor 1-alpha | TRIM24 | 78 |
| O15164 | Transcription intermediary factor 1-alpha | TRIM24 | 81 |
| O15164 | Transcription intermediary factor 1-alpha | TRIM24 | 127 |
| O15164 | Transcription intermediary factor 1-alpha | TRIM24 | 134 |
| O15164 | Transcription intermediary factor 1-alpha | TRIM24 | 223 |
| O15164 | Transcription intermediary factor 1-alpha | TRIM24 | 235 |
| O15164 | Transcription intermediary factor 1-alpha | TRIM24 | 238 |
| O15164 | Transcription intermediary factor 1-alpha | TRIM24 | 243 |
| O15164 | Transcription intermediary factor 1-alpha | TRIM24 | 246 |
| O15164 | Transcription intermediary factor 1-alpha | TRIM24 | 394 |
| O15164 | Transcription intermediary factor 1-alpha | TRIM24 | 629 |
| O15164 | Transcription intermediary factor 1-alpha | TRIM24 | 867 |
| O15164 | Transcription intermediary factor 1-alpha | TRIM24 | 870 |
| O15164 | Transcription intermediary factor 1-alpha | TRIM24 | 882 |
| Q14258 | E3 ubiquitin/ISG15 ligase TRIM25 | TRIM25 | 70 |
| Q14258 | E3 ubiquitin/ISG15 ligase TRIM25 | TRIM25 | 107 |
| Q14258 | E3 ubiquitin/ISG15 ligase TRIM25 | TRIM25 | 110 |
| Q14258 | E3 ubiquitin/ISG15 ligase TRIM25 | TRIM25 | 157 |
| Q14258 | E3 ubiquitin/ISG15 ligase TRIM25 | TRIM25 | 168 |
| Q14258 | E3 ubiquitin/ISG15 ligase TRIM25 | TRIM25 | 174 |
| Q14258 | E3 ubiquitin/ISG15 ligase TRIM25 | TRIM25 | 176 |
| Q14258 | E3 ubiquitin/ISG15 ligase TRIM25 | TRIM25 | 179 |
| Q14258 | E3 ubiquitin/ISG15 ligase TRIM25 | TRIM25 | 186 |
| Q14258 | E3 ubiquitin/ISG15 ligase TRIM25 | TRIM25 | 351 |
| Q14258 | E3 ubiquitin/ISG15 ligase TRIM25 | TRIM25 | 475 |
| Q14258 | E3 ubiquitin/ISG15 ligase TRIM25 | TRIM25 | 498 |
| Q14258 | E3 ubiquitin/ISG15 ligase TRIM25 | TRIM25 | 506 |
| Q14258 | E3 ubiquitin/ISG15 ligase TRIM25 | TRIM25 | 529 |
| Q12899 | Tripartite motif-containing protein 26 | TRIM26 | 19 |
| Q12899 | Tripartite motif-containing protein 26 | TRIM26 | 36 |
| Q12899 | Tripartite motif-containing protein 26 | TRIM26 | 39 |
| Q12899 | Tripartite motif-containing protein 26 | TRIM26 | 53 |
| Q12899 | Tripartite motif-containing protein 26 | TRIM26 | 56 |
| Q12899 | Tripartite motif-containing protein 26 | TRIM26 | 102 |
| Q12899 | Tripartite motif-containing protein 26 | TRIM26 | 113 |
| Q12899 | Tripartite motif-containing protein 26 | TRIM26 | 121 |
| Q12899 | Tripartite motif-containing protein 26 | TRIM26 | 334 |
| Q12899 | Tripartite motif-containing protein 26 | TRIM26 | 353 |
| P14373 | Zinc finger protein RFP | TRIM27 | 284 |
| P14373 | Zinc finger protein RFP | TRIM27 | 359 |
| P14373 | Zinc finger protein RFP | TRIM27 | 365 |
| P14373 | Zinc finger protein RFP | TRIM27 | 393 |
| P14373 | Zinc finger protein RFP | TRIM27 | 490 |
| Q13263 | Transcription intermediary factor 1-beta | TRIM28 | 65 |
| Q13263 | Transcription intermediary factor 1-beta | TRIM28 | 68 |
| Q13263 | Transcription intermediary factor 1-beta | TRIM28 | 83 |
| Q13263 | Transcription intermediary factor 1-beta | TRIM28 | 88 |
| Q13263 | Transcription intermediary factor 1-beta | TRIM28 | 91 |
| Q13263 | Transcription intermediary factor 1-beta | TRIM28 | 117 |
| Q13263 | Transcription intermediary factor 1-beta | TRIM28 | 120 |
| Q13263 | Transcription intermediary factor 1-beta | TRIM28 | 124 |
| Q13263 | Transcription intermediary factor 1-beta | TRIM28 | 153 |
| Q13263 | Transcription intermediary factor 1-beta | TRIM28 | 156 |
| Q13263 | Transcription intermediary factor 1-beta | TRIM28 | 166 |
| Q13263 | Transcription intermediary factor 1-beta | TRIM28 | 174 |
| Q13263 | Transcription intermediary factor 1-beta | TRIM28 | 177 |
| Q13263 | Transcription intermediary factor 1-beta | TRIM28 | 209 |
| Q13263 | Transcription intermediary factor 1-beta | TRIM28 | 221 |
| Q13263 | Transcription intermediary factor 1-beta | TRIM28 | 224 |
| Q13263 | Transcription intermediary factor 1-beta | TRIM28 | 229 |
| Q13263 | Transcription intermediary factor 1-beta | TRIM28 | 232 |
| Q13263 | Transcription intermediary factor 1-beta | TRIM28 | 628 |
| Q13263 | Transcription intermediary factor 1-beta | TRIM28 | 713 |
| Q13263 | Transcription intermediary factor 1-beta | TRIM28 | 717 |
| O75382 | Tripartite motif-containing protein 3 | TRIM3 | 25 |
| O75382 | Tripartite motif-containing protein 3 | TRIM3 | 37 |
| O75382 | Tripartite motif-containing protein 3 | TRIM3 | 42 |
| O75382 | Tripartite motif-containing protein 3 | TRIM3 | 45 |
| O75382 | Tripartite motif-containing protein 3 | TRIM3 | 227 |
| O75382 | Tripartite motif-containing protein 3 | TRIM3 | 252 |
| O75382 | Tripartite motif-containing protein 3 | TRIM3 | 509 |
| O75382 | Tripartite motif-containing protein 3 | TRIM3 | 597 |
| O75382 | Tripartite motif-containing protein 3 | TRIM3 | 598 |
| Q9BZY9 | E3 ubiquitin-protein ligase TRIM31 | TRIM31 | 48 |
| Q13049 | E3 ubiquitin-protein ligase TRIM32 | TRIM32 | 39 |
| Q13049 | E3 ubiquitin-protein ligase TRIM32 | TRIM32 | 123 |
| Q13049 | E3 ubiquitin-protein ligase TRIM32 | TRIM32 | 134 |
| Q13049 | E3 ubiquitin-protein ligase TRIM32 | TRIM32 | 239 |
| Q13049 | E3 ubiquitin-protein ligase TRIM32 | TRIM32 | 355 |
| Q13049 | E3 ubiquitin-protein ligase TRIM32 | TRIM32 | 464 |
| Q13049 | E3 ubiquitin-protein ligase TRIM32 | TRIM32 | 509 |
| Q13049 | E3 ubiquitin-protein ligase TRIM32 | TRIM32 | 511 |
| Q13049 | E3 ubiquitin-protein ligase TRIM32 | TRIM32 | 577 |
| Q9UPN9 | E3 ubiquitin-protein ligase TRIM33 | TRIM33 | 145 |
| Q9UPN9 | E3 ubiquitin-protein ligase TRIM33 | TRIM33 | 150 |
| Q9UPN9 | E3 ubiquitin-protein ligase TRIM33 | TRIM33 | 153 |
| Q9UPN9 | E3 ubiquitin-protein ligase TRIM33 | TRIM33 | 241 |
| Q9UPN9 | E3 ubiquitin-protein ligase TRIM33 | TRIM33 | 276 |
| Q9UPN9 | E3 ubiquitin-protein ligase TRIM33 | TRIM33 | 288 |
| Q9UPN9 | E3 ubiquitin-protein ligase TRIM33 | TRIM33 | 291 |
| Q9UPN9 | E3 ubiquitin-protein ligase TRIM33 | TRIM33 | 296 |
| Q9UPN9 | E3 ubiquitin-protein ligase TRIM33 | TRIM33 | 299 |
| Q9UPN9 | E3 ubiquitin-protein ligase TRIM33 | TRIM33 | 447 |
| Q9UPN9 | E3 ubiquitin-protein ligase TRIM33 | TRIM33 | 461 |
| Q9UPN9 | E3 ubiquitin-protein ligase TRIM33 | TRIM33 | 582 |
| Q9UPN9 | E3 ubiquitin-protein ligase TRIM33 | TRIM33 | 754 |
| Q9UPN9 | E3 ubiquitin-protein ligase TRIM33 | TRIM33 | 786 |
| Q9UPN9 | E3 ubiquitin-protein ligase TRIM33 | TRIM33 | 805 |
| Q9UPN9 | E3 ubiquitin-protein ligase TRIM33 | TRIM33 | 849 |
| Q9UPN9 | E3 ubiquitin-protein ligase TRIM33 | TRIM33 | 943 |
| Q9UPN9 | E3 ubiquitin-protein ligase TRIM33 | TRIM33 | 1035 |
| Q9UPQ4 | Tripartite motif-containing protein 35 | TRIM35 | 121 |
| Q9NQ86 | E3 ubiquitin-protein ligase TRIM36 | TRIM36 | 33 |
| Q9NQ86 | E3 ubiquitin-protein ligase TRIM36 | TRIM36 | 36 |
| Q9NQ86 | E3 ubiquitin-protein ligase TRIM36 | TRIM36 | 48 |
| Q9NQ86 | E3 ubiquitin-protein ligase TRIM36 | TRIM36 | 53 |
| Q9NQ86 | E3 ubiquitin-protein ligase TRIM36 | TRIM36 | 115 |
| Q9NQ86 | E3 ubiquitin-protein ligase TRIM36 | TRIM36 | 118 |
| Q9NQ86 | E3 ubiquitin-protein ligase TRIM36 | TRIM36 | 157 |
| Q9NQ86 | E3 ubiquitin-protein ligase TRIM36 | TRIM36 | 160 |
| Q9NQ86 | E3 ubiquitin-protein ligase TRIM36 | TRIM36 | 171 |
| Q9NQ86 | E3 ubiquitin-protein ligase TRIM36 | TRIM36 | 224 |
| Q9NQ86 | E3 ubiquitin-protein ligase TRIM36 | TRIM36 | 227 |
| Q9NQ86 | E3 ubiquitin-protein ligase TRIM36 | TRIM36 | 235 |
| O94972 | E3 ubiquitin-protein ligase TRIM37 | TRIM37 | 15 |
| O94972 | E3 ubiquitin-protein ligase TRIM37 | TRIM37 | 31 |
| O94972 | E3 ubiquitin-protein ligase TRIM37 | TRIM37 | 39 |
| O94972 | E3 ubiquitin-protein ligase TRIM37 | TRIM37 | 51 |
| O94972 | E3 ubiquitin-protein ligase TRIM37 | TRIM37 | 54 |
| O94972 | E3 ubiquitin-protein ligase TRIM37 | TRIM37 | 66 |
| O94972 | E3 ubiquitin-protein ligase TRIM37 | TRIM37 | 352 |
| O94972 | E3 ubiquitin-protein ligase TRIM37 | TRIM37 | 477 |
| O94972 | E3 ubiquitin-protein ligase TRIM37 | TRIM37 | 748 |
| O94972 | E3 ubiquitin-protein ligase TRIM37 | TRIM37 | 790 |
| O00635 | E3 ubiquitin-protein ligase TRIM38 | TRIM38 | 58 |
| O00635 | E3 ubiquitin-protein ligase TRIM38 | TRIM38 | 59 |
| O00635 | E3 ubiquitin-protein ligase TRIM38 | TRIM38 | 62 |
| O00635 | E3 ubiquitin-protein ligase TRIM38 | TRIM38 | 133 |
| O00635 | E3 ubiquitin-protein ligase TRIM38 | TRIM38 | 188 |
| O00635 | E3 ubiquitin-protein ligase TRIM38 | TRIM38 | 237 |
| O00635 | E3 ubiquitin-protein ligase TRIM38 | TRIM38 | 274 |
| O00635 | E3 ubiquitin-protein ligase TRIM38 | TRIM38 | 335 |
| O00635 | E3 ubiquitin-protein ligase TRIM38 | TRIM38 | 339 |
| Q9C037 | E3 ubiquitin-protein ligase TRIM4 | TRIM4 | 49 |
| Q9C037 | E3 ubiquitin-protein ligase TRIM4 | TRIM4 | 52 |
| Q9C037 | E3 ubiquitin-protein ligase TRIM4 | TRIM4 | 87 |
| Q9C037 | E3 ubiquitin-protein ligase TRIM4 | TRIM4 | 98 |
| Q9C037 | E3 ubiquitin-protein ligase TRIM4 | TRIM4 | 106 |
| Q9C037 | E3 ubiquitin-protein ligase TRIM4 | TRIM4 | 293 |
| Q9C037 | E3 ubiquitin-protein ligase TRIM4 | TRIM4 | 368 |
| Q8WV44 | E3 ubiquitin-protein ligase TRIM41 | TRIM41 | 180 |
| Q8WV44 | E3 ubiquitin-protein ligase TRIM41 | TRIM41 | 183 |
| Q8WV44 | E3 ubiquitin-protein ligase TRIM41 | TRIM41 | 186 |
| Q8WV44 | E3 ubiquitin-protein ligase TRIM41 | TRIM41 | 227 |
| Q8WV44 | E3 ubiquitin-protein ligase TRIM41 | TRIM41 | 405 |
| Q8WV44 | E3 ubiquitin-protein ligase TRIM41 | TRIM41 | 426 |
| Q7Z4K8 | Tripartite motif-containing protein 46 | TRIM46 | 48 |
| Q7Z4K8 | Tripartite motif-containing protein 46 | TRIM46 | 53 |
| Q7Z4K8 | Tripartite motif-containing protein 46 | TRIM46 | 175 |
| Q7Z4K8 | Tripartite motif-containing protein 46 | TRIM46 | 189 |
| Q7Z4K8 | Tripartite motif-containing protein 46 | TRIM46 | 338 |
| Q7Z4K8 | Tripartite motif-containing protein 46 | TRIM46 | 377 |
| Q96LD4 | Tripartite motif-containing protein 47 | TRIM47 | 54 |
| Q96LD4 | Tripartite motif-containing protein 47 | TRIM47 | 57 |
| Q96LD4 | Tripartite motif-containing protein 47 | TRIM47 | 182 |
| Q96LD4 | Tripartite motif-containing protein 47 | TRIM47 | 199 |
| Q96LD4 | Tripartite motif-containing protein 47 | TRIM47 | 201 |
| Q96LD4 | Tripartite motif-containing protein 47 | TRIM47 | 204 |
| Q96LD4 | Tripartite motif-containing protein 47 | TRIM47 | 347 |
| Q96LD4 | Tripartite motif-containing protein 47 | TRIM47 | 454 |
| Q96LD4 | Tripartite motif-containing protein 47 | TRIM47 | 468 |
| Q96LD4 | Tripartite motif-containing protein 47 | TRIM47 | 520 |
| Q96LD4 | Tripartite motif-containing protein 47 | TRIM47 | 521 |
| Q96LD4 | Tripartite motif-containing protein 47 | TRIM47 | 631 |
| Q9C035 | Tripartite motif-containing protein 5 | TRIM5 | 55 |
| Q9C035 | Tripartite motif-containing protein 5 | TRIM5 | 58 |
| Q9C035 | Tripartite motif-containing protein 5 | TRIM5 | 95 |
| Q9C035 | Tripartite motif-containing protein 5 | TRIM5 | 106 |
| Q9C035 | Tripartite motif-containing protein 5 | TRIM5 | 310 |
| Q9C035 | Tripartite motif-containing protein 5 | TRIM5 | 385 |
| Q9C035 | Tripartite motif-containing protein 5 | TRIM5 | 482 |
| Q9C035 | Tripartite motif-containing protein 5 | TRIM5 | 489 |
| Q9BYV6 | Tripartite motif-containing protein 55 | TRIM55 | 136 |
| Q9BYV6 | Tripartite motif-containing protein 55 | TRIM55 | 144 |
| Q9BRZ2 | E3 ubiquitin-protein ligase TRIM56 | TRIM56 | 24 |
| Q9BRZ2 | E3 ubiquitin-protein ligase TRIM56 | TRIM56 | 36 |
| Q9BRZ2 | E3 ubiquitin-protein ligase TRIM56 | TRIM56 | 41 |
| Q9BRZ2 | E3 ubiquitin-protein ligase TRIM56 | TRIM56 | 44 |
| Q9BRZ2 | E3 ubiquitin-protein ligase TRIM56 | TRIM56 | 56 |
| Q9BRZ2 | E3 ubiquitin-protein ligase TRIM56 | TRIM56 | 59 |
| Q9BRZ2 | E3 ubiquitin-protein ligase TRIM56 | TRIM56 | 101 |
| Q9BRZ2 | E3 ubiquitin-protein ligase TRIM56 | TRIM56 | 104 |
| Q9BRZ2 | E3 ubiquitin-protein ligase TRIM56 | TRIM56 | 120 |
| Q9BRZ2 | E3 ubiquitin-protein ligase TRIM56 | TRIM56 | 123 |
| Q9BRZ2 | E3 ubiquitin-protein ligase TRIM56 | TRIM56 | 128 |
| Q9BRZ2 | E3 ubiquitin-protein ligase TRIM56 | TRIM56 | 131 |
| Q9BRZ2 | E3 ubiquitin-protein ligase TRIM56 | TRIM56 | 169 |
| Q9BRZ2 | E3 ubiquitin-protein ligase TRIM56 | TRIM56 | 181 |
| Q9BRZ2 | E3 ubiquitin-protein ligase TRIM56 | TRIM56 | 184 |
| Q9BRZ2 | E3 ubiquitin-protein ligase TRIM56 | TRIM56 | 189 |
| Q9BRZ2 | E3 ubiquitin-protein ligase TRIM56 | TRIM56 | 202 |
| Q9BRZ2 | E3 ubiquitin-protein ligase TRIM56 | TRIM56 | 338 |
| Q9BRZ2 | E3 ubiquitin-protein ligase TRIM56 | TRIM56 | 347 |
| Q9BRZ2 | E3 ubiquitin-protein ligase TRIM56 | TRIM56 | 363 |
| Q9BRZ2 | E3 ubiquitin-protein ligase TRIM56 | TRIM56 | 514 |
| Q8IWR1 | Tripartite motif-containing protein 59 | TRIM59 | 25 |
| Q8IWR1 | Tripartite motif-containing protein 59 | TRIM59 | 30 |
| Q8IWR1 | Tripartite motif-containing protein 59 | TRIM59 | 208 |
| Q9C030 | Tripartite motif-containing protein 6 | TRIM6 | 97 |
| Q9C030 | Tripartite motif-containing protein 6 | TRIM6 | 335 |
| Q9C030 | Tripartite motif-containing protein 6 | TRIM6 | 342 |
| Q495X7 | Tripartite motif-containing protein 60 | TRIM60 | 59 |
| Q9BVG3 | E3 ubiquitin-protein ligase TRIM62 | TRIM62 | 34 |
| Q9BVG3 | E3 ubiquitin-protein ligase TRIM62 | TRIM62 | 50 |
| Q6PJ69 | Tripartite motif-containing protein 65 | TRIM65 | 15 |
| Q6PJ69 | Tripartite motif-containing protein 65 | TRIM65 | 47 |
| Q6PJ69 | Tripartite motif-containing protein 65 | TRIM65 | 50 |
| Q6PJ69 | Tripartite motif-containing protein 65 | TRIM65 | 106 |
| Q6PJ69 | Tripartite motif-containing protein 65 | TRIM65 | 112 |
| Q6PJ69 | Tripartite motif-containing protein 65 | TRIM65 | 114 |
| Q6PJ69 | Tripartite motif-containing protein 65 | TRIM65 | 117 |
| Q6PJ69 | Tripartite motif-containing protein 65 | TRIM65 | 174 |
| Q6PJ69 | Tripartite motif-containing protein 65 | TRIM65 | 284 |
| Q6PJ69 | Tripartite motif-containing protein 65 | TRIM65 | 320 |
| Q6PJ69 | Tripartite motif-containing protein 65 | TRIM65 | 421 |
| Q6PJ69 | Tripartite motif-containing protein 65 | TRIM65 | 426 |
| Q6PJ69 | Tripartite motif-containing protein 65 | TRIM65 | 500 |
| Q6ZTA4 | Tripartite motif-containing protein 67 | TRIM67 | 147 |
| Q6AZZ1 | E3 ubiquitin-protein ligase TRIM68 | TRIM68 | 352 |
| Q9C029 | E3 ubiquitin-protein ligase TRIM7 | TRIM7 | 56 |
| Q9C029 | E3 ubiquitin-protein ligase TRIM7 | TRIM7 | 78 |
| Q9C029 | E3 ubiquitin-protein ligase TRIM7 | TRIM7 | 130 |
| Q9C029 | E3 ubiquitin-protein ligase TRIM7 | TRIM7 | 141 |
| Q9C029 | E3 ubiquitin-protein ligase TRIM7 | TRIM7 | 149 |
| Q9C029 | E3 ubiquitin-protein ligase TRIM7 | TRIM7 | 152 |
| Q9C029 | E3 ubiquitin-protein ligase TRIM7 | TRIM7 | 378 |
| Q9C029 | E3 ubiquitin-protein ligase TRIM7 | TRIM7 | 501 |
| Q2Q1W2 | E3 ubiquitin-protein ligase TRIM71 | TRIM71 | 19 |
| Q2Q1W2 | E3 ubiquitin-protein ligase TRIM71 | TRIM71 | 66 |
| Q2Q1W2 | E3 ubiquitin-protein ligase TRIM71 | TRIM71 | 69 |
| Q2Q1W2 | E3 ubiquitin-protein ligase TRIM71 | TRIM71 | 94 |
| Q2Q1W2 | E3 ubiquitin-protein ligase TRIM71 | TRIM71 | 199 |
| Q2Q1W2 | E3 ubiquitin-protein ligase TRIM71 | TRIM71 | 212 |
| Q2Q1W2 | E3 ubiquitin-protein ligase TRIM71 | TRIM71 | 215 |
| Q2Q1W2 | E3 ubiquitin-protein ligase TRIM71 | TRIM71 | 223 |
| Q2Q1W2 | E3 ubiquitin-protein ligase TRIM71 | TRIM71 | 301 |
| Q2Q1W2 | E3 ubiquitin-protein ligase TRIM71 | TRIM71 | 380 |
| Q2Q1W2 | E3 ubiquitin-protein ligase TRIM71 | TRIM71 | 607 |
| Q2Q1W2 | E3 ubiquitin-protein ligase TRIM71 | TRIM71 | 636 |
| Q2Q1W2 | E3 ubiquitin-protein ligase TRIM71 | TRIM71 | 661 |
| Q2Q1W2 | E3 ubiquitin-protein ligase TRIM71 | TRIM71 | 778 |
| Q2Q1W2 | E3 ubiquitin-protein ligase TRIM71 | TRIM71 | 829 |
| Q6ZMU5 | Tripartite motif-containing protein 72 | TRIM72 | 56 |
| Q6ZMU5 | Tripartite motif-containing protein 72 | TRIM72 | 86 |
| Q6ZMU5 | Tripartite motif-containing protein 72 | TRIM72 | 97 |
| Q6ZMU5 | Tripartite motif-containing protein 72 | TRIM72 | 105 |
| Q6ZMU5 | Tripartite motif-containing protein 72 | TRIM72 | 108 |
| Q6ZMU5 | Tripartite motif-containing protein 72 | TRIM72 | 144 |
| Q6ZMU5 | Tripartite motif-containing protein 72 | TRIM72 | 242 |
| Q9BZR9 | E3 ubiquitin-protein ligase TRIM8 | TRIM8 | 8 |
| Q9BZR9 | E3 ubiquitin-protein ligase TRIM8 | TRIM8 | 38 |
| Q9BZR9 | E3 ubiquitin-protein ligase TRIM8 | TRIM8 | 92 |
| Q9BZR9 | E3 ubiquitin-protein ligase TRIM8 | TRIM8 | 95 |
| Q9BZR9 | E3 ubiquitin-protein ligase TRIM8 | TRIM8 | 110 |
| Q9BZR9 | E3 ubiquitin-protein ligase TRIM8 | TRIM8 | 114 |
| Q9BZR9 | E3 ubiquitin-protein ligase TRIM8 | TRIM8 | 145 |
| Q9BZR9 | E3 ubiquitin-protein ligase TRIM8 | TRIM8 | 262 |
| Q9BZR9 | E3 ubiquitin-protein ligase TRIM8 | TRIM8 | 315 |
| Q9BZR9 | E3 ubiquitin-protein ligase TRIM8 | TRIM8 | 367 |
| Q9C026 | E3 ubiquitin-protein ligase TRIM9 | TRIM9 | 92 |
| Q9C026 | E3 ubiquitin-protein ligase TRIM9 | TRIM9 | 131 |
| Q9C026 | E3 ubiquitin-protein ligase TRIM9 | TRIM9 | 168 |
| Q9C026 | E3 ubiquitin-protein ligase TRIM9 | TRIM9 | 370 |
| Q9C026 | E3 ubiquitin-protein ligase TRIM9 | TRIM9 | 451 |
| Q14669 | E3 ubiquitin-protein ligase TRIP12 | TRIP12 | 35 |
| Q14669 | E3 ubiquitin-protein ligase TRIP12 | TRIP12 | 156 |
| Q14669 | E3 ubiquitin-protein ligase TRIP12 | TRIP12 | 190 |
| Q14669 | E3 ubiquitin-protein ligase TRIP12 | TRIP12 | 332 |
| Q14669 | E3 ubiquitin-protein ligase TRIP12 | TRIP12 | 502 |
| Q14669 | E3 ubiquitin-protein ligase TRIP12 | TRIP12 | 535 |
| Q14669 | E3 ubiquitin-protein ligase TRIP12 | TRIP12 | 625 |
| Q14669 | E3 ubiquitin-protein ligase TRIP12 | TRIP12 | 703 |
| Q14669 | E3 ubiquitin-protein ligase TRIP12 | TRIP12 | 710 |
| Q14669 | E3 ubiquitin-protein ligase TRIP12 | TRIP12 | 737 |
| Q14669 | E3 ubiquitin-protein ligase TRIP12 | TRIP12 | 947 |
| Q14669 | E3 ubiquitin-protein ligase TRIP12 | TRIP12 | 1225 |
| Q14669 | E3 ubiquitin-protein ligase TRIP12 | TRIP12 | 1276 |
| Q14669 | E3 ubiquitin-protein ligase TRIP12 | TRIP12 | 1411 |
| Q14669 | E3 ubiquitin-protein ligase TRIP12 | TRIP12 | 1538 |
| Q14669 | E3 ubiquitin-protein ligase TRIP12 | TRIP12 | 1867 |
| Q14669 | E3 ubiquitin-protein ligase TRIP12 | TRIP12 | 1959 |
| Q8TEL6 | Short transient receptor potential channel 4-associated protein | TRPC4AP | 448 |
| Q8TEL6 | Short transient receptor potential channel 4-associated protein | TRPC4AP | 508 |
| Q8TEL6 | Short transient receptor potential channel 4-associated protein | TRPC4AP | 749 |
| P53804 | E3 ubiquitin-protein ligase TTC3 | TTC3 | 109 |
| P53804 | E3 ubiquitin-protein ligase TTC3 | TTC3 | 213 |
| P53804 | E3 ubiquitin-protein ligase TTC3 | TTC3 | 422 |
| P53804 | E3 ubiquitin-protein ligase TTC3 | TTC3 | 580 |
| P53804 | E3 ubiquitin-protein ligase TTC3 | TTC3 | 757 |
| P53804 | E3 ubiquitin-protein ligase TTC3 | TTC3 | 823 |
| P53804 | E3 ubiquitin-protein ligase TTC3 | TTC3 | 1107 |
| P53804 | E3 ubiquitin-protein ligase TTC3 | TTC3 | 1159 |
| P53804 | E3 ubiquitin-protein ligase TTC3 | TTC3 | 1168 |
| P53804 | E3 ubiquitin-protein ligase TTC3 | TTC3 | 1249 |
| P53804 | E3 ubiquitin-protein ligase TTC3 | TTC3 | 1293 |
| P53804 | E3 ubiquitin-protein ligase TTC3 | TTC3 | 1318 |
| P53804 | E3 ubiquitin-protein ligase TTC3 | TTC3 | 1857 |
| P53804 | E3 ubiquitin-protein ligase TTC3 | TTC3 | 2022 |
| Q9NRJ4 | Tubby-related protein 4 | TULP4 | 909 |
| Q9NRJ4 | Tubby-related protein 4 | TULP4 | 938 |
| Q9NRJ4 | Tubby-related protein 4 | TULP4 | 1098 |
| Q9BSL1 | Ubiquitin-associated domain-containing protein 1 | UBAC1 | 47 |
| Q9BSL1 | Ubiquitin-associated domain-containing protein 1 | UBAC1 | 322 |
| Q9C0C9 | (E3-independent) E2 ubiquitin-conjugating enzyme | UBE2O | 101 |
| Q9C0C9 | (E3-independent) E2 ubiquitin-conjugating enzyme | UBE2O | 182 |
| Q9C0C9 | (E3-independent) E2 ubiquitin-conjugating enzyme | UBE2O | 208 |
| Q9C0C9 | (E3-independent) E2 ubiquitin-conjugating enzyme | UBE2O | 230 |
| Q9C0C9 | (E3-independent) E2 ubiquitin-conjugating enzyme | UBE2O | 244 |
| Q9C0C9 | (E3-independent) E2 ubiquitin-conjugating enzyme | UBE2O | 314 |
| Q9C0C9 | (E3-independent) E2 ubiquitin-conjugating enzyme | UBE2O | 341 |
| Q9C0C9 | (E3-independent) E2 ubiquitin-conjugating enzyme | UBE2O | 370 |
| Q9C0C9 | (E3-independent) E2 ubiquitin-conjugating enzyme | UBE2O | 375 |
| Q9C0C9 | (E3-independent) E2 ubiquitin-conjugating enzyme | UBE2O | 400 |
| Q9C0C9 | (E3-independent) E2 ubiquitin-conjugating enzyme | UBE2O | 406 |
| Q9C0C9 | (E3-independent) E2 ubiquitin-conjugating enzyme | UBE2O | 585 |
| Q9C0C9 | (E3-independent) E2 ubiquitin-conjugating enzyme | UBE2O | 598 |
| Q9C0C9 | (E3-independent) E2 ubiquitin-conjugating enzyme | UBE2O | 910 |
| Q9C0C9 | (E3-independent) E2 ubiquitin-conjugating enzyme | UBE2O | 913 |
| Q9C0C9 | (E3-independent) E2 ubiquitin-conjugating enzyme | UBE2O | 1040 |
| Q9C0C9 | (E3-independent) E2 ubiquitin-conjugating enzyme | UBE2O | 1099 |
| Q9C0C9 | (E3-independent) E2 ubiquitin-conjugating enzyme | UBE2O | 1288 |
| Q05086 | Ubiquitin-protein ligase E3A | UBE3A | 7 |
| Q05086 | Ubiquitin-protein ligase E3A | UBE3A | 49 |
| Q05086 | Ubiquitin-protein ligase E3A | UBE3A | 57 |
| Q05086 | Ubiquitin-protein ligase E3A | UBE3A | 83 |
| Q05086 | Ubiquitin-protein ligase E3A | UBE3A | 108 |
| Q05086 | Ubiquitin-protein ligase E3A | UBE3A | 140 |
| Q05086 | Ubiquitin-protein ligase E3A | UBE3A | 198 |
| Q05086 | Ubiquitin-protein ligase E3A | UBE3A | 269 |
| Q05086 | Ubiquitin-protein ligase E3A | UBE3A | 310 |
| Q05086 | Ubiquitin-protein ligase E3A | UBE3A | 480 |
| Q05086 | Ubiquitin-protein ligase E3A | UBE3A | 843 |
| Q7Z3V4 | Ubiquitin-protein ligase E3B | UBE3B | 75 |
| Q7Z3V4 | Ubiquitin-protein ligase E3B | UBE3B | 307 |
| Q7Z3V4 | Ubiquitin-protein ligase E3B | UBE3B | 399 |
| Q7Z3V4 | Ubiquitin-protein ligase E3B | UBE3B | 1036 |
| Q15386 | Ubiquitin-protein ligase E3C | UBE3C | 140 |
| Q15386 | Ubiquitin-protein ligase E3C | UBE3C | 152 |
| Q15386 | Ubiquitin-protein ligase E3C | UBE3C | 158 |
| Q15386 | Ubiquitin-protein ligase E3C | UBE3C | 252 |
| Q15386 | Ubiquitin-protein ligase E3C | UBE3C | 395 |
| Q15386 | Ubiquitin-protein ligase E3C | UBE3C | 548 |
| Q15386 | Ubiquitin-protein ligase E3C | UBE3C | 622 |
| Q15386 | Ubiquitin-protein ligase E3C | UBE3C | 1018 |
| Q15386 | Ubiquitin-protein ligase E3C | UBE3C | 1034 |
| Q15386 | Ubiquitin-protein ligase E3C | UBE3C | 1051 |
| Q15386 | Ubiquitin-protein ligase E3C | UBE3C | 1076 |
| Q7Z6J8 | E3 ubiquitin-protein ligase E3D | UBE3D | 52 |
| Q7Z6J8 | E3 ubiquitin-protein ligase E3D | UBE3D | 68 |
| Q7Z6J8 | E3 ubiquitin-protein ligase E3D | UBE3D | 143 |
| Q7Z6J8 | E3 ubiquitin-protein ligase E3D | UBE3D | 144 |
| Q7Z6J8 | E3 ubiquitin-protein ligase E3D | UBE3D | 190 |
| Q7Z6J8 | E3 ubiquitin-protein ligase E3D | UBE3D | 197 |
| Q7Z6J8 | E3 ubiquitin-protein ligase E3D | UBE3D | 330 |
| Q14139 | Ubiquitin conjugation factor E4 A | UBE4A | 79 |
| Q14139 | Ubiquitin conjugation factor E4 A | UBE4A | 175 |
| Q14139 | Ubiquitin conjugation factor E4 A | UBE4A | 202 |
| Q14139 | Ubiquitin conjugation factor E4 A | UBE4A | 398 |
| Q14139 | Ubiquitin conjugation factor E4 A | UBE4A | 450 |
| Q14139 | Ubiquitin conjugation factor E4 A | UBE4A | 465 |
| Q14139 | Ubiquitin conjugation factor E4 A | UBE4A | 490 |
| Q14139 | Ubiquitin conjugation factor E4 A | UBE4A | 710 |
| O95155 | Ubiquitin conjugation factor E4 B | UBE4B | 113 |
| O95155 | Ubiquitin conjugation factor E4 B | UBE4B | 448 |
| O95155 | Ubiquitin conjugation factor E4 B | UBE4B | 464 |
| O95155 | Ubiquitin conjugation factor E4 B | UBE4B | 494 |
| O95155 | Ubiquitin conjugation factor E4 B | UBE4B | 563 |
| O95155 | Ubiquitin conjugation factor E4 B | UBE4B | 581 |
| O95155 | Ubiquitin conjugation factor E4 B | UBE4B | 826 |
| O95155 | Ubiquitin conjugation factor E4 B | UBE4B | 829 |
| O95155 | Ubiquitin conjugation factor E4 B | UBE4B | 1164 |
| O94941 | RING finger protein 37 | UBOX5 | 99 |
| O94941 | RING finger protein 37 | UBOX5 | 187 |
| O94941 | RING finger protein 37 | UBOX5 | 340 |
| O94941 | RING finger protein 37 | UBOX5 | 383 |
| O94941 | RING finger protein 37 | UBOX5 | 416 |
| Q8IWV7 | E3 ubiquitin-protein ligase UBR1 | UBR1 | 163 |
| Q8IWV7 | E3 ubiquitin-protein ligase UBR1 | UBR1 | 180 |
| Q8IWV7 | E3 ubiquitin-protein ligase UBR1 | UBR1 | 251 |
| Q8IWV7 | E3 ubiquitin-protein ligase UBR1 | UBR1 | 279 |
| Q8IWV7 | E3 ubiquitin-protein ligase UBR1 | UBR1 | 350 |
| Q8IWV7 | E3 ubiquitin-protein ligase UBR1 | UBR1 | 477 |
| Q8IWV7 | E3 ubiquitin-protein ligase UBR1 | UBR1 | 511 |
| Q8IWV7 | E3 ubiquitin-protein ligase UBR1 | UBR1 | 872 |
| Q8IWV7 | E3 ubiquitin-protein ligase UBR1 | UBR1 | 993 |
| Q8IWV7 | E3 ubiquitin-protein ligase UBR1 | UBR1 | 1197 |
| Q8IWV7 | E3 ubiquitin-protein ligase UBR1 | UBR1 | 1204 |
| Q8IWV7 | E3 ubiquitin-protein ligase UBR1 | UBR1 | 1577 |
| Q8IWV7 | E3 ubiquitin-protein ligase UBR1 | UBR1 | 1603 |
| Q8IWV7 | E3 ubiquitin-protein ligase UBR1 | UBR1 | 1685 |
| Q8IWV8 | E3 ubiquitin-protein ligase UBR2 | UBR2 | 19 |
| Q8IWV8 | E3 ubiquitin-protein ligase UBR2 | UBR2 | 252 |
| Q8IWV8 | E3 ubiquitin-protein ligase UBR2 | UBR2 | 280 |
| Q8IWV8 | E3 ubiquitin-protein ligase UBR2 | UBR2 | 602 |
| Q8IWV8 | E3 ubiquitin-protein ligase UBR2 | UBR2 | 837 |
| Q8IWV8 | E3 ubiquitin-protein ligase UBR2 | UBR2 | 1360 |
| Q8IWV8 | E3 ubiquitin-protein ligase UBR2 | UBR2 | 1619 |
| Q8IWV8 | E3 ubiquitin-protein ligase UBR2 | UBR2 | 1679 |
| Q8IWV8 | E3 ubiquitin-protein ligase UBR2 | UBR2 | 1691 |
| Q8IWV8 | E3 ubiquitin-protein ligase UBR2 | UBR2 | 1717 |
| Q6ZT12 | E3 ubiquitin-protein ligase UBR3 | UBR3 | 96 |
| Q6ZT12 | E3 ubiquitin-protein ligase UBR3 | UBR3 | 141 |
| Q6ZT12 | E3 ubiquitin-protein ligase UBR3 | UBR3 | 184 |
| Q6ZT12 | E3 ubiquitin-protein ligase UBR3 | UBR3 | 197 |
| Q6ZT12 | E3 ubiquitin-protein ligase UBR3 | UBR3 | 216 |
| Q6ZT12 | E3 ubiquitin-protein ligase UBR3 | UBR3 | 288 |
| Q6ZT12 | E3 ubiquitin-protein ligase UBR3 | UBR3 | 979 |
| Q6ZT12 | E3 ubiquitin-protein ligase UBR3 | UBR3 | 1289 |
| Q6ZT12 | E3 ubiquitin-protein ligase UBR3 | UBR3 | 1373 |
| Q6ZT12 | E3 ubiquitin-protein ligase UBR3 | UBR3 | 1471 |
| Q6ZT12 | E3 ubiquitin-protein ligase UBR3 | UBR3 | 1598 |
| Q6ZT12 | E3 ubiquitin-protein ligase UBR3 | UBR3 | 1629 |
| Q6ZT12 | E3 ubiquitin-protein ligase UBR3 | UBR3 | 1769 |
| Q6ZT12 | E3 ubiquitin-protein ligase UBR3 | UBR3 | 1787 |
| Q6ZT12 | E3 ubiquitin-protein ligase UBR3 | UBR3 | 1858 |
| Q5T4S7 | E3 ubiquitin-protein ligase UBR4 | UBR4 | 140 |
| Q5T4S7 | E3 ubiquitin-protein ligase UBR4 | UBR4 | 260 |
| Q5T4S7 | E3 ubiquitin-protein ligase UBR4 | UBR4 | 546 |
| Q5T4S7 | E3 ubiquitin-protein ligase UBR4 | UBR4 | 779 |
| Q5T4S7 | E3 ubiquitin-protein ligase UBR4 | UBR4 | 934 |
| Q5T4S7 | E3 ubiquitin-protein ligase UBR4 | UBR4 | 953 |
| Q5T4S7 | E3 ubiquitin-protein ligase UBR4 | UBR4 | 1080 |
| Q5T4S7 | E3 ubiquitin-protein ligase UBR4 | UBR4 | 1274 |
| Q5T4S7 | E3 ubiquitin-protein ligase UBR4 | UBR4 | 1458 |
| Q5T4S7 | E3 ubiquitin-protein ligase UBR4 | UBR4 | 1662 |
| Q5T4S7 | E3 ubiquitin-protein ligase UBR4 | UBR4 | 1682 |
| Q5T4S7 | E3 ubiquitin-protein ligase UBR4 | UBR4 | 1714 |
| Q5T4S7 | E3 ubiquitin-protein ligase UBR4 | UBR4 | 1724 |
| Q5T4S7 | E3 ubiquitin-protein ligase UBR4 | UBR4 | 1789 |
| Q5T4S7 | E3 ubiquitin-protein ligase UBR4 | UBR4 | 1900 |
| Q5T4S7 | E3 ubiquitin-protein ligase UBR4 | UBR4 | 1962 |
| Q5T4S7 | E3 ubiquitin-protein ligase UBR4 | UBR4 | 2222 |
| Q5T4S7 | E3 ubiquitin-protein ligase UBR4 | UBR4 | 2449 |
| Q5T4S7 | E3 ubiquitin-protein ligase UBR4 | UBR4 | 2489 |
| Q5T4S7 | E3 ubiquitin-protein ligase UBR4 | UBR4 | 2554 |
| Q5T4S7 | E3 ubiquitin-protein ligase UBR4 | UBR4 | 2618 |
| Q5T4S7 | E3 ubiquitin-protein ligase UBR4 | UBR4 | 2619 |
| Q5T4S7 | E3 ubiquitin-protein ligase UBR4 | UBR4 | 2688 |
| Q5T4S7 | E3 ubiquitin-protein ligase UBR4 | UBR4 | 2697 |
| Q5T4S7 | E3 ubiquitin-protein ligase UBR4 | UBR4 | 3075 |
| Q5T4S7 | E3 ubiquitin-protein ligase UBR4 | UBR4 | 3279 |
| Q5T4S7 | E3 ubiquitin-protein ligase UBR4 | UBR4 | 3430 |
| Q5T4S7 | E3 ubiquitin-protein ligase UBR4 | UBR4 | 3663 |
| Q5T4S7 | E3 ubiquitin-protein ligase UBR4 | UBR4 | 3677 |
| Q5T4S7 | E3 ubiquitin-protein ligase UBR4 | UBR4 | 3684 |
| Q5T4S7 | E3 ubiquitin-protein ligase UBR4 | UBR4 | 3700 |
| Q5T4S7 | E3 ubiquitin-protein ligase UBR4 | UBR4 | 3703 |
| Q5T4S7 | E3 ubiquitin-protein ligase UBR4 | UBR4 | 3706 |
| Q5T4S7 | E3 ubiquitin-protein ligase UBR4 | UBR4 | 3720 |
| Q5T4S7 | E3 ubiquitin-protein ligase UBR4 | UBR4 | 3721 |
| Q5T4S7 | E3 ubiquitin-protein ligase UBR4 | UBR4 | 3765 |
| Q5T4S7 | E3 ubiquitin-protein ligase UBR4 | UBR4 | 3804 |
| Q5T4S7 | E3 ubiquitin-protein ligase UBR4 | UBR4 | 3807 |
| Q5T4S7 | E3 ubiquitin-protein ligase UBR4 | UBR4 | 3864 |
| Q5T4S7 | E3 ubiquitin-protein ligase UBR4 | UBR4 | 3872 |
| Q5T4S7 | E3 ubiquitin-protein ligase UBR4 | UBR4 | 3875 |
| Q5T4S7 | E3 ubiquitin-protein ligase UBR4 | UBR4 | 3930 |
| Q5T4S7 | E3 ubiquitin-protein ligase UBR4 | UBR4 | 4049 |
| Q5T4S7 | E3 ubiquitin-protein ligase UBR4 | UBR4 | 4146 |
| Q5T4S7 | E3 ubiquitin-protein ligase UBR4 | UBR4 | 4272 |
| Q5T4S7 | E3 ubiquitin-protein ligase UBR4 | UBR4 | 4430 |
| Q5T4S7 | E3 ubiquitin-protein ligase UBR4 | UBR4 | 4487 |
| Q5T4S7 | E3 ubiquitin-protein ligase UBR4 | UBR4 | 4642 |
| Q5T4S7 | E3 ubiquitin-protein ligase UBR4 | UBR4 | 4663 |
| Q5T4S7 | E3 ubiquitin-protein ligase UBR4 | UBR4 | 4838 |
| Q5T4S7 | E3 ubiquitin-protein ligase UBR4 | UBR4 | 4839 |
| Q5T4S7 | E3 ubiquitin-protein ligase UBR4 | UBR4 | 4841 |
| Q5T4S7 | E3 ubiquitin-protein ligase UBR4 | UBR4 | 4890 |
| Q5T4S7 | E3 ubiquitin-protein ligase UBR4 | UBR4 | 4916 |
| Q5T4S7 | E3 ubiquitin-protein ligase UBR4 | UBR4 | 4935 |
| Q5T4S7 | E3 ubiquitin-protein ligase UBR4 | UBR4 | 4946 |
| Q5T4S7 | E3 ubiquitin-protein ligase UBR4 | UBR4 | 5121 |
| O95071 | E3 ubiquitin-protein ligase UBR5 | UBR5 | 373 |
| O95071 | E3 ubiquitin-protein ligase UBR5 | UBR5 | 535 |
| O95071 | E3 ubiquitin-protein ligase UBR5 | UBR5 | 572 |
| O95071 | E3 ubiquitin-protein ligase UBR5 | UBR5 | 617 |
| O95071 | E3 ubiquitin-protein ligase UBR5 | UBR5 | 691 |
| O95071 | E3 ubiquitin-protein ligase UBR5 | UBR5 | 730 |
| O95071 | E3 ubiquitin-protein ligase UBR5 | UBR5 | 739 |
| O95071 | E3 ubiquitin-protein ligase UBR5 | UBR5 | 883 |
| O95071 | E3 ubiquitin-protein ligase UBR5 | UBR5 | 1093 |
| O95071 | E3 ubiquitin-protein ligase UBR5 | UBR5 | 1215 |
| O95071 | E3 ubiquitin-protein ligase UBR5 | UBR5 | 1221 |
| O95071 | E3 ubiquitin-protein ligase UBR5 | UBR5 | 1232 |
| O95071 | E3 ubiquitin-protein ligase UBR5 | UBR5 | 1291 |
| O95071 | E3 ubiquitin-protein ligase UBR5 | UBR5 | 2084 |
| O95071 | E3 ubiquitin-protein ligase UBR5 | UBR5 | 2094 |
| O95071 | E3 ubiquitin-protein ligase UBR5 | UBR5 | 2267 |
| O95071 | E3 ubiquitin-protein ligase UBR5 | UBR5 | 2314 |
| O95071 | E3 ubiquitin-protein ligase UBR5 | UBR5 | 2546 |
| O95071 | E3 ubiquitin-protein ligase UBR5 | UBR5 | 2768 |
| Q8N806 | Putative E3 ubiquitin-protein ligase UBR7 | UBR7 | 35 |
| Q8N806 | Putative E3 ubiquitin-protein ligase UBR7 | UBR7 | 46 |
| Q8N806 | Putative E3 ubiquitin-protein ligase UBR7 | UBR7 | 61 |
| Q8N806 | Putative E3 ubiquitin-protein ligase UBR7 | UBR7 | 75 |
| Q8N806 | Putative E3 ubiquitin-protein ligase UBR7 | UBR7 | 78 |
| Q8N806 | Putative E3 ubiquitin-protein ligase UBR7 | UBR7 | 82 |
| Q8N806 | Putative E3 ubiquitin-protein ligase UBR7 | UBR7 | 99 |
| Q8N806 | Putative E3 ubiquitin-protein ligase UBR7 | UBR7 | 101 |
| Q8N806 | Putative E3 ubiquitin-protein ligase UBR7 | UBR7 | 111 |
| Q8N806 | Putative E3 ubiquitin-protein ligase UBR7 | UBR7 | 137 |
| Q8N806 | Putative E3 ubiquitin-protein ligase UBR7 | UBR7 | 158 |
| Q8N806 | Putative E3 ubiquitin-protein ligase UBR7 | UBR7 | 185 |
| Q8N806 | Putative E3 ubiquitin-protein ligase UBR7 | UBR7 | 260 |
| Q8N806 | Putative E3 ubiquitin-protein ligase UBR7 | UBR7 | 314 |
| Q8N806 | Putative E3 ubiquitin-protein ligase UBR7 | UBR7 | 316 |
| Q8N806 | Putative E3 ubiquitin-protein ligase UBR7 | UBR7 | 374 |
| Q8N806 | Putative E3 ubiquitin-protein ligase UBR7 | UBR7 | 424 |
| O94874 | E3 UFM1-protein ligase 1 | UFL1 | 32 |
| O94874 | E3 UFM1-protein ligase 1 | UFL1 | 143 |
| O94874 | E3 UFM1-protein ligase 1 | UFL1 | 372 |
| O94874 | E3 UFM1-protein ligase 1 | UFL1 | 708 |
| Q96T88 | E3 ubiquitin-protein ligase UHRF1 | UHRF1 | 97 |
| Q96T88 | E3 ubiquitin-protein ligase UHRF1 | UHRF1 | 98 |
| Q96T88 | E3 ubiquitin-protein ligase UHRF1 | UHRF1 | 170 |
| Q96T88 | E3 ubiquitin-protein ligase UHRF1 | UHRF1 | 302 |
| Q96T88 | E3 ubiquitin-protein ligase UHRF1 | UHRF1 | 305 |
| Q96T88 | E3 ubiquitin-protein ligase UHRF1 | UHRF1 | 316 |
| Q96T88 | E3 ubiquitin-protein ligase UHRF1 | UHRF1 | 318 |
| Q96T88 | E3 ubiquitin-protein ligase UHRF1 | UHRF1 | 321 |
| Q96T88 | E3 ubiquitin-protein ligase UHRF1 | UHRF1 | 403 |
| Q96T88 | E3 ubiquitin-protein ligase UHRF1 | UHRF1 | 410 |
| Q96T88 | E3 ubiquitin-protein ligase UHRF1 | UHRF1 | 497 |
| Q96T88 | E3 ubiquitin-protein ligase UHRF1 | UHRF1 | 512 |
| Q96T88 | E3 ubiquitin-protein ligase UHRF1 | UHRF1 | 726 |
| Q96T88 | E3 ubiquitin-protein ligase UHRF1 | UHRF1 | 727 |
| Q96T88 | E3 ubiquitin-protein ligase UHRF1 | UHRF1 | 739 |
| Q96T88 | E3 ubiquitin-protein ligase UHRF1 | UHRF1 | 747 |
| Q96T88 | E3 ubiquitin-protein ligase UHRF1 | UHRF1 | 759 |
| Q96T88 | E3 ubiquitin-protein ligase UHRF1 | UHRF1 | 762 |
| Q96PU4 | E3 ubiquitin-protein ligase UHRF2 | UHRF2 | 14 |
| Q96PU4 | E3 ubiquitin-protein ligase UHRF2 | UHRF2 | 42 |
| Q96PU4 | E3 ubiquitin-protein ligase UHRF2 | UHRF2 | 92 |
| Q96PU4 | E3 ubiquitin-protein ligase UHRF2 | UHRF2 | 171 |
| Q96PU4 | E3 ubiquitin-protein ligase UHRF2 | UHRF2 | 200 |
| Q96PU4 | E3 ubiquitin-protein ligase UHRF2 | UHRF2 | 331 |
| Q96PU4 | E3 ubiquitin-protein ligase UHRF2 | UHRF2 | 334 |
| Q96PU4 | E3 ubiquitin-protein ligase UHRF2 | UHRF2 | 342 |
| Q96PU4 | E3 ubiquitin-protein ligase UHRF2 | UHRF2 | 345 |
| Q96PU4 | E3 ubiquitin-protein ligase UHRF2 | UHRF2 | 347 |
| Q96PU4 | E3 ubiquitin-protein ligase UHRF2 | UHRF2 | 432 |
| Q96PU4 | E3 ubiquitin-protein ligase UHRF2 | UHRF2 | 439 |
| Q96PU4 | E3 ubiquitin-protein ligase UHRF2 | UHRF2 | 541 |
| Q96PU4 | E3 ubiquitin-protein ligase UHRF2 | UHRF2 | 671 |
| Q96PU4 | E3 ubiquitin-protein ligase UHRF2 | UHRF2 | 704 |
| Q96PU4 | E3 ubiquitin-protein ligase UHRF2 | UHRF2 | 768 |
| Q96PU4 | E3 ubiquitin-protein ligase UHRF2 | UHRF2 | 771 |
| Q9C0B0 | RING finger protein unkempt homolog | UNK | 45 |
| Q9C0B0 | RING finger protein unkempt homolog | UNK | 53 |
| Q9C0B0 | RING finger protein unkempt homolog | UNK | 106 |
| Q9C0B0 | RING finger protein unkempt homolog | UNK | 130 |
| Q9C0B0 | RING finger protein unkempt homolog | UNK | 140 |
| Q9C0B0 | RING finger protein unkempt homolog | UNK | 147 |
| Q9C0B0 | RING finger protein unkempt homolog | UNK | 221 |
| Q9C0B0 | RING finger protein unkempt homolog | UNK | 383 |
| Q9C0B0 | RING finger protein unkempt homolog | UNK | 675 |
| Q9C0B0 | RING finger protein unkempt homolog | UNK | 696 |
| Q9C0B0 | RING finger protein unkempt homolog | UNK | 772 |
| Q9C0B0 | RING finger protein unkempt homolog | UNK | 782 |
| Q9C0B0 | RING finger protein unkempt homolog | UNK | 788 |
| Q9C0B0 | RING finger protein unkempt homolog | UNK | 791 |
| Q9C0B0 | RING finger protein unkempt homolog | UNK | 797 |
| Q9C0B0 | RING finger protein unkempt homolog | UNK | 800 |
| Q9H9P5 | Putative E3 ubiquitin-protein ligase UNKL | UNKL | 36 |
| Q9H9P5 | Putative E3 ubiquitin-protein ligase UNKL | UNKL | 213 |
| Q9H9P5 | Putative E3 ubiquitin-protein ligase UNKL | UNKL | 226 |
| Q9H9P5 | Putative E3 ubiquitin-protein ligase UNKL | UNKL | 249 |
| Q9H9P5 | Putative E3 ubiquitin-protein ligase UNKL | UNKL | 270 |
| Q9H9P5 | Putative E3 ubiquitin-protein ligase UNKL | UNKL | 297 |
| Q9H9P5 | Putative E3 ubiquitin-protein ligase UNKL | UNKL | 303 |
| Q9H9P5 | Putative E3 ubiquitin-protein ligase UNKL | UNKL | 320 |
| Q9H9P5 | Putative E3 ubiquitin-protein ligase UNKL | UNKL | 595 |
| Q9H9P5 | Putative E3 ubiquitin-protein ligase UNKL | UNKL | 639 |
| Q9H9P5 | Putative E3 ubiquitin-protein ligase UNKL | UNKL | 642 |
| P40337 | von Hippel-Lindau disease tumor suppressor | VHL | 77 |
| P40337 | von Hippel-Lindau disease tumor suppressor | VHL | 162 |
| Q9H270 | Vacuolar protein sorting-associated protein 11 homolog | VPS11 | 44 |
| Q9H270 | Vacuolar protein sorting-associated protein 11 homolog | VPS11 | 52 |
| Q9H270 | Vacuolar protein sorting-associated protein 11 homolog | VPS11 | 128 |
| Q9H270 | Vacuolar protein sorting-associated protein 11 homolog | VPS11 | 231 |
| Q9H270 | Vacuolar protein sorting-associated protein 11 homolog | VPS11 | 317 |
| Q9H270 | Vacuolar protein sorting-associated protein 11 homolog | VPS11 | 460 |
| Q9H270 | Vacuolar protein sorting-associated protein 11 homolog | VPS11 | 568 |
| Q9H270 | Vacuolar protein sorting-associated protein 11 homolog | VPS11 | 586 |
| Q9H270 | Vacuolar protein sorting-associated protein 11 homolog | VPS11 | 660 |
| Q9H270 | Vacuolar protein sorting-associated protein 11 homolog | VPS11 | 734 |
| Q9H270 | Vacuolar protein sorting-associated protein 11 homolog | VPS11 | 890 |
| Q9P253 | Vacuolar protein sorting-associated protein 18 homolog | VPS18 | 22 |
| Q9P253 | Vacuolar protein sorting-associated protein 18 homolog | VPS18 | 421 |
| Q9P253 | Vacuolar protein sorting-associated protein 18 homolog | VPS18 | 433 |
| Q9P253 | Vacuolar protein sorting-associated protein 18 homolog | VPS18 | 445 |
| Q9P253 | Vacuolar protein sorting-associated protein 18 homolog | VPS18 | 522 |
| Q9P253 | Vacuolar protein sorting-associated protein 18 homolog | VPS18 | 704 |
| Q9P253 | Vacuolar protein sorting-associated protein 18 homolog | VPS18 | 713 |
| Q9P253 | Vacuolar protein sorting-associated protein 18 homolog | VPS18 | 741 |
| Q9P253 | Vacuolar protein sorting-associated protein 18 homolog | VPS18 | 776 |
| Q9P253 | Vacuolar protein sorting-associated protein 18 homolog | VPS18 | 780 |
| Q9P253 | Vacuolar protein sorting-associated protein 18 homolog | VPS18 | 806 |
| P49754 | Vacuolar protein sorting-associated protein 41 homolog | VPS41 | 299 |
| P49754 | Vacuolar protein sorting-associated protein 41 homolog | VPS41 | 638 |
| P49754 | Vacuolar protein sorting-associated protein 41 homolog | VPS41 | 794 |
| P49754 | Vacuolar protein sorting-associated protein 41 homolog | VPS41 | 814 |
| Q8N3P4 | Vacuolar protein sorting-associated protein 8 homolog | VPS 8 | 286 |
| Q8N3P4 | Vacuolar protein sorting-associated protein 8 homolog | VPS8 | 564 |
| Q8N3P4 | Vacuolar protein sorting-associated protein 8 homolog | VPS8 | 974 |
| Q8N3P4 | Vacuolar protein sorting-associated protein 8 homolog | VPS8 | 1309 |
| Q8N3P4 | Vacuolar protein sorting-associated protein 8 homolog | VPS8 | 1371 |
| Q9H7D7 | WD repeat-containing protein 26 | WDR26 | 153 |
| Q9H7D7 | WD repeat-containing protein 26 | WDR26 | 238 |
| Q9H7D7 | WD repeat-containing protein 26 | WDR26 | 259 |
| Q9H7D7 | WD repeat-containing protein 26 | WDR26 | 316 |
| Q9H7D7 | WD repeat-containing protein 26 | WDR26 | 338 |
| Q9H7D7 | WD repeat-containing protein 26 | WDR26 | 345 |
| Q9H7D7 | WD repeat-containing protein 26 | WDR26 | 355 |
| Q9H7D7 | WD repeat-containing protein 26 | WDR26 | 473 |
| Q9H7D7 | WD repeat-containing protein 26 | WDR26 | 490 |
| Q9H7D7 | WD repeat-containing protein 26 | WDR26 | 493 |
| Q9H7D7 | WD repeat-containing protein 26 | WDR26 | 656 |
| Q8N9V3 | WD repeat, SAM and U-box domain-containing protein 1 | WDSUB1 | 172 |
| Q8N9V3 | WD repeat, SAM and U-box domain-containing protein 1 | WDSUB1 | 316 |
| Q8N9V3 | WD repeat, SAM and U-box domain-containing protein 1 | WDSUB1 | 410 |
| Q8N5D0 | WD and tetratricopeptide repeats protein 1 | WDTC1 | 181 |
| Q8N5D0 | WD and tetratricopeptide repeats protein 1 | WDTC1 | 189 |
| Q8N5D0 | WD and tetratricopeptide repeats protein 1 | WDTC1 | 199 |
| Q8N5D0 | WD and tetratricopeptide repeats protein 1 | WDTC1 | 236 |
| Q8N5D0 | WD and tetratricopeptide repeats protein 1 | WDTC1 | 312 |
| Q8N5D0 | WD and tetratricopeptide repeats protein 1 | WDTC1 | 375 |
| Q8N5D0 | WD and tetratricopeptide repeats protein 1 | WDTC1 | 431 |
| Q8N5D0 | WD and tetratricopeptide repeats protein 1 | WDTC1 | 472 |
| Q8N5D0 | WD and tetratricopeptide repeats protein 1 | WDTC1 | 531 |
| Q8N5D0 | WD and tetratricopeptide repeats protein 1 | WDTC1 | 534 |
| Q8N5D0 | WD and tetratricopeptide repeats protein 1 | WDTC1 | 585 |
| Q8N5D0 | WD and tetratricopeptide repeats protein 1 | WDTC1 | 593 |
| Q9Y6I7 | WD repeat and SOCS box-containing protein 1 | WSB1 | 33 |
| Q9Y6I7 | WD repeat and SOCS box-containing protein 1 | WSB1 | 66 |
| Q9Y6I7 | WD repeat and SOCS box-containing protein 1 | WSB1 | 126 |
| Q9H0M0 | NEDD4-like E3 ubiquitin-protein ligase WWP1 | WWP1 | 335 |
| Q9H0M0 | NEDD4-like E3 ubiquitin-protein ligase WWP1 | WWP1 | 854 |
| Q9H0M0 | NEDD4-like E3 ubiquitin-protein ligase WWP1 | WWP1 | 890 |
| O00308 | NEDD4-like E3 ubiquitin-protein ligase WWP2 | WWP2 | 186 |
| O00308 | NEDD4-like E3 ubiquitin-protein ligase WWP2 | WWP2 | 750 |
| O00308 | NEDD4-like E3 ubiquitin-protein ligase WWP2 | WWP2 | 802 |
| O00308 | NEDD4-like E3 ubiquitin-protein ligase WWP2 | WWP2 | 822 |
| O00308 | NEDD4-like E3 ubiquitin-protein ligase WWP2 | WWP2 | 838 |
| P98170 | E3 ubiquitin-protein ligase XIAP | XIAP | 12 |
| P98170 | E3 ubiquitin-protein ligase XIAP | XIAP | 90 |
| P98170 | E3 ubiquitin-protein ligase XIAP | XIAP | 202 |
| P98170 | E3 ubiquitin-protein ligase XIAP | XIAP | 203 |
| P98170 | E3 ubiquitin-protein ligase XIAP | XIAP | 213 |
| P98170 | E3 ubiquitin-protein ligase XIAP | XIAP | 327 |
| P98170 | E3 ubiquitin-protein ligase XIAP | XIAP | 351 |
| P98170 | E3 ubiquitin-protein ligase XIAP | XIAP | 471 |
| P98170 | E3 ubiquitin-protein ligase XIAP | XIAP | 474 |
| P98170 | E3 ubiquitin-protein ligase XIAP | XIAP | 481 |
| P10074 | Telomere zinc finger-associated protein | ZBTB48 | 111 |
| P10074 | Telomere zinc finger-associated protein | ZBTB48 | 190 |
| P10074 | Telomere zinc finger-associated protein | ZBTB48 | 265 |
| P10074 | Telomere zinc finger-associated protein | ZBTB48 | 380 |
| Q86WB0 | Nuclear-interacting partner of ALK | ZC3HC1 | 102 |
| Q86WB0 | Nuclear-interacting partner of ALK | ZC3HC1 | 112 |
| Q86WB0 | Nuclear-interacting partner of ALK | ZC3HC1 | 117 |
| Q86WB0 | Nuclear-interacting partner of ALK | ZC3HC1 | 120 |
| Q86WB0 | Nuclear-interacting partner of ALK | ZC3HC1 | 125 |
| Q86WB0 | Nuclear-interacting partner of ALK | ZC3HC1 | 149 |
| Q86WB0 | Nuclear-interacting partner of ALK | ZC3HC1 | 156 |
| Q86WB0 | Nuclear-interacting partner of ALK | ZC3HC1 | 190 |
| Q86WB0 | Nuclear-interacting partner of ALK | ZC3HC1 | 208 |
| Q86WB0 | Nuclear-interacting partner of ALK | ZC3HC1 | 406 |
| Q86WB0 | Nuclear-interacting partner of ALK | ZC3HC1 | 429 |
| Q86WB0 | Nuclear-interacting partner of ALK | ZC3HC1 | 500 |
| Q86WB0 | Nuclear-interacting partner of ALK | ZC3HC1 | 502 |
| Q9H8U3 | AN1-type zinc finger protein 3 | ZFAND3 | 18 |
| Q9H8U3 | AN1-type zinc finger protein 3 | ZFAND3 | 20 |
| Q9H8U3 | AN1-type zinc finger protein 3 | ZFAND3 | 32 |
| Q9H8U3 | AN1-type zinc finger protein 3 | ZFAND3 | 35 |
| Q9H8U3 | AN1-type zinc finger protein 3 | ZFAND3 | 118 |
| Q9H8U3 | AN1-type zinc finger protein 3 | ZFAND3 | 157 |
| Q9H8U3 | AN1-type zinc finger protein 3 | ZFAND3 | 160 |
| Q9H8U3 | AN1-type zinc finger protein 3 | ZFAND3 | 174 |
| Q9H8U3 | AN1-type zinc finger protein 3 | ZFAND3 | 192 |
| O76080 | AN1-type zinc finger protein 5 | ZFAND5 | 30 |
| O76080 | AN1-type zinc finger protein 5 | ZFAND5 | 33 |
| O76080 | AN1-type zinc finger protein 5 | ZFAND5 | 76 |
| O76080 | AN1-type zinc finger protein 5 | ZFAND5 | 168 |
| O76080 | AN1-type zinc finger protein 5 | ZFAND5 | 170 |
| O76080 | AN1-type zinc finger protein 5 | ZFAND5 | 186 |
| Q6FIF0 | AN1-type zinc finger protein 6 | ZFAND6 | 30 |
| Q6FIF0 | AN1-type zinc finger protein 6 | ZFAND6 | 33 |
| Q6FIF0 | AN1-type zinc finger protein 6 | ZFAND6 | 163 |
| Q6FIF0 | AN1-type zinc finger protein 6 | ZFAND6 | 165 |
| Q6FIF0 | AN1-type zinc finger protein 6 | ZFAND6 | 170 |
| Q6FIF0 | AN1-type zinc finger protein 6 | ZFAND6 | 181 |
| Q96JP5 | E3 ubiquitin-protein ligase ZFP91 | ZFP91 | 115 |
| Q96JP5 | E3 ubiquitin-protein ligase ZFP91 | ZFP91 | 182 |
| Q96JP5 | E3 ubiquitin-protein ligase ZFP91 | ZFP91 | 313 |
| Q96JP5 | E3 ubiquitin-protein ligase ZFP91 | ZFP91 | 318 |
| Q96JP5 | E3 ubiquitin-protein ligase ZFP91 | ZFP91 | 344 |
| Q96JP5 | E3 ubiquitin-protein ligase ZFP91 | ZFP91 | 349 |
| Q96JP5 | E3 ubiquitin-protein ligase ZFP91 | ZFP91 | 409 |
| Q96JP5 | E3 ubiquitin-protein ligase ZFP91 | ZFP91 | 520 |
| O95159 | Zinc finger protein-like 1 | ZFPL1 | 16 |
| O95159 | Zinc finger protein-like 1 | ZFPL1 | 24 |
| O95159 | Zinc finger protein-like 1 | ZFPL1 | 27 |
| O95159 | Zinc finger protein-like 1 | ZFPL1 | 56 |
| O95159 | Zinc finger protein-like 1 | ZFPL1 | 70 |
| O95159 | Zinc finger protein-like 1 | ZFPL1 | 78 |
| O95159 | Zinc finger protein-like 1 | ZFPL1 | 97 |
| O95159 | Zinc finger protein-like 1 | ZFPL1 | 230 |
| Q9ULJ6 | Zinc finger MIZ domain-containing protein 1 | ZMIZ1 | 17 |
| Q9ULJ6 | Zinc finger MIZ domain-containing protein 1 | ZMIZ1 | 81 |
| Q9ULJ6 | Zinc finger MIZ domain-containing protein 1 | ZMIZ1 | 612 |
| Q9ULJ6 | Zinc finger MIZ domain-containing protein 1 | ZMIZ1 | 657 |
| Q9ULJ6 | Zinc finger MIZ domain-containing protein 1 | ZMIZ1 | 705 |
| Q9ULJ6 | Zinc finger MIZ domain-containing protein 1 | ZMIZ1 | 742 |
| Q9ULJ6 | Zinc finger MIZ domain-containing protein 1 | ZMIZ1 | 763 |
| Q9ULJ6 | Zinc finger MIZ domain-containing protein 1 | ZMIZ1 | 784 |
| Q8NF64 | Zinc finger MIZ domain-containing protein 2 | ZMIZ2 | 418 |
| Q8NF64 | Zinc finger MIZ domain-containing protein 2 | ZMIZ2 | 471 |
| Q8NF64 | Zinc finger MIZ domain-containing protein 2 | ZMIZ2 | 516 |
| Q8NF64 | Zinc finger MIZ domain-containing protein 2 | ZMIZ2 | 564 |
| Q8NF64 | Zinc finger MIZ domain-containing protein 2 | ZMIZ2 | 600 |
| Q8NF64 | Zinc finger MIZ domain-containing protein 2 | ZMIZ2 | 632 |
| Q8NF64 | Zinc finger MIZ domain-containing protein 2 | ZMIZ2 | 642 |
| Q9Y4E5 | E3 SUMO-protein ligase ZNF451 | ZNF451 | 133 |
| Q9Y4E5 | E3 SUMO-protein ligase ZNF451 | ZNF451 | 150 |
| Q9Y4E5 | E3 SUMO-protein ligase ZNF451 | ZNF451 | 268 |
| Q9Y4E5 | E3 SUMO-protein ligase ZNF451 | ZNF451 | 317 |
| Q9Y4E5 | E3 SUMO-protein ligase ZNF451 | ZNF451 | 320 |
| Q9Y4E5 | E3 SUMO-protein ligase ZNF451 | ZNF451 | 441 |
| Q9Y4E5 | E3 SUMO-protein ligase ZNF451 | ZNF451 | 461 |
| Q9Y4E5 | E3 SUMO-protein ligase ZNF451 | ZNF451 | 507 |
| Q9Y4E5 | E3 SUMO-protein ligase ZNF451 | ZNF451 | 611 |
| Q9Y4E5 | E3 SUMO-protein ligase ZNF451 | ZNF451 | 638 |
| Q9Y4E5 | E3 SUMO-protein ligase ZNF451 | ZNF451 | 641 |
| Q9Y4E5 | E3 SUMO-protein ligase ZNF451 | ZNF451 | 655 |
| Q9Y4E5 | E3 SUMO-protein ligase ZNF451 | ZNF451 | 730 |
| Q9Y4E5 | E3 SUMO-protein ligase ZNF451 | ZNF451 | 741 |
| Q9Y4E5 | E3 SUMO-protein ligase ZNF451 | ZNF451 | 813 |
| Q9Y4E5 | E3 SUMO-protein ligase ZNF451 | ZNF451 | 940 |
| Q9Y4E5 | E3 SUMO-protein ligase ZNF451 | ZNF451 | 959 |
| Q86UK7 | E3 ubiquitin-protein ligase ZNF598 | ZNF598 | 32 |
| Q86UK7 | E3 ubiquitin-protein ligase ZNF598 | ZNF598 | 33 |
| Q86UK7 | E3 ubiquitin-protein ligase ZNF598 | ZNF598 | 44 |
| Q86UK7 | E3 ubiquitin-protein ligase ZNF598 | ZNF598 | 49 |
| Q86UK7 | E3 ubiquitin-protein ligase ZNF598 | ZNF598 | 60 |
| Q86UK7 | E3 ubiquitin-protein ligase ZNF598 | ZNF598 | 65 |
| Q86UK7 | E3 ubiquitin-protein ligase ZNF598 | ZNF598 | 68 |
| Q86UK7 | E3 ubiquitin-protein ligase ZNF598 | ZNF598 | 119 |
| Q86UK7 | E3 ubiquitin-protein ligase ZNF598 | ZNF598 | 122 |
| Q86UK7 | E3 ubiquitin-protein ligase ZNF598 | ZNF598 | 146 |
| Q86UK7 | E3 ubiquitin-protein ligase ZNF598 | ZNF598 | 147 |
| Q86UK7 | E3 ubiquitin-protein ligase ZNF598 | ZNF598 | 189 |
| Q86UK7 | E3 ubiquitin-protein ligase ZNF598 | ZNF598 | 242 |
| Q86UK7 | E3 ubiquitin-protein ligase ZNF598 | ZNF598 | 247 |
| Q86UK7 | E3 ubiquitin-protein ligase ZNF598 | ZNF598 | 269 |
| Q86UK7 | E3 ubiquitin-protein ligase ZNF598 | ZNF598 | 426 |
| Q86UK7 | E3 ubiquitin-protein ligase ZNF598 | ZNF598 | 456 |
| Q86UK7 | E3 ubiquitin-protein ligase ZNF598 | ZNF598 | 610 |
| Q86UK7 | E3 ubiquitin-protein ligase ZNF598 | ZNF598 | 662 |
| Q86UK7 | E3 ubiquitin-protein ligase ZNF598 | ZNF598 | 676 |
| Q86UK7 | E3 ubiquitin-protein ligase ZNF598 | ZNF598 | 725 |
| Q86UK7 | E3 ubiquitin-protein ligase ZNF598 | ZNF598 | 835 |
| Q86UK7 | E3 ubiquitin-protein ligase ZNF598 | ZNF598 | 864 |
| Q86UK7 | E3 ubiquitin-protein ligase ZNF598 | ZNF598 | 867 |
| Q8ND25 | E3 ubiquitin-protein ligase ZNRF1 | ZNRF1 | 148 |
| Q8ND25 | E3 ubiquitin-protein ligase ZNRF1 | ZNRF1 | 164 |
| Q8ND25 | E3 ubiquitin-protein ligase ZNRF1 | ZNRF1 | 202 |
| Q8ND25 | E3 ubiquitin-protein ligase ZNRF1 | ZNRF1 | 204 |
| Q8ND25 | E3 ubiquitin-protein ligase ZNRF1 | ZNRF1 | 210 |
| Q8ND25 | E3 ubiquitin-protein ligase ZNRF1 | ZNRF1 | 221 |
| Q8NHG8 | E3 ubiquitin-protein ligase ZNRF2 | ZNRF2 | 160 |
| Q8NHG8 | E3 ubiquitin-protein ligase ZNRF2 | ZNRF2 | 163 |
| Q8NHG8 | E3 ubiquitin-protein ligase ZNRF2 | ZNRF2 | 179 |
| Q8NHG8 | E3 ubiquitin-protein ligase ZNRF2 | ZNRF2 | 217 |
| Q8NHG8 | E3 ubiquitin-protein ligase ZNRF2 | ZNRF2 | 219 |
| Q8NHG8 | E3 ubiquitin-protein ligase ZNRF2 | ZNRF2 | 236 |
| Q9ULT6 | E3 ubiquitin-protein ligase ZNRF3 | ZNRF3 | 311 |
| Q9ULT6 | E3 ubiquitin-protein ligase ZNRF3 | ZNRF3 | 433 |
| Q9ULT6 | E3 ubiquitin-protein ligase ZNRF3 | ZNRF3 | 779 |
| Q9ULT6 | E3 ubiquitin-protein ligase ZNRF3 | ZNRF3 | 882 |
| Q8NEG5 | E3 ubiquitin-protein ligase ZSWIM2 | ZSWIM2 | 605 |
| Q9C0D3 | Protein zyg-11 homolog B | ZYG11B | 43 |
| Q9C0D3 | Protein zyg-11 homolog B | ZYG11B | 643 |
| Q9C0D3 | Protein zyg-11 homolog B | ZYG11B | 735 |

TABLE 2B

| UniProt | KB02¹ | KB02² | KB03³ | KB03⁴ | KB05⁵ | KB05⁶ |
|---|---|---|---|---|---|---|
| Q969K4 | 83 | | 72 | | 53 | |
| Q969K4 | 63 | 17 | 34 | 6 | 53 | |
| Q969K4 | 53 | 28 | 43 | 18 | 38 | 3 |
| Q969K4 | 39 | 6 | 32 | | 65 | 3 |
| Q969K4 | 60 | | 62 | | 71 | |
| Q8N961 | 50 | | 51 | | 62 | |
| Q8N961 | 93 | 52 | 83 | 70 | 89 | 50 |
| Q8N961 | 87 | 60 | 44 | 15 | 42 | 10 |
| Q8N961 | 4 | | | | 7 | |
| Q8N961 | 20 | | 7 | | 25 | 28 |
| Q8N961 | | | | | 10 | |
| Q9C0C7 | 64 | | 52 | | 64 | |
| Q9C0C7 | 83 | | 71 | | 72 | |
| Q9C0C7 | 54 | | 19 | | 36 | |
| Q9C0C7 | 90 | 48 | 88 | 59 | 81 | 23 |
| Q9C0C7 | 72 | 49 | 81 | 73 | 79 | 25 |
| Q9C0C7 | 37 | | 5 | | 43 | |
| Q9UKV5 | 19 | 4 | 48 | | 18 | 5 |
| Q9H1A4 | 18 | | 29 | | 22 | |
| Q9H1A4 | 21 | | 5 | | 25 | |
| Q9H1A4 | 74 | | 57 | 30 | 76 | |
| Q9H1A4 | 29 | 24 | 58 | | 35 | |
| Q9H1A4 | 56 | 21 | 46 | 34 | 43 | 29 |
| Q9H1A4 | 38 | | 16 | | 51 | |
| Q9UM13 | 53 | 1 | 37 | 11 | 62 | 18 |
| Q9NYG5 | 63 | 41 | 79 | 51 | 37 | 27 |
| Q9NYG5 | 38 | | 20 | | 40 | |
| Q9NYG5 | 76 | | 75 | | 77 | |
| Q9NYG5 | 51 | | 49 | | 54 | |
| P60006 | 93 | 44 | 65 | 49 | 74 | 24 |
| Q96DE5 | 85 | 46 | 82 | 52 | 83 | 37 |
| Q9UJX6 | 37 | | 16 | | 56 | |
| Q9UJX6 | 94 | 40 | 97 | 86 | 76 | 23 |
| Q9UJX6 | 67 | 7 | 69 | 46 | 72 | 8 |
| Q9UJX6 | 84 | 47 | 73 | 11 | 81 | 35 |
| Q9UJX6 | 58 | 14 | 84 | 63 | 59 | |
| Q9UJX6 | 54 | | 73 | 58 | 71 | 24 |
| Q9UJX6 | 57 | | 37 | | 59 | |
| Q9UJX5 | 7 | | 29 | | 20 | 6 |
| Q9UJX4 | 64 | 35 | 62 | 31 | 74 | |
| Q9UJX4 | 62 | | 16 | | 70 | |
| Q9UJX4 | 71 | 16 | 50 | | 73 | |
| Q9UJX4 | 74 | | 42 | | 70 | |
| Q9UJX4 | 41 | | 27 | | 60 | |
| Q9UJX4 | 40 | 1 | 45 | | 58 | 9 |
| Q9UJX4 | 51 | 15 | 53 | | | |
| Q9UJX3 | 56 | 24 | 41 | 20 | 86 | 64 |
| Q9UJX3 | 87 | | 42 | | 40 | |
| Q9UJX3 | 6 | 16 | 47 | | 61 | 36 |
| Q9UJX3 | 67 | 30 | 39 | 28 | 53 | 58 |
| Q9UJX3 | 77 | 62 | 79 | 68 | 77 | 71 |
| Q9UJX3 | 73 | | 62 | | 79 | |
| Q9UJX3 | 84 | 48 | 75 | | 49 | |
| Q9UJX3 | 79 | | 54 | | 59 | |
| Q9UJX3 | 8 | | | | 32 | 9 |
| Q9UJX3 | 57 | 50 | 76 | 68 | 60 | 62 |
| Q9P2R3 | 68 | | 53 | | 73 | |
| Q9P2R3 | 21 | 14 | 50 | 33 | 76 | 26 |
| Q9P2R3 | 76 | 51 | 87 | 65 | 75 | 63 |
| Q9P2R3 | 63 | 4 | 36 | | 54 | 21 |
| Q9P2R3 | 44 | 14 | 74 | 55 | 62 | 41 |
| Q9P2R3 | 48 | | 51 | | 64 | |
| Q9P2R3 | 40 | 41 | 42 | 39 | 41 | |
| Q9P2R3 | 76 | 12 | 76 | | 79 | 38 |
| Q9P2R3 | 28 | | 34 | | 43 | |
| Q9P2R3 | 44 | | 52 | 39 | 37 | |
| Q9P2R3 | 66 | | 48 | | 57 | |
| Q9P2R3 | 27 | | 51 | | 52 | |
| Q9P2R3 | 46 | | 50 | | 57 | |
| Q9P2R3 | 62 | | 32 | | 57 | |
| Q9P2R3 | 68 | | 53 | | 60 | |
| Q9P2R3 | 43 | 12 | 73 | | 40 | |
| Q9P2R3 | 52 | | 43 | | 57 | |
| Q9P2R3 | 47 | | 51 | | 61 | |
| Q9P2G1 | 28 | | 16 | | 26 | |
| Q9P2G1 | 20 | | 14 | | 42 | |
| Q9P2G1 | 50 | | 26 | | 38 | |
| Q9P2G1 | 51 | | 40 | | 34 | |
| Q9P2G1 | 39 | 11 | 60 | 29 | 43 | 41 |
| Q9P2G1 | 32 | 7 | 58 | | | |
| Q9P2G1 | 66 | | 39 | | 66 | |
| Q9P2G1 | 52 | | 49 | | 68 | |
| Q9P2G1 | 20 | 17 | 36 | | 32 | |
| Q9P2G1 | 80 | 46 | 69 | | 55 | 22 |
| Q92624 | 100 | 83 | 84 | 56 | 79 | 34 |
| Q92624 | | 11 | 24 | | 16 | |
| O15033 | 96 | 77 | 87 | 57 | 92 | 69 |
| Q9Y4X5 | 82 | 36 | 46 | 23 | 67 | 41 |
| Q9Y4X5 | 46 | | 18 | | | |
| Q9Y4X5 | 64 | | 45 | | 59 | |
| Q9Y4X5 | 24 | | 22 | | 14 | |
| Q9Y4X5 | 64 | 36 | 75 | 63 | 55 | |
| Q9Y4X5 | 32 | 16 | 24 | | 36 | |
| Q9Y4X5 | 70 | 46 | 56 | 34 | 51 | 7 |
| Q9Y4X5 | 68 | | 33 | | 60 | |
| Q9Y4X5 | 54 | 8 | 40 | | 56 | |
| Q9Y4X5 | 67 | | 25 | | 54 | |
| Q9Y4X5 | 51 | 12 | 26 | | 51 | 21 |
| Q9Y4X5 | | | | | 8 | |
| Q9Y4X5 | 18 | 2 | 12 | | 31 | |
| Q9Y4X5 | 46 | | 20 | | 51 | |
| Q9Y4X5 | 15 | | 16 | | 22 | |
| Q9Y4X5 | 29 | | 26 | | 32 | |
| Q9Y4X5 | 69 | | 81 | | 83 | |
| Q9Y4X5 | 10 | | 11 | | 41 | |
| O95376 | 59 | 41 | 30 | | 42 | |
| O95376 | 77 | 21 | 67 | 4 | 77 | 16 |
| O95376 | 47 | 16 | 46 | 29 | 43 | 9 |
| O95376 | 72 | 10 | 63 | | 77 | |
| O95376 | 77 | | 67 | | 79 | |
| O95376 | 89 | 28 | 100 | | 87 | |
| O95376 | 100 | 44 | 100 | | 87 | |
| O95376 | 88 | 3 | 79 | | 80 | |
| O95376 | 89 | | 78 | | 79 | |
| O95376 | 71 | 22 | 47 | 34 | 63 | 26 |
| O95376 | 75 | 25 | 39 | 16 | 69 | 13 |
| O95376 | 65 | 3 | 45 | | 67 | |
| O95376 | 64 | | 56 | | 64 | |
| O95376 | 16 | 14 | 24 | | 46 | 9 |
| O95376 | 7 | 21 | 24 | | 28 | |
| O95376 | 26 | 19 | 48 | | 26 | |
| O95376 | 23 | 21 | 29 | | 38 | |
| O95376 | | | 28 | 21 | | |
| O95376 | | 5 | 24 | | | |
| O95376 | 42 | 5 | 28 | 20 | 37 | 25 |
| O95376 | | 6 | 20 | | 30 | 7 |
| Q96C12 | 35 | | 34 | | 54 | |
| Q96C12 | 41 | | 33 | | 30 | |
| Q96C12 | 81 | | 73 | | 74 | |
| Q96C12 | 51 | | 27 | | 57 | |
| Q96C12 | 96 | 62 | 63 | 40 | 65 | 23 |
| Q9Y576 | 48 | 35 | 33 | | 62 | 9 |
| Q9Y576 | 45 | 14 | 57 | | 60 | |
| Q9Y576 | 65 | 31 | 47 | | 61 | 14 |
| Q9Y576 | 60 | 35 | 73 | 51 | 86 | 20 |
| Q9Y576 | 22 | | 16 | | 53 | |
| Q9Y576 | 24 | 10 | 19 | | 23 | 14 |
| Q8WXK3 | 44 | 63 | 40 | 7 | | |
| Q8WXK3 | 30 | | 72 | | | |
| Q8WXK3 | 36 | | | | 33 | |
| Q8WXK3 | | 6 | 49 | | | |
| Q8WXK3 | | | 11 | | 2 | |
| Q96Q27 | 19 | | 10 | | 21 | |
| Q96Q27 | 96 | 54 | 77 | | 88 | 36 |
| Q96Q27 | 45 | | | | 23 | |
| Q96Q27 | 39 | 16 | 45 | | 36 | |
| Q9NWX5 | 46 | 38 | 75 | 72 | 88 | 51 |
| Q9NWX5 | 49 | 8 | 87 | 68 | 55 | 11 |
| Q9NWX5 | 82 | | 83 | | 46 | |
| Q9NWX5 | 33 | 2 | 44 | 19 | | |
| Q9H765 | 54 | 19 | 40 | | 37 | |
| Q96DX5 | 64 | 30 | 44 | 33 | 37 | 12 |
| Q96DX5 | | | | | 8 | |
| Q96DX5 | 16 | | 11 | | 32 | |
| P46100 | 31 | | 14 | | 23 | |
| P46100 | | | 7 | | 10 | 19 |
| P46100 | 12 | | 79 | | | |
| P46100 | 74 | 36 | 82 | 67 | 49 | 18 |
| P46100 | 21 | | 34 | | 29 | |
| P46100 | 22 | | 16 | | | |
| P46100 | 33 | | 38 | | 40 | |
| P46100 | | | | 13 | | |
| P46100 | 47 | 27 | 37 | 13 | 40 | 4 |
| P46100 | 79 | 29 | 43 | 12 | 40 | 13 |
| P46100 | 75 | 54 | 49 | 42 | 61 | 44 |
| P46100 | 42 | 9 | 26 | 29 | 19 | 1 |
| P46100 | 64 | | 42 | | 43 | |
| P46100 | 18 | 14 | 21 | | | |
| P46100 | 23 | | | | 9 | |
| P46100 | 36 | | | | 35 | 5 |
| P46100 | 33 | 23 | | | 19 | |
| P46100 | 65 | 17 | 23 | | 28 | |
| P46100 | 46 | | | | | |
| P46100 | 31 | | | | 16 | |
| P46100 | 67 | 37 | 55 | 33 | 88 | 23 |
| P46100 | 39 | 36 | 68 | | | |
| P46100 | 82 | | 68 | | 78 | |
| P46100 | 80 | | 59 | | 69 | |
| P46100 | | | | | 80 | 52 |
| P46100 | 74 | 43 | 90 | 74 | 87 | 47 |
| P46100 | 55 | 12 | 36 | | 63 | |
| P46100 | 75 | | 60 | | 89 | |
| P46100 | 67 | 47 | 85 | 25 | | |
| P46100 | 82 | | 73 | | 57 | |
| P46100 | 57 | | 50 | | 57 | |
| P46100 | 30 | 11 | 41 | 15 | 54 | 22 |
| Q9NXR7 | 50 | 10 | 37 | 26 | 41 | 1 |
| Q9NXR7 | 60 | 11 | 64 | 46 | 64 | |
| Q9NXR7 | 48 | 2 | 46 | | 58 | |
| Q9NXR7 | 63 | 44 | 64 | 53 | 73 | 17 |
| Q9NXR7 | 51 | | 57 | 29 | 60 | |
| Q9NXR7 | 54 | 37 | 38 | | 51 | |
| O14867 | 64 | | 49 | | 70 | |
| O14867 | 75 | 17 | 48 | | 83 | |
| O14867 | 80 | | 54 | | 80 | |
| O14867 | 75 | | 38 | | 76 | 23 |
| O14867 | 89 | 62 | 92 | 83 | 92 | 36 |
| O14867 | 60 | | 39 | | 53 | |
| O14867 | 50 | | | | 14 | |
| O14867 | 19 | | 2 | | 16 | |
| O14867 | 66 | | 51 | | 69 | |
| O14867 | 68 | | 45 | | 84 | 12 |
| O14867 | 81 | 35 | 70 | 61 | 67 | 29 |
| O14867 | 66 | | 42 | | 25 | |
| O14867 | 55 | | 32 | | 15 | |
| O14867 | 32 | | 13 | | 35 | |
| O14867 | 54 | | 76 | 64 | 62 | |
| O14867 | 97 | 91 | 95 | 92 | 91 | 62 |
| O14867 | 79 | 36 | 62 | 21 | 45 | |
| O14867 | 39 | | 1 | | 21 | |
| Q9BYV9 | 41 | 18 | 48 | | | |
| Q9BYV9 | 35 | 3 | 42 | | | |
| Q9BYV9 | 61 | 26 | 44 | 35 | 59 | 22 |
| Q9BYV9 | 63 | 14 | 43 | 20 | 63 | |
| Q9BYV9 | 61 | 27 | 65 | 3 | 61 | 10 |
| Q9BYV9 | 86 | 47 | 81 | 38 | 75 | 17 |
| Q9BYV9 | 56 | 19 | 49 | | 44 | 10 |
| Q9BYV9 | 49 | 6 | 40 | 19 | 48 | |
| Q9BYV9 | 50 | 19 | 58 | | 54 | |
| Q9BYV9 | 63 | 24 | 57 | 9 | 70 | 17 |
| Q9BYV9 | 56 | 26 | 57 | 3 | 55 | 13 |
| Q9BYV9 | 22 | 10 | 48 | | 34 | |
| Q9BYV9 | 83 | 13 | 80 | | 100 | 27 |
| Q9BYV9 | 87 | 41 | 100 | | | |
| Q9BYV9 | 33 | 7 | 23 | | | |
| Q9BYV9 | 19 | | 11 | | | |
| Q99728 | 47 | 22 | 68 | | 71 | |
| Q99728 | 45 | | 16 | | 59 | |
| Q99728 | 69 | 29 | 55 | | 40 | |
| Q99728 | 66 | 35 | 52 | | 57 | |
| Q99728 | 37 | 53 | 47 | 31 | 55 | |
| Q99728 | 66 | 33 | 31 | 38 | 68 | 31 |
| P41182 | 18 | 25 | | | | |
| P41182 | 55 | 28 | 39 | 19 | 74 | 9 |
| P41182 | 67 | 9 | 52 | 35 | 65 | 7 |
| P41182 | 67 | 20 | 53 | | | |
| P41182 | 58 | 17 | 37 | 20 | 41 | |
| P41182 | 43 | 8 | 49 | | | |
| P41182 | 48 | 20 | 42 | | | |
| P41182 | 42 | 13 | 49 | 15 | 41 | |
| P41182 | 17 | 10 | 31 | | 31 | |
| P41182 | 28 | 18 | 41 | | | |
| P41182 | 6 | 2 | 29 | | | |
| Q13490 | 99 | 99 | 98 | 91 | 87 | 44 |
| Q13490 | 36 | 33 | 33 | 46 | | |
| Q13490 | 37 | | 34 | 26 | 40 | 10 |
| Q13490 | 57 | | 44 | | 54 | |
| Q13490 | 75 | | 55 | | 63 | |
| Q13490 | 21 | | 19 | | 42 | 2 |
| Q13490 | 8 | 4 | | | 10 | |
| Q13490 | 7 | | 6 | | 23 | |
| Q13490 | 16 | | 14 | | 36 | |
| Q13490 | 55 | | 56 | | 58 | |
| Q13489 | 89 | 44 | 61 | 42 | 48 | 37 |
| Q13489 | 36 | 33 | 33 | 46 | | |
| Q13489 | 37 | | 34 | 26 | 40 | 10 |
| Q13489 | 26 | 17 | 46 | | | |
| Q13489 | 91 | 65 | 97 | 88 | 84 | 52 |
| Q13489 | 57 | | 44 | | 54 | |
| Q13489 | 67 | | 57 | | 61 | |
| Q13489 | 21 | | 19 | | 42 | 2 |
| Q13489 | 38 | 13 | 40 | | 24 | |
| Q13489 | 3 | 1 | 28 | | | |
| Q13489 | 16 | | 14 | | 36 | |
| Q13489 | 55 | | 56 | | 58 | |
| Q9NR09 | 17 | | 4 | | 13 | |
| Q9NR09 | 31 | | 10 | | 16 | |
| Q9NR09 | 90 | 92 | 94 | | | |
| Q9NR09 | 75 | 11 | 66 | | 49 | |
| Q9NR09 | 29 | 24 | 35 | 5 | 55 | |
| Q9NR09 | 37 | | 18 | | 25 | |
| Q9NR09 | 30 | 29 | 67 | 48 | 36 | 36 |
| Q9NR09 | | | 22 | | | |
| Q9NR09 | 17 | | 30 | | 33 | |
| Q9NR09 | 32 | 20 | 34 | 19 | 37 | 25 |
| Q9NR09 | 32 | 35 | 60 | | 23 | |
| Q9NR09 | 23 | | 16 | | 29 | |
| Q9NR09 | 64 | 46 | 54 | 36 | 64 | 38 |
| Q9NR09 | 38 | | 17 | | 49 | |
| Q9NR09 | 30 | 18 | 13 | | | |
| Q9NR09 | | | 14 | | | |
| Q9NR09 | 23 | | 40 | | 54 | |
| Q9NR09 | 58 | 46 | 55 | 26 | 57 | 29 |
| Q9NR09 | 54 | | 46 | 3 | 59 | 7 |
| Q9NR09 | 30 | | 20 | | 42 | |
| Q9NR09 | 37 | 2 | 26 | | | |
| Q9NR09 | 86 | 9 | 89 | 70 | 34 | 4 |
| Q9NR09 | 86 | | 58 | | 84 | |
| Q9NR09 | 2 | | | | 19 | |
| Q9NR09 | 27 | | 29 | | | |
| Q9NR09 | 81 | 41 | 78 | 71 | 81 | 35 |
| Q9NR09 | 50 | 24 | 57 | 35 | 63 | 42 |
| Q9NR09 | 61 | 18 | 31 | 18 | 47 | 43 |
| Q9NR09 | 51 | 15 | | | 57 | 27 |
| Q9NR09 | 28 | | 22 | | 57 | |
| Q9NR09 | 84 | 70 | 87 | 83 | 89 | 80 |
| Q9NR09 | 92 | | 69 | 11 | 38 | |
| Q9NR09 | 70 | 24 | 4 | | 59 | 13 |
| Q9NR09 | 73 | 33 | 77 | 44 | 90 | 37 |
| Q9NR09 | 39 | 3 | 19 | | 59 | |
| Q9NR09 | 53 | 12 | 43 | | 65 | |
| Q9NR09 | 45 | | 22 | | 51 | |
| Q9NR09 | 50 | 19 | 38 | | 39 | |
| Q96P09 | 38 | | 21 | | 45 | |
| Q7Z569 | 37 | 22 | 16 | 10 | 71 | 33 |
| Q7Z569 | 26 | 26 | 18 | | 32 | |
| Q7Z569 | | 6 | | | | |
| Q7Z569 | 30 | | 29 | | 38 | |
| Q7Z569 | 30 | | 36 | | 50 | |
| Q7Z569 | 60 | | 13 | | 43 | |
| Q7Z569 | 35 | | 30 | | 51 | |
| Q7Z569 | | | 1 | | | |
| Q7Z569 | 38 | | 33 | | 53 | |
| Q7Z569 | 1 | 5 | 16 | | 1 | |
| Q7Z569 | 42 | | 30 | | 30 | |
| Q7Z569 | 26 | 26 | 43 | | 30 | |
| P38398 | 62 | 38 | 96 | | 78 | |
| P38398 | 75 | 40 | 94 | | 68 | |
| P38398 | 71 | 32 | 85 | | 70 | |
| P38398 | 55 | 27 | 73 | | 65 | |
| P38398 | 80 | 21 | 75 | 14 | 79 | 18 |
| P38398 | 85 | 37 | 89 | | 72 | |
| P38398 | 44 | | 7 | | 32 | |
| P38398 | 35 | | 40 | 36 | 24 | |
| P38398 | 18 | | | | 1 | |
| P38398 | 62 | 35 | 43 | 19 | 66 | 16 |
| P38398 | 31 | 18 | 31 | | 11 | |
| P38398 | 53 | 19 | 34 | | 37 | |
| P38398 | 71 | 20 | 31 | | 63 | 8 |
| P38398 | 40 | 8 | 38 | | 46 | |
| P38398 | 63 | 30 | 54 | | 73 | |
| P38398 | 58 | 22 | 55 | | 36 | |
| P38398 | 47 | 5 | 43 | 35 | 45 | |
| P38398 | 69 | 31 | 44 | 5 | 63 | 16 |
| P38398 | 63 | 28 | 44 | 1 | 70 | 20 |
| P38398 | 62 | 35 | 48 | 18 | 55 | 14 |
| P38398 | 19 | | | | | |
| P38398 | 44 | 5 | 22 | | 39 | |
| P38398 | 65 | 36 | 65 | 47 | 58 | 11 |
| P38398 | 45 | 20 | 37 | | 40 | |
| P38398 | 64 | 19 | 53 | | 67 | |
| P38398 | 42 | 12 | 36 | 16 | 38 | 10 |
| P38398 | 27 | | 24 | | 38 | |
| P38398 | 45 | | 39 | | 52 | |
| P38398 | 50 | | 28 | | | |
| P38398 | 72 | | 34 | | 57 | |
| Q9H0C5 | 36 | | 13 | | 56 | |
| Q9H0C5 | 14 | 9 | 17 | | | |
| Q9H0C5 | 42 | 9 | 25 | | 36 | |
| Q9BSF8 | 45 | 8 | 13 | | 37 | |
| Q9BSF8 | 34 | 11 | 13 | | 37 | |
| Q9BSF8 | 56 | 26 | 38 | 29 | 53 | 27 |
| A6QL63 | 39 | 9 | 53 | 62 | 61 | 13 |
| A6QL63 | 50 | | 22 | | 49 | |
| A6QL63 | 7 | | | | 20 | |
| A6QL63 | | | | | 10 | |
| A6QL63 | 17 | | 46 | 29 | 79 | 28 |
| Q9BX70 | | 4 | 40 | | | |
| Q9P203 | 12 | | 13 | | 31 | |
| Q9P203 | 17 | | | | 8 | |
| Q5XKL5 | 31 | | | | 37 | |
| Q5XKL5 | 67 | 27 | 18 | | 55 | |
| Q96Q07 | 54 | | 29 | | 65 | |
| Q96Q07 | 45 | | 12 | | | |
| Q9Y297 | 69 | 8 | 48 | | 66 | |
| Q9Y297 | 69 | | 15 | | 65 | |
| Q9Y297 | 53 | | 28 | | 60 | |
| Q9Y297 | 26 | | 11 | | 35 | |
| Q9Y297 | 62 | | 30 | | 52 | |
| Q9Y297 | 55 | 36 | 67 | 54 | 63 | 43 |
| Q9Y297 | 100 | | 67 | | 85 | |
| Q9Y297 | 70 | | 61 | | 68 | |
| Q86VP6 | 56 | 47 | 42 | 46 | 43 | |
| Q86VP6 | | | | | 4 | |
| Q86VP6 | 11 | | 36 | | 26 | 4 |
| Q86VP6 | 65 | 27 | 26 | | 31 | |
| Q86VP6 | 59 | | 32 | | 10 | 7 |
| Q86VP6 | | | 9 | | 31 | |
| Q86VP6 | 9 | | 17 | | 49 | |
| Q86VP6 | 35 | 21 | 53 | 28 | 37 | 43 |
| Q86VP6 | 24 | 27 | | | | |
| Q86VP6 | 44 | 18 | 69 | 53 | 74 | 64 |
| Q86VP6 | 67 | 38 | 68 | 51 | 78 | 66 |
| Q86VP6 | | | | | | 4 |
| Q86VP6 | 47 | 12 | 43 | | 63 | |
| Q86VP6 | 11 | | 40 | | | |
| Q86VP6 | 21 | 39 | 59 | 17 | 41 | 39 |
| Q86VP6 | 57 | 21 | 51 | 37 | 73 | 18 |
| Q86VP6 | 62 | 24 | 73 | 57 | 65 | 55 |
| Q86VP6 | 46 | 10 | 53 | 17 | 75 | 47 |
| Q86VP6 | 54 | 30 | 79 | 48 | 77 | 60 |
| O75155 | 34 | | 33 | | 53 | |
| O75155 | 56 | | 67 | | 68 | |
| O75155 | 36 | | 63 | | 65 | |
| O75155 | 34 | | 60 | | 67 | |
| O75155 | 3 | | 39 | | 56 | |
| O75155 | 44 | | 46 | | 64 | |
| O75155 | 76 | | 78 | | 82 | |
| O75155 | 79 | | 83 | | 83 | |
| O75155 | 57 | | 72 | | 86 | |
| O75155 | 73 | | 85 | | 90 | |
| O75155 | 68 | | 87 | | 83 | |
| O75155 | 38 | | 26 | | 59 | 19 |
| O75155 | 50 | | 40 | | 61 | |
| O75155 | 29 | | 43 | | 51 | |
| O75155 | 50 | | 62 | | 57 | |
| O75155 | 62 | 24 | 43 | 31 | 61 | 5 |
| P22681 | 14 | | 22 | | 33 | |
| P22681 | 17 | | 41 | 11 | 18 | |
| P22681 | | | 30 | | 3 | |
| P22681 | | 4 | 4 | | 6 | |
| P22681 | 54 | 22 | 44 | 30 | 48 | 46 |
| P22681 | 87 | 76 | 59 | 51 | 65 | 14 |
| P22681 | 88 | 70 | 67 | 54 | 69 | 17 |
| P22681 | 57 | 27 | 31 | 21 | 38 | 9 |
| P22681 | 59 | 29 | 36 | 13 | 65 | 29 |
| Q13191 | 14 | | 22 | | 33 | |
| Q13191 | | | 5 | | 19 | |
| Q13191 | | | 30 | | 3 | |
| Q13191 | 67 | | 25 | | 77 | |
| Q13191 | 60 | | 27 | | 76 | |
| Q13191 | 53 | 41 | 51 | 34 | 63 | 22 |
| Q13191 | 65 | 46 | 41 | 27 | 51 | 43 |
| Q13191 | 74 | 67 | 69 | 57 | 61 | 12 |
| Q13191 | 56 | 65 | 72 | 58 | | |
| Q13191 | 84 | 78 | 87 | 80 | 76 | 21 |
| Q13191 | 45 | 28 | 26 | 13 | 42 | 35 |
| Q13191 | 72 | | 17 | | 51 | |
| Q13191 | 25 | 14 | 9 | | 48 | 5 |
| Q9ULV8 | 100 | | 76 | | 100 | |
| Q9ULV8 | 100 | 31 | 67 | | 66 | |
| Q9ULV8 | | | 84 | 57 | 77 | 24 |
| Q9ULV8 | 85 | 26 | 41 | 18 | 41 | 29 |
| Q9ULV8 | 51 | | 47 | 12 | 37 | |
| Q75N03 | 53 | 18 | 32 | 27 | 37 | 9 |
| Q75N03 | | | 9 | | 5 | |
| Q75N03 | 49 | 14 | 53 | 6 | 39 | |
| Q75N03 | 26 | 3 | 36 | | 25 | |
| Q75N03 | 55 | 6 | 62 | | 67 | |
| Q75N03 | 57 | 6 | 47 | | 49 | |
| Q75N03 | 40 | 9 | 35 | | 42 | |
| Q75N03 | 49 | 12 | 47 | | 65 | 26 |
| O00257 | 54 | 23 | 45 | 23 | 45 | 7 |
| O00257 | 71 | 43 | 70 | 41 | 81 | 20 |
| O00257 | 71 | 42 | 73 | 39 | 81 | 25 |
| O00257 | 53 | 16 | 65 | 37 | 51 | 7 |
| O60826 | 39 | | 22 | | 46 | |
| O60826 | 71 | 32 | 54 | | 63 | |
| O60826 | 38 | 26 | 30 | 7 | 29 | 22 |
| P55774 | | | | | 51 | 5 |
| P55774 | | | | | 51 | 5 |
| P41002 | 65 | 19 | 49 | | 37 | |
| P41002 | 83 | 45 | 66 | | 83 | 49 |
| P41002 | 75 | 21 | 62 | | 77 | |
| Q13042 | 14 | 9 | 43 | | | |
| Q13042 | 47 | 8 | 47 | | 63 | |
| Q13042 | 50 | 43 | 62 | 49 | 42 | 52 |
| Q13042 | 66 | 10 | 44 | | 61 | |
| Q13042 | 72 | 34 | 68 | 48 | 57 | 27 |
| Q13042 | 58 | | 51 | | 74 | |
| Q13042 | 44 | 40 | 41 | 49 | 59 | 38 |
| Q12834 | 66 | 36 | 83 | 71 | 73 | 9 |
| Q12834 | 98 | | 91 | | 93 | |
| Q12834 | 83 | 9 | 65 | 16 | 87 | 22 |
| Q12834 | 55 | 5 | 52 | | 66 | |
| Q9UJX2 | 28 | | 16 | | 43 | |
| Q9UJX2 | 45 | | 6 | | 24 | |
| Q9UJX2 | | 6 | 16 | | 32 | |
| Q9UJX2 | 78 | 6 | 54 | | 78 | |
| Q9UJX2 | 11 | | 9 | | 39 | |
| Q9UJX2 | 82 | | 56 | | 78 | |
| Q9UJX2 | 64 | 8 | 44 | | 61 | |
| Q9UJX2 | 76 | 30 | 69 | 18 | 75 | 9 |
| Q9UJX2 | 76 | 33 | 79 | 71 | 68 | 12 |
| P30260 | 49 | | 40 | | 52 | |
| P30260 | 43 | | 34 | | 51 | 10 |
| P30260 | | | 15 | | | |
| P30260 | 49 | | 57 | | 41 | |
| P30260 | 62 | | 34 | | 69 | |
| P30260 | 39 | 17 | 51 | 45 | 62 | 20 |
| P30260 | 79 | 59 | 69 | 52 | 82 | 20 |
| P30260 | 100 | 66 | 79 | 57 | 90 | 35 |
| P30260 | 90 | 59 | 95 | 87 | 33 | 28 |
| P30260 | 82 | 5 | 92 | 85 | 76 | |
| P30260 | 35 | | 18 | | 35 | |
| P30260 | 57 | 6 | 34 | | 44 | |
| P30260 | 43 | | 17 | | 61 | |
| P30260 | 40 | | 29 | | 53 | |
| Q6P1J9 | 48 | 18 | 35 | 24 | 42 | 33 |
| Q6P1J9 | 13 | 26 | 17 | | 13 | |
| Q99675 | 34 | 19 | 34 | | 74 | |
| Q99675 | 63 | 23 | 83 | | | |
| Q99675 | 61 | 34 | 71 | | | |
| Q99675 | 63 | 21 | 73 | | 29 | |
| Q99675 | 42 | | 24 | | 36 | |
| Q99675 | 30 | | 35 | | 28 | |
| Q96EP1 | | | 16 | | | |
| Q96EP1 | | | 8 | | 34 | |
| Q96EP1 | 54 | 27 | 66 | | 66 | |
| Q96EP1 | 38 | 4 | 22 | | 26 | |
| Q96EP1 | 45 | 3 | 35 | | 12 | |
| Q96EP1 | 63 | | 27 | | 28 | |
| Q96EP1 | 65 | | 35 | | 48 | |
| Q96EP1 | 36 | | 35 | | 35 | |
| Q96EP1 | 84 | 27 | 49 | | 29 | |
| Q96EP1 | 32 | 35 | 66 | | | |
| Q96EP1 | 51 | | 65 | | | |
| Q96EP1 | 9 | | 21 | | 31 | |
| Q9NSE2 | 31 | 41 | 44 | 21 | 33 | 31 |
| Q9NSE2 | 54 | 13 | 46 | 16 | 77 | 34 |
| Q9NSE2 | 18 | | 31 | | 57 | |
| Q9NSE2 | 16 | | 22 | | | |
| P10909 | 57 | | 32 | | 53 | |
| P10909 | 72 | 54 | 75 | 61 | 61 | 15 |
| P10909 | 45 | | 17 | | 37 | |
| P10909 | 81 | 69 | 42 | | 74 | 17 |
| P10909 | 70 | 53 | 72 | 58 | 75 | 24 |
| O95628 | 39 | | 31 | | 35 | |
| O95628 | | 6 | 20 | | | |
| O95628 | 20 | 6 | 39 | 13 | 97 | 94 |
| O95628 | | | 19 | | | |
| O95628 | 2 | | | | 16 | |
| Q86X83 | 83 | 31 | 72 | 35 | 78 | 38 |
| Q9UBI1 | 33 | | 29 | | 39 | |
| Q9UBI1 | 57 | | 29 | | 36 | |
| Q9GZQ3 | 11 | | 24 | | 56 | |
| Q7Z4G1 | 30 | 22 | 51 | 33 | 32 | 33 |
| Q7Z4G1 | 48 | | 25 | | 59 | |
| Q9P000 | 54 | 25 | 67 | 30 | 64 | 19 |
| Q9P000 | 100 | 83 | 12 | | 7 | |
| Q8NHY2 | 65 | 34 | 54 | 27 | 65 | 43 |
| Q8NHY2 | 62 | | 28 | | 48 | |
| Q8NHY2 | 58 | | 14 | | 35 | |
| Q8NHY2 | 57 | | 37 | | 59 | |
| Q8NHY2 | 37 | 11 | 29 | | 40 | |
| Q8NHY2 | 15 | | 20 | | 58 | |
| Q8NHY2 | 35 | | 35 | | 43 | |
| Q8NHY2 | 64 | | 51 | | 59 | |
| Q8NHY2 | 31 | | 15 | | 31 | |
| Q8NHY2 | 52 | 15 | 58 | | 47 | |
| Q13616 | 31 | 6 | 22 | | 33 | |
| Q13616 | 54 | 22 | 49 | | 51 | |
| Q13616 | 58 | 34 | 43 | 31 | 58 | 4 |
| Q13616 | 34 | | 23 | | 50 | |
| Q13616 | | 3 | 20 | | | |
| Q13616 | 30 | | 18 | | 41 | |
| Q13616 | | 1 | 17 | | | |
| Q13616 | 20 | | 9 | | 37 | |
| Q13617 | 58 | 39 | 27 | | 46 | 4 |
| Q13617 | 45 | 4 | 33 | | 59 | |
| Q13617 | 44 | 1 | 33 | | 51 | |
| Q13617 | 45 | 16 | 49 | 30 | 42 | 37 |
| Q13617 | 33 | 2 | 39 | | 51 | |
| Q13617 | 38 | 12 | 22 | | 53 | |
| Q13617 | | | | | 16 | |
| Q13618 | 39 | 38 | 47 | 35 | 53 | 46 |
| Q13618 | 48 | | 34 | | 20 | |
| Q13618 | | | 9 | | | |
| Q13618 | 29 | | 22 | | 47 | 11 |
| Q13618 | 23 | 16 | 35 | | | |
| Q13618 | 4 | | 27 | | | |
| Q13618 | 51 | 29 | 19 | 5 | 8 | |
| Q13618 | 55 | 33 | 43 | 39 | 46 | 47 |
| Q13618 | 33 | | 34 | | 57 | 3 |
| Q13618 | 37 | 25 | 41 | 18 | 54 | 40 |
| Q13619 | 32 | | 28 | | 52 | |
| Q13619 | 48 | | 33 | | 77 | |
| Q13619 | 72 | | 50 | | 70 | |
| Q13619 | 95 | 68 | 71 | 58 | 41 | |
| Q13619 | 59 | | 64 | | 62 | |
| Q13619 | 56 | 13 | 57 | 41 | 61 | 45 |
| Q13619 | 45 | 25 | 44 | 36 | 37 | 28 |
| Q13620 | 49 | 5 | 45 | 19 | 47 | 23 |
| Q13620 | 38 | | 23 | | 56 | |
| Q13620 | 16 | 4 | 36 | | 56 | |
| Q13620 | 47 | | 27 | | 49 | |
| Q13620 | 56 | 13 | 57 | 41 | 61 | 45 |
| Q13620 | 45 | 25 | 44 | 36 | 37 | 28 |
| Q13620 | 27 | | 24 | | 52 | |
| Q93034 | 41 | | 47 | | 41 | |
| Q93034 | 73 | 27 | 60 | 13 | 70 | 12 |
| Q93034 | 62 | 31 | 89 | 67 | 63 | 47 |
| Q93034 | 24 | | 14 | | 38 | |
| Q93034 | 11 | | 59 | | 61 | |
| Q14999 | 45 | | 6 | | 54 | |
| Q14999 | 24 | | 10 | | | |
| Q14999 | | | | 12 | | |
| Q14999 | 53 | | 25 | | 49 | |
| Q14999 | 60 | | 57 | | 75 | |
| Q14999 | 33 | | 27 | | 40 | |
| Q14999 | 27 | | 5 | | 38 | 12 |
| Q14999 | 30 | | 21 | | 32 | |
| Q14999 | 100 | 99 | 97 | 80 | 99 | 66 |
| Q14999 | 100 | 70 | 89 | 79 | 91 | 63 |
| Q14999 | 37 | | 22 | | 48 | |
| Q14999 | 40 | 27 | 49 | 20 | 30 | |
| Q8IWT3 | 25 | | 10 | | 35 | |
| Q8IWT3 | | | | 12 | | |
| Q8IWT3 | 65 | | 56 | | 71 | |
| Q8IWT3 | 34 | | 32 | | | |
| Q8IWT3 | 43 | | 14 | | 63 | |
| Q8IWT3 | 70 | 40 | 73 | 55 | 60 | 42 |
| Q8IWT3 | 72 | 46 | 19 | 6 | 66 | 32 |
| Q8IWT3 | 52 | | 15 | | 54 | |
| Q8IWT3 | 82 | 53 | | | | |
| Q8IWT3 | 82 | 53 | | | | |
| Q8IWT3 | 54 | 9 | 41 | | 50 | |
| Q9Y4B6 | 93 | 57 | 68 | 61 | 91 | 16 |
| Q9Y4B6 | 87 | 3 | 91 | | 66 | |
| Q9Y4B6 | 65 | | 45 | | 75 | |
| Q9Y4B6 | 56 | 26 | 56 | 33 | 61 | 56 |
| Q9Y4B6 | 80 | 31 | 57 | 38 | 74 | 50 |
| Q9Y4B6 | 69 | | 51 | 36 | 66 | 3 |
| Q9Y4B6 | 65 | | 37 | | 70 | |
| Q9Y4B6 | 3 | | 35 | | 65 | |
| Q9Y4B6 | 12 | 17 | 17 | | 17 | 6 |
| Q9Y4B6 | 44 | 22 | 38 | 38 | 37 | 8 |
| Q5QP82 | 47 | 6 | 35 | | 50 | |
| Q5QP82 | 61 | 38 | 46 | 23 | 70 | 34 |
| Q5QP82 | 65 | | 47 | | 50 | |
| Q5QP82 | 65 | | 66 | | 76 | |
| Q5QP82 | 83 | | 80 | | 82 | |
| Q5QP82 | 49 | | 69 | | 70 | |
| Q5QP82 | 58 | 39 | 48 | 48 | 70 | 21 |
| Q5QP82 | 49 | | 59 | | 66 | |
| Q5QP82 | 49 | | 42 | | 60 | |
| Q5QP82 | 73 | 26 | 68 | | 80 | |
| Q5QP82 | 79 | | 64 | | 83 | |
| Q5QP82 | 81 | | 66 | | 84 | |
| Q5QP82 | 85 | 61 | 91 | 84 | 95 | 57 |
| Q8TEB1 | 54 | 31 | 61 | | 56 | |
| Q8TEB1 | 55 | 15 | 64 | | 74 | |
| Q8TEB1 | 66 | | 54 | | 68 | |
| Q8TEB1 | 50 | | 41 | | 52 | |
| Q8TEB1 | 72 | | 57 | 43 | 81 | 38 |
| Q5T6F0 | 16 | | 36 | | | |
| Q5T6F0 | | 15 | 39 | | | |
| Q5T6F0 | 44 | | 31 | | 23 | |
| Q5T6F0 | 46 | 14 | 36 | 11 | 47 | 31 |
| Q5T6F0 | 33 | | 43 | | | |
| Q5VU92 | | | 24 | 10 | 11 | |
| Q5VU92 | 29 | 25 | 36 | 24 | 28 | |
| Q9NV06 | 65 | 24 | 67 | | 76 | 25 |
| Q9NV06 | 60 | 27 | 51 | 46 | 69 | 22 |
| Q9NV06 | 43 | 29 | 40 | 16 | 51 | 34 |
| Q9NV06 | 59 | | 48 | | 77 | |
| Q9NV06 | 48 | 26 | 55 | 48 | 67 | 40 |
| Q9NV06 | 65 | 46 | 77 | 62 | 78 | 32 |
| Q9NV06 | 35 | 6 | 37 | | 61 | |
| Q9NV06 | 71 | 2 | 62 | | 76 | |
| Q66K64 | 20 | | | | 46 | |
| Q66K64 | 57 | | 32 | | 64 | |
| Q66K64 | | | | | 8 | |
| Q66K64 | 73 | 73 | 55 | 31 | 43 | 11 |
| Q66K64 | 59 | 2 | 36 | | 58 | |
| Q9NXF7 | 85 | 66 | 81 | 68 | 88 | 51 |
| Q9NXF7 | 100 | 25 | 88 | 85 | 85 | 26 |
| Q9NXF7 | 59 | 45 | 79 | 58 | 80 | 31 |
| Q9NXF7 | 78 | 38 | 57 | 29 | 75 | 11 |
| Q9NXF7 | | | 16 | | 43 | 20 |
| Q9NXF7 | | | 7 | | 72 | 17 |
| Q5H9S7 | 93 | 69 | 57 | 39 | 77 | 54 |
| Q5H9S7 | 32 | | 13 | | | |
| Q5H9S7 | 91 | | 70 | | 64 | |
| Q8WV16 | 32 | 16 | 28 | | 22 | 26 |
| Q8WV16 | 14 | | 30 | | 32 | |
| Q96JK2 | 70 | 18 | 91 | 79 | 92 | 2 |
| Q96JK2 | 60 | | 89 | | 56 | |
| Q96JK2 | 57 | | 60 | | 68 | |
| Q96JK2 | 63 | | 33 | | 52 | |
| Q96JK2 | 38 | 32 | 18 | | 39 | 10 |
| Q96JK2 | 56 | 48 | 45 | 29 | 41 | 7 |
| Q96JK2 | 22 | | | | 16 | |
| Q96JK2 | 63 | 44 | 40 | 32 | 48 | 25 |
| Q96JK2 | 60 | 56 | 45 | 29 | 31 | 20 |
| Q96JK2 | 20 | | | | | |
| Q58WW2 | 79 | | 64 | | 79 | |
| Q58WW2 | 67 | | 50 | | 70 | |
| Q58WW2 | 81 | | 56 | | 80 | |
| Q58WW2 | 78 | | 73 | | 89 | |
| Q58WW2 | 40 | 23 | 17 | | 34 | 3 |
| Q58WW2 | 63 | 66 | 72 | 67 | 47 | 12 |
| P61962 | 35 | 11 | 67 | 54 | 53 | 15 |
| P61962 | 59 | 38 | 92 | 78 | 73 | 17 |
| P61962 | 57 | 38 | 34 | 33 | 52 | 24 |
| P61962 | 67 | 19 | 67 | 55 | 68 | 8 |
| P61962 | 35 | | 24 | | 23 | |
| A6NGE4 | 55 | | 41 | | 63 | |
| P0C7V8 | 55 | | 41 | | 63 | |
| Q96GG9 | 19 | 7 | 19 | | 12 | |
| Q96GG9 | 54 | 23 | 50 | 26 | 42 | 10 |
| Q16531 | 16 | 14 | 30 | | 34 | |
| Q16531 | 11 | 25 | 38 | 24 | 28 | 10 |
| Q16531 | | 7 | 53 | 43 | 60 | 19 |
| Q16531 | 29 | 16 | 55 | 43 | 49 | 20 |
| Q16531 | 63 | 52 | 56 | 50 | 55 | 25 |
| Q16531 | 12 | | 25 | | 34 | 12 |
| Q16531 | 18 | 4 | 26 | | 27 | |
| Q16531 | 27 | | 21 | | 38 | |
| Q16531 | 61 | 50 | 56 | 55 | 46 | 23 |
| Q92466 | 88 | 43 | 78 | 63 | 72 | 53 |
| Q92466 | 90 | 36 | 51 | 30 | 59 | 12 |
| Q92466 | 80 | | 47 | 42 | 57 | 29 |
| Q92466 | 31 | | 37 | 24 | 33 | 22 |
| Q92466 | 32 | 16 | 47 | 25 | 31 | |
| Q92466 | 63 | 58 | 80 | 74 | 73 | 47 |
| Q92466 | 54 | 30 | 50 | 32 | 52 | 7 |
| Q92782 | | | 19 | | 26 | |
| Q92782 | 77 | 48 | 63 | 44 | 66 | 8 |
| Q92782 | 32 | 27 | 27 | | 14 | |
| Q92782 | 43 | 20 | 40 | 32 | 32 | |
| Q92782 | 28 | | | | 23 | |
| Q92782 | 40 | | 14 | | 32 | |
| Q92782 | 40 | | 17 | | 24 | 3 |
| Q92782 | 37 | 17 | 41 | 18 | 30 | |
| Q92782 | 24 | 20 | 31 | 22 | 33 | 7 |
| Q9NZJ0 | 78 | 51 | 69 | 38 | 80 | 42 |
| Q9NZJ0 | 100 | 96 | 96 | 88 | 87 | 59 |
| Q9NZJ0 | 77 | 30 | 56 | 45 | 66 | 8 |
| Q9NZJ0 | 73 | | 65 | | 74 | |
| Q9NZJ0 | 70 | | 64 | | 66 | |
| Q9NZJ0 | 54 | 11 | 68 | 53 | 68 | 17 |
| Q9NZJ0 | 71 | 5 | 54 | | 67 | |
| Q9NZJ0 | 71 | 64 | 3 | | 65 | 9 |
| Q9NZJ0 | 61 | 14 | 67 | 57 | 65 | |
| Q9NZJ0 | 60 | 49 | 69 | 32 | 56 | 25 |
| Q9NZJ0 | 71 | 78 | 68 | 39 | 61 | 27 |
| Q9NZJ0 | 66 | | 10 | | 55 | |
| Q9NZJ0 | 58 | | 27 | | 82 | |
| Q9NZJ0 | 67 | | 24 | | 88 | |
| Q9NZJ0 | 39 | | | | 27 | |
| Q9NZJ0 | 62 | | | | 59 | |
| Q9NZJ0 | 85 | 66 | 81 | 56 | 85 | 34 |
| Q9NZJ0 | 27 | | 20 | | 48 | |
| Q86Y01 | 80 | 24 | 81 | 48 | 55 | 17 |
| Q86Y01 | 56 | 21 | 26 | 3 | 18 | |
| Q86Y01 | 73 | 43 | | | | |
| Q86Y01 | 73 | 43 | | | | |
| Q86Y01 | 74 | 35 | 59 | | | |
| Q86UW9 | | | | | 16 | |
| Q86UW9 | 38 | | 23 | | 48 | |
| Q86UW9 | 42 | | 15 | | 41 | 3 |
| Q86UW9 | 34 | 28 | 30 | 8 | 27 | 16 |
| Q86UW9 | 61 | | 43 | | 87 | |
| Q86UW9 | 39 | | 49 | | 58 | |
| Q8N9I9 | 76 | | 29 | | 75 | |
| Q8N9I9 | 39 | | 20 | | 54 | |
| Q8N9I9 | 26 | | | | 6 | |
| Q8N9I9 | 22 | | 7 | | 27 | 9 |
| Q8N9I9 | 33 | | 19 | 10 | 23 | |
| Q8N9I9 | 27 | | 27 | | 30 | |
| Q8N9I9 | 35 | | 16 | | 27 | |
| Q8N9I9 | 30 | | 27 | | 12 | |
| Q8N9I9 | 48 | 8 | 25 | | 29 | 3 |
| Q8N9I9 | 9 | | 7 | | | |
| Q8TDB6 | 29 | | 2 | | 49 | |
| Q8TDB6 | 57 | 18 | 58 | 38 | 56 | |
| Q8TDB6 | 66 | 38 | 65 | 53 | 78 | 48 |
| Q8TDB6 | 54 | 16 | 45 | 30 | 59 | 46 |
| Q8TDB6 | 9 | 13 | | 5 | 33 | 51 |
| Q8TDB6 | 24 | 12 | 20 | 23 | 39 | 41 |
| Q8TDB6 | 21 | | | 15 | 39 | 31 |
| Q8TDB6 | 15 | 15 | 7 | 16 | 23 | 46 |
| Q8TDB6 | 57 | | 37 | | 49 | |
| Q8TDB6 | 12 | | 11 | | 36 | |
| Q8TDB6 | 37 | 12 | 40 | 20 | 34 | 42 |
| Q8TDB6 | 69 | 30 | 79 | 47 | 60 | 31 |
| Q8TDB6 | 66 | 30 | 77 | 41 | 74 | 52 |
| Q8TDB6 | 73 | 8 | 36 | | 71 | 13 |
| Q8TDB6 | 20 | | 60 | 31 | 68 | 35 |
| Q8TDB6 | 29 | 5 | 11 | | 43 | |
| Q86Y13 | 27 | | 25 | | 60 | |
| Q86Y13 | 44 | | 46 | 46 | 57 | 24 |
| Q86Y13 | 46 | 11 | 33 | | 50 | |
| Q66K89 | 22 | | | | 18 | |
| Q66K89 | 43 | 20 | 30 | 26 | 27 | 8 |
| Q66K89 | 69 | | 39 | | 53 | |
| P11161 | 77 | | 62 | | 72 | |
| P11161 | 60 | | 13 | | 52 | |
| P11161 | 51 | | 2 | | 52 | |
| Q14241 | 44 | 23 | 47 | 44 | 48 | 1 |
| Q14241 | 28 | 12 | 41 | 17 | 67 | 12 |
| Q14241 | 72 | 33 | 51 | 28 | 52 | 41 |
| Q14241 | 52 | 24 | 35 | 37 | 20 | |
| Q14241 | 27 | | 26 | | 31 | |
| Q15370 | 54 | 31 | 63 | 36 | 52 | 32 |
| Q15370 | 21 | 21 | 3 | 7 | 48 | 22 |
| Q15369 | 62 | 72 | 48 | 41 | 78 | 44 |
| Q15369 | 58 | 32 | 45 | 33 | 55 | 23 |
| O14682 | 82 | | 79 | | 85 | |
| O14682 | 28 | | 40 | | 38 | |
| O14682 | 36 | 29 | 39 | 20 | 37 | 20 |
| O14682 | 24 | | 30 | | 29 | |
| O14682 | 16 | | 2 | | 32 | |
| O14682 | 69 | | 47 | | 65 | |
| O14682 | 29 | | 7 | | 47 | |
| O14682 | 34 | | 34 | | 57 | |
| O14682 | 17 | | 13 | | 20 | |
| O14682 | 24 | | 38 | | 51 | |
| Q13216 | 54 | | 31 | | 56 | |
| Q13216 | 57 | | 34 | | 57 | |
| Q13216 | 54 | 3 | 51 | | 66 | |
| Q13216 | 59 | | 43 | | 52 | |
| Q13216 | 73 | 2 | 52 | 16 | 69 | |
| Q13216 | 55 | | 54 | | 71 | |
| Q13216 | 44 | | 41 | | 54 | |
| Q13216 | 66 | 15 | 57 | | 81 | 16 |
| Q13216 | 87 | | 72 | | 63 | |
| Q13216 | 61 | | 42 | | 55 | |
| Q9NW38 | 86 | 44 | 80 | | 85 | |
| Q9NW38 | 73 | | 47 | | 43 | |
| Q8NFZ0 | 45 | | 25 | | 34 | |
| Q8NFZ0 | 69 | | 58 | | 78 | |
| Q8NFZ0 | 30 | | 22 | | 40 | |
| Q8NFZ0 | 38 | 24 | 61 | | 46 | |
| Q8NFZ0 | 56 | 15 | 41 | | 59 | |
| Q8NFZ0 | 76 | 6 | 61 | | 62 | |
| Q8NFZ0 | 73 | | 55 | | 73 | |
| Q8NFZ0 | 78 | | 55 | | 66 | |
| Q9NXK8 | 81 | 72 | 81 | 84 | 80 | 70 |
| Q9NXK8 | 39 | 38 | 58 | 53 | 48 | 31 |
| Q9NXK8 | 55 | 39 | 78 | 81 | 56 | 18 |
| Q9NXK8 | 61 | 41 | 55 | | 20 | |
| Q9NXK8 | | 10 | 33 | | | |
| Q9NXK8 | 71 | 41 | 50 | | 22 | |
| Q8N1E6 | 1 | | 12 | | 22 | |
| Q8N1E6 | 6 | | 50 | | | |
| Q9H469 | 13 | 21 | 35 | | | |
| Q9H469 | 65 | | 66 | | 63 | |
| Q9H469 | 64 | | 67 | | 25 | |
| Q9H469 | | | | | 14 | |
| Q9H469 | 49 | 1 | 56 | | 51 | 13 |
| Q8N461 | 6 | | 11 | | 12 | |
| Q9UF56 | 72 | | 53 | | 54 | |
| Q9UF56 | 74 | | 37 | | 45 | |
| Q9UF56 | 52 | | 26 | | 20 | |
| Q9UF56 | 89 | | 76 | | 76 | |
| Q96ME1 | 3 | | 13 | | 60 | |
| Q96ME1 | 70 | | 45 | | 55 | |
| Q96ME1 | 57 | | 51 | | 57 | |
| Q96ME1 | 55 | | | | 42 | |
| Q96ME1 | 76 | | 71 | | 66 | |
| Q96ME1 | 59 | | 44 | | 56 | |
| Q96ME1 | 75 | | 66 | | 92 | |
| Q96ME1 | 69 | | 71 | | 79 | |
| Q96ME1 | | | | | 25 | 5 |
| Q96ME1 | 39 | 9 | 42 | | 49 | |
| Q96ME1 | 58 | 18 | 43 | 14 | 77 | 30 |
| Q96ME1 | 54 | | 37 | | 57 | 13 |
| Q96ME1 | 91 | 87 | 85 | 76 | 88 | 38 |
| Q96ME1 | 100 | 86 | 82 | 76 | 87 | 39 |
| Q96ME1 | 85 | | 51 | | 69 | |
| Q96ME1 | 61 | | 88 | | 62 | |
| Q96ME1 | 48 | | 50 | | 54 | |
| Q6PCT2 | 14 | 11 | 32 | | 13 | |
| Q6PCT2 | 38 | 3 | 44 | 21 | 48 | 5 |
| Q6PCT2 | | | 56 | 41 | 10 | |
| Q6PCT2 | 43 | 41 | 55 | 42 | 27 | 14 |
| Q6PCT2 | 54 | | 38 | | 47 | |
| Q6PCT2 | 44 | | 14 | | 43 | |
| Q6PCT2 | 46 | 11 | 15 | | 36 | |
| Q6PCT2 | 75 | 17 | 72 | | 52 | |
| Q6PCT2 | 57 | | 66 | | 62 | |
| Q6PCT2 | 24 | | 31 | | 29 | |
| Q9UKC9 | 42 | | 56 | | 58 | |
| Q96IG2 | 21 | 1 | 50 | | 43 | |
| Q96IG2 | 11 | | 44 | | | |
| Q96IG2 | 77 | | 24 | | 34 | |
| Q96IG2 | 42 | | 56 | | 58 | |
| Q96IG2 | 48 | | 29 | | 58 | |
| Q9UKT7 | 47 | | 45 | | 49 | |
| Q9UKT7 | 46 | 16 | 51 | | 56 | |
| Q9UKT7 | 85 | 34 | 100 | | | |
| Q9UKT7 | | 72 | | 91 | 95 | |
| Q9UKT7 | 23 | 1 | 43 | | | |
| Q9UKT7 | 31 | | 66 | 23 | 91 | 32 |
| Q9UKA2 | 65 | 29 | 70 | | 52 | |
| Q9UKA2 | 36 | 35 | 41 | 9 | 25 | |
| Q9UKA2 | 43 | 10 | 59 | | | |
| Q9UKA1 | 39 | | 27 | | 69 | |
| Q8N531 | 58 | 15 | 40 | | 73 | |
| Q8N531 | 73 | 64 | 84 | 78 | 54 | |
| Q8N531 | 62 | 19 | 27 | | 61 | |
| Q8N531 | 63 | 37 | 65 | | 57 | 21 |
| Q8N531 | 65 | 51 | 72 | 74 | 81 | 43 |
| Q96CD0 | 30 | | 24 | | 47 | |
| Q96CD0 | | | | | 8 | |
| Q96CD0 | 20 | | 27 | | 26 | |
| Q9UK96 | 51 | 8 | 13 | | 42 | |
| Q9UK96 | 51 | | 15 | | 21 | |
| Q9UK96 | 37 | | 3 | | 36 | |
| Q9UK96 | 26 | | 16 | | 32 | |
| Q9UK96 | 46 | 1 | 42 | | | |
| Q86XK2 | 56 | 38 | 27 | 25 | 40 | 35 |
| Q86XK2 | 98 | 63 | 31 | 15 | 25 | 22 |
| Q96EF6 | 57 | | 47 | | 60 | |
| Q96EF6 | 26 | | 14 | | 20 | |
| Q96EF6 | 64 | | 36 | | 67 | |
| Q9UK22 | 34 | 32 | 27 | | 13 | |
| Q9UK22 | 27 | 16 | 12 | | 27 | |
| Q9UK22 | 4 | | 7 | | 11 | |
| Q9UK22 | 66 | | 44 | | 41 | |
| Q9UK22 | 74 | | 51 | | 54 | |
| O94952 | | | | | 11 | |
| O94952 | 25 | | 11 | | 24 | |
| Q8NEZ5 | 46 | | 28 | | 43 | |
| Q8NEZ5 | 51 | | 26 | | 52 | |
| Q8NEZ5 | 51 | | 12 | | 48 | |
| Q8NEZ5 | 50 | 53 | 35 | 22 | 84 | 37 |
| Q8NEZ5 | 41 | 27 | 40 | 25 | 40 | 35 |
| Q8NI29 | 47 | | 38 | | 57 | |
| Q8NI29 | | | | | 21 | |
| Q9UK99 | 39 | 18 | 37 | | | |
| Q9UK99 | 41 | | 13 | | 30 | |
| Q8TB52 | 42 | | 54 | | 48 | |
| Q8TB52 | 69 | | 60 | | 64 | |
| Q8TB52 | 37 | | 31 | 10 | 33 | |
| Q8TB52 | 78 | | 79 | | 71 | |
| Q8TB52 | 89 | 11 | 77 | | 77 | |
| Q8TB52 | 30 | 6 | 29 | 19 | 21 | |
| Q8TB52 | 47 | 16 | 39 | 40 | 44 | |
| Q8TB52 | 55 | | 24 | | 48 | |
| Q8TB52 | 44 | | 44 | | 57 | |
| Q8TB52 | 94 | 4 | 82 | 56 | 77 | |
| Q8TB52 | 56 | 33 | 78 | 55 | 55 | 13 |
| Q8TB52 | 54 | 42 | 45 | 31 | 42 | 26 |
| Q8TB52 | 54 | 32 | 47 | 36 | 63 | 25 |
| Q8TB52 | 46 | 3 | 48 | | 41 | 12 |
| Q8TB52 | 47 | | 24 | | 37 | |
| Q8TB52 | 85 | 22 | 49 | | 100 | 43 |
| Q8TB52 | 88 | 58 | 87 | 64 | 80 | 24 |
| Q5XUX0 | 31 | 14 | 31 | | | |
| Q5XUX0 | 90 | 30 | 76 | | 62 | |
| Q5XUX0 | 34 | | 24 | | 50 | |
| Q5XUX0 | | | 2 | | | |
| Q5XUX0 | 29 | 28 | 35 | | | |
| Q969P5 | | | | | 25 | |
| Q7Z6M2 | 57 | | 52 | | 71 | |
| Q9NWN3 | 50 | | 23 | | 62 | |
| Q9NWN3 | 46 | | 24 | | 55 | |
| Q9NWN3 | 47 | 1 | 46 | | | |
| Q9NWN3 | 8 | | 21 | | | |
| Q9NWN3 | 59 | 18 | 47 | | 44 | |
| Q6PIJ6 | 71 | 39 | | | | |
| Q6PIJ6 | 59 | | 42 | | 57 | |
| Q6PIJ6 | 89 | 7 | 60 | | 64 | |
| Q6PIJ6 | 68 | | 50 | | 64 | |
| Q6PIJ6 | 35 | 24 | 46 | 34 | 31 | 36 |
| Q6PIJ6 | 90 | | 76 | | 70 | |
| Q6PIJ6 | 67 | | 22 | | 16 | |
| Q6PIJ6 | 63 | 58 | 38 | 27 | 18 | |
| Q6PIJ6 | 36 | 1 | 47 | 31 | 45 | |
| Q6PIJ6 | 57 | 11 | 46 | 40 | 45 | |
| Q6PIJ6 | 49 | 3 | 47 | 42 | 38 | |
| Q6PIJ6 | 74 | | 36 | | 38 | |
| Q6PIJ6 | 51 | 45 | 36 | 25 | 41 | 6 |
| Q6PIJ6 | 88 | 69 | 49 | 44 | 50 | 29 |
| Q6PIJ6 | 24 | | 5 | | 16 | |
| Q6PIJ6 | 55 | | 50 | | 64 | |
| Q9UKT5 | | | 54 | 33 | 36 | 3 |
| Q9UKT5 | 36 | 27 | 43 | 28 | 27 | 8 |
| Q9UKT5 | 38 | | 53 | | 67 | |
| Q6P3S6 | | 2 | 34 | | | |
| Q6P3S6 | 86 | 51 | 70 | 40 | 81 | 38 |
| Q6P3S6 | 84 | 20 | 60 | 40 | 54 | |
| Q9H4M3 | 62 | | 52 | | 61 | |
| Q9H4M3 | 13 | | 3 | | 17 | |
| P0C2W1 | | | 14 | | | |
| P0C2W1 | 24 | 1 | 33 | | 22 | |
| P0C2W1 | | 4 | 23 | | | |
| P0C2W1 | 80 | 33 | 57 | 42 | 43 | 16 |
| Q6PJ61 | 59 | 8 | 32 | | 49 | |
| Q6PJ61 | 40 | 3 | 27 | | 48 | |
| Q6PJ61 | 47 | 18 | 32 | | | |
| Q6PJ61 | 54 | | 31 | | 37 | |
| Q6PJ61 | | | 2 | | | |
| Q6PJ61 | 8 | | 21 | | | |
| Q6PJ61 | 13 | | | | 8 | |
| Q6PJ61 | 65 | 25 | 56 | 38 | 47 | 4 |
| Q9UKT4 | 68 | 28 | 62 | 53 | 45 | 37 |
| Q9UKT4 | 39 | 17 | 57 | | 36 | 7 |
| Q9UKT4 | 83 | 26 | 80 | 58 | 77 | 14 |
| Q9UKT4 | 78 | 25 | 71 | 38 | | |
| Q9UKT4 | 80 | 21 | 65 | | 56 | |
| Q9UKT4 | 80 | | 34 | | 56 | |
| Q9UKT4 | 61 | | 26 | | 56 | |
| Q9UKT4 | 68 | 12 | 33 | | 58 | |
| Q9UKT4 | 71 | | 54 | | 74 | |
| Q9UKT4 | 43 | 46 | 68 | 34 | 65 | 45 |
| Q9UKT4 | 64 | 27 | 68 | | 62 | 33 |
| Q9UKT4 | 41 | 8 | 59 | 56 | 42 | |
| Q9UKT4 | 51 | 16 | 44 | | 41 | |
| Q9UKT4 | 55 | | | | 17 | |
| Q9UKT4 | 41 | | 43 | | 54 | |
| Q9UKT4 | 49 | 17 | 68 | | | |
| Q9UKT4 | 48 | | | | 17 | |
| Q9UKT4 | 28 | | 19 | | 31 | |
| Q9UKT4 | 26 | | | | 24 | |
| Q9NRD1 | 62 | | 52 | | 61 | |
| Q9Y3I1 | 71 | 11 | 77 | 18 | 81 | |
| Q9Y3I1 | 52 | 18 | 49 | 38 | 65 | 59 |
| Q9UK97 | 78 | 23 | 37 | | 57 | 11 |
| Q9UKB1 | 51 | | 53 | | 63 | |
| Q9UKB1 | 76 | 39 | 92 | | | |
| Q9UKB1 | 57 | | 34 | | 67 | |
| Q9UKB1 | 50 | | 49 | | 63 | |
| Q9UKB1 | 62 | | 30 | | 52 | |
| Q9UKB1 | 55 | 36 | 67 | 54 | 63 | 43 |
| Q9UKB1 | 100 | | 67 | | 85 | |
| Q9UKB1 | 83 | | 50 | | 66 | |
| Q9UKT8 | 34 | 15 | 48 | 13 | 44 | 9 |
| P57775 | 83 | 39 | 57 | | 73 | 13 |
| P57775 | 33 | | 26 | | 49 | |
| Q969U6 | 37 | | 19 | | 33 | |
| Q969U6 | 4 | 6 | 34 | | | |
| Q969H0 | 73 | | 45 | | 27 | |
| Q969H0 | 42 | 18 | 24 | | | |
| Q8N3Y1 | 45 | | 29 | | 51 | |
| Q8N3Y1 | 4 | | | | 17 | |
| Q8N3Y1 | 70 | 33 | 73 | 68 | 46 | 16 |
| Q8N3Y1 | 93 | 49 | 60 | 27 | 89 | 30 |
| Q5XUX1 | 61 | 28 | 79 | | 45 | |
| Q5XUX1 | 65 | | 65 | | 64 | |
| Q5XUX1 | 82 | 36 | 73 | 44 | 67 | 25 |
| Q5XUX1 | 38 | 21 | 65 | | | |
| Q5XUX1 | 34 | 5 | 31 | | 46 | 7 |
| Q9BSK4 | 79 | 41 | 62 | 38 | 79 | 29 |
| Q9UK73 | 90 | 60 | 79 | | 77 | 28 |
| Q9UK73 | | | 17 | | | |
| Q9UK73 | | | | | 8 | |
| Q7L622 | 70 | | 70 | | 73 | |
| Q7L622 | 52 | 43 | 62 | | | |
| Q7L622 | 22 | | 3 | | 23 | |
| Q9H2C0 | 19 | | 21 | | 34 | |
| Q9H2C0 | 80 | 35 | 84 | 67 | 92 | 38 |
| Q9H2C0 | 1 | 9 | 35 | | 20 | |
| Q9H2C0 | 6 | 8 | 51 | 36 | 46 | 13 |
| Q96IK5 | 66 | | 35 | | 67 | |
| Q96IK5 | 62 | | 26 | | 56 | |
| Q13888 | 82 | | 67 | | 91 | |
| Q13888 | 51 | | 19 | | 54 | |
| Q13888 | 60 | | 46 | | 67 | |
| Q13888 | 44 | 32 | 57 | 38 | 54 | 31 |
| Q13888 | 51 | 20 | 43 | 10 | 53 | 36 |
| Q13888 | 21 | | | | 11 | |
| Q13888 | 83 | | 79 | | 41 | |
| Q13888 | 9 | 11 | 42 | | | |
| Q9H116 | 50 | 16 | 47 | | 36 | |
| Q9H116 | 55 | 10 | 51 | | 45 | |
| Q9H116 | 17 | 8 | 47 | | | |
| Q9H116 | 17 | 8 | 47 | | | |
| Q9H116 | | 6 | 22 | | | |
| Q8IYU2 | 30 | | 25 | | 12 | |
| Q8IYU2 | 4 | 6 | 29 | | | |
| Q8IYU2 | 70 | 43 | 76 | | 74 | |
| Q8IYU2 | 2 | | 67 | 62 | 26 | |
| Q8IYU2 | 32 | | 13 | | 28 | |
| Q9ULT8 | 29 | 11 | 23 | | 45 | 11 |
| Q9ULT8 | 48 | | 37 | | 49 | |
| Q9ULT8 | | | 30 | | 6 | |
| Q9ULT8 | 37 | 42 | 36 | | | |
| Q9ULT8 | | | | | 8 | |
| Q9ULT8 | 66 | 35 | 65 | 44 | 63 | 15 |
| Q9ULT8 | | | 14 | | | |
| Q9ULT8 | 7 | | | | 26 | |
| Q9ULT8 | 34 | 19 | 23 | 4 | 16 | 6 |
| Q9ULT8 | 51 | 25 | 35 | 24 | 51 | |
| Q9ULT8 | 24 | | 4 | | 25 | |
| Q9ULT8 | 48 | 34 | 48 | 30 | 68 | 39 |
| Q9ULT8 | 68 | 32 | 54 | 32 | 86 | 29 |
| Q9ULT8 | 52 | 56 | 51 | 30 | 35 | 14 |
| Q9ULT8 | 68 | 33 | 35 | 11 | 46 | 1 |
| Q9ULT8 | 17 | | 20 | | 35 | |
| Q9ULT8 | 98 | 51 | 95 | 86 | 100 | 48 |
| Q9ULT8 | | 11 | 6 | | 17 | |
| Q9ULT8 | 40 | 19 | 25 | 14 | 35 | 4 |
| Q9ULT8 | 33 | 10 | 83 | 38 | 55 | 23 |
| Q9ULT8 | 73 | 48 | 70 | | 74 | 32 |
| Q9ULT8 | 40 | | 31 | | 47 | |
| Q9ULT8 | 14 | | 28 | | 53 | |
| Q9ULT8 | 34 | 25 | 71 | 50 | 100 | 46 |
| Q9ULT8 | 15 | | 11 | | | |
| Q9ULT8 | 73 | 30 | 54 | 30 | 44 | 54 |
| Q5T447 | 54 | 28 | 26 | 6 | 27 | 3 |
| Q5T447 | 44 | 40 | 68 | | 23 | |
| Q5T447 | 53 | 36 | 66 | 42 | 44 | 49 |
| Q5T447 | 26 | 7 | 20 | | 40 | |
| Q5T447 | 43 | 17 | 39 | 20 | 33 | |
| Q5T447 | 12 | | 15 | | | |
| Q5T447 | 56 | 21 | 66 | 40 | 44 | 23 |
| Q5T447 | 12 | 1 | 23 | | | |
| Q9Y4D8 | 35 | | 20 | | 45 | |
| Q9Y4D8 | 87 | | 45 | | 89 | |
| Q9Y4D8 | 46 | 12 | 29 | 7 | 53 | |
| Q9Y4D8 | 66 | 33 | 45 | 9 | 98 | 52 |
| Q9Y4D8 | 42 | | 27 | | 39 | |
| Q9Y4D8 | 48 | | 49 | 34 | 30 | 2 |
| Q9Y4D8 | 50 | 15 | 31 | | 46 | |
| Q9Y4D8 | | | 29 | 12 | | |
| Q9Y4D8 | | | 46 | 42 | | |
| Q9Y4D8 | 100 | 48 | | | 84 | 3 |
| Q9Y4D8 | 57 | | 17 | | 53 | |
| Q9Y4D8 | 16 | 4 | | | 26 | |
| Q9Y4D8 | 26 | | | | 22 | |
| Q9Y4D8 | 55 | 29 | 38 | | 36 | |
| Q9Y4D8 | 89 | 58 | 45 | 24 | 30 | |
| Q76N89 | 29 | 6 | | | 41 | |
| Q76N89 | 76 | 33 | 76 | 71 | 53 | 52 |
| Q9P2P5 | | | | | 10 | |
| Q9P2P5 | | | | | 2 | |
| Q9P2P5 | 22 | | | | 4 | |
| Q9P2P5 | 63 | | 36 | 25 | 62 | 23 |
| Q9P2P5 | 76 | 33 | 76 | 71 | 53 | 52 |
| Q15751 | 97 | 90 | 86 | 90 | 96 | 54 |
| Q15751 | 30 | | 2 | | 33 | |
| Q15751 | 44 | | 28 | | | |
| Q15751 | 44 | | 20 | | 54 | |
| Q15751 | 58 | 28 | 50 | 25 | 60 | 19 |
| Q15751 | 23 | | 17 | | 53 | |
| Q15751 | 82 | 45 | 73 | 42 | 64 | 12 |
| Q15751 | 63 | 32 | 79 | | | |
| Q15751 | 99 | 89 | 93 | 83 | 77 | 31 |
| Q15751 | 42 | 12 | 42 | | 28 | |
| Q15751 | 66 | 28 | 64 | | 23 | 5 |
| Q15751 | | | | | 24 | |
| Q15751 | 12 | | 16 | | 34 | |
| Q15751 | 47 | 23 | 41 | 5 | 64 | |
| Q15751 | 43 | 23 | 54 | | 74 | 8 |
| Q15751 | | 8 | 10 | | | |
| Q15751 | | | 10 | | 14 | |
| Q15751 | | | 37 | | 9 | |
| Q15751 | 61 | 26 | 58 | 34 | 62 | 15 |
| Q15751 | 29 | 21 | 34 | | 18 | |
| O95714 | 28 | 11 | 28 | | | |
| O95714 | 86 | 49 | 29 | | 55 | |
| O95714 | 55 | | 37 | | 62 | 4 |
| O95714 | 86 | 25 | 70 | 57 | 98 | 47 |
| O95714 | 77 | 47 | 81 | 67 | 63 | 40 |
| O95714 | 77 | 55 | 84 | 75 | 88 | 70 |
| O95714 | 74 | 61 | 94 | 91 | 100 | 76 |
| O95714 | 10 | | 15 | | 26 | |
| O95714 | 57 | 31 | 39 | | | |
| O95714 | 81 | 24 | 52 | | 50 | |
| O95714 | 67 | 34 | 75 | 44 | 76 | 29 |
| O95714 | 40 | | 54 | 1 | 53 | |
| O95714 | 94 | 48 | 95 | 85 | 79 | 16 |
| O95714 | 15 | 3 | 36 | | | |
| O95714 | | | 9 | | | |
| O95714 | | | 75 | 38 | 97 | 85 |
| O95714 | 74 | 57 | 95 | 93 | 93 | 83 |
| O95714 | | | 8 | | | |
| O95714 | | | 27 | | 22 | |
| O95714 | 66 | 67 | 72 | 73 | 66 | 29 |
| O95714 | 68 | 49 | 81 | | 8 | |
| O95714 | | | 18 | | | |
| O95714 | 28 | | 23 | | 33 | |
| O95714 | 60 | 13 | 45 | | 58 | |
| O95714 | 79 | 35 | 58 | 8 | 75 | 19 |
| O95714 | 55 | 18 | 49 | 11 | 44 | |
| O95714 | 100 | 60 | 100 | 76 | 49 | 14 |
| O95714 | 5 | 22 | 45 | 47 | 27 | |
| Q15034 | 55 | | 40 | | 48 | |
| Q15034 | 33 | | 58 | 6 | 53 | |
| Q15034 | 87 | 53 | 78 | 48 | | |
| Q5GLZ8 | 31 | 5 | 36 | | 41 | 15 |
| Q5GLZ8 | 63 | 22 | 76 | 49 | 63 | 48 |
| Q5GLZ8 | 82 | 55 | 78 | 69 | 49 | 55 |
| Q5GLZ8 | 63 | | 51 | | 57 | |
| Q5GLZ8 | 22 | 22 | 40 | 29 | 47 | 53 |
| Q5GLZ8 | 47 | | 41 | | 48 | |
| Q5GLZ8 | 61 | | 56 | | 64 | |
| Q5GLZ8 | 40 | | 15 | | 34 | |
| Q5GLZ8 | 64 | 33 | 59 | 50 | 47 | |
| Q5GLZ8 | 84 | 24 | 63 | 34 | 24 | |
| Q5GLZ8 | 18 | 29 | 38 | 21 | 38 | 26 |
| Q5GLZ8 | 53 | 21 | 56 | 31 | 50 | 34 |
| Q5GLZ8 | | 8 | 4 | | 27 | 7 |
| Q5GLZ8 | 12 | 26 | 48 | 35 | 43 | 30 |
| Q9UII4 | 62 | | 6 | | 30 | |
| Q9UII4 | | | 15 | | | |
| Q9UII4 | 69 | | 54 | | 59 | |
| Q9UII4 | 19 | | 51 | | 54 | |
| Q9UII4 | 85 | 41 | 97 | 92 | 72 | 50 |
| Q8IVU3 | 56 | | | | 20 | |
| Q8IVU3 | 33 | | | | 43 | |
| Q8IVU3 | 45 | | 20 | | 70 | |
| Q8IVU3 | 21 | | | | 33 | |
| P49773 | 27 | 39 | 60 | 30 | 55 | 23 |
| P49773 | 47 | 29 | 52 | 29 | 56 | 30 |
| Q14527 | 53 | 30 | 63 | 42 | 60 | 34 |
| Q14527 | 66 | 32 | 74 | 41 | 50 | 29 |
| Q14527 | 53 | 38 | 48 | 27 | 53 | 14 |
| Q14527 | 69 | | 80 | | 59 | 5 |
| Q14527 | 71 | 13 | 49 | | 60 | |
| Q14527 | 59 | | 44 | | 50 | |
| Q14527 | 14 | | 83 | 21 | 44 | |
| Q14527 | 28 | | 25 | | 37 | |
| Q14527 | 16 | 27 | 23 | | | |
| Q7Z6Z7 | 26 | | 24 | | 50 | 3 |
| Q7Z6Z7 | 23 | 3 | 44 | | 56 | |
| Q7Z6Z7 | 75 | 32 | 79 | 67 | 73 | |
| Q7Z6Z7 | 75 | 32 | 79 | 67 | 55 | |
| Q7Z6Z7 | 37 | | 39 | | 49 | 17 |
| Q7Z6Z7 | 74 | 40 | 51 | 57 | 61 | 41 |
| Q7Z6Z7 | 21 | 26 | 39 | 47 | 39 | |
| Q7Z6Z7 | 78 | 7 | 70 | | 58 | 1 |
| Q7Z6Z7 | 56 | | 43 | | 32 | |
| Q7Z6Z7 | 53 | 24 | 21 | 5 | 51 | |
| Q7Z6Z7 | 42 | | 44 | | 63 | |
| Q7Z6Z7 | | | | | 27 | |
| Q7Z6Z7 | 26 | | | | | |
| Q7Z6Z7 | 61 | | 37 | 37 | 35 | |
| Q7Z6Z7 | 41 | 44 | 18 | 17 | 36 | 13 |
| Q7Z6Z7 | 68 | 11 | 58 | | 78 | |
| Q7Z6Z7 | 10 | | 17 | 3 | 52 | |
| Q7Z6Z7 | 53 | 49 | 20 | 16 | 64 | 43 |
| Q7Z6Z7 | 40 | 27 | 53 | 29 | 52 | 38 |
| Q7Z6Z7 | 11 | | 26 | | 41 | |
| Q7Z6Z7 | 54 | 4 | 33 | | 55 | |
| Q7Z6Z7 | 58 | 43 | 52 | 38 | 51 | 46 |
| Q7Z6Z7 | 57 | 22 | 64 | 58 | 69 | 10 |
| Q7Z6Z7 | 36 | 43 | 29 | 6 | 14 | |
| Q7Z6Z7 | 88 | | 43 | 3 | 53 | |
| Q7Z6Z7 | 100 | 76 | 96 | 90 | 90 | 68 |
| Q7Z6Z7 | 91 | 53 | 88 | 74 | 85 | 16 |
| Q7Z6Z7 | 61 | 5 | 32 | | 55 | |
| Q7Z6Z7 | 51 | | 45 | | 43 | |
| Q7Z6Z7 | 41 | 27 | 56 | 9 | 38 | 24 |
| Q7Z6Z7 | 34 | 8 | 13 | | 50 | 41 |
| Q7Z6Z7 | 51 | 31 | 64 | 28 | 59 | 45 |
| Q7Z6Z7 | 53 | 28 | 55 | 32 | 63 | 43 |
| Q7Z6Z7 | 40 | 46 | 69 | 60 | 33 | 8 |
| Q7Z6Z7 | 71 | | 49 | | 57 | |
| Q7Z6Z7 | 56 | 52 | 53 | 42 | 46 | 44 |
| Q7Z6Z7 | 88 | 52 | 86 | 54 | 74 | 31 |
| Q7Z6Z7 | 88 | 46 | 79 | 46 | 93 | 44 |
| Q7Z6Z7 | 100 | 46 | 72 | 55 | 87 | 35 |
| Q7Z6Z7 | 39 | 18 | 67 | 48 | 52 | 3 |
| Q7Z6Z7 | 38 | 30 | 40 | 27 | 49 | 52 |
| Q7Z6Z7 | 40 | | 24 | | 26 | 1 |
| Q7Z6Z7 | 19 | | 27 | | | |
| Q7Z6Z7 | 16 | 9 | 49 | 37 | 48 | 14 |
| Q7Z6Z7 | 32 | 35 | 72 | 58 | 47 | 8 |
| Q7Z6Z7 | 64 | 50 | 63 | 44 | 50 | 23 |
| Q9P2D0 | 71 | | 73 | | 88 | |
| Q9P2D0 | 73 | | 66 | | 86 | |
| Q9P2D0 | 60 | | 34 | | 52 | |
| Q9P2D0 | 56 | | 65 | | 54 | |
| Q9P2D0 | 35 | | 16 | | 30 | |
| Q9P2D0 | 38 | | 39 | | 42 | |
| Q9P2D0 | 23 | | 28 | | 22 | |
| Q9P2D0 | 17 | 19 | 43 | | | |
| Q9P2D0 | 56 | 31 | 27 | 2 | 47 | 7 |
| Q9Y573 | 69 | | 43 | | | |
| Q9Y573 | 81 | 28 | 54 | | 50 | |
| Q9Y573 | 56 | 38 | 42 | 28 | 51 | 18 |
| Q8IU81 | 1 | | 13 | | 14 | |
| Q8IU81 | 51 | 24 | 80 | 63 | 43 | 22 |
| Q8IU81 | | 2 | 23 | | 9 | 5 |
| Q8IU81 | 30 | 32 | 45 | 29 | 30 | |
| Q8IU81 | 84 | 39 | 71 | 43 | 49 | 20 |
| Q8IU81 | 90 | 31 | 71 | 60 | 46 | 23 |
| Q8IU81 | 37 | 27 | 57 | 31 | 50 | 28 |
| Q8IU81 | 23 | | 21 | | | |
| Q8IU81 | 14 | 8 | 44 | 35 | 33 | 7 |
| Q8IU81 | 23 | 24 | 50 | 31 | 44 | 18 |
| Q8IU81 | 35 | 26 | 51 | 40 | 40 | 46 |
| Q7Z5L9 | 42 | 28 | 66 | 52 | 52 | 27 |
| Q7Z5L9 | 45 | 31 | 78 | 60 | 56 | 32 |
| Q7Z5L9 | 36 | 10 | 58 | 19 | 19 | 30 |
| Q7Z5L9 | 48 | 22 | 54 | 17 | 16 | |
| Q7Z5L9 | 54 | 54 | 60 | 61 | 66 | 50 |
| Q7Z5L9 | 11 | 18 | 13 | | | |
| Q7Z5L9 | 27 | 24 | 67 | 33 | 54 | 18 |
| Q7Z5L9 | 23 | 24 | 50 | 31 | 44 | 18 |
| Q7Z5L9 | 35 | 26 | 51 | 40 | 40 | 46 |
| Q7Z5L9 | | | 85 | 65 | 49 | |
| Q9H1B7 | 42 | 28 | 66 | 52 | 52 | 27 |
| Q9H1B7 | 45 | 31 | 78 | 60 | 56 | 32 |
| Q9H1B7 | 9 | | 30 | | 29 | |
| Q9H1B7 | 3 | | 12 | | | |
| Q9H1B7 | 65 | 56 | 78 | 72 | 71 | 41 |
| Q9H1B7 | 45 | 24 | 23 | | 14 | |
| Q9H1B7 | 38 | | 21 | 31 | 27 | |
| Q9H1B7 | 100 | 24 | 86 | 44 | 39 | 5 |
| Q9H1B7 | 3 | | | | | |
| Q9H1B7 | 27 | 24 | 67 | 33 | 54 | 18 |
| Q9H1B7 | 23 | 24 | 50 | 31 | 44 | 18 |
| Q9H1B7 | 35 | 26 | 51 | 40 | 40 | 46 |
| Q9H1B7 | 17 | | | | 39 | |
| Q9H1B7 | | | 88 | 68 | 55 | |
| Q96J02 | 50 | 48 | 33 | 26 | 32 | 45 |
| Q96J02 | 55 | 35 | 81 | 61 | 85 | 36 |
| Q96J02 | 59 | 22 | 34 | | 80 | 25 |
| Q9Y6Y0 | 33 | | 33 | | 44 | |
| Q9Y6Y0 | 27 | | 19 | | 34 | |
| Q9Y6Y0 | 28 | | 24 | | 45 | |
| Q9Y6Y0 | 40 | | 23 | | 46 | |
| Q9Y6Y0 | 23 | | 15 | | 35 | |
| Q9Y6Y0 | 70 | | 31 | | 68 | |
| Q9Y6Y0 | 82 | 62 | 68 | 55 | 71 | 20 |
| Q9Y6Y0 | 17 | | 10 | | 24 | |
| Q9Y6Y0 | 46 | 23 | 31 | 13 | 45 | 54 |
| Q9Y6Y0 | | | 14 | | | |
| Q9Y6Y0 | 22 | 10 | 20 | 7 | 26 | 15 |
| Q9Y6Y0 | | | 15 | 3 | 34 | |
| Q9Y6Y0 | 69 | 16 | 43 | 23 | 44 | 39 |
| O94819 | 18 | 24 | 3 | | 29 | |
| O94819 | 10 | 26 | 33 | 17 | 21 | 6 |
| O94819 | 19 | | 35 | 30 | 31 | 15 |
| O94819 | 63 | 24 | 74 | 43 | 65 | 7 |
| O94819 | 25 | 25 | 11 | | 6 | |
| O94819 | 11 | 22 | 23 | | 30 | |
| O94819 | 17 | 9 | 81 | 69 | 36 | 12 |
| Q8IY47 | 27 | | 37 | | 32 | |
| Q8IY47 | 3 | | 9 | | 30 | |
| Q8IY47 | 46 | 5 | 32 | | 39 | |
| Q8IY47 | 6 | 2 | 12 | | 16 | |
| Q8NAB2 | 57 | | 21 | | 39 | 12 |
| Q9NVX7 | 88 | | 77 | | 36 | |
| Q9NVX7 | 4 | 7 | 29 | 12 | | |
| Q9NVX7 | 52 | 38 | 48 | 54 | 42 | 19 |
| Q9NVX7 | 59 | | 28 | | 44 | |
| Q9NVX7 | 34 | | 26 | | 51 | |
| Q9NVX7 | 2 | 25 | 50 | | | |
| Q86V97 | 65 | | 41 | | 56 | |
| Q86V97 | 83 | 17 | 79 | | 80 | |
| Q86V97 | 15 | | 30 | | | |
| Q86V97 | 50 | 39 | 45 | 8 | 43 | |
| Q86V97 | 28 | 7 | 43 | | | |
| Q8WVZ9 | 29 | | 34 | | 25 | |
| Q8WVZ9 | 83 | 17 | 79 | | 80 | |
| Q8WVZ9 | 15 | | 30 | | | |
| Q8NFY9 | 79 | 38 | 48 | 28 | 52 | 18 |
| Q8NFY9 | 62 | 18 | 42 | | 56 | |
| Q8NFY9 | 42 | 7 | 33 | 29 | 62 | |
| Q8NFY9 | | 1 | 11 | | | |
| Q8NFY9 | | | 11 | | | |
| Q8NFY9 | | | 27 | | | |
| Q8NFY9 | | | 20 | | | |
| Q8NFY9 | 86 | 32 | 49 | 36 | 59 | 13 |
| Q8NFY9 | | 13 | 21 | | | |
| Q8NFY9 | | | | | 18 | 4 |
| Q9P0J7 | 28 | 15 | 21 | 7 | 29 | 28 |
| Q9P0J7 | 19 | 10 | 90 | 90 | 33 | 6 |
| Q9P0J7 | 4 | | 1 | | 20 | |
| Q719H9 | | | | | 6 | |
| Q719H9 | | | | | 14 | |
| Q9H3F6 | 15 | | | | 2 | |
| Q9H3F6 | 50 | | 23 | | 31 | |
| Q9H3F6 | 9 | 8 | 21 | | 10 | 7 |
| Q9H3F6 | | 4 | 20 | | | |
| Q96CX2 | 45 | 31 | 62 | 21 | 34 | 17 |
| Q96CX2 | 15 | 17 | 17 | | 27 | |
| Q8WZ19 | 9 | 8 | 21 | | 10 | 7 |
| Q8WZ19 | | 4 | 20 | | | |
| Q96SI1 | 44 | | 23 | | 44 | |
| Q96SI1 | 33 | | | | 22 | |
| Q96SI1 | 42 | | 33 | 17 | 21 | 22 |
| Q7Z5Y7 | | | | | 2 | |
| Q7Z5Y7 | 48 | 12 | 31 | | 50 | |
| Q7Z5Y7 | 4 | | 4 | | | |
| Q7Z5Y7 | 58 | 47 | 58 | 40 | 45 | 36 |
| Q4G0X4 | 51 | | 38 | | 33 | |
| Q9Y597 | 47 | | 56 | | 68 | |
| Q9Y597 | 24 | | 23 | | 48 | |
| Q9Y597 | 95 | 89 | 96 | 87 | 90 | 84 |
| Q9Y597 | 51 | | 51 | 19 | 36 | |
| Q9Y597 | 73 | 58 | 73 | 56 | 77 | 21 |
| Q9Y597 | 43 | 31 | 35 | 7 | 39 | 6 |
| Q8WVF5 | 18 | | 4 | | 11 | |
| Q9NXV2 | 46 | 23 | 41 | 17 | 44 | 20 |
| Q9NXV2 | 33 | 26 | 42 | 16 | 39 | 42 |
| Q9NXV2 | | | 2 | | 9 | |
| Q8NC69 | 47 | | 18 | | 39 | |
| Q96MP8 | 100 | 77 | 98 | 79 | 79 | 17 |
| Q6ZWB6 | 15 | | 12 | | 53 | |
| Q6ZWB6 | 34 | | | | 49 | |
| Q7L273 | 26 | | | | 43 | |
| Q7L273 | 17 | 41 | 22 | 8 | 28 | 2 |
| Q7L273 | 27 | 9 | 23 | 23 | 61 | |
| Q7L273 | 49 | | 50 | | 49 | |
| Q7L273 | 21 | | 5 | | 21 | |
| Q7L273 | 32 | | 11 | | | |
| Q9Y2K7 | 33 | 11 | 37 | | 42 | |
| Q9Y2K7 | 62 | 12 | 26 | | 50 | |
| Q9Y2K7 | 55 | 19 | 22 | | 34 | |
| Q9Y2K7 | 63 | | 50 | | 40 | |
| Q9Y2K7 | 5 | 4 | 30 | | | |
| Q9Y2K7 | 48 | | 15 | | 49 | 23 |
| Q9Y2K7 | 48 | | 30 | | 27 | |
| Q9Y2K7 | 54 | 11 | 41 | | 45 | 6 |
| Q9Y2K7 | 41 | | 26 | | 40 | |
| Q9Y2K7 | 31 | 12 | 62 | 47 | 34 | 24 |
| Q9Y2K7 | 45 | 22 | 49 | 36 | 46 | 11 |
| Q9Y2K7 | 29 | | 38 | | 44 | |
| Q9Y2K7 | 34 | | 63 | | 44 | |
| Q9Y2K7 | 64 | | 44 | | 56 | |
| Q9Y2K7 | 18 | 2 | 34 | 8 | 26 | 7 |
| Q9Y2K7 | 45 | 27 | 43 | 48 | 42 | |
| Q9Y2K7 | 62 | 37 | 48 | 26 | 58 | 57 |
| Q8NHM5 | 31 | 8 | 38 | | 51 | |
| Q8NHM5 | 42 | | 40 | | 32 | |
| Q8NHM5 | 35 | | 29 | | 34 | |
| Q8NHM5 | 21 | 17 | 39 | 3 | 25 | |
| Q8NHM5 | 53 | 15 | 21 | | 70 | |
| Q8NHM5 | 46 | 18 | 56 | 38 | 52 | 18 |
| Q8NHM5 | 61 | 8 | 36 | | | |
| Q8NHM5 | 52 | 15 | 52 | | 62 | 22 |
| Q8NHM5 | 58 | | 51 | | 73 | |
| Q8NHM5 | 5 | | | | 20 | |
| Q14145 | 81 | 19 | 88 | | 68 | |
| Q14145 | 89 | 7 | 80 | | 81 | |
| Q14145 | 98 | 97 | 98 | 98 | 96 | 97 |
| Q14145 | 72 | | 58 | | 57 | |
| Q14145 | 94 | | 84 | | 93 | |
| Q14145 | 88 | 57 | 89 | | | |
| Q14145 | 98 | 87 | 99 | 99 | 99 | 85 |
| Q14145 | 88 | 76 | 93 | 89 | 92 | 79 |
| Q14145 | 78 | 56 | 91 | 77 | 100 | 53 |
| Q14145 | 50 | 14 | 54 | 24 | 50 | 36 |
| Q14145 | 95 | 90 | 78 | 73 | 97 | 78 |
| Q14145 | 12 | | 26 | | | |
| Q14145 | 37 | | 92 | 82 | 69 | |
| Q9HCI6 | 60 | | 25 | | 62 | |
| Q9HCI6 | 33 | | | | 26 | |
| Q9HCI6 | 31 | | 1 | | 12 | |
| Q6PID8 | 79 | 32 | 78 | | 59 | |
| Q6PID8 | 13 | 1 | 18 | | 17 | |
| Q6PID8 | | | | | 55 | 10 |
| Q9Y2U9 | 60 | | 29 | | 45 | |
| Q9Y2U9 | 40 | | 30 | | 52 | |
| Q9Y2U9 | 12 | | 24 | | 35 | |
| Q9Y2U9 | 22 | 4 | 30 | | 45 | |
| Q9BQ90 | 52 | 36 | 47 | | 33 | |
| Q9BQ90 | 67 | 35 | 67 | 46 | 37 | 36 |
| Q9BQ90 | | 17 | 31 | | | |
| Q9BQ90 | 92 | 56 | 76 | 51 | 49 | 47 |
| Q9BQ90 | | 1 | 17 | | 32 | |
| Q9BQ90 | 30 | 6 | 37 | 39 | 25 | 8 |
| Q9NR64 | 35 | | 31 | | 27 | |
| Q9NR64 | 44 | 11 | 31 | | | |
| Q9NVR0 | 46 | | 32 | | 33 | |
| Q9NVR0 | 46 | 3 | 42 | | 37 | |
| Q9NVR0 | 48 | 38 | 59 | 40 | 59 | 32 |
| Q9NVR0 | 28 | 34 | 18 | | 37 | 7 |
| Q9NVR0 | 53 | 29 | 51 | | 36 | |
| Q9NVR0 | 11 | | 27 | | | |
| Q9NVR0 | | | 20 | | | |
| Q53G59 | | | | | 6 | |
| Q53G59 | 64 | | 38 | | 43 | |
| Q53G59 | 7 | | 11 | | | |
| Q9P2N7 | | | | | 21 | 3 |
| Q9P2N7 | 44 | 34 | 25 | 23 | 35 | 15 |
| Q9P2G3 | | | 34 | 35 | | |
| Q9P2G3 | | | 34 | 35 | | |
| Q9P2G3 | 32 | | 14 | | 28 | |
| Q9P2G3 | 30 | | | | 35 | |
| O94889 | 17 | | 17 | | 36 | |
| O94889 | 30 | | 24 | | 12 | |
| Q9Y2M5 | 56 | 22 | 48 | 30 | 53 | |
| Q9Y2M5 | 92 | 42 | 25 | 6 | 82 | 7 |
| Q53GT1 | 61 | 29 | 42 | 29 | 45 | |
| Q8NBE8 | 32 | | 28 | | 48 | |
| Q8NBE8 | 42 | | 15 | | 21 | |
| Q6TFL4 | 56 | | 22 | | 32 | |
| Q6TFL4 | 26 | | 21 | | 35 | |
| Q6TFL4 | 73 | 48 | 70 | | 4 | |
| Q6TFL4 | 79 | | 64 | | 72 | |
| Q9H0H3 | 32 | | 22 | | 38 | |
| Q9H0H3 | 37 | 13 | 17 | | 53 | |
| Q9H0H3 | 25 | | 13 | | | |
| Q9H0H3 | 5 | | | | | |
| Q9H0H3 | 40 | | 29 | | 55 | |
| Q53HC5 | 58 | 11 | 86 | | 49 | |
| Q53HC5 | 39 | | 60 | | 64 | |
| Q53HC5 | 31 | 15 | 38 | | 57 | |
| Q53HC5 | 92 | 53 | 91 | 91 | 91 | 40 |
| Q53HC5 | 89 | | 85 | | 100 | |
| Q53HC5 | 44 | | 24 | | 59 | |
| Q0D2K2 | 70 | | 18 | | 21 | |
| Q8N4N3 | 7 | 8 | 37 | 19 | 18 | |
| Q8N4N3 | 1 | 5 | 4 | 18 | 7 | |
| Q8N4N3 | 35 | 17 | 36 | 3 | 36 | |
| Q8N4N3 | 83 | | 79 | | 36 | |
| Q9C0H6 | 55 | | 5 | | 43 | |
| Q9C0H6 | 44 | | 41 | | 45 | |
| Q9C0H6 | 48 | | 41 | | 55 | |
| Q9C0H6 | 47 | | 46 | | 57 | |
| Q9C0H6 | 44 | | | 1 | 34 | 13 |
| Q9C0H6 | 22 | 17 | 37 | | 17 | |
| Q9P2K6 | 7 | | | | 60 | 4 |
| Q96PQ7 | 48 | | 41 | | 55 | |
| Q96PQ7 | 32 | | 26 | | 39 | |
| Q96PQ7 | 35 | | 31 | | 27 | |
| Q96PQ7 | 22 | 17 | 37 | | 17 | |
| Q8WZ60 | 55 | 62 | 53 | 50 | 57 | 10 |
| Q8WZ60 | 31 | 5 | 40 | | 36 | |
| Q8WZ60 | 28 | 17 | 54 | | | |
| Q8WZ60 | | 2 | 33 | | | |
| Q8WZ60 | | | 25 | | 41 | |
| Q8WZ60 | 23 | 9 | 16 | 37 | 1 | 5 |
| Q8WZ60 | 35 | 13 | 44 | | | |
| Q8WZ60 | 21 | 22 | 70 | | | |
| Q8IXQ5 | 66 | 13 | 49 | | 18 | |
| Q8IXQ5 | | | 18 | | | |
| Q9P2G9 | 29 | | 29 | | 60 | |
| Q9P2G9 | 43 | 40 | 42 | 29 | 53 | 1 |
| Q9P2J3 | 66 | | 60 | | 58 | |
| Q9P2J3 | | | | | 21 | 3 |
| Q9P2J3 | 44 | 34 | 25 | 23 | 35 | 15 |
| Q08380 | 47 | | 6 | | | |
| Q08380 | | | 26 | | 91 | 54 |
| Q08380 | 45 | | 29 | | 29 | |
| Q08380 | 72 | 37 | 75 | 69 | 74 | 30 |
| Q8TBB1 | 60 | | 56 | | 48 | |
| Q8TBB1 | 88 | 31 | 63 | 40 | 78 | 59 |
| Q8N448 | 23 | | 42 | | 19 | |
| Q8N448 | 18 | | 11 | | 17 | |
| Q8N448 | 51 | | 30 | | 15 | |
| Q8N448 | | | | | 8 | |
| Q8N448 | 64 | | 52 | | 32 | |
| Q1L5Z9 | 2 | | 11 | | 15 | |
| Q1L5Z9 | 97 | 68 | 94 | 84 | 86 | 37 |
| Q1L5Z9 | 98 | 46 | 81 | | 68 | |
| Q1L5Z9 | | | 45 | 10 | 39 | 17 |
| Q1L5Z9 | 73 | | 72 | | 57 | |
| Q1L5Z9 | 20 | 2 | | | 31 | |
| Q1L5Z9 | 98 | 27 | 66 | | 68 | |
| Q1L5Z9 | 93 | 88 | 72 | 36 | 59 | 27 |
| Q1L5Z9 | 65 | | 59 | 24 | 56 | |
| Q1L5Z9 | 94 | 58 | 94 | 78 | 83 | 12 |
| Q1L5Z9 | 30 | | 6 | | 39 | |
| Q496Y0 | 55 | | 26 | | 47 | |
| Q496Y0 | 9 | | | | 52 | 26 |
| Q496Y0 | 31 | | | | | |
| Q96L50 | 41 | 12 | 46 | | 28 | |
| Q96L50 | 23 | 9 | 31 | | 39 | 7 |
| Q96L50 | 61 | 23 | 66 | 39 | 63 | 9 |
| Q96L50 | | | 40 | | 31 | |
| Q96L50 | 95 | 49 | 40 | 23 | 40 | 20 |
| Q96L50 | 44 | | 8 | | 25 | |
| Q15345 | 77 | 31 | 80 | 43 | 56 | 30 |
| Q15345 | 83 | 75 | 72 | 59 | 81 | 28 |
| Q15345 | 47 | 17 | 45 | | 6 | |
| Q15345 | 64 | 38 | 73 | 48 | 66 | 49 |
| Q15345 | | | | | 7 | |
| Q15345 | 50 | 5 | 52 | | 51 | |
| Q15345 | 21 | | 22 | | 40 | |
| Q15345 | 57 | | 29 | | 58 | |
| Q15345 | | | 36 | 16 | | |
| Q6UWE0 | 24 | 22 | 33 | 17 | 35 | 26 |
| Q6UWE0 | 80 | | 80 | | 76 | |
| Q6UWE0 | 68 | 43 | 38 | 40 | 53 | 36 |
| Q6UWE0 | 78 | | 76 | | 85 | 10 |
| Q6UWE0 | 84 | | 55 | | 57 | |
| Q6UWE0 | 41 | | 13 | | 79 | |
| Q6UWE0 | 58 | 33 | 55 | 37 | 74 | 23 |
| Q6UWE0 | 23 | | | | 4 | |
| Q6UWE0 | | | | | 16 | |
| Q6UWE0 | 42 | 44 | 34 | | 16 | |
| Q6UWE0 | 42 | 44 | 37 | | 10 | |
| O94822 | 29 | | 8 | | 38 | |
| O94822 | | | | | 1 | |
| O94822 | 24 | | 33 | | 47 | |
| O94822 | 16 | | 34 | | 48 | |
| O94822 | 25 | | 1 | | 60 | |
| O94822 | 19 | 19 | 25 | | 34 | 1 |
| O94822 | 42 | | 31 | 6 | 20 | 13 |
| O94822 | 47 | 29 | 63 | 28 | 59 | 20 |
| O94822 | 34 | 9 | 55 | 34 | 46 | |
| O94822 | 49 | | 35 | | 60 | |
| O94822 | 66 | 53 | 77 | 65 | 81 | 14 |
| O94822 | 77 | 62 | 52 | 29 | 53 | 39 |
| O94822 | 51 | | 50 | | 52 | |
| O94822 | 92 | | 87 | | 86 | |
| O94822 | 66 | 14 | 69 | | 71 | |
| O94822 | 48 | | 50 | | 56 | |
| O94822 | 75 | | 64 | | 74 | |
| O94822 | 60 | | 32 | | 34 | |
| O94822 | 87 | | 75 | | 85 | |
| O94822 | 89 | | 76 | | 77 | |
| O94822 | 85 | | 76 | | 72 | |
| Q8N653 | | | | | 3 | |
| Q8N653 | 24 | 11 | 26 | | 7 | |
| Q8N653 | 64 | | 57 | | 67 | |
| Q8N653 | 64 | | 57 | | 67 | |
| Q8N653 | 64 | | 57 | | 67 | |
| Q8N653 | 21 | | 27 | | 15 | |
| Q8N653 | 11 | | | | 13 | |
| Q8N653 | 52 | 15 | 27 | | 44 | 11 |
| Q8N653 | | 1 | | | | |
| Q13233 | 56 | 36 | 53 | | 49 | |
| Q13233 | 21 | 8 | 37 | | | |
| Q13233 | 35 | | 63 | | | |
| Q13233 | 37 | 12 | 37 | | 44 | |
| Q13233 | 51 | 28 | 46 | 19 | 82 | 31 |
| Q13233 | 49 | 1 | 29 | | 36 | |
| Q13233 | 72 | 35 | 62 | | 38 | |
| Q13233 | 26 | 21 | 43 | | | |
| Q13233 | 61 | 23 | 42 | | | |
| Q8TCQ1 | 74 | 30 | 78 | | | |
| Q8TCQ1 | | 3 | 39 | | | |
| Q8TCQ1 | 78 | 16 | 92 | | | |
| Q8TCQ1 | 44 | 16 | 49 | | | |
| Q8TCQ1 | 39 | 13 | 58 | | | |
| Q8TCQ1 | 34 | | 33 | | | |
| Q86UD3 | 77 | 34 | 66 | | | |
| Q9NX47 | | 7 | 34 | | | |
| Q9NX47 | | | 15 | | | |
| Q9NX47 | 36 | 6 | 37 | | 54 | |
| Q9NX47 | 77 | 12 | 68 | | 42 | |
| Q9NX47 | | | | | 27 | |
| Q9NX47 | 28 | | 40 | 30 | 31 | |
| O60337 | 68 | | 82 | 33 | 74 | 19 |
| O60337 | 85 | 35 | 96 | 72 | 83 | 36 |
| O60337 | 55 | 18 | 96 | | 24 | |
| O60337 | 86 | 44 | 94 | 89 | 72 | 48 |
| Q9H992 | 10 | 20 | 46 | | 43 | |
| Q9H992 | 34 | 17 | 22 | | | |
| Q9H992 | 58 | | 41 | | 66 | |
| Q9H992 | 9 | 18 | 47 | | | |
| Q9H992 | | | | | 28 | |
| Q9H992 | 71 | 32 | 84 | | 53 | |
| Q9H992 | 62 | | 50 | | 58 | |
| Q9H992 | 60 | | 56 | | 65 | |
| Q9H992 | 86 | 35 | 98 | 82 | 71 | 21 |
| Q9H992 | 62 | | 35 | | 23 | |
| Q00987 | 52 | | 48 | | 81 | |
| Q00987 | 61 | 26 | 60 | | | |
| Q00987 | 35 | 8 | 35 | | 18 | |
| Q00987 | 15 | | 26 | | | |
| Q00987 | 23 | | 54 | | | |
| Q00987 | 54 | | 34 | | 37 | |
| Q00987 | 72 | | 67 | | 59 | |
| Q00987 | 74 | 27 | 82 | | | |
| O15151 | 29 | | 23 | | 55 | |
| O15151 | 28 | | | | 41 | |
| O15151 | 37 | | 30 | | 41 | |
| Q96G25 | 47 | 29 | 13 | 8 | 31 | 10 |
| Q96G25 | 23 | 31 | 26 | | 40 | |
| A1L020 | 35 | | 27 | | 37 | 19 |
| A1L020 | 65 | 37 | 75 | 52 | 73 | 10 |
| A1L020 | | | | | 16 | 12 |
| A1L020 | 38 | 13 | 13 | | 38 | |
| A1L020 | 67 | | 39 | | 55 | |
| A1L020 | 52 | | 19 | | 39 | |
| A1L020 | 7 | | 47 | | 21 | |
| Q6ZN04 | 51 | | 28 | | 51 | |
| Q5U5Q3 | 19 | | 26 | | 6 | |
| Q5U5Q3 | 28 | | 4 | | 37 | |
| Q5U5Q3 | 46 | 5 | 74 | | | |
| Q86XN8 | 41 | 35 | 12 | | 29 | |
| Q86XN8 | 16 | 15 | 59 | 48 | 62 | |
| Q86XN8 | 54 | | 47 | | 47 | |
| Q86XN8 | 49 | | 32 | | 52 | |
| O60291 | 38 | | 24 | | 42 | |
| O60291 | 56 | | 29 | | 34 | |
| O60291 | 69 | | 62 | | 76 | |
| O60291 | 73 | | 83 | | 82 | |
| O60291 | 83 | 9 | 74 | | 65 | |
| O60291 | 84 | | 55 | | 49 | |
| O60291 | 53 | 35 | 66 | 45 | 52 | 13 |
| O60291 | 32 | 40 | 24 | 3 | 37 | 20 |
| Q86YT6 | 38 | 8 | 19 | 22 | | |
| Q86YT6 | 44 | 25 | 33 | 19 | 17 | 31 |
| Q86YT6 | | | 5 | | | |
| Q86YT6 | 10 | 1 | | | 17 | |
| Q86YT6 | 36 | | 23 | | 31 | |
| Q86YT6 | 6 | | 27 | | 25 | |
| Q86YT6 | 7 | 2 | 6 | | 14 | |
| Q86YT6 | 18 | | 20 | | 34 | |
| Q86YT6 | 17 | | 17 | | 17 | |
| Q86YT6 | 23 | 13 | 46 | | | |
| Q86YT6 | 36 | | | | 24 | |
| Q86YT6 | 36 | | | | 24 | |
| Q86YT6 | 52 | | 6 | | 39 | |
| Q86YT6 | 4 | | | | 18 | |
| Q96AX9 | 20 | 2 | 35 | | 26 | |
| Q96AX9 | 62 | 5 | 59 | | 61 | |
| Q96AX9 | 74 | | 43 | | 69 | |
| Q96AX9 | 60 | 21 | 66 | | 57 | 11 |
| Q96AX9 | 67 | | 30 | | 52 | |
| Q96AX9 | 66 | 9 | 56 | | 66 | |
| Q96AX9 | 66 | 19 | 72 | | 56 | 9 |
| Q96AX9 | 71 | | 33 | | 74 | |
| Q96AX9 | 100 | | 87 | | 100 | |
| O15344 | 21 | | 19 | | 48 | |
| O15344 | 13 | | 17 | | 36 | |
| O15344 | 28 | 21 | | | 48 | 60 |
| O15344 | 24 | | 3 | | 47 | |
| O15344 | 26 | | 9 | | 24 | |
| O15344 | 26 | | 26 | | 13 | |
| O15344 | 24 | | 5 | | 36 | |
| O15344 | 18 | | 6 | | 57 | |
| O15344 | 22 | 3 | 20 | | 23 | |
| O15344 | 34 | | 43 | 13 | 27 | |
| O15344 | 58 | 16 | 53 | 26 | 31 | 6 |
| O15344 | 49 | | 15 | 4 | 66 | 25 |
| O15344 | 44 | 9 | 28 | 27 | 84 | 59 |
| Q9UJV3 | 8 | | 5 | | 8 | |
| Q9UJV3 | 82 | | 24 | | 55 | |
| Q9UHC7 | 34 | 10 | 31 | | 46 | |
| Q9UHC7 | | 3 | | 2 | | 8 |
| Q9UHC7 | 47 | 3 | 32 | | 50 | 4 |
| Q9UHC7 | 43 | 14 | 27 | 14 | 28 | |
| Q9UHC7 | 69 | 27 | 67 | 40 | 67 | 31 |
| Q9UHC7 | 66 | 23 | 53 | | 49 | |
| Q9UHC7 | 28 | | 14 | | 41 | |
| Q9UHC7 | 63 | | 35 | 14 | 58 | |
| Q9UHC7 | 62 | 7 | 46 | | 57 | |
| Q9UHC7 | 82 | 44 | 69 | 48 | 78 | 33 |
| Q9UHC7 | 75 | | 44 | 43 | 73 | 11 |
| Q9UHC7 | 67 | 29 | 84 | 74 | 72 | 45 |
| Q9UHC7 | 59 | 21 | 56 | 15 | 69 | 28 |
| Q9UHC7 | 59 | | 53 | 29 | 44 | |
| Q9H000 | 16 | 15 | 42 | 2 | 44 | 17 |
| Q9H000 | 26 | 8 | 55 | 29 | 47 | 7 |
| Q9H000 | 38 | 24 | 32 | 14 | 66 | 10 |
| Q9H000 | 78 | 31 | 92 | 80 | 68 | 23 |
| Q9H000 | 86 | | 42 | | 35 | |
| Q9H000 | 71 | 4 | 76 | | 50 | |
| Q9H000 | 51 | 27 | 60 | 26 | 70 | 29 |
| Q9H000 | 46 | | 73 | | 73 | |
| Q9H000 | 64 | | 73 | | 76 | |
| Q9H000 | 78 | 32 | 65 | 39 | 83 | 30 |
| Q9H000 | 85 | 47 | 43 | | 63 | |
| Q9H000 | 67 | 29 | 84 | 74 | 72 | 45 |
| Q9H000 | 59 | 21 | 56 | 15 | 69 | 28 |
| Q9H000 | 66 | 24 | 69 | 50 | 55 | 41 |
| Q13064 | | 2 | | | | |
| Q13064 | 37 | | 16 | | 23 | |
| P51948 | 46 | 3 | 49 | | 54 | |
| P51948 | 19 | | 33 | | 36 | |
| P51948 | 66 | | 35 | | 70 | |
| Q68DK7 | 57 | 34 | 31 | 23 | 52 | 2 |
| Q68DK7 | 39 | 29 | 23 | | 24 | |
| Q68DK7 | 42 | 27 | 39 | 26 | 61 | 1 |
| Q68DK7 | 70 | 45 | 94 | 69 | 96 | 37 |
| Q68DK7 | 73 | 37 | 47 | 14 | 57 | 11 |
| Q68DK7 | 68 | | 17 | | 32 | |
| Q68DK7 | 72 | 13 | 34 | | 57 | 8 |
| Q68DK7 | 56 | 37 | 64 | 57 | 51 | 8 |
| Q68DK7 | 56 | 33 | 39 | 30 | 60 | 26 |
| Q68DK7 | 58 | 40 | 28 | | 47 | 1 |
| Q9HCI7 | 69 | 47 | 47 | | 80 | 15 |
| Q9HCI7 | 68 | | 56 | | 88 | |
| Q9HCI7 | 34 | | 36 | | 72 | |
| Q9HCI7 | | | 32 | | | |
| Q9HCI7 | 69 | 12 | 63 | 49 | 45 | 11 |
| Q9HCI7 | | | 41 | | | |
| Q969V5 | 56 | 25 | 60 | 38 | 61 | 40 |
| Q969V5 | | | | | 46 | 10 |
| Q969V5 | 36 | 35 | 46 | | 61 | 24 |
| Q969V5 | 45 | | 21 | | 74 | |
| Q969V5 | 16 | | 14 | | 36 | |
| Q969V5 | 55 | | 56 | | 58 | |
| O75592 | 58 | | 34 | | 42 | |
| O75592 | 56 | 7 | 42 | | 46 | |
| O75592 | 50 | | 25 | | 37 | 9 |
| O75592 | 59 | | 55 | | 69 | 3 |
| O75592 | | | 8 | | | |
| O75592 | 18 | 15 | | | | |
| O75592 | 18 | 15 | | 2 | | |
| O75592 | | | | 2 | | |
| O75592 | 18 | 15 | | 2 | | |
| O75592 | 44 | 12 | 15 | | 29 | |
| O75592 | 49 | | 48 | | 43 | |
| O75592 | 8 | 12 | 34 | | | |
| O75592 | 39 | | 38 | | 35 | |
| O75592 | 83 | 55 | 58 | 39 | 65 | 53 |
| O75592 | 12 | | 32 | | | |
| O75592 | | | 27 | | | |
| O75592 | | | 31 | | | |
| O75592 | 30 | 16 | 38 | 3 | 38 | 2 |
| O75592 | | | 70 | 4 | 32 | 4 |
| O75592 | 100 | | 30 | 15 | 42 | 24 |
| O75592 | 94 | | 14 | | 32 | |
| O75592 | 3 | 10 | 46 | 36 | 26 | |
| O75592 | | 4 | 30 | | | |
| O75592 | 46 | | 22 | | 33 | |
| O75592 | 62 | 18 | 42 | | 60 | 27 |
| O75592 | 56 | 7 | 25 | | 61 | |
| O75592 | 59 | 25 | 23 | 27 | 28 | 47 |
| O75592 | 53 | 19 | 32 | 29 | 59 | 7 |
| O75592 | 42 | | | | 36 | |
| O75592 | | | 2 | | 26 | |
| O75592 | 79 | 33 | 41 | 37 | 56 | 44 |
| O75592 | 33 | | | | 7 | |
| O75592 | 17 | 3 | 19 | | 19 | |
| O75592 | 64 | 13 | 57 | 37 | 61 | 33 |
| O75592 | 43 | 32 | 44 | 25 | 30 | |
| O75592 | 2 | | 26 | | | |
| O75592 | 66 | 15 | 60 | | 51 | |
| O75592 | 44 | | 41 | | 38 | |
| O75592 | | | 19 | | | |
| O75592 | 19 | 1 | 35 | | 40 | 40 |
| O75592 | | | 24 | | | |
| O75592 | | | 40 | | 51 | 9 |
| O75592 | | | 5 | | | |
| O75592 | | | 4 | | | |
| O75592 | 56 | 27 | | | | |
| O75592 | 56 | 27 | | | | |
| O75592 | | | 14 | | | |
| O75592 | | | 40 | 4 | 8 | |
| O75592 | | | 34 | | 22 | |
| O75592 | 50 | 24 | 35 | 19 | 39 | |
| O75592 | 67 | 4 | 28 | | 32 | |
| O75592 | 30 | 7 | 51 | | 49 | |
| O75592 | | | 19 | | | |
| O75592 | 3 | | 52 | 53 | 12 | |
| O75592 | 19 | | 29 | | | |
| O75592 | 47 | 5 | 44 | | | |
| O75592 | 60 | 29 | 37 | 27 | 36 | 45 |
| Q9NPC7 | 60 | 18 | 38 | | 58 | |
| Q9NPC7 | 23 | | 21 | | 42 | |
| Q9NPC7 | 64 | 69 | 43 | 30 | 43 | 1 |
| Q96RE7 | 37 | | 51 | | 73 | |
| Q96RE7 | 44 | | 26 | | 45 | |
| Q96RE7 | 41 | 38 | 46 | 40 | 42 | 1 |
| Q96RE7 | 65 | 47 | 74 | 53 | | |
| Q96RE7 | 50 | | 45 | | 55 | 10 |
| Q96RE7 | 38 | 32 | 72 | 33 | 56 | 25 |
| Q96RE7 | 67 | 47 | 66 | 56 | 70 | 9 |
| Q96RE7 | 41 | 6 | 26 | | 46 | |
| Q96BF6 | 46 | 38 | 37 | 13 | 47 | |
| Q96BF6 | 32 | | 10 | | 47 | |
| Q96BF6 | 38 | 32 | 72 | 33 | 56 | 25 |
| P46934 | 44 | 9 | | | | |
| P46934 | 76 | 33 | 76 | 71 | 53 | 52 |
| Q96PU5 | 27 | | 2 | | 32 | 8 |
| Q96PU5 | 88 | 30 | 75 | 53 | 61 | 19 |
| Q96PU5 | 68 | 33 | 76 | 71 | 57 | 52 |
| A8MQ27 | 48 | | 26 | | 44 | |
| A8MQ27 | 52 | | 17 | | 34 | |
| A8MQ27 | 17 | | 16 | | 40 | |
| A8MQ27 | 5 | | | | | |
| Q9BR09 | 32 | | | | 3 | |
| Q96EH8 | | | | | 44 | |
| Q96EH8 | 40 | | 26 | | 52 | |
| Q96EH8 | 54 | | 56 | | 56 | |
| Q12986 | 44 | 29 | 42 | 10 | 42 | 1 |
| Q12986 | 71 | 42 | 57 | 38 | 57 | 14 |
| Q12986 | 60 | 23 | 41 | 17 | 44 | |
| Q12986 | 65 | 28 | 37 | 10 | 48 | 10 |
| Q12986 | 36 | 15 | 35 | 27 | 22 | |
| Q12986 | 41 | | 13 | | 29 | |
| Q12986 | | | | | 7 | |
| Q12986 | 26 | | 25 | | 45 | |
| Q12986 | 49 | | 28 | | 24 | |
| Q12986 | | | 11 | | | |
| Q12986 | 46 | | 36 | | 34 | |
| Q12986 | 76 | | 73 | | 79 | |
| Q12986 | 83 | | 62 | | 77 | |
| Q12986 | 82 | | 65 | | 72 | |
| Q12986 | 60 | 14 | 59 | 6 | 40 | |
| Q12986 | | | 63 | 53 | | |
| Q12986 | 46 | | 41 | | 64 | |
| Q12986 | 66 | | 56 | | 73 | |
| Q12986 | 74 | | 62 | | 76 | |
| Q12986 | 60 | 1 | 54 | | 70 | |
| Q12986 | 72 | | 68 | | 83 | |
| Q12986 | 56 | | 47 | | 73 | |
| Q12986 | 53 | | 52 | | 56 | |
| Q12986 | 43 | 13 | 38 | 26 | 36 | 3 |
| Q12986 | 33 | 9 | 25 | | 30 | |
| Q12986 | 46 | 5 | 44 | | 49 | |
| Q12986 | 56 | | 38 | | 47 | |
| Q12986 | 35 | | 3 | | 37 | |
| Q12986 | 59 | | 39 | | 52 | |
| Q12986 | 65 | | 53 | | 62 | |
| Q12986 | 69 | 10 | 66 | | 52 | 8 |
| Q12986 | 49 | | 42 | | 58 | |
| Q12986 | 67 | | 38 | | 63 | |
| Q12986 | 70 | | 52 | | 72 | 2 |
| Q12986 | 2 | | 20 | | 12 | |
| Q12986 | 58 | | 63 | | 63 | |
| Q12986 | 66 | | 64 | | 57 | |
| Q12986 | 57 | | 43 | | 62 | |
| Q12986 | 45 | | 26 | | 47 | |
| Q12986 | 30 | | 35 | | 38 | |
| Q12986 | 36 | | 26 | | 38 | |
| Q12986 | 51 | | 40 | | 44 | |
| Q12986 | 67 | | 49 | | 64 | |
| Q12986 | 70 | | 38 | | 53 | |
| Q12986 | 72 | | 73 | | 76 | |
| Q12986 | 75 | | 73 | | 83 | |
| Q12986 | 82 | | 77 | | 85 | |
| Q12986 | 36 | | 34 | | 55 | |
| Q12986 | 76 | 10 | 61 | | 68 | |
| Q12986 | 60 | | 53 | | 85 | |
| Q12986 | 22 | 1 | 19 | | 22 | |
| Q12986 | 36 | | 20 | | 17 | |
| Q6ZNB6 | 34 | | 36 | | 27 | |
| Q6ZNB6 | | | | | 68 | 21 |
| Q6ZNB6 | 53 | | 62 | | 68 | 21 |
| Q6ZNB6 | 50 | 12 | 37 | 20 | 56 | 36 |
| Q6ZNB6 | 33 | 5 | 34 | | 39 | |
| Q6ZNB6 | 38 | | 30 | | 32 | |
| Q6ZNB6 | 46 | 14 | 59 | | 55 | |
| Q6ZNB6 | 65 | | 38 | | 41 | |
| Q6ZNB6 | 65 | | 57 | | 81 | |
| Q6ZNB6 | 59 | | 41 | | 61 | |
| Q6ZNB6 | 41 | | 23 | | 36 | |
| Q6ZNB6 | 40 | | 17 | | 41 | |
| Q6ZNB6 | 64 | | 37 | | 68 | |
| Q6ZNB6 | 62 | | 59 | | 43 | |
| Q6ZNB6 | 60 | | 58 | | 59 | |
| Q6ZNB6 | 33 | | 45 | | 43 | |
| Q6ZNB6 | 57 | | 20 | | 43 | |
| Q6ZNB6 | 45 | | 45 | | 32 | |
| Q6ZNB6 | 21 | | 5 | | 18 | |
| Q6ZNB6 | 18 | | | | 24 | |
| Q6ZNB6 | 55 | | 29 | | 53 | |
| Q6ZNB6 | 47 | | 20 | | 45 | |
| Q6ZNB6 | 42 | | 16 | | 48 | |
| Q6ZNB6 | 40 | | 9 | | 23 | |
| Q6ZNB6 | 87 | | 61 | | 62 | |
| Q6ZNB6 | 70 | | 35 | | 36 | |
| Q6ZNB6 | 46 | | 24 | | 42 | 3 |
| Q6ZNB6 | 57 | | 35 | | 38 | |
| Q6ZNB6 | 38 | | 27 | | 33 | |
| Q6ZNB6 | 53 | | 31 | | 50 | |
| Q6ZNB6 | 45 | | 11 | | 32 | |
| Q6ZNB6 | 41 | | 34 | | 58 | |
| Q6ZNB6 | 7 | | | | 4 | |
| Q6ZNB6 | 40 | | 8 | | 27 | |
| Q6ZNB6 | | | 26 | | 23 | |
| Q6ZNB6 | 36 | | 27 | | 56 | |
| Q6ZNB6 | | | 29 | | | |
| Q6ZNB6 | 57 | | 15 | | 50 | |
| Q6ZNB6 | 37 | 32 | 25 | | 38 | |
| Q6ZNB6 | 60 | | 39 | | 53 | |
| Q6ZNB6 | 59 | | 56 | | 46 | |
| Q6ZNB6 | 37 | | 25 | | 43 | |
| Q6ZNB6 | 32 | | 20 | | 42 | |
| Q6ZNB6 | 45 | | 19 | | 44 | |
| Q6ZNB6 | 31 | | 13 | | 29 | |
| Q6ZNB6 | 77 | | 47 | | 48 | |
| Q6ZNB6 | 62 | | 31 | | 49 | |
| Q6ZNB6 | 53 | | 22 | | 45 | |
| Q6ZNB6 | 85 | | 60 | | 81 | |
| Q6ZNB6 | 88 | | 66 | | 86 | |
| Q6ZNB6 | 94 | | 65 | | 84 | |
| Q6ZNB6 | 77 | | 55 | | 74 | |
| Q6ZNB6 | 75 | | 56 | | 74 | |
| Q6ZNB6 | 71 | | 45 | | 72 | |
| Q6ZNB6 | 64 | | 67 | 62 | 40 | |
| Q6ZNB6 | 76 | | 51 | | 80 | |
| Q6ZNB6 | 67 | | 47 | | 72 | |
| Q6ZNB6 | 68 | | 41 | | 67 | |
| Q6ZNB6 | 72 | | 45 | | 65 | |
| Q6ZNB6 | 47 | 20 | 42 | 33 | 43 | |
| Q6ZNB6 | 74 | | 43 | | 65 | |
| Q6ZNB6 | 74 | 2 | 46 | | 64 | |
| Q6ZNB6 | 69 | 44 | 68 | 57 | 76 | 32 |
| Q6ZNB6 | 84 | 44 | 78 | | 81 | |
| Q6ZNB6 | 87 | 59 | 81 | 72 | 89 | 40 |
| Q6ZNB6 | 89 | | 79 | | 89 | |
| Q6ZNB6 | 100 | | 88 | | 100 | |
| Q6ZNB6 | 75 | 1 | 81 | | 87 | |
| Q6ZNB6 | 77 | | 55 | | 81 | |
| Q6ZNB6 | 85 | 4 | 78 | | 92 | |
| Q6ZNB6 | 38 | 25 | 50 | 45 | 45 | 4 |
| Q6ZNB6 | | | 45 | 24 | | |
| Q6ZNB6 | 86 | | 69 | | 60 | |
| Q6ZNB6 | 78 | 2 | 59 | | 72 | |
| Q6ZNB6 | 77 | | 30 | | 67 | 3 |
| Q6ZNB6 | 55 | | 20 | | 44 | |
| Q6ZNB6 | 52 | | 29 | | 51 | |
| Q6ZNB6 | 17 | | 33 | | 39 | |
| Q6ZNB6 | 58 | 19 | 36 | 15 | 44 | 5 |
| Q6ZNB6 | 37 | | 3 | | 26 | |
| Q6VVB1 | 9 | | | | 15 | |
| Q9Y314 | 63 | 30 | 51 | 28 | 53 | 28 |
| Q9Y314 | 49 | | 29 | | 55 | |
| Q9Y314 | 74 | 47 | 89 | 68 | 69 | 32 |
| Q9Y314 | 78 | 40 | 88 | 62 | 78 | 36 |
| Q9Y314 | 84 | 63 | 65 | 54 | 67 | 21 |
| Q9Y314 | 61 | 46 | 49 | 39 | 58 | 39 |
| Q9Y314 | 83 | 39 | 45 | 45 | 66 | 43 |
| Q9Y314 | 44 | 29 | 74 | 20 | 53 | 22 |
| O96028 | 65 | 38 | 68 | 23 | 64 | |
| O96028 | 57 | 49 | 60 | 44 | 45 | 4 |
| O96028 | 29 | 15 | 31 | | 31 | |
| O96028 | 55 | 18 | 37 | 28 | 43 | 41 |
| O96028 | 88 | | 78 | | 55 | |
| O96028 | 54 | 36 | 48 | 19 | 60 | 20 |
| O96028 | 38 | 26 | 29 | 16 | 24 | 16 |
| O96028 | 51 | 29 | 35 | 18 | 50 | 30 |
| O96028 | | 8 | 22 | | 15 | |
| O96028 | 50 | 24 | 47 | 22 | 48 | 25 |
| O96028 | 75 | | 82 | | 100 | |
| O96028 | 78 | | 72 | | 74 | |
| O96028 | 75 | | 78 | | 74 | |
| O96028 | 54 | | 50 | | 72 | |
| O96028 | 76 | | 56 | | 69 | |
| O96028 | 56 | | 47 | | 57 | |
| O96028 | 75 | | 59 | | 79 | |
| O96028 | 72 | 8 | 67 | | 76 | |
| O96028 | 73 | 5 | 68 | | 77 | |
| O96028 | 51 | | 54 | | 72 | |
| O96028 | 86 | | 75 | | 80 | |
| O96028 | 80 | | 76 | | 79 | |
| O96028 | 56 | | 58 | | 72 | |
| O96028 | | | | | 10 | |
| O96028 | 69 | 10 | | 65 | | 10 |
| O96028 | 67 | 42 | 100 | 84 | 81 | 23 |
| O96028 | 39 | 3 | 36 | 16 | 46 | |
| O96028 | 69 | 17 | 70 | | 71 | |
| O96028 | 81 | 20 | 79 | | 89 | |
| O96028 | 90 | | 78 | | 84 | |
| O96028 | 85 | 5 | 76 | | 89 | |
| O96028 | 55 | | 34 | | 50 | |
| O96028 | 76 | | 62 | | 76 | |
| O96028 | 57 | 17 | 68 | | 65 | |
| O96028 | 25 | | 35 | | 36 | |
| O96028 | 54 | 31 | 58 | 51 | 46 | 49 |
| O96028 | 59 | 3 | 50 | | 61 | 26 |
| O96028 | 60 | 11 | 77 | | | |
| O96028 | 41 | 10 | 52 | 4 | 66 | 40 |
| O96028 | 43 | 39 | 64 | 50 | 60 | 4 |
| O96028 | 68 | 56 | 74 | 55 | 49 | 19 |
| O96028 | 27 | 2 | 24 | | 38 | |
| O96028 | 40 | | 53 | | 52 | |
| O96028 | 49 | | 60 | | 70 | |
| O96028 | 38 | 31 | 78 | 69 | 68 | 35 |
| O96028 | 81 | | 58 | | 76 | |
| O96028 | 44 | | 38 | | 42 | |
| O96028 | 42 | 27 | 59 | 43 | 58 | 39 |
| O96028 | 30 | 34 | 16 | 21 | 39 | 32 |
| Q8WV22 | 56 | 20 | 46 | 2 | 64 | 13 |
| Q8WV22 | 74 | 8 | 56 | | 66 | |
| Q8WV22 | 2 | | 23 | | | |
| Q96MF7 | 38 | 8 | 20 | | 46 | |
| Q96MF7 | 55 | 8 | 33 | | 37 | |
| Q96MF7 | 35 | 5 | 38 | 20 | 37 | 5 |
| Q96MF7 | 58 | 29 | 56 | 30 | 62 | 39 |
| Q96MF7 | 54 | 30 | 33 | | 48 | 17 |
| Q86WC4 | 68 | | 58 | | 66 | |
| Q8TE49 | 28 | | 56 | 52 | 23 | |
| Q6GQQ9 | 48 | | 39 | | 45 | |
| Q6GQQ9 | 11 | 13 | 56 | 52 | 36 | 14 |
| Q6GQQ9 | 55 | | 44 | | 57 | |
| Q6GQQ9 | 72 | 21 | 40 | 19 | 57 | |
| Q6GQQ9 | 60 | 4 | 43 | 29 | 66 | 21 |
| Q6GQQ9 | 51 | | 19 | | 63 | 12 |
| Q6GQQ9 | 79 | 39 | 52 | 24 | 70 | 20 |
| Q6GQQ9 | 72 | 40 | 49 | 9 | 74 | 16 |
| Q6GQQ9 | 78 | | 16 | 2 | 62 | |
| Q6GQQ9 | 73 | 13 | 66 | 32 | 55 | 32 |
| Q9HBE1 | 60 | 1 | 34 | | | |
| Q9HBE1 | 75 | 44 | 84 | 72 | 86 | 45 |
| Q9HBE1 | 43 | | 28 | | 39 | |
| Q9HBE1 | 29 | | 32 | | 31 | |
| Q9HBE1 | 34 | 19 | 40 | | 39 | |
| Q9HBE1 | 93 | | 53 | | 43 | |
| Q15366 | 45 | 9 | 45 | 7 | 26 | 24 |
| Q15366 | 50 | 26 | 39 | 17 | 48 | 36 |
| Q15366 | 39 | 10 | 64 | 4 | 55 | |
| Q15366 | 36 | 23 | 58 | 45 | 65 | 44 |
| Q15366 | 36 | 14 | 34 | 6 | 54 | 16 |
| Q15366 | 44 | 27 | 64 | 27 | 67 | 49 |
| Q15366 | 48 | 21 | 42 | 17 | 55 | 15 |
| P35227 | 60 | | 41 | | 47 | |
| P35227 | 38 | | 50 | | 42 | |
| P35227 | 66 | 1 | 74 | | 48 | 28 |
| P35227 | 80 | 42 | 66 | 37 | 82 | 29 |
| Q3KNV8 | 52 | | 31 | | 58 | |
| Q3KNV8 | 57 | 19 | 29 | 6 | 50 | 14 |
| Q86SE9 | | 1 | 19 | | | |
| Q86SE9 | 37 | | 20 | | 53 | |
| Q86SE9 | 31 | 12 | 33 | | | |
| Q86SE9 | 19 | 27 | 49 | | | |
| Q86SE9 | 41 | 27 | 51 | 25 | 58 | 9 |
| Q9BYE7 | 37 | 1 | 10 | | | |
| Q9BYE7 | 29 | 15 | 61 | 41 | 33 | |
| Q9BYE7 | 34 | | 43 | | 48 | |
| Q96JY6 | 31 | 23 | 36 | 37 | 36 | 33 |
| Q96JY6 | 22 | | 20 | | 32 | |
| Q96JY6 | | | 10 | 5 | 32 | |
| Q96JY6 | 6 | | | | 23 | |
| Q96JY6 | 24 | 23 | 12 | 4 | 43 | |
| Q96JY6 | | | | 6 | 43 | |
| Q9UPQ7 | 55 | 22 | | | | |
| Q9UPQ7 | 28 | | 19 | | 25 | |
| Q9UPQ7 | 56 | 44 | | | | |
| Q9UPQ7 | 82 | 39 | | | 65 | 20 |
| Q9UPQ7 | 82 | 36 | 81 | 43 | 69 | 24 |
| Q9UPQ7 | 51 | 13 | 35 | 5 | 36 | |
| Q6ZMN7 | | | | | 1 | |
| Q96FA3 | 18 | | 9 | | 10 | |
| Q96FA3 | 14 | 3 | 34 | | | |
| Q96FA3 | 82 | 31 | 86 | 57 | 85 | 2 |
| Q9HAT8 | 66 | 15 | 87 | 68 | 63 | 15 |
| Q9HAT8 | 12 | 2 | 24 | 14 | | |
| Q9HAT8 | 19 | 4 | | | | |
| O60683 | 93 | 51 | 96 | | 88 | 37 |
| O60683 | 53 | | 39 | | 52 | |
| O60683 | 49 | | 31 | | 42 | |
| O00623 | 61 | | 51 | | 40 | |
| O00623 | | | | | 14 | |
| Q9BUL5 | 29 | | 14 | | 27 | |
| Q9BUL5 | 47 | | 21 | | 29 | |
| Q9BUL5 | 6 | 13 | 35 | 16 | 30 | 29 |
| Q9BUL5 | | | 17 | | | |
| Q9P1Y6 | 26 | | 27 | | 24 | |
| Q9P1Y6 | 36 | | 37 | | 39 | |
| Q9P1Y6 | 72 | | 68 | | 61 | |
| Q9P1Y6 | 46 | 27 | 81 | 39 | 62 | 9 |
| Q9P1Y6 | 48 | 28 | 57 | 33 | 59 | 6 |
| Q9P1Y6 | 51 | 14 | 72 | 49 | 71 | 17 |
| Q9P1Y6 | 39 | | 7 | | 49 | |
| Q9P1Y6 | 55 | 14 | 55 | 32 | 73 | 17 |
| Q9P1Y6 | 48 | 37 | 53 | 30 | 61 | 7 |
| Q9P1Y6 | 45 | 17 | 62 | 31 | 68 | 20 |
| Q9P1Y6 | 49 | 15 | 39 | | 41 | 2 |
| Q9P1Y6 | 42 | | 56 | 35 | 39 | |
| Q9P1Y6 | 15 | 1 | 14 | | 12 | |
| O75925 | 7 | 15 | 33 | | 32 | 11 |
| O75925 | 44 | | 24 | | 52 | |
| O75925 | 38 | | 41 | | 64 | |
| O75925 | 71 | 39 | 48 | 37 | 40 | 12 |
| O75925 | 18 | | 40 | | 52 | |
| O75925 | 71 | 28 | 68 | | 72 | |
| O75925 | 33 | 15 | 49 | | | |
| O75925 | 22 | 14 | 43 | | | |
| O75925 | 23 | 2 | 36 | | 32 | |
| O75925 | 37 | 4 | 60 | 15 | 41 | |
| O75925 | 62 | 28 | 74 | 47 | 76 | 26 |
| O75928 | 13 | | 29 | | 20 | |
| O75928 | 33 | 6 | 27 | | 10 | |
| O75928 | 43 | 27 | 39 | | 37 | |
| O75928 | 42 | 30 | 54 | | 55 | |
| O75928 | 67 | 24 | 35 | 15 | 46 | 5 |
| O75928 | 18 | | 40 | | 52 | |
| O75928 | | | 30 | | 53 | |
| O75928 | 51 | | 66 | | 76 | |
| O75928 | 13 | | 38 | | 48 | |
| O75928 | 52 | 32 | 43 | 28 | 43 | 36 |
| O75928 | 64 | 29 | 66 | 58 | 56 | 15 |
| O75928 | 77 | 23 | 75 | 42 | 59 | 2 |
| Q9Y6X2 | 13 | | | | 31 | |
| Q9Y6X2 | 55 | 1 | 45 | | | |
| Q9Y6X2 | 67 | 24 | 35 | 15 | 46 | 5 |
| Q9Y6X2 | 4 | 11 | 33 | | 38 | |
| Q9Y6X2 | 24 | | | | 7 | |
| Q9Y6X2 | 47 | 14 | 39 | 18 | 65 | 12 |
| Q9Y6X2 | 59 | | 63 | 27 | 71 | 10 |
| Q9Y6X2 | 25 | | 67 | | 50 | |
| Q8N2W9 | 16 | 3 | 55 | | 26 | |
| Q8N2W9 | 51 | | 45 | | 63 | |
| Q8N2W9 | 8 | | | | 39 | |
| Q8N2W9 | | 4 | 34 | | 68 | 16 |
| Q8N2W9 | 97 | 96 | 91 | 80 | 25 | 43 |
| Q8N2W9 | 32 | | 56 | | 41 | |
| Q8N2W9 | 77 | | 81 | | 74 | |
| Q8N2W9 | 11 | | 44 | | 47 | |
| Q8N2W9 | 76 | 34 | 69 | | 8 | |
| Q8NG27 | 45 | 11 | 28 | | 28 | |
| Q8NG27 | 47 | 20 | 27 | 16 | 26 | |
| Q8NG27 | 26 | | | | 18 | |
| Q8NG27 | 55 | 2 | 35 | | 44 | |
| Q8NG27 | 36 | 9 | 62 | | 13 | |
| O43164 | 64 | 32 | 51 | 22 | 60 | 1 |
| O43164 | 6 | 2 | | | 4 | |
| O43164 | 74 | | 19 | | 23 | |
| O43164 | 88 | | 66 | | 39 | |
| O43164 | 55 | 2 | 35 | | 44 | |
| O43164 | 36 | 9 | 62 | | 13 | |
| P29590 | 58 | 22 | 47 | 17 | 49 | 40 |
| P29590 | 42 | 21 | 66 | 45 | 50 | 44 |
| P29590 | 50 | 1 | 44 | | 36 | |
| P29590 | 48 | 25 | | | 20 | 24 |
| P29590 | | | 72 | 62 | 12 | 26 |
| P29590 | | | 30 | | 21 | 12 |
| P29590 | 37 | 16 | 84 | 36 | 37 | 33 |
| P29590 | 33 | 13 | 24 | 3 | | |
| P29590 | 26 | 13 | 37 | 10 | 17 | |
| P29590 | 17 | | 26 | | 15 | |
| P29590 | 19 | 23 | 58 | 30 | 46 | 30 |
| P29590 | 43 | 39 | 60 | 43 | 39 | 26 |
| P29590 | 39 | 23 | 39 | 31 | 57 | 36 |
| P29590 | 60 | 33 | 78 | 50 | 66 | 21 |
| P29590 | 79 | 33 | 74 | 51 | 77 | 33 |
| P29590 | 69 | 33 | 92 | 66 | 60 | 39 |
| P29590 | 83 | 39 | 58 | 30 | 88 | 48 |
| P29590 | 60 | | 46 | | 44 | 20 |
| P29590 | 39 | 23 | 38 | 23 | 37 | 26 |
| P29590 | 41 | 19 | 69 | 24 | 73 | 32 |
| P29590 | 87 | 67 | 83 | 62 | 87 | 50 |
| P29590 | 85 | 63 | 69 | 44 | 85 | 43 |
| P29590 | 35 | | 35 | | 56 | |
| Q13356 | 68 | 30 | 65 | 40 | 69 | 43 |
| Q13356 | 77 | | 62 | | 72 | |
| Q13356 | 60 | 11 | 39 | | 60 | 3 |
| Q13356 | 37 | 16 | 35 | 5 | 52 | 31 |
| O60260 | 92 | | 91 | | 93 | |
| Q9UMS4 | 72 | 18 | 49 | 55 | 61 | 5 |
| Q9UMS4 | 41 | 20 | 32 | 11 | 32 | 34 |
| Q9UMS4 | 42 | 22 | 45 | 27 | 36 | 39 |
| Q9UMS4 | 81 | 46 | 33 | 34 | 47 | 22 |
| Q9UMS4 | 82 | 1 | 64 | 19 | 81 | 8 |
| Q9UJ41 | | | 7 | | 12 | |
| Q9UJ41 | 28 | | 14 | | 17 | |
| Q9UJ41 | 9 | | 29 | | 14 | |
| Q9UJ41 | | 12 | 22 | | 11 | |
| Q9UJ41 | 33 | | 7 | | 26 | |
| Q9UJ41 | 9 | | 18 | | 46 | |
| Q9UJ41 | | | 7 | | 30 | |
| Q9UJ41 | 54 | 12 | 50 | | 65 | |
| Q9UJ41 | 39 | | 27 | | 45 | |
| Q9UJ41 | 47 | 27 | 43 | 20 | 39 | 33 |
| Q9NS91 | 69 | 1 | 60 | 26 | 71 | 54 |
| Q9NS91 | 59 | 26 | 48 | 51 | 53 | 33 |
| Q9NS91 | 42 | 3 | 38 | | 47 | |
| Q9NS91 | 30 | | 14 | | 28 | |
| Q9NS91 | 25 | 15 | 21 | | 27 | |
| Q9NS91 | 29 | 46 | 30 | 5 | 27 | 2 |
| P49792 | 31 | 15 | 47 | | 33 | |
| P49792 | 37 | 20 | 60 | 30 | 56 | 23 |
| P49792 | 40 | 26 | 79 | 58 | 60 | 17 |
| P49792 | 31 | 26 | 61 | 38 | 55 | 26 |
| P49792 | 52 | 16 | 68 | 43 | 72 | 53 |
| P49792 | 79 | 53 | 79 | 73 | 76 | 51 |
| P49792 | 87 | 75 | 62 | 6 | 42 | 4 |
| P49792 | 42 | 31 | 73 | 54 | 67 | 17 |
| P49792 | 76 | 42 | 75 | 46 | 92 | 51 |
| P49792 | 75 | 44 | 73 | 47 | 95 | 42 |
| P49792 | 91 | 68 | 90 | 88 | 100 | 68 |
| P49792 | 78 | 41 | 78 | 69 | 87 | 60 |
| P49792 | 65 | 24 | 59 | | 75 | 20 |
| P49792 | 60 | 28 | 73 | 52 | 79 | 42 |
| P49792 | 33 | | | | 33 | |
| P49792 | 47 | 25 | 79 | 52 | 81 | 58 |
| P49792 | 11 | 12 | 23 | | | |
| P49792 | 25 | | 17 | | 33 | |
| P49792 | 16 | 22 | 29 | 11 | 17 | 21 |
| P49792 | 36 | 16 | 29 | 6 | 40 | |
| P49792 | 32 | 29 | 48 | 21 | 52 | 19 |
| P49792 | 32 | 30 | 50 | 33 | 75 | 48 |
| P49792 | 55 | 28 | 42 | 35 | 41 | |
| P49792 | 36 | 12 | 49 | 36 | 87 | 32 |
| P49792 | | 8 | 50 | 42 | 20 | |
| P49792 | 42 | 31 | 51 | 9 | 62 | 20 |
| P49792 | 51 | 43 | 37 | 5 | 40 | 11 |
| P49792 | 52 | 14 | 42 | | 40 | |
| P49792 | 28 | 20 | 64 | 12 | 39 | |
| P49792 | 17 | | | | 5 | |
| P49792 | 47 | 15 | 50 | 37 | 49 | 79 |
| P49792 | 41 | 29 | 49 | 35 | 56 | 45 |
| P49792 | 59 | 51 | 46 | 29 | 47 | 3 |
| P49792 | 54 | 47 | 46 | 19 | 52 | 28 |
| P49792 | 64 | 47 | 42 | 27 | 60 | 11 |
| P49792 | 56 | 44 | 54 | 35 | 60 | 29 |
| P49792 | 35 | 12 | 42 | 18 | 45 | |
| P49792 | 25 | 32 | 40 | 18 | 29 | 13 |
| P49792 | 54 | 9 | 37 | 22 | 51 | 10 |
| P49792 | 40 | 7 | 39 | 24 | 61 | 6 |
| P49792 | 46 | 19 | 49 | | 45 | 38 |
| P49792 | 23 | | 29 | 20 | 58 | 41 |
| P49792 | 25 | 22 | 35 | 9 | 53 | 20 |
| P49792 | 57 | 32 | 62 | | 41 | |
| P49792 | 50 | 29 | 55 | | 50 | |
| P49792 | 41 | 18 | 57 | 47 | 43 | 37 |
| P49792 | 26 | | | | 23 | |
| P49792 | 36 | | 46 | 27 | 56 | |
| P49792 | 29 | | 19 | | 42 | |
| P49792 | 20 | 15 | 25 | | 22 | |
| P49792 | 50 | 27 | 33 | 25 | 56 | 28 |
| P49792 | 41 | 26 | 63 | 31 | 43 | 19 |
| P49792 | 55 | 17 | 48 | 22 | 66 | 44 |
| P49792 | 61 | 26 | 45 | 17 | 59 | 36 |
| P49792 | 20 | 30 | 16 | 10 | 14 | |
| P49792 | 45 | 33 | 36 | 19 | 42 | 49 |
| P49792 | 62 | 17 | 33 | 12 | 54 | 8 |
| P49792 | 52 | 23 | 32 | | 50 | |
| P49792 | 46 | 20 | 37 | | 48 | |
| P49792 | 21 | 29 | 32 | 24 | 32 | |
| P49792 | 42 | 32 | 43 | 23 | 60 | 11 |
| P49792 | 32 | 27 | 49 | 26 | 36 | 39 |
| P49792 | 46 | | 36 | | 70 | |
| P49792 | 40 | 28 | 50 | 34 | 67 | 14 |
| P49792 | 61 | 31 | 62 | 44 | 57 | 37 |
| P49792 | 51 | | 31 | | 57 | |
| P49792 | 21 | | 22 | | 32 | |
| Q7Z6E9 | 17 | 23 | 21 | 2 | 14 | 10 |
| Q7Z6E9 | 13 | | 11 | | 20 | |
| Q7Z6E9 | 33 | 21 | 40 | | 49 | 31 |
| Q7Z6E9 | 15 | | 34 | | | |
| Q7Z6E9 | 23 | 20 | 35 | | | |
| Q7Z6E9 | | | | | 16 | |
| Q7Z6E9 | 17 | | 4 | | 16 | |
| Q7Z6E9 | 50 | 26 | 31 | 18 | 50 | 5 |
| Q9BYM8 | 50 | 39 | 50 | 34 | 52 | 23 |
| Q9BYM8 | 32 | | 13 | | 18 | |
| Q9BYM8 | 5 | | | | 12 | |
| Q9BYM8 | 13 | | | | 3 | |
| Q9BYM8 | 24 | 34 | 65 | 39 | 32 | 3 |
| Q9BYM8 | 54 | 28 | 41 | | 49 | |
| Q9BYM8 | 37 | 29 | 48 | 18 | 42 | 14 |
| Q9BYM8 | 25 | | 13 | | 52 | 13 |
| Q9BYM8 | 43 | | 48 | | | |
| Q9BYM8 | 21 | | 64 | | 35 | |
| Q9BYM8 | 32 | | 8 | | 19 | |
| Q9BYM8 | 28 | | 31 | | 35 | |
| Q9BYM8 | 51 | | 49 | | 64 | |
| Q9BYM8 | 63 | | 26 | | 47 | |
| Q9BYM8 | 10 | | 13 | | 14 | |
| Q9BYM8 | 69 | | 27 | | 28 | |
| P62877 | 78 | | 64 | | 77 | |
| P62877 | 77 | 28 | 74 | 40 | 67 | 25 |
| Q5TC82 | | | | | 50 | |
| Q5TC82 | 40 | | 23 | | 41 | |
| Q5TC82 | 24 | | 5 | | 26 | |
| Q5TC82 | 54 | 28 | 56 | 24 | 50 | |
| Q5TC82 | 43 | 13 | 25 | | 37 | |
| Q9HBD1 | 13 | | 20 | | | |
| Q9HBD1 | 48 | 27 | 43 | 12 | 65 | |
| Q9HBD1 | 71 | 22 | 57 | 38 | 58 | 8 |
| Q9HBD1 | 47 | 37 | 37 | 23 | 31 | |
| Q8NDN9 | 28 | | 19 | | 39 | |
| Q8NDN9 | 85 | 22 | 32 | 2 | 94 | |
| Q8NDN9 | 16 | 10 | 38 | | | |
| O95199 | 57 | 5 | 42 | | 35 | |
| O95199 | 16 | 10 | 38 | | | |
| O95199 | | | 18 | | | |
| Q96PM5 | 22 | 27 | 55 | | 38 | |
| Q96PM5 | | | | | 38 | |
| Q96PM5 | 39 | | 46 | | 56 | |
| Q96PM5 | 3 | | 26 | | 15 | |
| Q96PM5 | 19 | | | | 14 | |
| Q96PM5 | | | 1 | | 1 | |
| Q96PM5 | 30 | | 15 | | 45 | |
| Q96PM5 | 20 | | 19 | | 24 | |
| Q96PM5 | 28 | 44 | 22 | 8 | 19 | |
| Q96PM5 | 3 | 4 | 28 | | 18 | |
| Q96PM5 | 30 | 20 | 58 | 31 | 23 | 23 |
| Q96PM5 | 34 | | 2 | | 40 | |
| Q96PM5 | 12 | | 27 | 31 | 18 | |
| Q96PM5 | | | 74 | 36 | 35 | 8 |
| Q96PM5 | 16 | | 24 | | 25 | |
| Q96PM5 | | 5 | 18 | | | |
| Q96PM5 | 82 | 26 | 79 | | | |
| Q8WZ73 | 28 | | 35 | | 44 | |
| Q8WZ73 | 20 | 15 | 30 | 17 | 39 | 35 |
| Q8WZ73 | | | 44 | 29 | 1 | |
| Q8WZ73 | 25 | | 25 | | 38 | |
| Q8WZ73 | | 1 | 4 | | 39 | |
| Q8WZ73 | 15 | | 32 | | 24 | |
| Q8WZ73 | 30 | | | | 25 | |
| Q8WZ73 | 33 | | 17 | | 45 | |
| Q8WZ73 | 18 | | 3 | | 49 | |
| Q8WZ73 | 27 | | 27 | | 17 | |
| Q8WZ73 | 43 | | 20 | | 34 | |
| Q6PCD5 | 67 | 8 | 59 | | 75 | |
| Q6PCD5 | 52 | 12 | 56 | | 71 | 32 |
| Q6PCD5 | 67 | | 54 | | 55 | |
| Q6PCD5 | 76 | | 51 | | 65 | |
| Q6PCD5 | 67 | 4 | 73 | 58 | 71 | 13 |
| Q6PCD5 | 45 | 15 | 45 | 34 | 77 | 23 |
| Q6PCD5 | 35 | | 63 | | 61 | |
| Q6PCD5 | 93 | 73 | 97 | 95 | 96 | 45 |
| Q6PCD5 | 97 | 82 | 96 | 97 | 97 | 73 |
| Q06587 | 69 | 30 | 75 | 52 | 74 | 29 |
| Q06587 | 63 | 29 | 77 | 38 | 44 | 13 |
| Q06587 | 18 | 20 | 39 | 18 | 20 | 11 |
| Q06587 | 83 | 50 | 83 | 35 | 70 | 22 |
| Q06587 | 52 | 4 | 47 | | 75 | 33 |
| Q06587 | 77 | 46 | 86 | 71 | 62 | 38 |
| Q06587 | 57 | 9 | 48 | 21 | 41 | 12 |
| Q06587 | | 9 | | | 10 | |
| Q06587 | 13 | 25 | | 7 | 6 | |
| Q9NVW2 | 24 | | 45 | | 53 | |
| Q9NVW2 | 81 | | 85 | | 59 | |
| Q9NVW2 | 74 | | 67 | | 71 | |
| Q9NVW2 | 74 | | 67 | | 70 | |
| Q8N5U6 | 37 | | 35 | | 28 | |
| Q8N5U6 | 100 | | 70 | | 88 | |
| Q8N5U6 | 54 | | 51 | | 54 | |
| Q8N5U6 | 36 | 37 | 51 | 16 | 38 | |
| Q8N5U6 | 37 | | 9 | | 7 | |
| Q8N5U6 | 28 | | | | 54 | |
| Q6ZNA4 | 14 | 8 | 28 | | 25 | |
| Q6ZNA4 | 45 | | 8 | | 30 | |
| Q6ZNA4 | 60 | 36 | 71 | 44 | 47 | 8 |
| Q6ZNA4 | 69 | | 42 | | 65 | |
| Q6ZNA4 | 31 | | 17 | | 20 | |
| Q6ZNA4 | 16 | | 14 | | 36 | |
| Q6ZNA4 | 55 | | 56 | | 58 | |
| Q9ULX5 | | | | | 39 | 6 |
| O15541 | 61 | 15 | 28 | 15 | 40 | 21 |
| O15541 | 69 | 6 | 14 | | 38 | 8 |
| O15541 | 58 | 6 | 13 | | 13 | |
| O15541 | 32 | | 26 | | 25 | |
| O15541 | 29 | 16 | 35 | 25 | 30 | 2 |
| O15541 | 4 | | 12 | | | |
| O15541 | 59 | 21 | 47 | | 32 | 8 |
| O15541 | 29 | | 20 | 5 | 33 | 16 |
| Q8IZP6 | 32 | | 26 | | 25 | |
| Q9Y508 | 47 | 21 | 20 | 17 | 28 | 23 |
| Q9Y508 | 36 | | 12 | | 32 | |
| Q9Y508 | 18 | | 25 | | 10 | |
| Q9Y508 | 46 | 11 | 49 | 35 | 47 | 17 |
| Q9Y508 | 42 | 8 | 57 | 32 | 44 | 29 |
| Q9Y508 | 38 | | | | 29 | 14 |
| Q9Y508 | 43 | 4 | 28 | | 45 | |
| Q9Y508 | 17 | 3 | 35 | 22 | 41 | 3 |
| Q9Y508 | 37 | | 32 | 4 | 35 | 25 |
| Q9Y508 | 44 | 10 | 43 | 18 | 48 | 8 |
| Q9Y508 | 72 | 85 | 43 | 84 | 48 | 14 |
| Q9Y508 | 70 | 38 | 61 | 52 | 71 | 32 |
| Q9Y508 | 72 | 3 | 53 | | 62 | |
| Q9Y508 | 56 | 12 | 36 | | 30 | |
| Q9Y508 | 27 | 6 | 21 | | 31 | |
| Q9Y508 | 9 | | 44 | 29 | 16 | |
| Q9Y508 | 60 | 29 | 78 | | 59 | |
| Q9H920 | 58 | | 59 | | 70 | |
| Q9H920 | 18 | | 38 | | 24 | |
| Q9H920 | 67 | | 35 | | 56 | |
| Q9H920 | 40 | | 31 | | 43 | |
| Q5XPI4 | | | | | 6 | |
| Q5XPI4 | 28 | | 15 | | 40 | |
| Q5XPI4 | 44 | 21 | 26 | | 22 | |
| Q5XPI4 | | | 1 | | 20 | |
| Q9BV68 | 51 | 30 | 42 | | 52 | |
| Q9BV68 | 82 | 29 | 51 | 18 | 75 | 6 |
| Q9BV68 | 37 | 11 | 14 | | 44 | |
| Q9BV68 | 23 | 29 | 32 | 19 | 49 | 14 |
| Q9BV68 | | | 3 | | 14 | |
| Q9BV68 | 44 | | 14 | | 47 | |
| Q9BV68 | 18 | | 38 | 16 | 31 | 6 |
| Q9BV68 | 47 | | 66 | | 47 | |
| Q8TEB7 | 52 | | 39 | | 52 | |
| Q8TEB7 | 53 | | 42 | | 56 | |
| Q8TEB7 | 63 | | 45 | | 63 | |
| Q8TEB7 | 77 | | 68 | | 71 | |
| O43567 | 75 | 30 | 68 | 19 | 68 | |
| O43567 | 75 | 12 | 74 | 42 | 61 | 8 |
| O43567 | 57 | 7 | 61 | | 52 | |
| O43567 | 73 | 29 | 64 | | 68 | |
| O43567 | 37 | 14 | | | 35 | |
| Q86XS8 | 60 | 20 | 37 | | 71 | |
| Q8IUD6 | 22 | | 34 | | 22 | |
| Q8IUD6 | 35 | 17 | | | 26 | |
| Q8IUD6 | 40 | | 39 | | 41 | |
| Q8IUD6 | | | 88 | 52 | 86 | 33 |
| Q8IUD6 | 85 | 62 | 82 | 73 | 87 | 28 |
| Q8IUD6 | 9 | | | | 8 | |
| Q8IUD6 | | | 4 | | | |
| Q8WVD3 | | 1 | 49 | | 14 | |
| Q8WVD3 | | | 34 | | | |
| Q8WVD3 | 15 | 14 | 60 | 46 | 78 | 30 |
| Q8WVD3 | 38 | 19 | 52 | 7 | 34 | |
| Q8WVD3 | | | 14 | | | |
| Q8WVD3 | 7 | | 34 | | 27 | 15 |
| Q8WVD3 | 37 | | 25 | | 48 | |
| Q8WVD3 | 23 | | | | 4 | |
| Q9UBS8 | 45 | 17 | 50 | 18 | 48 | |
| Q9UBS8 | | | | | 32 | |
| Q9UBS8 | 64 | | 54 | | 67 | |
| Q9UBS8 | 75 | | 66 | | 79 | |
| Q9UBS8 | 39 | | 50 | | 60 | |
| Q9UBS8 | 17 | 10 | 31 | | 23 | |
| Q9UBS8 | 59 | 4 | 50 | | 60 | |
| Q9UBS8 | 88 | 58 | 86 | 82 | 81 | 14 |
| Q9UBS8 | 36 | | 34 | | 49 | |
| Q9UBS8 | 84 | 19 | 49 | 24 | 66 | 6 |
| Q9UBS8 | 51 | | 20 | | 32 | |
| Q9UBS8 | 63 | 4 | 42 | | 57 | |
| Q9UBS8 | 69 | | 54 | | 73 | |
| Q8WVD5 | | | 19 | | 37 | |
| Q8WVD5 | 16 | 9 | 42 | | 50 | |
| Q8WVD5 | | | | | 3 | |
| Q8WVD5 | 31 | | 54 | | 30 | |
| Q7Z419 | 58 | 23 | 67 | | 33 | |
| Q7Z419 | 5 | | 3 | | 37 | |
| Q7Z419 | 31 | | 46 | | 62 | |
| Q7Z419 | 63 | 17 | 74 | | | |
| Q7Z419 | 67 | 34 | 63 | | | |
| Q7Z419 | 40 | 20 | 47 | | | |
| Q7Z419 | 53 | 21 | 58 | | 18 | |
| Q7Z419 | 24 | | 53 | | | |
| Q7Z419 | 67 | 41 | 75 | | | |
| Q7Z419 | 65 | 28 | 56 | | | |
| Q7Z419 | 81 | 31 | 65 | | 30 | |
| Q7Z419 | 74 | 27 | 57 | 11 | 24 | |
| Q9NTX7 | 34 | | 37 | 3 | 12 | |
| Q9NTX7 | 40 | | 9 | | 24 | |
| Q9NTX7 | | | 38 | 19 | 36 | |
| Q9NTX7 | 29 | 3 | 39 | 4 | 25 | |
| Q9NTX7 | 15 | | 9 | | 21 | |
| Q9NTX7 | 25 | | | | 28 | |
| Q9NTX7 | 20 | 18 | 40 | 43 | 41 | |
| Q8N7C7 | 63 | | 45 | | 63 | |
| Q8N7C7 | 77 | | 68 | | 71 | |
| Q8NC42 | 32 | | | | 31 | |
| Q8NC42 | 62 | 50 | 83 | 41 | 71 | 7 |
| Q8NC42 | 63 | | 45 | | 63 | |
| Q8NC42 | 77 | | 68 | | 71 | |
| Q9ULK6 | 63 | | 45 | | 63 | |
| Q9ULK6 | 77 | | 68 | | 71 | |
| Q96PX1 | 19 | | 42 | | 43 | |
| Q96PX1 | 38 | | 24 | | 42 | |
| Q96PX1 | 73 | | 83 | | 82 | |
| Q96PX1 | 83 | 9 | 74 | | 65 | |
| Q6ZSG1 | 16 | | 14 | | 36 | |
| Q6ZSG1 | 55 | | 56 | | 58 | |
| Q96A37 | 41 | | 29 | | 39 | |
| Q96A37 | 58 | 26 | 32 | | 42 | 20 |
| Q9H6Y7 | 62 | 22 | 70 | 51 | 59 | 19 |
| Q9H6Y7 | 2 | | | | 18 | |
| Q9H6Y7 | 68 | | 37 | | 71 | |
| Q9H6Y7 | 64 | 12 | 42 | | 60 | 1 |
| Q8IYW5 | 22 | 22 | 49 | | | |
| Q8IYW5 | 37 | 8 | 69 | | 16 | |
| Q8IYW5 | 42 | 40 | 81 | 25 | 35 | |
| Q8IYW5 | 61 | 14 | 47 | 21 | 29 | |
| Q8IYW5 | 73 | 25 | 46 | 16 | 40 | |
| Q8IYW5 | 36 | 7 | 39 | | | |
| Q8IYW5 | 30 | 3 | 25 | | 45 | |
| Q8NCN4 | 60 | 12 | 51 | 29 | 85 | 31 |
| Q8NCN4 | 14 | 8 | 36 | | 19 | 2 |
| Q8NCN4 | 49 | 21 | 37 | | 38 | |
| Q8NCN4 | 42 | 21 | 49 | 24 | 49 | 27 |
| Q8NCN4 | 56 | 30 | 72 | 38 | 62 | 9 |
| Q8NCN4 | 50 | | | | 69 | |
| Q8NCN4 | 45 | | 2 | | 71 | |
| Q8NCN4 | 48 | | 5 | | 33 | |
| Q8NCN4 | 49 | 21 | 43 | | 51 | |
| Q8NCN4 | 42 | 22 | 50 | 8 | 19 | |
| Q9BXT8 | | | 23 | 1 | | |
| Q9BXT8 | | | 63 | 40 | | |
| Q8N4F7 | 18 | | 38 | | 24 | |
| Q8N4F7 | 67 | | 35 | | 56 | |
| Q8N4F7 | 40 | | 31 | | 43 | |
| Q9P0P0 | 62 | | 41 | | 60 | |
| Q96GF1 | 12 | | 18 | | 36 | |
| Q96GF1 | 42 | 21 | 64 | | 23 | |
| Q9NXI6 | 69 | 22 | 68 | 25 | 52 | |
| Q5TA31 | 2 | 8 | 37 | | | |
| Q5TA31 | 30 | 25 | 57 | | 10 | |
| Q5TA31 | 76 | 34 | 88 | 43 | 42 | |
| Q5TA31 | 64 | 32 | 75 | | 42 | |
| Q9NV58 | 37 | | 25 | | 47 | |
| Q9NV58 | 63 | | 47 | | 61 | |
| Q9NV58 | 72 | | 55 | | 62 | |
| Q9NV58 | 70 | | 24 | | 57 | |
| Q9NV58 | 64 | | 57 | | 88 | |
| Q9NV58 | 30 | | | | | |
| Q6ZMZ0 | 61 | | 71 | | 57 | |
| Q6ZMZ0 | 72 | 44 | 56 | | | |
| Q6ZMZ0 | 72 | | 51 | | 44 | |
| Q6ZMZ0 | 64 | 20 | 82 | | | |
| Q6ZMZ0 | 45 | 6 | 11 | | 9 | |
| Q99496 | 69 | 30 | 75 | 52 | 74 | 29 |
| Q99496 | 63 | 29 | 77 | 38 | 44 | 13 |
| Q99496 | 37 | 29 | 35 | 21 | 15 | 29 |
| Q99496 | 44 | 1 | 57 | | 50 | 24 |
| Q99496 | 52 | 4 | 47 | | 75 | 33 |
| Q99496 | 77 | 46 | 86 | 71 | 62 | 38 |
| Q99496 | 23 | 5 | 20 | 5 | 31 | |
| Q5VTR2 | 50 | 20 | 22 | 11 | 36 | 34 |
| Q5VTR2 | 94 | 62 | 69 | 42 | 73 | 53 |
| Q5VTR2 | 33 | 21 | 32 | 21 | 18 | |
| Q5VTR2 | | | | | 2 | |
| Q5VTR2 | 57 | 19 | 56 | 31 | 38 | 24 |
| Q5VTR2 | 70 | | 18 | | 33 | 9 |
| Q5VTR2 | | | | | 10 | |
| Q5VTR2 | | | 25 | | | |
| Q5VTR2 | 21 | | 22 | | 38 | 7 |
| Q63HN8 | 88 | | 62 | | 77 | |
| Q63HN8 | 92 | 51 | | | 25 | 27 |
| Q63HN8 | 74 | 65 | 69 | 73 | 80 | 72 |
| Q63HN8 | 58 | | 49 | | 46 | |
| Q63HN8 | 12 | 17 | 59 | 29 | 46 | 46 |
| Q63HN8 | 97 | 74 | 100 | 96 | 97 | 67 |
| Q63HN8 | 61 | | 55 | | 46 | |
| Q63HN8 | 78 | 42 | 87 | 82 | 82 | 76 |
| Q63HN8 | 100 | 16 | 67 | 38 | 86 | 44 |
| Q63HN8 | 67 | | 63 | | 79 | 26 |
| Q63HN8 | 58 | | 60 | | 74 | |
| Q63HN8 | 64 | 36 | 43 | | 64 | |
| Q63HN8 | 50 | 71 | 53 | 63 | 49 | 77 |
| Q63HN8 | 43 | 18 | 43 | | 71 | 48 |
| Q63HN8 | 47 | | 30 | | 27 | |
| Q63HN8 | 65 | 24 | 82 | 62 | 86 | 71 |
| Q63HN8 | 50 | | 33 | | 54 | |
| Q63HN8 | 36 | | 26 | | 48 | |
| Q63HN8 | 37 | 15 | 61 | 35 | 44 | 38 |
| Q63HN8 | 100 | 93 | 84 | 56 | 73 | 53 |
| Q63HN8 | 38 | 21 | 32 | 30 | 42 | 50 |
| Q63HN8 | 66 | 32 | 89 | 79 | 73 | 55 |
| Q63HN8 | 35 | 27 | 63 | 43 | 75 | 62 |
| Q63HN8 | | | | | 8 | |
| Q63HN8 | 38 | 7 | 32 | | 42 | |
| Q63HN8 | 48 | | | | | |
| Q63HN8 | 54 | 10 | 76 | 64 | 71 | 44 |
| Q63HN8 | 63 | | 57 | | 66 | |
| Q63HN8 | 89 | 73 | 80 | | 84 | 24 |
| Q63HN8 | 78 | 36 | 71 | 20 | 82 | 15 |
| Q63HN8 | 58 | | 47 | | 64 | |
| Q63HN8 | 44 | 16 | 43 | 17 | 47 | 46 |
| Q63HN8 | 12 | | 16 | 6 | 40 | 39 |
| Q63HN8 | 71 | 32 | 65 | | 75 | |
| Q63HN8 | 55 | | 29 | | 60 | |
| Q63HN8 | 31 | 11 | 49 | 38 | 43 | 66 |
| Q63HN8 | 52 | 11 | 72 | 51 | 72 | 52 |
| Q63HN8 | 51 | | 34 | | 64 | |
| Q63HN8 | 44 | 23 | 76 | 51 | 94 | 65 |
| Q63HN8 | 61 | | 33 | | 58 | 1 |
| Q63HN8 | 41 | 23 | 39 | | 45 | |
| Q63HN8 | 61 | 27 | 65 | | 39 | |
| Q63HN8 | 50 | | 43 | | 58 | 15 |
| Q63HN8 | 70 | | 46 | | 52 | |
| Q63HN8 | 46 | 11 | 36 | 17 | 63 | 39 |
| Q63HN8 | 51 | | 39 | | 45 | |
| Q63HN8 | 39 | 36 | 62 | 24 | 53 | 59 |
| Q63HN8 | 53 | 17 | 92 | 67 | 55 | 32 |
| Q63HN8 | | 2 | | 5 | 71 | |
| Q63HN8 | 48 | | 37 | | 52 | |
| Q63HN8 | 68 | 24 | 72 | 51 | 83 | 60 |
| Q63HN8 | 76 | 6 | 86 | 40 | 88 | |
| Q63HN8 | 89 | | 84 | | 90 | |
| Q63HN8 | 79 | 20 | 79 | 18 | 84 | 52 |
| Q63HN8 | 88 | | 92 | 69 | 83 | |
| Q63HN8 | 94 | | 80 | | 89 | 72 |
| Q63HN8 | 91 | | 84 | 74 | 87 | |
| Q63HN8 | 81 | 2 | 77 | 26 | 71 | |
| Q63HN8 | 100 | | 87 | | 63 | |
| Q63HN8 | 65 | 40 | 63 | 35 | 63 | 48 |
| Q63HN8 | 55 | | 44 | | 60 | |
| Q63HN8 | 53 | | 42 | | 58 | 16 |
| Q63HN8 | 95 | 69 | 73 | 43 | 74 | 52 |
| Q63HN8 | 28 | | 53 | 31 | 45 | |
| Q63HN8 | 84 | | 77 | | 77 | |
| Q63HN8 | 99 | 84 | 50 | 17 | 99 | 99 |
| Q63HN8 | 26 | | 2 | | 31 | |
| Q63HN8 | 37 | 24 | 21 | 8 | 35 | 34 |
| Q63HN8 | 69 | 26 | 83 | | 63 | 43 |
| Q63HN8 | 54 | | 32 | | 47 | |
| Q63HN8 | 100 | 16 | 69 | 57 | 83 | |
| Q63HN8 | 66 | | 46 | | 58 | |
| Q8ND24 | 19 | | | | 1 | |
| Q8ND24 | 62 | 38 | 35 | 17 | 41 | 25 |
| Q8ND24 | 45 | 34 | 32 | 16 | 36 | 10 |
| Q8ND24 | 31 | 33 | 44 | 24 | 38 | 11 |
| Q8ND24 | 17 | 5 | 13 | | 33 | |
| Q8ND24 | 83 | 50 | 96 | 66 | 84 | 54 |
| Q8ND24 | 37 | 6 | 33 | | 40 | |
| Q8ND24 | 29 | | 34 | | 34 | |
| Q8ND24 | 42 | | 40 | | 44 | |
| Q8ND24 | 50 | | 47 | | 55 | |
| Q8ND24 | 57 | 3 | 56 | | 36 | |
| Q9NWF9 | 57 | | 61 | | 74 | |
| Q9NWF9 | 60 | 14 | 80 | | 28 | |
| Q9NWF9 | 69 | 5 | 66 | | 39 | |
| Q9NWF9 | 35 | 15 | 13 | | 42 | |
| Q9NWF9 | 53 | | 24 | | 34 | |
| Q9NWF9 | 11 | | | | 7 | |
| Q9NWF9 | 33 | | 7 | | 10 | |
| Q9NWF9 | | | 9 | | 2 | |
| Q9NWF9 | 38 | | 16 | | 66 | |
| Q8TC41 | 78 | | 58 | | 80 | |
| Q5W0B1 | 73 | 10 | 74 | | 75 | |
| Q5W0B1 | 77 | 6 | 82 | | 62 | |
| Q5W0B1 | 28 | | 38 | | 34 | |
| Q5W0B1 | | 6 | 14 | | | |
| Q5W0B1 | 49 | | 51 | 26 | 43 | |
| Q5W0B1 | 56 | 30 | 47 | 33 | 46 | 4 |
| Q5W0B1 | 56 | 34 | 57 | 41 | 59 | 13 |
| Q5W0B1 | 84 | 40 | 82 | | 65 | |
| Q5W0B1 | 76 | 41 | 70 | | 70 | |
| Q5W0B1 | 86 | 67 | 78 | 70 | 86 | 17 |
| Q5W0B1 | 34 | | 22 | | 36 | |
| Q5W0B1 | 54 | 33 | 55 | 32 | 57 | 5 |
| Q5W0B1 | 59 | 31 | 61 | 40 | 70 | 35 |
| Q5W0B1 | 54 | 17 | 28 | | 48 | |
| Q5VTB9 | 53 | 18 | 33 | | | |
| Q5VTB9 | 53 | | 23 | | 14 | |
| Q5VTB9 | 69 | 38 | 32 | 17 | 34 | 2 |
| Q5VTB9 | 41 | 18 | 24 | | 10 | |
| Q5VTB9 | 35 | | 27 | | 42 | |
| Q5VTB9 | 67 | 55 | 46 | 46 | | |
| Q5VTB9 | 64 | | 51 | | 67 | |
| Q5VTB9 | 50 | 11 | 30 | | 37 | |
| E7ERA6 | 35 | | 19 | | 38 | |
| E7ERA6 | 38 | | 22 | | 18 | |
| E7ERA6 | | | 15 | | | |
| P0DH78 | 67 | 17 | | | | |
| P0DH78 | 67 | 17 | | | | |
| P0DH78 | 67 | 17 | | | | |
| Q96BH1 | 11 | | 15 | | | |
| Q96BH1 | 11 | | 24 | | | |
| Q96BH1 | 22 | 3 | 36 | | 46 | 11 |
| Q96BH1 | 26 | 5 | 32 | | | |
| Q96BH1 | 27 | | 32 | | 48 | |
| Q96BH1 | 15 | | 26 | | 10 | |
| Q96BH1 | 53 | 21 | 36 | 8 | 39 | 15 |
| Q96BH1 | 48 | 25 | 24 | 4 | 40 | 16 |
| Q96BH1 | 78 | 34 | 62 | 14 | 71 | 10 |
| Q9BY78 | 31 | | 54 | | 30 | |
| Q96EP0 | 45 | 30 | 37 | 17 | 58 | 42 |
| Q96EP0 | | | 63 | 23 | 42 | |
| Q96EP0 | 52 | 31 | 89 | 63 | 39 | 6 |
| Q96EP0 | | | | | 7 | |
| Q96EP0 | | | | | 41 | 2 |
| Q96EP0 | 31 | 21 | 68 | 6 | 14 | |
| Q96EP0 | 20 | 1 | 11 | | 35 | |
| Q96EP0 | 45 | 20 | 48 | 19 | 50 | 3 |
| Q96EP0 | 17 | | | | 33 | |
| Q96EP0 | 48 | | 31 | | 33 | 3 |
| Q96EP0 | 41 | | 47 | | 39 | |
| Q96EP0 | 69 | | 60 | 53 | 47 | |
| Q96EP0 | 51 | | 23 | | 51 | |
| Q96EP0 | 79 | 19 | 60 | | 70 | |
| Q96EP0 | 24 | 8 | | 9 | 40 | 20 |
| Q96EP0 | 28 | | 15 | | 17 | |
| Q96EP0 | 47 | | 41 | | 54 | |
| Q96EP0 | 25 | | 16 | | 23 | |
| Q96EP0 | 22 | 5 | 31 | | 49 | |
| Q96EP0 | 55 | 17 | 25 | 5 | 41 | |
| Q96EP0 | 32 | | 1 | | 28 | |
| Q96EP0 | 54 | | 34 | | 43 | |
| Q96EP0 | 10 | | 26 | | 24 | |
| Q96EP0 | 54 | | 50 | 17 | 65 | 11 |
| Q96EP0 | | | | | 37 | |
| Q96EP0 | 24 | | 18 | | 40 | |
| Q9H0A6 | 42 | 44 | 34 | | 16 | |
| Q9H0A6 | 42 | 44 | 37 | | 10 | |
| Q969K3 | | | 48 | | | |
| Q969K3 | 55 | 33 | 36 | 18 | 28 | 8 |
| Q969K3 | 18 | 20 | 46 | | | |
| Q969K3 | | | 47 | | | |
| Q969K3 | 33 | | 51 | | 43 | |
| Q969K3 | | | 20 | | 21 | |
| Q969K3 | 7 | | 12 | | 17 | |
| Q969K3 | 20 | | | | 7 | |
| Q969K3 | 41 | 4 | 43 | | 35 | |
| Q969K3 | 4 | | | | 18 | |
| Q9H0F5 | 24 | | 14 | | | |
| Q9H0F5 | 42 | 44 | 34 | | 16 | |
| Q9H0F5 | 42 | 44 | 37 | | 10 | |
| P78317 | 4 | | 25 | 16 | 24 | |
| P78317 | 20 | | 26 | | 35 | |
| P78317 | 25 | | 18 | | 21 | |
| P78317 | 40 | | 34 | | 25 | |
| O75150 | 24 | 3 | 37 | 36 | 52 | 10 |
| O75150 | 24 | 12 | 26 | | 51 | 14 |
| O75150 | 94 | 84 | 65 | 37 | 78 | 50 |
| O75150 | 63 | 16 | 58 | | 47 | |
| O75150 | 44 | 32 | 47 | 30 | 49 | 48 |
| O75150 | 61 | 14 | 59 | 40 | 41 | 5 |
| O75150 | 56 | 18 | 47 | | 53 | 4 |
| O75150 | 15 | | 22 | | 30 | |
| Q9H4P4 | 63 | | 61 | | 64 | |
| Q9H4P4 | 52 | | 44 | | 47 | |
| Q9H4P4 | 42 | | 13 | | 37 | |
| Q68DV7 | 41 | 13 | 12 | 7 | 42 | |
| Q68DV7 | 44 | | 23 | | | |
| Q68DV7 | 44 | | 7 | | 40 | |
| Q68DV7 | 59 | 21 | 81 | | | |
| Q7L0R7 | 41 | | 25 | | 32 | |
| Q7L0R7 | 42 | 44 | 34 | | 16 | |
| Q7L0R7 | 42 | 44 | 37 | | 10 | |
| Q99942 | 56 | 21 | 62 | 16 | 55 | 14 |
| Q99942 | 30 | | 19 | | 34 | |
| Q99942 | 66 | | 37 | | 45 | |
| Q99942 | 80 | | 68 | | 68 | |
| Q99942 | 64 | 8 | 48 | 7 | 65 | |
| Q99942 | 68 | 26 | 65 | 19 | 56 | 15 |
| Q9Y252 | 38 | | 9 | | 51 | |
| Q9UBF6 | 64 | | | | | |
| Q9UBF6 | 67 | 57 | 65 | 34 | 52 | 33 |
| Q9UBF6 | 90 | | 38 | | 32 | 3 |
| Q9UBF6 | | | | | 52 | 20 |
| Q9UBF6 | 100 | 23 | 90 | 69 | 50 | 5 |
| Q9UBF6 | 96 | 67 | 88 | 71 | 52 | 20 |
| Q9UBF6 | 100 | 75 | 86 | 61 | 42 | |
| Q9UBF6 | 71 | 15 | 74 | | 75 | |
| Q9UBF6 | 69 | | 73 | | 67 | |
| O76064 | 65 | 19 | 17 | | 34 | |
| O76064 | 52 | 22 | 25 | | 35 | |
| O76064 | 82 | 44 | 61 | | 68 | |
| O76064 | 2 | 4 | 18 | | | |
| Q5M7Z0 | 62 | | 34 | | 56 | |
| Q5M7Z0 | 63 | | 23 | | 52 | |
| Q5M7Z0 | 54 | 3 | 49 | | 61 | |
| Q5M7Z0 | 71 | | 59 | | 73 | |
| Q5M7Z0 | | | 60 | | 45 | |
| Q5M7Z0 | 42 | 44 | 34 | | 16 | |
| Q5M7Z0 | 42 | 44 | 37 | | 10 | |
| Q96EX2 | 42 | 44 | 34 | | 16 | |
| Q96EX2 | 42 | 44 | 37 | | 29 | 17 |
| Q96DX4 | 17 | | 18 | | 24 | |
| Q96DX4 | | | 21 | | | |
| Q96T51 | 51 | 17 | 29 | 29 | 31 | 12 |
| Q96T51 | 66 | 24 | 56 | 35 | 82 | 45 |
| Q96T51 | 68 | 33 | 80 | 33 | 57 | |
| Q96T51 | 29 | | 18 | | 38 | |
| Q96T51 | 57 | 41 | 51 | 40 | 81 | 71 |
| Q96T51 | 15 | 22 | 20 | 5 | 25 | |
| Q96T51 | 18 | 20 | 26 | 14 | 51 | 34 |
| Q96T51 | 18 | | | | 37 | |
| Q96T51 | 36 | 9 | 36 | | 52 | 4 |
| Q96T51 | | | 15 | | 12 | |
| Q96T51 | 6 | | | | 10 | |
| Q96T51 | 27 | 19 | 36 | 12 | 51 | 15 |
| Q96T51 | 35 | | 32 | | 30 | |
| Q96T51 | | | | | 15 | |
| Q96T51 | 27 | 6 | 22 | | 38 | |
| Q96T51 | 48 | 20 | 15 | 16 | 59 | 41 |
| Q7Z6J0 | 6 | | 47 | 14 | 24 | |
| Q7Z6J0 | 53 | | 39 | | 38 | |
| Q7Z6J0 | 58 | 42 | 52 | 28 | 60 | 3 |
| Q7Z6J0 | 52 | 26 | 28 | 16 | 43 | 3 |
| Q7Z6J0 | 9 | | | | 14 | |
| Q7Z6J0 | | | | | 12 | |
| Q7Z6J0 | 4 | | | | 22 | |
| Q7Z6J0 | 18 | | | | 13 | |
| Q7Z6J0 | 12 | | | | | |
| Q7Z6J0 | 73 | 42 | 45 | 26 | 66 | 4 |
| Q7Z6J0 | 70 | 28 | 65 | 43 | 46 | |
| Q8TEC5 | 84 | | 65 | | 76 | |
| Q8TEC5 | 81 | | 74 | | 73 | |
| Q8TEC5 | 2 | | 3 | | 10 | |
| Q8TEC5 | 25 | | | | 30 | |
| Q8TEC5 | 51 | | 23 | | 64 | |
| Q8TEJ3 | | | 3 | | | |
| Q8TEJ3 | 50 | | 18 | | 69 | |
| Q8TEJ3 | | | | | 10 | |
| Q8TEJ3 | | | | | 12 | |
| Q8TEJ3 | 15 | | 26 | | 39 | |
| Q9H0F6 | 16 | 3 | 16 | | 19 | |
| Q9H0F6 | 62 | | 53 | | 72 | 25 |
| Q9H0F6 | | | 4 | | | |
| Q9H0F6 | 35 | | 29 | | 37 | |
| Q9H0F6 | 37 | | 29 | | 42 | |
| Q8TBC3 | 81 | 34 | 66 | | 68 | |
| Q8TBC3 | 57 | | 34 | | 41 | |
| Q8TBC3 | 45 | 9 | 56 | 45 | 55 | 28 |
| Q8TBC3 | 100 | 62 | 99 | 99 | 99 | 44 |
| Q8TBC3 | 55 | 11 | 39 | 18 | 51 | 17 |
| Q149N8 | 55 | | 27 | 19 | 52 | |
| Q149N8 | 69 | 35 | 50 | | | |
| Q149N8 | 32 | 13 | 32 | | | |
| Q149N8 | 32 | | 12 | | 41 | |
| Q149N8 | 31 | | 17 | | 32 | |
| Q149N8 | 50 | | 20 | | 37 | |
| Q149N8 | 61 | 20 | 51 | | 58 | |
| Q149N8 | 2 | | 25 | | 70 | 41 |
| Q149N8 | 62 | | 59 | | 2 | |
| Q149N8 | 62 | | | | 2 | |
| Q149N8 | 67 | 35 | 59 | 42 | 35 | |
| Q149N8 | 20 | 16 | 58 | | | |
| Q149N8 | 54 | | 46 | | 38 | |
| Q149N8 | 14 | 32 | 57 | | | |
| Q149N8 | 71 | | 64 | | 86 | |
| Q149N8 | | | 4 | | | |
| Q149N8 | 31 | 1 | 28 | | | |
| Q149N8 | 3 | | 34 | | | |
| Q149N8 | 45 | | 66 | | 35 | |
| Q8IUQ4 | 24 | 2 | 20 | 10 | 33 | 9 |
| Q8IUQ4 | 58 | 41 | 43 | 25 | 69 | 17 |
| Q8IUQ4 | 61 | 35 | 43 | 18 | | |
| O43255 | 47 | 31 | 49 | | 28 | |
| O43255 | 14 | 20 | | | | |
| O43255 | 32 | | 12 | | 30 | |
| O43255 | 47 | | 32 | | 34 | |
| P63208 | 5 | | 7 | | 10 | |
| P63208 | 19 | 13 | 23 | 2 | 15 | 15 |
| P63208 | 20 | 18 | 16 | 7 | 23 | 14 |
| Q13309 | | | 43 | | | |
| Q13309 | 64 | 16 | 69 | 42 | 70 | 10 |
| Q13309 | 52 | 18 | 55 | | 58 | |
| Q13309 | 42 | | 52 | | 59 | |
| Q13309 | 16 | | 14 | | 24 | |
| Q13309 | 31 | 6 | 38 | 2 | 34 | |
| Q8IY92 | 30 | 7 | 21 | | 39 | |
| Q8IY92 | | | 13 | | | |
| Q8IY92 | 14 | | 14 | | 7 | |
| Q8IY92 | 17 | 12 | 21 | | 32 | |
| Q8IY92 | 35 | 12 | 20 | | | |
| Q8IY92 | 82 | 49 | 68 | | 40 | |
| Q8IY92 | 37 | 4 | 18 | | 49 | |
| Q8IY92 | 42 | 20 | 39 | | 7 | |
| Q8IY92 | 30 | 23 | 23 | | 51 | |
| Q8IY92 | 52 | | 12 | | 43 | |
| Q8IY92 | 68 | 28 | 37 | 8 | 65 | 10 |
| Q9HCE7 | | 5 | | | 13 | |
| Q9HCE7 | | | | | 1 | |
| Q9HCE7 | 53 | 18 | 34 | | 50 | |
| Q9HCE7 | 13 | | | | 25 | |
| Q9HCE7 | 76 | 33 | 76 | 71 | 53 | 52 |
| Q9HAU4 | | 5 | | | 13 | |
| Q9HAU4 | 47 | 10 | 32 | | 52 | 1 |
| Q9HAU4 | 38 | 12 | 7 | | 44 | |
| Q9HAU4 | 61 | | 61 | 47 | 37 | |
| Q9HAU4 | 64 | | 6 | | 35 | |
| Q9HAU4 | 72 | 37 | 80 | 49 | 56 | 24 |
| Q9HAU4 | 76 | 33 | 76 | 71 | 53 | 52 |
| Q9HAU4 | 32 | 6 | 17 | | | |
| O15524 | 38 | | 35 | | 56 | |
| O14508 | 21 | | | | 48 | |
| O14508 | 86 | 14 | 66 | 30 | 91 | 48 |
| O14508 | | | 41 | | 82 | |
| O14508 | 35 | | 28 | | 53 | |
| O14543 | 45 | | 30 | | 62 | |
| O14543 | 90 | 40 | 96 | 91 | 89 | |
| O14543 | 42 | | 21 | | 68 | |
| O14544 | 26 | | | | 46 | |
| O14544 | 24 | | 33 | | 36 | |
| O14544 | 64 | 35 | 80 | 83 | 85 | 59 |
| O14512 | 100 | | 85 | | 89 | |
| O14512 | 16 | | 22 | | | |
| O43791 | | | 20 | | 36 | 6 |
| O43791 | 19 | | 19 | | 27 | |
| O43791 | 29 | | 29 | | 40 | |
| O43791 | 94 | 49 | 78 | 57 | 67 | 54 |
| Q6IQ16 | 14 | | 22 | | 44 | |
| Q6IQ16 | 58 | 16 | 47 | | 57 | 20 |
| Q6PJ21 | | | 52 | 25 | | |
| Q6PJ21 | 8 | 5 | | | 30 | 4 |
| Q9UNE7 | 51 | 15 | 33 | 5 | 49 | 3 |
| Q9UNE7 | 39 | 16 | 22 | 13 | 53 | |
| Q9UNE7 | 29 | 30 | 33 | | | |
| Q9UNE7 | 14 | | 3 | | 9 | |
| Q9UNE7 | 42 | 16 | 13 | 10 | 34 | 20 |
| Q9UNE7 | 26 | 31 | 14 | 12 | 39 | |
| Q9UNE7 | 28 | | 29 | | 33 | |
| Q9Y2Z0 | 19 | 20 | 36 | 9 | 31 | 31 |
| Q9Y2Z0 | 55 | 27 | 30 | 13 | 38 | 16 |
| Q9Y2Z0 | 49 | 24 | 28 | 11 | 39 | 23 |
| Q9Y2Z0 | 40 | 23 | 23 | 9 | 45 | 25 |
| Q9Y2Z0 | 28 | | 25 | 19 | 60 | |
| Q86TM6 | 21 | | 22 | | 35 | |
| Q86TM6 | 3 | | 30 | 19 | 37 | |
| Q86TM6 | | | 20 | | 20 | |
| Q86TM6 | 26 | | 24 | | 57 | |
| A0AVI4 | 44 | 14 | 58 | | | 15 |
| Q13829 | 15 | | | | 2 | |
| Q13829 | 9 | 8 | 21 | | 10 | 7 |
| Q13829 | | 4 | 20 | | | |
| P21580 | 100 | 90 | 99 | 98 | 99 | 86 |
| P21580 | 98 | 65 | 90 | 56 | 76 | 35 |
| P21580 | 44 | 25 | 86 | 70 | 78 | 30 |
| P21580 | 39 | | 44 | 24 | 50 | 16 |
| P21580 | 43 | | 42 | | 56 | |
| P21580 | 55 | | 33 | 14 | 36 | |
| P21580 | 63 | | 42 | | 42 | |
| P21580 | 67 | | 52 | | 58 | |
| P21580 | 67 | | 49 | | 35 | |
| P21580 | 50 | 21 | 57 | 30 | 53 | 37 |
| P21580 | 48 | | 33 | | 52 | |
| P21580 | 39 | | 22 | | 38 | |
| P21580 | 47 | | 24 | | 46 | |
| P21580 | 39 | | 27 | | 46 | |
| P21580 | 51 | | 31 | | 57 | |
| P21580 | 100 | 100 | 62 | 53 | 50 | 45 |
| P21580 | 49 | 33 | 57 | 32 | 53 | 33 |
| P21580 | 61 | 30 | 68 | 42 | 55 | 4 |
| P21580 | 60 | 9 | 45 | | 60 | |
| P21580 | 69 | 40 | 63 | 38 | 64 | 42 |
| P21580 | 54 | | 27 | | 40 | 19 |
| P21580 | 89 | 40 | 56 | 32 | 18 | |
| P21580 | 60 | | 22 | | 49 | |
| P21580 | 49 | 22 | 65 | 42 | 46 | |
| P21580 | 45 | 4 | 68 | 34 | 52 | |
| P21580 | 61 | 14 | 17 | | 53 | |
| P21580 | 61 | 31 | 44 | 39 | 57 | 26 |
| P21580 | 52 | 4 | 24 | | 30 | |
| P21580 | 64 | 31 | 68 | 40 | 31 | 8 |
| P21580 | 79 | 29 | 70 | 37 | 25 | 16 |
| P21580 | 72 | | 32 | | 47 | |
| P21580 | 79 | | 36 | 26 | 37 | |
| Q15025 | 31 | | 26 | 3 | 49 | |
| Q15025 | 75 | | 37 | | 100 | |
| Q15025 | 73 | | 23 | | 69 | |
| Q9NS56 | 50 | 46 | 73 | 55 | 69 | 13 |
| Q9NS56 | 81 | | 52 | | 53 | |
| Q9NS56 | 64 | 13 | 46 | | 66 | |
| Q9NS56 | 65 | 18 | 100 | 66 | 79 | 11 |
| Q9NS56 | 51 | | 21 | | 64 | |
| Q9NS56 | 39 | | 21 | | 23 | |
| Q13077 | 48 | 19 | 56 | 11 | 62 | 41 |
| Q13077 | 82 | 37 | 49 | 31 | 44 | |
| Q13077 | 64 | | 19 | | 46 | 2 |
| Q13077 | 46 | | 16 | | 33 | |
| Q13077 | 59 | 25 | 29 | 14 | 40 | 42 |
| Q13077 | 71 | | 53 | 26 | 54 | 17 |
| Q13077 | 69 | | 61 | | 54 | |
| Q13077 | 45 | 5 | 23 | | 32 | |
| Q13077 | 70 | 26 | 55 | 38 | 77 | 68 |
| Q13077 | 55 | 3 | 53 | 38 | 81 | 9 |
| Q13077 | 44 | | 35 | | 35 | |
| Q12933 | 59 | 31 | 66 | 13 | 62 | 2 |
| Q12933 | 34 | 8 | 36 | 21 | 28 | 38 |
| Q12933 | 34 | 13 | 39 | 18 | 40 | 36 |
| Q12933 | 27 | 11 | 40 | 13 | 25 | 36 |
| Q12933 | 29 | 23 | 34 | 22 | 35 | 3 |
| Q12933 | 39 | 6 | 35 | | 37 | |
| Q12933 | 30 | | 18 | | 43 | |
| Q12933 | 37 | 42 | 23 | 29 | 68 | 6 |
| Q12933 | 54 | 22 | 49 | 24 | 50 | 39 |
| Q12933 | 64 | 21 | 52 | 33 | 45 | 13 |
| Q12933 | 17 | 14 | 34 | | 29 | 20 |
| Q12933 | 23 | 9 | 12 | | 31 | 7 |
| Q12933 | 35 | 14 | 17 | | 30 | 1 |
| Q12933 | 4 | | 40 | | | |
| Q12933 | 2 | 17 | 11 | | | |
| Q12933 | 48 | 22 | 83 | 69 | 99 | 67 |
| Q12933 | 29 | 22 | 34 | 8 | 37 | |
| Q12933 | 18 | | | | 29 | |
| Q13114 | 29 | | 21 | | 28 | |
| Q13114 | 79 | | 35 | | 54 | |
| Q13114 | 38 | 33 | 7 | | 23 | |
| Q13114 | 28 | | | | 22 | |
| Q13114 | 58 | | 23 | | 53 | |
| Q13114 | 42 | | 13 | | 54 | |
| Q9BUZ4 | 32 | 8 | 38 | | 32 | |
| Q9BUZ4 | 57 | 16 | 32 | | 40 | |
| Q9BUZ4 | 8 | | 8 | | 15 | |
| Q9BUZ4 | 48 | | 45 | 30 | 34 | |
| Q9BUZ4 | 24 | 2 | 34 | | 12 | |
| Q9BUZ4 | 19 | | 23 | | 22 | |
| Q9BUZ4 | 55 | | 18 | | 41 | |
| Q9BUZ4 | | 2 | 31 | | | |
| Q9BUZ4 | 44 | | 24 | | 44 | |
| Q9BUZ4 | | | 25 | | | |
| O00463 | 59 | 10 | 36 | | 60 | |
| O00463 | 42 | | 57 | | 51 | |
| O00463 | 36 | | 52 | | 61 | |
| O00463 | 42 | 4 | 62 | | 42 | |
| O00463 | 56 | 24 | 57 | 37 | 40 | 35 |
| O00463 | 38 | 16 | 55 | 21 | 50 | 39 |
| O00463 | 35 | 13 | 52 | | | |
| O00463 | 59 | 9 | 60 | | 70 | |
| O00463 | 22 | 4 | 23 | | 45 | |
| O00463 | | 16 | 29 | | 54 | 17 |
| O00463 | | 9 | 51 | | | |
| O00463 | 40 | 23 | 62 | 13 | 45 | |
| O00463 | 10 | 10 | 27 | | 14 | |
| O00463 | | 1 | 13 | | | |
| Q9Y4K3 | 39 | 12 | 61 | 15 | 31 | 6 |
| Q9Y4K3 | 21 | | 33 | 8 | 30 | |
| Q9Y4K3 | | | 5 | | | |
| Q9Y4K3 | 29 | 12 | 22 | 3 | 32 | |
| Q9Y4K3 | | 11 | | | | |
| Q9Y4K3 | 18 | | 18 | | 27 | |
| Q9Y4K3 | 45 | 24 | 24 | 14 | 34 | 58 |
| Q9Y4K3 | | | 11 | | | |
| Q6Q0C0 | 42 | | 27 | | 68 | |
| Q6Q0C0 | 62 | | 55 | | 71 | |
| Q6Q0C0 | 57 | | 55 | | 72 | |
| Q6Q0C0 | 62 | | 58 | | 60 | 5 |
| Q6Q0C0 | 69 | | 70 | | 61 | |
| Q6Q0C0 | 56 | | 29 | | 45 | |
| Q6Q0C0 | 68 | 1 | 39 | | 55 | 20 |
| Q6Q0C0 | | | | | 9 | |
| Q6Q0C0 | 30 | | | | 10 | |
| Q6Q0C0 | 47 | | 38 | | 50 | |
| Q6Q0C0 | 30 | 3 | 22 | | 9 | |
| Q6Q0C0 | 29 | | | | 30 | |
| Q9BWF2 | 36 | | 71 | | 22 | |
| Q9BWF2 | 52 | 10 | 42 | | 29 | |
| Q96F44 | 49 | | 25 | | 41 | |
| Q96F44 | 63 | | 62 | | 71 | |
| Q96F44 | 33 | | 30 | | 57 | |
| Q96F44 | 53 | 51 | 83 | | 61 | |
| Q96F44 | 83 | | 66 | | 69 | |
| Q96F44 | 54 | | 59 | | 53 | |
| Q96F44 | 53 | 4 | 39 | | 41 | |
| Q96F44 | | | 9 | | | |
| O60858 | 62 | | 58 | | 48 | |
| O60858 | 45 | | 32 | | 46 | |
| O60858 | 4 | | 6 | | 8 | |
| Q9C019 | 1 | | 7 | | 3 | |
| Q9C019 | 35 | | 33 | | 40 | |
| Q9C019 | 26 | | 33 | 18 | 34 | |
| Q9C019 | 16 | | 13 | | 19 | |
| Q9C019 | 23 | | 8 | | 24 | |
| Q9C040 | 32 | | 33 | | 23 | |
| Q9C040 | 49 | | 44 | | 40 | |
| Q9C040 | 44 | 20 | 33 | | 36 | |
| Q9C040 | | | 5 | | 24 | |
| Q9C040 | 49 | | 19 | | 61 | |
| P19474 | 32 | 12 | 24 | | 33 | |
| P19474 | 25 | 5 | 58 | 39 | 8 | |
| P19474 | | | 14 | 11 | 20 | |
| P19474 | 66 | | 50 | | 60 | |
| P19474 | 61 | 23 | 22 | 2 | 22 | |
| P19474 | | | 40 | 5 | | |
| P19474 | 56 | 34 | 55 | 25 | 57 | 39 |
| P19474 | 1 | | 34 | | | |
| P19474 | 41 | | 16 | | 18 | |
| Q8IYM9 | | | 62 | 25 | | |
| Q8IYM9 | 39 | | 47 | | 34 | |
| Q8IYM9 | 37 | | 23 | | 29 | |
| Q8IYM9 | 45 | 59 | 65 | | | |
| Q8IYM9 | 69 | | 77 | | 57 | |
| Q8IYM9 | 47 | 21 | 51 | 17 | 67 | 24 |
| Q8IYM9 | 52 | 2 | 38 | 25 | 58 | 20 |
| Q8IYM9 | 16 | 9 | 34 | | 62 | |
| Q8IYM9 | 5 | 11 | 39 | | 22 | |
| Q8IYM9 | 28 | 23 | 22 | | 20 | 4 |
| P36406 | 10 | | | | | |
| P36406 | 73 | | 17 | | 61 | |
| O15164 | 40 | | 41 | | 39 | |
| O15164 | 20 | | 17 | 16 | 37 | |
| O15164 | 64 | 17 | 41 | 6 | 50 | 20 |
| O15164 | 46 | 34 | 58 | 30 | 77 | 58 |
| O15164 | 43 | | 17 | | 40 | |
| O15164 | 42 | | 8 | | 28 | |
| O15164 | 54 | 20 | 32 | | 23 | |
| O15164 | 55 | 6 | 55 | | 47 | |
| O15164 | 40 | | 30 | | 46 | |
| O15164 | 67 | | 53 | | 65 | |
| O15164 | | | 33 | | 26 | |
| O15164 | 43 | 25 | 57 | 29 | 55 | 42 |
| O15164 | | | 54 | 29 | | |
| O15164 | | | 21 | | | |
| O15164 | 46 | | 19 | | 12 | |
| O15164 | 44 | | 27 | | 43 | |
| O15164 | 58 | 22 | 19 | | 48 | 5 |
| Q14258 | 62 | 21 | 58 | 31 | 27 | 41 |
| Q14258 | 72 | 18 | 88 | 61 | 62 | 9 |
| Q14258 | 72 | 66 | 62 | 47 | 72 | 8 |
| Q14258 | 59 | 10 | 43 | 27 | 57 | 21 |
| Q14258 | 60 | 50 | 63 | 36 | 62 | 5 |
| Q14258 | 51 | 36 | 65 | 35 | 59 | 30 |
| Q14258 | 50 | | 44 | | 55 | |
| Q14258 | 46 | | 51 | | 53 | |
| Q14258 | 42 | 22 | 71 | 41 | 64 | 24 |
| Q14258 | 12 | | | | 25 | |
| Q14258 | 33 | 32 | 32 | 16 | 35 | 5 |
| Q14258 | 34 | 25 | 42 | 28 | 44 | 37 |
| Q14258 | 52 | 35 | 28 | 7 | 45 | |
| Q14258 | 82 | 49 | 94 | 85 | 94 | 56 |
| Q12899 | 44 | | 58 | | 31 | |
| Q12899 | 16 | | 17 | | 35 | |
| Q12899 | 24 | | 40 | | 26 | |
| Q12899 | 37 | | 31 | | 46 | |
| Q12899 | 44 | | 35 | | 59 | |
| Q12899 | | 7 | | | 29 | |
| Q12899 | 74 | | 55 | | 60 | |
| Q12899 | 68 | | 30 | | 55 | |
| Q12899 | | | 22 | | | |
| Q12899 | 18 | 36 | 18 | 56 | 9 | 28 |
| P14373 | 25 | | 20 | | 21 | |
| P14373 | 24 | | 60 | | 57 | 1 |
| P14373 | 37 | 5 | 71 | | 23 | |
| P14373 | 58 | 25 | 43 | 13 | 52 | 11 |
| P14373 | 23 | | 39 | | 28 | |
| Q13263 | 50 | 29 | 34 | 17 | 71 | 57 |
| Q13263 | 58 | 12 | 43 | 21 | 46 | 12 |
| Q13263 | 26 | | 40 | 19 | 50 | 28 |
| Q13263 | 32 | 29 | 53 | 42 | 44 | 35 |
| Q13263 | 77 | 26 | 84 | 61 | 62 | 34 |
| Q13263 | 23 | 6 | 38 | 10 | 52 | 23 |
| Q13263 | 40 | 14 | 50 | 42 | 46 | 6 |
| Q13263 | 44 | 25 | 51 | 24 | 68 | 57 |
| Q13263 | 26 | 19 | 24 | | 45 | 9 |
| Q13263 | | | | | 33 | |
| Q13263 | | | 35 | 6 | | |
| Q13263 | | | 43 | 1 | 29 | 20 |
| Q13263 | 47 | | | | 43 | |
| Q13263 | 40 | 25 | 47 | 41 | 57 | 38 |
| Q13263 | 65 | 24 | 50 | 36 | 79 | 42 |
| Q13263 | 50 | 22 | 59 | 41 | 58 | 30 |
| Q13263 | 53 | 19 | 87 | 47 | 68 | 7 |
| Q13263 | 36 | 37 | 58 | 33 | 52 | 40 |
| Q13263 | 18 | | 7 | | 46 | 19 |
| Q13263 | 64 | 23 | 58 | 43 | 62 | 33 |
| Q13263 | 44 | 30 | 53 | 40 | 56 | 53 |
| O75382 | 57 | | 66 | | 39 | |
| O75382 | 49 | | 44 | | 40 | |
| O75382 | 44 | 20 | 33 | | 36 | |
| O75382 | | | 18 | | 6 | |
| O75382 | 47 | | 39 | | 70 | |
| O75382 | 82 | 47 | 83 | 45 | 87 | |
| O75382 | 14 | | | | | |
| O75382 | 43 | | 31 | | 26 | |
| O75382 | | | 2 | | 3 | |
| Q9BZY9 | 68 | | 48 | | 71 | |
| Q13049 | 60 | | 64 | | 61 | |
| Q13049 | 22 | | 20 | | 33 | |
| Q13049 | 36 | | 40 | 14 | 40 | 5 |
| Q13049 | 55 | | 39 | | 58 | |
| Q13049 | 78 | | 58 | | 79 | |
| Q13049 | 9 | | 31 | | 46 | |
| Q13049 | 25 | | 14 | | 28 | |
| Q13049 | 63 | | 51 | | 43 | |
| Q13049 | 10 | | 11 | | 18 | |
| Q9UPN9 | 53 | 25 | 46 | | 24 | |
| Q9UPN9 | 68 | 28 | 74 | 50 | 47 | 37 |
| Q9UPN9 | 72 | 63 | 86 | 87 | 47 | 35 |
| Q9UPN9 | 45 | 21 | 30 | | 46 | 17 |
| Q9UPN9 | 26 | | 41 | 23 | 38 | |
| Q9UPN9 | 53 | 35 | 50 | | 51 | |
| Q9UPN9 | 55 | 23 | 53 | 32 | 55 | 36 |
| Q9UPN9 | 45 | | 27 | | 39 | |
| Q9UPN9 | 43 | 25 | 57 | 29 | 55 | 42 |
| Q9UPN9 | 36 | 16 | 63 | 43 | 79 | 19 |
| Q9UPN9 | 64 | 14 | 50 | 5 | 67 | |
| Q9UPN9 | 85 | 44 | 86 | 64 | 78 | 57 |
| Q9UPN9 | 57 | 38 | 40 | 26 | 56 | 40 |
| Q9UPN9 | 60 | 42 | 49 | 27 | 30 | 8 |
| Q9UPN9 | 51 | 45 | 51 | 26 | 56 | 11 |
| Q9UPN9 | 27 | 4 | 13 | 2 | 18 | 3 |
| Q9UPN9 | 3 | 5 | 32 | 2 | 34 | 12 |
| Q9UPN9 | 35 | 42 | 10 | 63 | 7 | |
| Q9UPQ4 | 27 | 10 | 32 | | | |
| Q9NQ86 | | | | | 16 | |
| Q9NQ86 | 32 | | 33 | | 21 | |
| Q9NQ86 | 56 | 27 | 30 | | 35 | |
| Q9NQ86 | 52 | | 14 | | 26 | |
| Q9NQ86 | 73 | | 65 | | 71 | |
| Q9NQ86 | 71 | | 65 | | 67 | |
| Q9NQ86 | 57 | | 39 | | 44 | |
| Q9NQ86 | 49 | 24 | 26 | 1 | 24 | |
| Q9NQ86 | 89 | | 62 | | 88 | |
| Q9NQ86 | 88 | 31 | | | | |
| Q9NQ86 | 88 | 41 | | | | |
| Q9NQ86 | 38 | | 25 | 11 | 51 | |
| O94972 | | | 64 | 36 | | |
| O94972 | 57 | | 57 | | 52 | |
| O94972 | 68 | | 2 | | 89 | |
| O94972 | 69 | | 63 | | 66 | |
| O94972 | 70 | 25 | 72 | | 67 | |
| O94972 | 67 | 28 | 71 | | 59 | |
| O94972 | 7 | | | | 28 | |
| O94972 | 43 | 11 | 38 | | 34 | |
| O94972 | 49 | 13 | 44 | | 72 | 18 |
| O94972 | 37 | 6 | 28 | | 28 | |
| O00635 | 60 | 8 | 39 | | 16 | 7 |
| O00635 | 51 | 7 | 32 | | 58 | |
| O00635 | 31 | | 50 | | | |
| O00635 | 68 | | 46 | 7 | 70 | 7 |
| O00635 | 44 | | 38 | | 43 | |
| O00635 | 45 | | 44 | 18 | 50 | 21 |
| O00635 | 49 | | 45 | | 63 | |
| O00635 | 35 | | 12 | | 43 | |
| O00635 | 19 | | | | 27 | |
| Q9C037 | 24 | 3 | 44 | | | |
| Q9C037 | 62 | | 37 | | 38 | |
| Q9C037 | 36 | | 31 | | 33 | |
| Q9C037 | 75 | | 59 | | 70 | |
| Q9C037 | 78 | | 60 | | 80 | |
| Q9C037 | 57 | 24 | 42 | | 55 | 11 |
| Q9C037 | 79 | 18 | 74 | | 77 | |
| Q8WV44 | 40 | | 39 | 31 | 50 | |
| Q8WV44 | 30 | | | | 39 | |
| Q8WV44 | 56 | | 43 | | 55 | |
| Q8WV44 | 47 | | 31 | | 42 | |
| Q8WV44 | 29 | | | | | |
| Q8WV44 | 59 | 15 | 43 | | 56 | |
| Q7Z4K8 | 60 | | 49 | | 56 | |
| Q7Z4K8 | 63 | | 48 | | 37 | |
| Q7Z4K8 | 22 | | 31 | | 37 | |
| Q7Z4K8 | 75 | | 64 | | 75 | |
| Q7Z4K8 | 44 | | 28 | | 38 | |
| Q7Z4K8 | 63 | | 66 | | 68 | |
| Q96LD4 | 59 | | 18 | | 44 | |
| Q96LD4 | | | | | 13 | |
| Q96LD4 | 45 | | 39 | 21 | 38 | 11 |
| Q96LD4 | 55 | 48 | 61 | 36 | 45 | 11 |
| Q96LD4 | 45 | 26 | 66 | 31 | 43 | 17 |
| Q96LD4 | 58 | 39 | 81 | 47 | 53 | 31 |
| Q96LD4 | 75 | 22 | 28 | 7 | 41 | |
| Q96LD4 | 52 | 28 | 36 | 14 | 38 | 5 |
| Q96LD4 | 27 | | 44 | | 44 | |
| Q96LD4 | 36 | | 33 | | 39 | |
| Q96LD4 | 44 | | 33 | | 42 | |
| Q96LD4 | 43 | 27 | 65 | 48 | 45 | 11 |
| Q9C035 | 21 | | 22 | | 48 | |
| Q9C035 | 33 | | 34 | | 29 | |
| Q9C035 | 42 | 13 | 46 | | 46 | |
| Q9C035 | 67 | | 44 | | 66 | |
| Q9C035 | 32 | 13 | 24 | | 34 | |
| Q9C035 | 58 | | 14 | | 69 | |
| Q9C035 | 8 | | 23 | 18 | 27 | 4 |
| Q9C035 | 53 | | 32 | | 49 | |
| Q9BYV6 | 1 | | | | | |
| Q9BYV6 | 1 | | | | | |
| Q9BRZ2 | 66 | 55 | 61 | 32 | 58 | 39 |
| Q9BRZ2 | 64 | 19 | 78 | 34 | 64 | 16 |
| Q9BRZ2 | 78 | 33 | 93 | 61 | 64 | 47 |
| Q9BRZ2 | 27 | | 32 | 4 | 32 | 14 |
| Q9BRZ2 | 34 | | 55 | | 44 | |
| Q9BRZ2 | 39 | 16 | 66 | | | |
| Q9BRZ2 | 66 | 36 | 93 | 57 | 73 | 32 |
| Q9BRZ2 | 58 | 27 | 86 | 70 | 58 | 31 |
| Q9BRZ2 | 28 | | 20 | | 18 | |
| Q9BRZ2 | 62 | | 59 | | 47 | |
| Q9BRZ2 | 54 | | 41 | | | |
| Q9BRZ2 | 43 | 26 | 49 | 33 | 27 | |
| Q9BRZ2 | 31 | | 47 | 29 | 43 | 11 |
| Q9BRZ2 | 80 | | 57 | | 76 | |
| Q9BRZ2 | 77 | 48 | 75 | 62 | 80 | 23 |
| Q9BRZ2 | 35 | 51 | 38 | 5 | 53 | 24 |
| Q9BRZ2 | 39 | 20 | 51 | | 51 | |
| Q9BRZ2 | 91 | 61 | 85 | 66 | 89 | 67 |
| Q9BRZ2 | 86 | | 59 | | 82 | |
| Q9BRZ2 | 83 | 49 | 89 | 67 | 85 | 35 |
| Q9BRZ2 | 67 | 41 | 80 | | 75 | 15 |
| Q8IWR1 | 58 | | 46 | | 49 | |
| Q8IWR1 | | | 70 | 27 | 37 | |
| Q8IWR1 | 66 | 46 | 92 | 90 | 72 | 38 |
| Q9C030 | 46 | | 43 | | 53 | |
| Q9C030 | 73 | | 28 | | 62 | |
| Q9C030 | 54 | | 39 | | 49 | |
| Q495X7 | 44 | 15 | 43 | 27 | | |
| Q9BVG3 | 58 | | 59 | | 64 | |
| Q9BVG3 | 13 | 1 | | | 26 | |
| Q6PJ69 | 38 | | 45 | 16 | 45 | |
| Q6PJ69 | | | 16 | | | |
| Q6PJ69 | 13 | | 14 | | 14 | |
| Q6PJ69 | 76 | 59 | 75 | 56 | 70 | 44 |
| Q6PJ69 | 75 | 36 | 89 | 69 | 69 | 35 |
| Q6PJ69 | 66 | 34 | 68 | 50 | 75 | 27 |
| Q6PJ69 | 52 | 26 | 45 | 5 | 86 | 32 |
| Q6PJ69 | 72 | 35 | 69 | 47 | 79 | 26 |
| Q6PJ69 | 90 | | 57 | | 63 | |
| Q6PJ69 | 89 | 58 | 81 | 56 | 88 | 51 |
| Q6PJ69 | 66 | 21 | 42 | 5 | 53 | 27 |
| Q6PJ69 | 66 | | 67 | | 51 | |
| Q6PJ69 | 1 | | 24 | | 19 | |
| Q6ZTA4 | 44 | | 6 | | 37 | |
| Q6AZZ1 | 44 | | 18 | | 36 | |
| Q9C029 | 66 | 23 | 45 | | 54 | |
| Q9C029 | 66 | | 33 | | 37 | |
| Q9C029 | 48 | | 52 | | 58 | |
| Q9C029 | 60 | | 44 | | 55 | |
| Q9C029 | 55 | | 36 | | 52 | |
| Q9C029 | 68 | 22 | 39 | | 56 | |
| Q9C029 | | | | | 19 | |
| Q9C029 | 92 | 1 | | | | |
| Q2Q1W2 | 19 | | 23 | | 17 | |
| Q2Q1W2 | 67 | | 41 | | 43 | |
| Q2Q1W2 | 73 | | 38 | | 50 | |
| Q2Q1W2 | 7 | | 14 | | 12 | |
| Q2Q1W2 | 19 | | 3 | | 28 | |
| Q2Q1W2 | 46 | | 20 | | 38 | |
| Q2Q1W2 | 44 | | | | 38 | |
| Q2Q1W2 | 51 | | 25 | | 45 | |
| Q2Q1W2 | 3 | | | | 14 | |
| Q2Q1W2 | 34 | | 11 | | 37 | |
| Q2Q1W2 | 10 | | 19 | | 21 | |
| Q2Q1W2 | 87 | 39 | 64 | 17 | 59 | 5 |
| Q2Q1W2 | 53 | | 37 | | 33 | |
| Q2Q1W2 | 20 | | 8 | | 37 | |
| Q2Q1W2 | | | 8 | | 12 | |
| Q6ZMU5 | | | | | 18 | 6 |
| Q6ZMU5 | | | 61 | 41 | | |
| Q6ZMU5 | 46 | 36 | 74 | 55 | 47 | |
| Q6ZMU5 | | | 77 | 64 | | |
| Q6ZMU5 | 35 | 41 | 54 | 38 | 50 | |
| Q6ZMU5 | 27 | 34 | 61 | 22 | 57 | |
| Q6ZMU5 | 4 | 24 | 11 | 6 | 31 | 8 |
| Q9BZR9 | | | | | 34 | |
| Q9BZR9 | 20 | 11 | 24 | | | |
| Q9BZR9 | 73 | 44 | 63 | | 67 | |
| Q9BZR9 | 71 | | 58 | | 61 | |
| Q9BZR9 | 86 | | 53 | | 72 | |
| Q9BZR9 | 78 | | 66 | | 74 | |
| Q9BZR9 | 67 | | 64 | 39 | 70 | 10 |
| Q9BZR9 | 17 | 7 | 38 | | 28 | |
| Q9BZR9 | 39 | 19 | 47 | | 54 | 16 |
| Q9BZR9 | 68 | 25 | 48 | | 57 | |
| Q9C026 | | | 9 | | 57 | 30 |
| Q9C026 | | | 5 | 13 | 18 | 4 |
| Q9C026 | | | 21 | | 45 | |
| Q9C026 | 64 | 3 | | | 4 | 39 |
| Q9C026 | 69 | 24 | | | | |
| Q14669 | 24 | | 17 | 14 | 46 | 1 |
| Q14669 | 47 | 31 | 44 | 10 | 30 | 16 |
| Q14669 | 27 | | | | 34 | 9 |
| Q14669 | 53 | 34 | 49 | 33 | 51 | 53 |
| Q14669 | 63 | | 52 | | 74 | |
| Q14669 | 42 | 31 | 92 | 64 | 59 | 61 |
| Q14669 | 66 | 4 | 59 | | 67 | |
| Q14669 | 46 | 12 | 46 | 21 | 36 | 1 |
| Q14669 | 41 | 26 | 57 | 36 | 44 | 2 |
| Q14669 | 43 | 33 | 65 | 33 | 50 | 38 |
| Q14669 | 54 | | 43 | | 49 | |
| Q14669 | 9 | | 37 | | | |
| Q14669 | 38 | 11 | 29 | | 35 | |
| Q14669 | 58 | | 31 | | 42 | |
| Q14669 | 62 | 56 | 71 | 55 | 72 | 24 |
| Q14669 | | | | | 63 | 21 |
| Q14669 | 80 | 34 | 86 | 52 | 63 | 51 |
| Q8TEL6 | 81 | | 54 | | 77 | |
| Q8TEL6 | 69 | 1 | 55 | | 61 | |
| Q8TEL6 | 56 | | 23 | | 61 | |
| P53804 | 91 | | 85 | | 91 | |
| P53804 | | | | | 49 | 9 |
| P53804 | 44 | | 22 | | 52 | |
| P53804 | 35 | | 82 | | 46 | |
| P53804 | 56 | 12 | 52 | | 60 | |
| P53804 | 71 | 36 | 54 | 32 | 69 | 26 |
| P53804 | 50 | | 32 | | 62 | |
| P53804 | 83 | 59 | 89 | 89 | 86 | 38 |
| P53804 | 28 | | 16 | | 39 | |
| P53804 | 28 | | 15 | | 40 | |
| P53804 | 62 | | 25 | | 60 | |
| P53804 | 60 | | 35 | | 65 | |
| P53804 | 40 | | | | 20 | |
| P53804 | 38 | | 8 | | 45 | |
| Q9NRJ4 | 75 | | 15 | | 45 | |
| Q9NRJ4 | 59 | 28 | 37 | | 24 | |
| Q9NRJ4 | 39 | | 17 | | 32 | |
| Q9BSL1 | 17 | 20 | 29 | 36 | 48 | 36 |
| Q9BSL1 | 66 | 2 | 38 | | 57 | |
| Q9C0C9 | 27 | | 1 | | 20 | |
| Q9C0C9 | 58 | 51 | 50 | 24 | 65 | 19 |
| Q9C0C9 | 9 | | | 15 | 35 | |
| Q9C0C9 | 36 | | 43 | | 56 | |
| Q9C0C9 | 58 | 30 | 66 | 53 | 35 | 17 |
| Q9C0C9 | 70 | 40 | 49 | 24 | 80 | 21 |
| Q9C0C9 | 43 | 19 | 45 | 37 | 17 | |
| Q9C0C9 | 96 | 89 | 94 | 84 | 87 | 43 |
| Q9C0C9 | 73 | 52 | 70 | 38 | 65 | 49 |
| Q9C0C9 | 84 | 79 | 74 | 62 | 79 | 30 |
| Q9C0C9 | 56 | 42 | 60 | 37 | 77 | 53 |
| Q9C0C9 | 25 | 16 | 29 | | 27 | |
| Q9C0C9 | 35 | 27 | 30 | 24 | 41 | 19 |
| Q9C0C9 | 84 | 74 | 59 | 45 | 71 | 31 |
| Q9C0C9 | 78 | 54 | 61 | 46 | 76 | 20 |
| Q9C0C9 | 32 | 18 | 50 | 29 | 25 | 11 |
| Q9C0C9 | 63 | 42 | 57 | 34 | 62 | 51 |
| Q9C0C9 | 21 | | 10 | | 20 | 11 |
| Q05086 | 58 | 30 | 35 | | 57 | |
| Q05086 | 66 | 50 | 60 | 42 | 61 | 16 |
| Q05086 | 58 | 9 | 39 | | 21 | |
| Q05086 | 78 | 52 | 32 | 36 | 29 | 36 |
| Q05086 | 56 | 21 | 51 | 27 | 59 | 41 |
| Q05086 | 45 | 11 | 23 | | 5 | |
| Q05086 | 51 | 13 | 43 | 42 | 34 | 34 |
| Q05086 | 20 | | 34 | | 50 | 8 |
| Q05086 | 35 | | 43 | | 55 | |
| Q05086 | 72 | 2 | 53 | 35 | 51 | 8 |
| Q05086 | 57 | 27 | 61 | 43 | 27 | 35 |
| Q7Z3V4 | 86 | 42 | 56 | 33 | 64 | 8 |
| Q7Z3V4 | 50 | | 38 | | 56 | |
| Q7Z3V4 | | | 13 | | | |
| Q7Z3V4 | 45 | | 24 | | 36 | |
| Q15386 | 56 | 9 | 59 | | 59 | 10 |
| Q15386 | 3 | | | | | 2 |
| Q15386 | 72 | | 50 | | 52 | |
| Q15386 | 79 | | 40 | | 69 | 9 |
| Q15386 | 49 | 11 | 34 | | 56 | |
| Q15386 | 32 | 12 | 7 | | 30 | |
| Q15386 | 38 | | 14 | | 34 | |
| Q15386 | 50 | 48 | 68 | 16 | 53 | 8 |
| Q15386 | 54 | | 41 | | 47 | |
| Q15386 | 28 | | 36 | | 42 | |
| Q15386 | | | 12 | | 12 | |
| Q7Z6J8 | 19 | 4 | 16 | | 19 | |
| Q7Z6J8 | 59 | 10 | 41 | | 57 | |
| Q7Z6J8 | 62 | 43 | 25 | | 17 | |
| Q7Z6J8 | 80 | 16 | 50 | | 82 | 13 |
| Q7Z6J8 | 52 | | 25 | | 53 | |
| Q7Z6J8 | 75 | | 54 | | 65 | |
| Q7Z6J8 | 42 | 16 | 50 | | 51 | |
| Q14139 | 90 | 34 | 63 | 29 | 48 | 66 |
| Q14139 | 6 | | | | 31 | |
| Q14139 | 31 | | 32 | | 54 | |
| Q14139 | 58 | 40 | 43 | 17 | 21 | |
| Q14139 | 51 | 26 | 37 | | 58 | 16 |
| Q14139 | 56 | 39 | 71 | 57 | 64 | 31 |
| Q14139 | | 3 | 23 | | 15 | 2 |
| Q14139 | 25 | | 55 | 55 | 42 | |
| O95155 | 25 | 3 | 21 | 28 | 37 | 20 |
| O95155 | 88 | 86 | 78 | 69 | 62 | 31 |
| O95155 | 45 | | 49 | | 71 | |
| O95155 | 56 | | 59 | | 60 | |
| O95155 | 54 | | 58 | | 66 | |
| O95155 | 54 | 5 | 48 | 23 | 52 | 2 |
| O95155 | 96 | 72 | 99 | | 57 | |
| O95155 | 61 | 16 | 68 | | 70 | |
| O95155 | 39 | 3 | 47 | 15 | 42 | 7 |
| O94941 | 46 | 24 | 24 | | 36 | |
| O94941 | 4 | | 23 | | 20 | |
| O94941 | 70 | 47 | 72 | 63 | 86 | 40 |
| O94941 | 21 | | | | 36 | |
| O94941 | 9 | | | | 12 | |
| Q8IWV7 | 40 | | 2 | | 13 | |
| Q8IWV7 | 64 | 44 | 62 | 34 | 55 | 44 |
| Q8IWV7 | 37 | | 42 | | 35 | |
| Q8IWV7 | 51 | 8 | 13 | | 65 | |
| Q8IWV7 | 61 | | 62 | | 63 | |
| Q8IWV7 | | | 8 | | 1 | |
| Q8IWV7 | 50 | | 28 | | 50 | |
| Q8IWV7 | 68 | | 65 | | 57 | |
| Q8IWV7 | 33 | 32 | 39 | 22 | 32 | |
| Q8IWV7 | | 5 | 34 | | | |
| Q8IWV7 | 12 | | 48 | | 43 | |
| Q8IWV7 | | | | | 16 | |
| Q8IWV7 | 89 | 63 | 85 | 64 | 95 | 59 |
| Q8IWV7 | | | 13 | | | |
| Q8IWV8 | 17 | 1 | 18 | | 33 | |
| Q8IWV8 | 40 | 3 | 25 | | 35 | |
| Q8IWV8 | | | 11 | | | |
| Q8IWV8 | 31 | | 28 | | 7 | |
| Q8IWV8 | 22 | 3 | 20 | | 24 | |
| Q8IWV8 | 65 | 26 | 57 | 39 | 44 | 50 |
| Q8IWV8 | 28 | 15 | 64 | 19 | 61 | 8 |
| Q8IWV8 | 74 | | 72 | | 86 | |
| Q8IWV8 | 88 | | 54 | | 64 | |
| Q8IWV8 | 93 | 72 | 83 | 67 | 77 | 49 |
| Q6ZT12 | 54 | | 48 | | 74 | |
| Q6ZT12 | 35 | | 12 | | 44 | |
| Q6ZT12 | 19 | | 16 | | 36 | |
| Q6ZT12 | 31 | | 24 | | 38 | |
| Q6ZT12 | 54 | | 29 | | 17 | |
| Q6ZT12 | 39 | 16 | 23 | | 67 | |
| Q6ZT12 | 62 | 14 | 52 | 21 | 61 | |
| Q6ZT12 | 45 | | 39 | | 51 | |
| Q6ZT12 | 62 | | 34 | | 46 | |
| Q6ZT12 | 24 | 7 | 26 | | 33 | |
| Q6ZT12 | 8 | | | | 17 | |
| Q6ZT12 | 26 | 25 | 37 | | 38 | |
| Q6ZT12 | 82 | | 54 | | | |
| Q6ZT12 | 89 | | 58 | | 70 | |
| Q6ZT12 | 70 | 41 | 59 | 40 | 56 | 41 |
| Q5T4S7 | 34 | 34 | 39 | 18 | 39 | 14 |
| Q5T4S7 | 69 | | 58 | | 44 | |
| Q5T4S7 | 48 | 9 | 44 | | 56 | |
| Q5T4S7 | | 8 | 22 | | | |
| Q5T4S7 | 64 | 24 | 57 | 31 | 55 | 40 |
| Q5T4S7 | 65 | 24 | 53 | | 64 | |
| Q5T4S7 | 63 | 19 | 43 | | 73 | |
| Q5T4S7 | 62 | 17 | 55 | 2 | 54 | 7 |
| Q5T4S7 | 22 | | 73 | 60 | 71 | 31 |
| Q5T4S7 | 33 | | 26 | | 35 | |
| Q5T4S7 | 38 | 6 | 33 | | 47 | |
| Q5T4S7 | | 12 | 49 | | | |
| Q5T4S7 | 63 | | 37 | | 70 | |
| Q5T4S7 | 44 | 11 | 32 | | 57 | 29 |
| Q5T4S7 | 56 | | 32 | | 37 | |
| Q5T4S7 | 51 | 18 | 33 | | 15 | |
| Q5T4S7 | 78 | 53 | 80 | 49 | 77 | 56 |
| Q5T4S7 | 32 | 10 | 42 | 32 | | |
| Q5T4S7 | | 11 | 38 | | 30 | |
| Q5T4S7 | 50 | 16 | 77 | 58 | 97 | 56 |
| Q5T4S7 | 50 | 7 | 40 | 8 | 93 | 3 |
| Q5T4S7 | 51 | 36 | 76 | 46 | 100 | 64 |
| Q5T4S7 | 29 | 25 | 67 | 36 | 52 | 51 |
| Q5T4S7 | 68 | | 61 | | 66 | |
| Q5T4S7 | 57 | 50 | 49 | 33 | 58 | 30 |
| Q5T4S7 | 26 | 4 | 39 | | 44 | |
| Q5T4S7 | 58 | 35 | 58 | 17 | 35 | 36 |
| Q5T4S7 | 45 | | 36 | | 58 | |
| Q5T4S7 | 35 | | 21 | | 47 | |
| Q5T4S7 | 62 | | 40 | | 42 | |
| Q5T4S7 | 65 | 4 | 69 | | 66 | |
| Q5T4S7 | 56 | | 42 | | 63 | |
| Q5T4S7 | 70 | 4 | 62 | 48 | 60 | |
| Q5T4S7 | 51 | 30 | 32 | 29 | 78 | 23 |
| Q5T4S7 | 29 | 26 | 58 | 38 | 81 | 43 |
| Q5T4S7 | 33 | 44 | 56 | 37 | 50 | 25 |
| Q5T4S7 | 41 | | 14 | | 34 | |
| Q5T4S7 | 37 | | 47 | | 67 | |
| Q5T4S7 | 67 | 30 | 61 | 48 | 72 | 45 |
| Q5T4S7 | 52 | 21 | 50 | 34 | 52 | 26 |
| Q5T4S7 | 49 | | 35 | 20 | 36 | 1 |
| Q5T4S7 | 78 | 38 | 38 | | 34 | 29 |
| Q5T4S7 | 82 | 28 | 49 | 24 | 51 | 39 |
| Q5T4S7 | 23 | 12 | 35 | | 42 | |
| Q5T4S7 | 33 | 23 | 33 | 19 | 34 | 46 |
| Q5T4S7 | 36 | | 6 | | 43 | |
| Q5T4S7 | 67 | 33 | 57 | 20 | 66 | 27 |
| Q5T4S7 | 51 | | 38 | | 50 | |
| Q5T4S7 | 53 | | 33 | | 54 | |
| Q5T4S7 | 77 | | 69 | | 75 | |
| Q5T4S7 | 62 | | 50 | | 60 | |
| Q5T4S7 | 69 | | 46 | | 63 | |
| Q5T4S7 | 9 | | 14 | | 6 | |
| Q5T4S7 | 59 | 33 | 44 | 27 | 58 | 29 |
| Q5T4S7 | 6 | | 14 | | | |
| Q5T4S7 | 49 | | 21 | | 38 | |
| Q5T4S7 | 33 | | 46 | | 43 | |
| O95071 | | | | | 24 | |
| O95071 | 76 | 22 | 49 | | | |
| O95071 | | | 34 | | | |
| O95071 | 34 | 6 | 18 | | 44 | 4 |
| O95071 | 46 | 12 | 30 | | 58 | |
| O95071 | 56 | 24 | 52 | 39 | 50 | 52 |
| O95071 | 34 | 11 | 32 | 14 | 33 | |
| O95071 | | | 21 | | | |
| O95071 | 2 | | 18 | | 47 | |
| O95071 | | 2 | 38 | | | |
| O95071 | 41 | 23 | 48 | | 23 | |
| O95071 | 75 | 4 | 48 | | 65 | |
| O95071 | 39 | 7 | 33 | 25 | 43 | 4 |
| O95071 | 78 | 40 | 70 | 23 | 71 | 71 |
| O95071 | 78 | 34 | 66 | 16 | 73 | 11 |
| O95071 | 65 | 38 | 57 | 38 | 60 | 51 |
| O95071 | 76 | 28 | 59 | 34 | 55 | 52 |
| O95071 | 24 | | 29 | | 42 | 15 |
| O95071 | | | 73 | 51 | | |
| Q8N806 | | 20 | 55 | 34 | 14 | 25 |
| Q8N806 | 51 | 8 | 33 | 6 | 47 | |
| Q8N806 | 81 | | 67 | | 79 | |
| Q8N806 | 76 | | 61 | | 80 | |
| Q8N806 | 78 | | 60 | | 80 | |
| Q8N806 | 78 | | 58 | | 80 | |
| Q8N806 | 50 | | 33 | | 45 | |
| Q8N806 | 75 | | 67 | | 78 | |
| Q8N806 | 57 | 17 | 46 | | 64 | 4 |
| Q8N806 | 56 | | 40 | | 91 | |
| Q8N806 | 2 | 24 | 12 | | 14 | |
| Q8N806 | | | 12 | | | |
| Q8N806 | 24 | 19 | 5 | 2 | 16 | 14 |
| Q8N806 | 4 | 14 | 23 | | 21 | |
| Q8N806 | | 16 | 4 | | 6 | |
| Q8N806 | 51 | 30 | 77 | 62 | 57 | 18 |
| Q8N806 | 6 | 2 | 10 | | | |
| O94874 | 62 | 34 | 67 | 32 | 76 | 12 |
| O94874 | 23 | | 9 | | 29 | |
| O94874 | 30 | 9 | 29 | | 44 | |
| O94874 | 60 | 41 | 70 | 59 | 66 | 45 |
| Q96T88 | 54 | | | | 40 | |
| Q96T88 | 52 | | | | 28 | |
| Q96T88 | 44 | 26 | 21 | 12 | 21 | 35 |
| Q96T88 | 47 | 33 | 33 | 25 | 42 | 51 |
| Q96T88 | | 19 | 53 | 6 | 21 | 28 |
| Q96T88 | 63 | 42 | 61 | 47 | 40 | 18 |
| Q96T88 | 47 | 37 | 36 | | 44 | 7 |
| Q96T88 | 30 | 26 | 35 | 26 | 36 | 31 |
| Q96T88 | 57 | 32 | 44 | 30 | 45 | 33 |
| Q96T88 | 51 | 34 | 29 | 12 | 59 | 16 |
| Q96T88 | 39 | 20 | 23 | 20 | 72 | 5 |
| Q96T88 | 57 | 7 | 15 | | 40 | 11 |
| Q96T88 | 21 | 5 | 28 | | | |
| Q96T88 | | | 19 | | | |
| Q96T88 | 30 | | 35 | | 24 | |
| Q96T88 | 17 | 12 | 40 | | | |
| Q96T88 | 58 | 32 | 50 | 28 | 42 | 4 |
| Q96T88 | 35 | 12 | 61 | 64 | 48 | 17 |
| Q96PU4 | | | 4 | | 1 | |
| Q96PU4 | 33 | 11 | 29 | 8 | 16 | |
| Q96PU4 | 32 | | | | 9 | |
| Q96PU4 | 48 | 15 | 21 | 14 | 43 | 7 |
| Q96PU4 | | | 5 | | 7 | |
| Q96PU4 | 14 | | 4 | 6 | 29 | |
| Q96PU4 | | | | | 9 | |
| Q96PU4 | 39 | | 32 | | 54 | |
| Q96PU4 | 51 | 50 | 46 | 31 | 45 | |
| Q96PU4 | 51 | | 27 | | 54 | |
| Q96PU4 | 57 | 32 | 44 | 30 | 45 | 33 |
| Q96PU4 | 23 | 39 | 27 | 14 | 32 | |
| Q96PU4 | 11 | | 21 | | | |
| Q96PU4 | 46 | 42 | 32 | 13 | 32 | |
| Q96PU4 | 19 | 20 | 42 | 48 | 20 | |
| Q96PU4 | 58 | 32 | 50 | 28 | 42 | 4 |
| Q96PU4 | 35 | 12 | 61 | 64 | 48 | 17 |
| Q9C0B0 | 57 | | 67 | | 70 | |
| Q9C0B0 | 21 | | 10 | | 54 | |
| Q9C0B0 | 50 | | 49 | | 32 | |
| Q9C0B0 | 31 | | 19 | | 30 | |
| Q9C0B0 | 32 | | 28 | | 50 | |
| Q9C0B0 | 31 | | 29 | | 32 | |
| Q9C0B0 | | | | | 3 | |
| Q9C0B0 | 34 | | 20 | 15 | 34 | |
| Q9C0B0 | 64 | | 34 | | 54 | |
| Q9C0B0 | 78 | 17 | 56 | 21 | 67 | 15 |
| Q9C0B0 | 31 | 25 | | | 35 | |
| Q9C0B0 | 83 | 43 | 63 | 52 | 53 | 23 |
| Q9C0B0 | 76 | | 40 | | 45 | |
| Q9C0B0 | 57 | 44 | 41 | 25 | 27 | |
| Q9C0B0 | 44 | | 18 | | 33 | |
| Q9C0B0 | 59 | | 76 | 43 | 46 | |
| Q9H9P5 | 81 | | 76 | | 63 | |
| Q9H9P5 | | | 28 | | 14 | |
| Q9H9P5 | 46 | | 22 | | 37 | |
| Q9H9P5 | 3 | 6 | 26 | | 11 | |
| Q9H9P5 | 60 | 20 | 21 | | 27 | |
| Q9H9P5 | | | 27 | | | |
| Q9H9P5 | 6 | | 44 | | 21 | |
| Q9H9P5 | 42 | | 6 | | 37 | |
| Q9H9P5 | 48 | | 30 | | 67 | |
| Q9H9P5 | 45 | | 17 | | 36 | |
| Q9H9P5 | 55 | 9 | 31 | | 37 | |
| P40337 | 38 | 12 | 32 | 19 | 32 | 15 |
| P40337 | 23 | | 19 | | | |
| Q9H270 | 56 | 42 | 50 | 38 | 26 | |
| Q9H270 | 99 | 58 | 92 | 80 | 26 | 1 |
| Q9H270 | | 6 | 14 | | | |
| Q9H270 | 44 | | 34 | | 48 | |
| Q9H270 | 21 | 13 | 26 | 9 | 37 | |
| Q9H270 | 20 | | 17 | | 38 | |
| Q9H270 | 46 | | 36 | | 60 | |
| Q9H270 | 15 | 14 | 18 | | 30 | |
| Q9H270 | 28 | 23 | 42 | 34 | 39 | |
| Q9H270 | 36 | | 34 | | 46 | |
| Q9H270 | 47 | 24 | 81 | 55 | 52 | 43 |
| Q9P253 | 73 | 30 | 81 | 56 | 74 | 43 |
| Q9P253 | 41 | 10 | 53 | 36 | 51 | 12 |
| Q9P253 | 63 | | 34 | | 47 | |
| Q9P253 | 72 | | 30 | | 54 | |
| Q9P253 | 90 | | 55 | | 86 | 17 |
| Q9P253 | 39 | | | | 20 | |
| Q9P253 | 46 | 15 | 33 | | 47 | |
| Q9P253 | 24 | 4 | 31 | | 17 | |
| Q9P253 | 49 | 8 | 67 | | 64 | |
| Q9P253 | 90 | 30 | 94 | | 84 | |
| Q9P253 | 89 | 64 | 92 | 85 | 97 | 85 |
| P49754 | 33 | 17 | 34 | 1 | 39 | |
| P49754 | 76 | 12 | 79 | 10 | 62 | 10 |
| P49754 | | 55 | | | | |
| P49754 | 91 | | 34 | | 55 | |
| Q8N3P4 | 8 | 3 | 16 | | 16 | |
| Q8N3P4 | 27 | 1 | 16 | | 21 | |
| Q8N3P4 | 62 | 35 | 87 | 44 | 45 | 55 |
| Q8N3P4 | 38 | 19 | 37 | | 40 | |
| Q8N3P4 | 48 | 9 | 48 | | 36 | |
| Q9H7D7 | 79 | | 52 | | 41 | |
| Q9H7D7 | 39 | 39 | 43 | 21 | 20 | |
| Q9H7D7 | 23 | | 32 | | 41 | |
| Q9H7D7 | 65 | 54 | 87 | 78 | 74 | 32 |
| Q9H7D7 | 72 | 58 | 55 | 42 | 60 | 32 |
| Q9H7D7 | 18 | 9 | 29 | | 18 | |
| Q9H7D7 | 73 | 49 | 63 | 33 | 74 | |
| Q9H7D7 | 18 | | 36 | | 37 | |
| Q9H7D7 | 33 | | 29 | | 51 | |
| Q9H7D7 | 37 | | 29 | | 41 | |
| Q9H7D7 | 28 | 31 | 27 | 30 | 32 | 22 |
| Q8N9V3 | 41 | 20 | 41 | | | |
| Q8N9V3 | 66 | 30 | 58 | 36 | 65 | 40 |
| Q8N9V3 | 44 | 17 | 28 | 2 | 22 | 16 |
| Q8N5D0 | 45 | | 42 | | 46 | |
| Q8N5D0 | 100 | 58 | 96 | 87 | 95 | 46 |
| Q8N5D0 | 74 | | 45 | | 73 | |
| Q8N5D0 | 68 | 40 | 61 | 29 | 69 | 31 |
| Q8N5D0 | 83 | 49 | 92 | 79 | 85 | 70 |
| Q8N5D0 | 89 | | 51 | | 61 | |
| Q8N5D0 | 79 | 28 | 68 | 20 | 81 | 20 |
| Q8N5D0 | 70 | | 72 | | 87 | |
| Q8N5D0 | 80 | | 57 | | 87 | |
| Q8N5D0 | 75 | | 49 | | 72 | |
| Q8N5D0 | 79 | | 76 | | 82 | |
| Q8N5D0 | 79 | | 83 | | 87 | |
| Q9Y6I7 | 61 | | 10 | | 61 | |
| Q9Y6I7 | 56 | | 43 | | 66 | |
| Q9Y6I7 | 29 | 5 | 22 | | 36 | |
| Q9H0M0 | 22 | 7 | 17 | | 24 | |
| Q9H0M0 | 55 | 35 | 81 | 61 | 85 | 36 |
| Q9H0M0 | 59 | 22 | 34 | | 80 | 25 |
| O00308 | 55 | 26 | 40 | 32 | 54 | 37 |
| O00308 | 62 | 9 | 48 | | 55 | |
| O00308 | 55 | 35 | 81 | 61 | 85 | 36 |
| O00308 | 59 | 23 | 54 | | 56 | |
| O00308 | 59 | 22 | 34 | | 80 | 25 |
| P98170 | 34 | 20 | 13 | 12 | 30 | 8 |
| P98170 | 46 | | 22 | | 25 | |
| P98170 | 55 | 15 | 70 | 58 | 48 | 24 |
| P98170 | 74 | 27 | 65 | 37 | 37 | 12 |
| P98170 | 27 | 25 | 21 | | 33 | 5 |
| P98170 | 38 | | 21 | | 45 | |
| P98170 | 43 | 23 | 50 | 25 | 40 | 21 |
| P98170 | 42 | | 45 | | 55 | |
| P98170 | 40 | | 38 | | 45 | |
| P98170 | 65 | | 44 | | 62 | |
| P10074 | 50 | 26 | 60 | | 74 | |
| P10074 | 59 | | 15 | | 69 | |
| P10074 | 45 | 31 | 38 | 24 | 40 | |
| P10074 | | | | | 47 | |
| Q86WB0 | 69 | 21 | 64 | 45 | 71 | 14 |
| Q86WB0 | 38 | 20 | 47 | 15 | 52 | 26 |
| Q86WB0 | 80 | | 69 | | 80 | |
| Q86WB0 | 69 | | 81 | | 75 | |
| Q86WB0 | 69 | 27 | 80 | 48 | 81 | 20 |
| Q86WB0 | 45 | 15 | 61 | 38 | 78 | 33 |
| Q86WB0 | 67 | 13 | 65 | | 76 | 23 |
| Q86WB0 | 66 | | 73 | | 85 | |
| Q86WB0 | 49 | 11 | 53 | 40 | 55 | 18 |
| Q86WB0 | 78 | 50 | 54 | 39 | 64 | 15 |
| Q86WB0 | 81 | 39 | 77 | 62 | 68 | 26 |
| Q86WB0 | 33 | 8 | 28 | | | |
| Q86WB0 | 34 | | 28 | | 73 | |
| Q9H8U3 | 44 | | 43 | 19 | 35 | 6 |
| Q9H8U3 | 72 | 39 | 59 | 42 | 33 | 3 |
| Q9H8U3 | | | 15 | | 18 | |
| Q9H8U3 | 64 | 31 | 26 | 17 | 18 | 24 |
| Q9H8U3 | 41 | 41 | 35 | 19 | 35 | 12 |
| Q9H8U3 | 54 | | 30 | | 59 | |
| Q9H8U3 | 49 | | 13 | | 40 | |
| Q9H8U3 | 63 | 37 | 45 | | 20 | |
| Q9H8U3 | 42 | 7 | 28 | | | |
| O76080 | 29 | 7 | 11 | | 18 | 5 |
| O76080 | 31 | | 18 | | 11 | |
| O76080 | 37 | 16 | 14 | 14 | 26 | |
| O76080 | 15 | 10 | 24 | | 18 | 8 |
| O76080 | 1 | | 17 | | 16 | |
| O76080 | 50 | | 13 | | 48 | 3 |
| Q6FIF0 | 29 | 7 | 11 | | 18 | 5 |
| Q6FIF0 | 31 | | 18 | | 11 | |
| Q6FIF0 | 15 | 16 | 32 | 8 | 36 | 23 |
| Q6FIF0 | | 1 | 6 | | 14 | |
| Q6FIF0 | 1 | | 16 | | 22 | |
| Q6FIF0 | | | 5 | | 12 | |
| Q96JP5 | 34 | 22 | 22 | 4 | 42 | 17 |
| Q96JP5 | 33 | 22 | 16 | 17 | 40 | 36 |
| Q96JP5 | 67 | 66 | 75 | 70 | 73 | 26 |
| Q96JP5 | 63 | 44 | 61 | 40 | 55 | 12 |
| Q96JP5 | 49 | | 20 | | 33 | |
| Q96JP5 | 57 | | 36 | | 49 | 10 |
| Q96JP5 | 28 | | 22 | | 7 | 5 |
| Q96JP5 | 65 | 68 | 67 | 43 | 66 | 32 |
| O95159 | 68 | 20 | 73 | | 52 | 4 |
| O95159 | 59 | 6 | 61 | | 59 | |
| O95159 | 62 | 7 | 52 | | 58 | |
| O95159 | 43 | 41 | 51 | | 50 | 13 |
| O95159 | 66 | | 55 | | 57 | |
| O95159 | 65 | 18 | 61 | | 66 | |
| O95159 | 100 | | 59 | | 70 | |
| O95159 | 26 | 40 | 22 | 15 | 54 | 48 |
| Q9ULJ6 | 47 | 11 | 34 | 16 | 42 | 42 |
| Q9ULJ6 | 11 | | 3 | | 19 | |
| Q9ULJ6 | 51 | 4 | 32 | | 58 | 7 |
| Q9ULJ6 | 59 | 24 | 54 | | 69 | |
| Q9ULJ6 | 47 | 22 | 34 | 15 | 80 | 59 |
| Q9ULJ6 | 47 | 37 | 31 | 3 | 23 | 8 |
| Q9ULJ6 | | | 7 | | 62 | |
| Q9ULJ6 | 41 | | 41 | | 40 | |
| Q8NF64 | 46 | 41 | 53 | 20 | 41 | |
| Q8NF64 | 51 | 4 | 32 | | 58 | 7 |
| Q8NF64 | 59 | 24 | 54 | | 69 | |
| Q8NF64 | 47 | 22 | 34 | 15 | 80 | 59 |
| Q8NF64 | 47 | 37 | 31 | 3 | 23 | 8 |
| Q8NF64 | 41 | | 8 | | 34 | |
| Q8NF64 | 41 | | 41 | | 40 | |
| Q9Y4E5 | 40 | 7 | 35 | 13 | 56 | 11 |
| Q9Y4E5 | 67 | 35 | 52 | 13 | 66 | 17 |
| Q9Y4E5 | 58 | | 34 | | 49 | |
| Q9Y4E5 | | | 30 | 15 | | |
| Q9Y4E5 | | | 40 | | | |
| Q9Y4E5 | 49 | 11 | 33 | | 50 | |
| Q9Y4E5 | 54 | 15 | 65 | 64 | 70 | 45 |
| Q9Y4E5 | 27 | | 36 | | 40 | |
| Q9Y4E5 | | | 18 | | | |
| Q9Y4E5 | 38 | | 33 | | 58 | |
| Q9Y4E5 | 65 | | 49 | | 60 | |
| Q9Y4E5 | | | 25 | | | |
| Q9Y4E5 | 62 | 24 | | | 43 | |
| Q9Y4E5 | 15 | | 5 | | 25 | |
| Q9Y4E5 | 33 | | 24 | | 15 | |
| Q9Y4E5 | 43 | 22 | 59 | 25 | 50 | 12 |
| Q9Y4E5 | 34 | 29 | 65 | | 3 | |
| Q86UK7 | 66 | 51 | 63 | 50 | 67 | 24 |
| Q86UK7 | 3 | | 54 | 21 | 32 | |
| Q86UK7 | 49 | | 57 | | 57 | |
| Q86UK7 | 47 | 22 | 48 | | 45 | 1 |
| Q86UK7 | 72 | 9 | 73 | 27 | 44 | 22 |
| Q86UK7 | 17 | | 33 | | 30 | |
| Q86UK7 | 51 | | 47 | | 44 | |
| Q86UK7 | 46 | 22 | 47 | | 32 | 25 |
| Q86UK7 | 76 | | 37 | | 56 | |
| Q86UK7 | | | 51 | | | |
| Q86UK7 | 4 | 3 | 53 | | | |
| Q86UK7 | 6 | 2 | | | 11 | |
| Q86UK7 | 45 | 9 | 37 | | 48 | |
| Q86UK7 | 70 | 43 | 16 | | 67 | |
| Q86UK7 | 25 | 8 | 18 | | 23 | |
| Q86UK7 | 30 | 13 | 15 | | 30 | |
| Q86UK7 | 27 | 15 | 25 | 23 | 32 | 32 |
| Q86UK7 | 57 | 21 | 36 | 2 | 27 | |
| Q86UK7 | 66 | 39 | 75 | 56 | 66 | 18 |
| Q86UK7 | 68 | 26 | 75 | 59 | 68 | 15 |
| Q86UK7 | 63 | 6 | 70 | 42 | 58 | 29 |
| Q86UK7 | 35 | | 61 | 44 | 34 | |
| Q86UK7 | 21 | 1 | 16 | | 8 | |
| Q86UK7 | 1 | | 3 | | | |
| Q8ND25 | 42 | | 15 | | 28 | |
| Q8ND25 | 32 | | 2 | | 23 | |
| Q8ND25 | 45 | 18 | 56 | 20 | 45 | 9 |
| Q8ND25 | 43 | 3 | 59 | 48 | 29 | |
| Q8ND25 | | | 47 | | 13 | |
| Q8ND25 | 27 | | 28 | | 43 | |
| Q8NHG8 | | | | | 11 | |
| Q8NHG8 | | | | | 24 | |
| Q8NHG8 | 35 | | 8 | | | |
| Q8NHG8 | 45 | 18 | 56 | 20 | 45 | 9 |
| Q8NHG8 | 43 | 3 | 59 | 48 | 29 | |
| Q8NHG8 | | 4 | | | 13 | |
| Q9ULT6 | 39 | | 16 | | 36 | |
| Q9ULT6 | 53 | | 39 | | 49 | |
| Q9ULT6 | 79 | | 42 | | 55 | |
| Q9ULT6 | 44 | | 29 | | 51 | |
| Q8NEG5 | 35 | | 8 | | 16 | |
| Q9C0D3 | 50 | 12 | 44 | | 37 | |
| Q9C0D3 | 46 | | 20 | | 48 | |
| Q9C0D3 | 72 | 34 | 58 | 38 | 64 | 48 |

¹KB02_500 uM_percent_inhibition
²KB02_100 uM_percent_inhibition
³KB03_500 uM_percent_inhibition
⁴KB03_100 uM_percent_inhibition
⁵KB05_500 uM_percent_inhibition
⁶KB05_100 uM_percent_inhibition

### Example 2

The following 96-well sample prep protocol was used for sample preparation.

Cells were resuspended in cold PBS on ice and sonicated with a probe sonicator to achieve lysis. 200 μL of lysate containing 5 mg/ml proteome was treated with 2 μL of 100× compound stock in a 2-mL deep-well plate. The treated lysate was subsequently incubate at 25° C. for 1 hr with shaking at 600 rpm. The treated lysate was further incubated with 2 μL of 10 mM desthiobiotin iodoacetamide probe at 25° C. for 1 hr with shaking at 600 rpm. Next, the lysate was further treat with 20 μL of solution of PBS containing 11 mM MgSO₄ and 2.5% Turbonuclease stock and incubated at 25° C. for 20 minutes with shaking at 600 rpm. 1.7 mL ice-cold acetone was added to each well and incubated at −20° C. for 2 hr, followed by max speed (4200 rpm) spin for 45 min. Acetone was then decanted and the plates were blotted to remove acetone. The plates were then dried in open air for 20 minutes. After drying, the plates were then covered with foil seal and stored at −80° C. overnight.

The next day, samples were re-suspended in 90 μL of solution of 9M urea, 50 mM ammonium bicarbonate and 10 mM DTT by incubating at 65C for 20 min with shaking at 1500 rpm. Next, samples were cooled to 37° C. and then 10 μL of 500 mM iodoacetamide solution (92.48 mg/ml) was added. The samples were then incubated at 37° C. for 30 min with shaking at 600 rpm.

ZEBA desalting plates were equilibrated 4 times using 250 μL of 2M urea, 50 mM ammonium bicarbonate solution followed by centrifugation at 1500 rpm for 2 minutes. After equilibration was complete, samples were applied to ZEBA desalting plate and spin at 1500 rpm for 2 min on top of 1 mL 96-well deep-well collection plate to buffer exchange samples. 4 μL of solution containing 25 mM CaCl₂ and 0.25 mg/mL trypsin was added to the plate and the plate was then incubated at 37° C. for 2 hrs with shaking at 600 rpm. 300 μL of solution containing 5% high-capacity streptavidin agarose slurry in 25 mM Tris-HCl pH 7.5, 150 mM NaCl, and 0.1% NP-40 then added. The plate was incubated at 25° C. for 2 hrs with shaking at 600 rpm. Samples were transferred to 25 μm filter plate and spin at 1000 rpm for 2 min. Samples were then washed 3 times with 0.75 mL of wash buffer (25 mM Tris-HCl pH 7.5, 150 mM NaCl, and 0.1% NP-40) followed by washing 3 times with 0.75 mL PBS, and then 4 times with 0.75 mL water. After the washing step, samples were eluted into polypropylene 96-well plate by addition of 250 L of 50% MeCN/water, 0.1% formic acid and allowed to gravity drip for 10 min, followed by centrifugation at 1000 rpm for 2 min. Speedvac was used to dry the plates at about 45° C. for −5 hrs. Then the plates were covered with foil seal and stored at −20° C.

Samples were resuspended by addition of 20 μl of 12.5% ACN, 0.1% formic acid solution, covered with foil seal, and incubated at 42° C. for 10 minutes with shaking at 600 rpm. Then add 30 μL of 0.1% formic acid solution was added and the plate was covered and incubated at 42° C. for 10 minutes with shaking at 600 rpm. Seal plate was then sealed with a 96-well silicon mat for analysis.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A protein-probe adduct wherein the probe binds to a cysteine residue of a protein illustrated in Table 1A or Table 2A, wherein the probe has a structure represented by Formula (I): 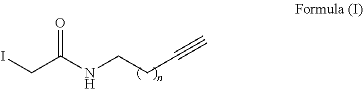
wherein,
n is 0-8.

2. The protein-probe adduct of claim 1, wherein the protein is selected from Ankyrin repeat and BTB/POZ domain-containing protein 1, Ankyrin repeat and BTB/POZ domain-containing protein 2, Activating molecule in BECN1-regulated autophagy protein 1, Anaphase-promoting complex subunit 11, Anaphase-promoting complex subunit 15, Anaphase-promoting complex subunit 16, Anaphase-promoting complex subunit 2, Anaphase-promoting complex subunit 7, Rabankyrin-5, Ankyrin repeat and IBR domain-containing protein 1, Amyloid protein-binding protein 2, Apoptosis-resistant E3 ubiquitin protein ligase 1, E3 ubiquitin-protein ligase ARIH1, E3 ubiquitin-protein ligase ARIH2, Armadillo repeat-containing protein 5, Ankyrin repeat and SOCS box protein 2, Ankyrin repeat and SOCS box protein 6, Transcriptional regulator ATRX, Transcription regulator protein BACH1, Transcription regulator protein BACH2, Baculoviral IAP repeat-containing protein 2, Baculoviral IAP repeat-containing protein 3, Baculoviral IAP repeat-containing protein 6, Breast cancer type 1 susceptibility protein, F-box/WD repeat-containing protein 1A, Cullin-associated NEDD8-dissociated protein 1, Cullin-associated NEDD8-dissociated protein 2, E3 ubiquitin-protein ligase CBL, E3 ubiquitin-protein ligase CBL-B, E3 ubiquitin-protein ligase CBL-C, Cyclin-F, Cell division cycle protein 20 homolog, Cell division cycle protein 23 homolog, Cell division cycle protein 27 homolog, Cell growth regulator with RING finger domain protein 1, E3 ubiquitin-protein ligase CHFR, Clusterin, CCR4-NOT transcription complex subunit 4, COMM domain-containing protein 2, COMM domain-containing protein 9, Cullin-4A, Cullin-5, Cullin-7, Cullin-9, DDB1- and CUL4-associated factor 1, DDB1- and CUL4-associated factor 10, DDB1- and CUL4-associated factor 13, DDB1- and CUL4-associated factor 16, DDB1- and CUL4-associated factor 17, DDB1- and CUL4-associated factor 5, DDB1- and CUL4-associated factor 6, DDB1- and CUL4-associated factor 7, DNA damage-binding protein 2, Zinc finger protein neuro-d4, Denticleless protein homolog, E3 ubiquitin-protein ligase DTX1, Probable E3 ubiquitin-protein ligase DTX3, E3 ubiquitin-protein ligase DTX3L, E3 SUMO-protein ligase EGR2, Ectoderm-neural cortex protein 1, DNA excision repair protein ERCC-8, E3 ubiquitin-protein ligase FANCL, F-box DNA helicase 1, F-box/LRR-repeat protein 12, F-box/LRR-repeat protein 17, F-box/LRR-repeat protein 18, F-box/LRR-repeat protein 20, F-box/LRR-repeat protein 3, F-box/LRR-repeat protein 6, F-box only protein 11, F-box only protein 30, F-box only protein 31, F-box only protein 38, F-box only protein 42, F-box/SPRY domain-containing protein 1, F-box only protein 5, F-box only protein 7, F-box only protein 9, F-box/WD repeat-containing protein 11, F-box/WD repeat-containing protein 4, F-box/WD repeat-containing protein 8, F-box/WD repeat-containing protein 9, Protein fem-1 homolog A, Protein fem-1 homolog B, Gigaxonin, General transcription factor IIH subunit 2, E3 ubiquitin-protein ligase HACE1, E3 ubiquitin-protein ligase HECTD1, Probable E3 ubiquitin-protein ligase HECTD4, E3 ubiquitin-protein ligase HECW1, E3 ubiquitin-protein ligase HECW2, Probable E3 ubiquitin-protein ligase HERC1, E3 ubiquitin-protein ligase HERC2, Probable E3 ubiquitin-protein ligase HERC3, Probable E3 ubiquitin-protein ligase HERC4, E3 ISG15-protein ligase HERC5, Helicase-like transcription factor, E3 ubiquitin-protein ligase HUWE1, Actin-binding protein IPP, Interferon regulatory factor 2-binding protein 1, Interferon regulatory factor 2-binding protein 2, Interferon regulatory factor 2-binding protein-like, E3 ubiquitin-protein ligase Itchy homolog, Influenza virus NS1A-binding protein, Kelch repeat and BTB domain-containing protein 11, Kelch repeat and BTB domain-containing protein 4, Kelch repeat and BTB domain-containing protein 6, Kelch repeat and BTB domain-containing protein 7, Kelch repeat and BTB domain-containing protein 8, E3 ubiquitin-protein ligase KCMF1, BTB/POZ domain-containing protein KCTD3, BTB/POZ domain-containing protein KCTD7, Kelch-like ECH-associated protein 1, Kelch domain-containing protein 10, Kelch domain-containing protein 3, Kelch-like protein 20, Kelch-like protein 24, Kelch-like protein 26, Kelch-like protein 36, E3 ubiquitin-protein ligase LNX, LON peptidase N-terminal domain and RING finger protein 2, Leucine-rich repeat protein 1, Leucine-rich repeat-containing protein 41, E3 ubiquitin-protein ligase LRSAM1, E3 ubiquitin-protein ligase listerin, E3 ubiquitin-protein ligase MARCH1, E3 ubiquitin-protein ligase MARCH3, E3 ubiquitin-protein ligase MARCH5, E3 ubiquitin-protein ligase MARCH6, E3 ubiquitin-protein ligase MARCH7, E3 ubiquitin-protein ligase Mdm2, E3 ubiquitin-protein ligase MGRN1, E3 ubiquitin-protein ligase MIB2, Probable E3 ubiquitin-protein ligase MID2, E3 ubiquitin-protein ligase makorin-1, Probable E3 ubiquitin-protein ligase makorin-2, Male-specific lethal 1 homolog, E3 ubiquitin-protein ligase MYCBP2, E3 ubiquitin-protein ligase NEDD4, E3 ubiquitin-protein ligase NEDD4-like, Transcriptional repressor NF-X1, NF-X1-type zinc finger protein NFXL1, Nitric oxide synthase-interacting protein, Histone-lysine N-methyltransferase NSD2, OTU domain-containing protein 7B, POZ-, AT hook-, and zinc finger-containing protein 1, Polycomb group RING finger protein 2, E3 ubiquitin-protein ligase PDZRN3, E3 ubiquitin-protein ligase pellino homolog 1, E3 ubiquitin-protein ligase pellino homolog 2, Peroxisome biogenesis factor 10, PHD and RING finger domain-containing protein 1, E3 SUMO-protein ligase PIAS2, E3 SUMO-protein ligase PIAS4, E3 ubiquitin-protein ligase Praja-2, Protein PML, RING-type E3 ubiquitin-protein ligase PPIL2, E3 ubiquitin-protein ligase parkin, Pre-mRNA-processing factor 19, E3 SUMO-protein ligase RanBP2, E3 ubiquitin-protein ligase RBX1, RCC1 and BTB domain-containing protein 1, RING finger and CHY zinc finger domain-containing protein 1, E3 ubiquitin-protein ligase RFWD3, E3 ubiquitin-protein ligase RING1, E3 ubiquitin-protein ligase RLIM, RING finger protein 10, E3 ubiquitin-protein ligase RNF114, E3 ubiquitin-protein ligase RNF126, E3 ubiquitin-protein ligase RNF128, E3 ubiquitin-protein ligase RNF135, E3 ubiquitin-protein ligase RNF14, E3 ubiquitin-protein ligase RNF144B, RING finger protein 148, E3 ubiquitin-protein ligase RNF149, RING finger protein 150, E3 ubiquitin ligase RNF157, E3 ubiquitin-protein ligase RNF168, E3 ubiquitin-protein ligase RNF187, E3 ubiquitin-protein ligase RNF19B, E3 ubiquitin-protein ligase RING2, E3 ubiquitin-protein ligase BRE1A, E3 ubiquitin-protein ligase RNF213, RING finger protein 214, E3 ubiquitin-protein ligase RNF216, Probable E3 ubiquitin-protein ligase RNF217, RING finger protein 219, E3 ubiquitin-protein ligase RNF25, E3 ubiquitin-protein ligase RNF31, E3 ubiquitin-protein ligase BRE1B, E3 ubiquitin-protein ligase RNF43, E3 ubiquitin-protein ligase RNF5, RING-box protein 2, E3 ubiquitin-protein ligase RNF8, RUN and FYVE domain-containing protein 1, E3 ubiquitin-protein ligase SH3RF2, SH3KBP1-binding protein 1, Structure-specific endonuclease subunit SLX4, E3 ubiquitin-protein ligase SMURF1, E3 ubiquitin-protein ligase SMURF2, Suppressor of cytokine signaling 2, Suppressor of cytokine signaling 3, Suppressor of cytokine signaling 6, Suppressor of cytokine signaling 7, Speckle-type POZ protein, Tumor necrosis factor alpha-induced protein 3, E3 ubiquitin-protein ligase Topors, TNF receptor-associated factor 1, TNF receptor-associated factor 2, TNF receptor-associated factor 3, E3 ubiquitin-protein ligase TRIM11, E3 ubiquitin-protein ligase TRIM22, E3 ubiquitin/ISG15 ligase TRIM25, Transcription intermediary factor 1-beta, Tripartite motif-containing protein 3, E3 ubiquitin-protein ligase TRIM32, E3 ubiquitin-protein ligase TRIM33, E3 ubiquitin-protein ligase TRIM36, E3 ubiquitin-protein ligase TRIM4, Tripartite motif-containing protein 47, E3 ubiquitin-protein ligase TRIM56, Tripartite motif-containing protein 59, Tripartite motif-containing protein 65, E3 ubiquitin-protein ligase TRIM7, E3 ubiquitin-protein ligase TRIM71, Tripartite motif-containing protein 72, E3 ubiquitin-protein ligase TRIM8, E3 ubiquitin-protein ligase TRIP12, Short transient receptor potential channel 4-associated protein, E3 ubiquitin-protein ligase TTC3, (E3-independent) E2 ubiquitin-conjugating enzyme, Ubiquitin-protein ligase E3A, Ubiquitin-protein ligase E3B, Ubiquitin-protein ligase E3C, E3 ubiquitin-protein ligase E3D, Ubiquitin conjugation factor E4 A, Ubiquitin conjugation factor E4 B, E3 ubiquitin-protein ligase UBR1, E3 ubiquitin-protein ligase UBR2, E3 ubiquitin-protein ligase UBR3, E3 ubiquitin-protein ligase UBR4, E3 ubiquitin-protein ligase UBR5, Putative E3 ubiquitin-protein ligase UBR7, RING finger protein unkempt homolog, Putative E3 ubiquitin-protein ligase UNKL, Vacuolar protein sorting-associated protein 11 homolog, Vacuolar protein sorting-associated protein 18 homolog, Vacuolar protein sorting-associated protein 41 homolog, Vacuolar protein sorting-associated protein 8 homolog, WD repeat-containing protein 26, WD and tetratricopeptide repeats protein 1, NEDD4-like E3 ubiquitin-protein ligase WWP1, NEDD4-like E3 ubiquitin-protein ligase WWP2, Nuclear-interacting partner of ALK, Zinc finger protein-like 1, E3 ubiquitin-protein ligase ZNF598, and E3 ubiquitin-protein ligase ZNRF3.

3. The protein-probe adduct of claim 1, wherein the protein is B-cell lymphoma 6 protein and the cysteine residue is C121, C175, C232, C254, C296, C339, C348, C354, C414, C548, or C663, wherein the numbering of the amino acid position corresponds to the amino acid position with the UniProt Identifier P41182.

4. The protein-probe adduct of claim 1, wherein the protein is Polycomb group RING finger protein 6 and the cysteine residue is C56, C137, or C155, wherein the numbering of the amino acid position corresponds to the amino acid position with the UniProt Identifier Q9BYE7.

5. The protein-probe adduct of claim 1, wherein the protein is E3 ubiquitin-protein ligase CBL-B and the cysteine residue is C60, C345, C376, C435, C436, C470, C523, C535, C594, C607, C686, C741, or C895, wherein the numbering of the amino acid position corresponds to the amino acid position with the UniProt Identifier Q13191.

6. The protein-probe adduct of claim 1, wherein the protein is Elongin-B and the cysteine residue is C60 or C89, wherein the numbering of the amino acid position corresponds to the amino acid position with the UniProt Identifier Q15370.

7. The protein-probe adduct of claim 1, wherein the protein is Elongin-C and the cysteine residue is C11 or C74, wherein the numbering of the amino acid position corresponds to the amino acid position with the UniProt Identifier Q15369.

8. The protein-probe adduct of claim 1, wherein the protein is F-box only protein 22 and the cysteine residue is C47, C117, C227, C228, or C378, wherein the numbering of the amino acid position corresponds to the amino acid position with the UniProt Identifier Q8NEZ5.

9. The protein-probe adduct of claim 1, wherein the protein is Kelch repeat and BTB domain-containing protein 4 and the cysteine residue is C68, C201, C274, C301, C455, or C472, wherein the numbering of the amino acid position corresponds to the amino acid position with the UniProt Identifier Q9NVX7.

10. The protein-probe adduct of claim 1, wherein the protein is Kelch-like ECH-associated protein 1 and the cysteine residue is C23, C38, C151, C226, C241, C257, C288, C297, C319, C434, C613, C622, or C624 wherein the numbering of the amino acid position corresponds to the amino acid position with the UniProt Identifier Q14145.

11. The protein-probe adduct of claim 1, wherein the protein is E3 ubiquitin-protein ligase pellino homolog 11 and the cysteine residue is C61, C212, or C282 wherein the numbering of the amino acid position corresponds to the amino acid position with the UniProt Identifier Q96FA3.

12. The protein-probe adduct of claim 1, wherein the protein is E3 ubiquitin-protein ligase RNF128 and the cysteine residue is C295, C303, C314, or C317 wherein the numbering of the amino acid position corresponds to the amino acid position with the UniProt Identifier Q8TEB7.

13. The protein-probe adduct of claim 1, wherein the protein is TNF receptor-associated factor 6 and the cysteine residue is C85, C105, C134, C139, C182, C235, C349, or C366, wherein the numbering of the amino acid position corresponds to the amino acid position with the UniProt Identifier Q9Y4K3.

14. The protein-probe adduct of claim 1, wherein the protein is (E3-independent) E2 ubiquitin-conjugating enzyme and the cysteine residue is C101, C182, C208, C230, C244, C314, C341, C370, C375, C400, C406, C585, C598, C910, C913, C1040, C1099, or C1288, wherein the numbering of the amino acid position corresponds to the amino acid position with the UniProt Identifier Q9C0C9.

15. The protein-probe adduct of claim 1, wherein the protein is E3 ubiquitin-protein ligase CBL-B and the cysteine residue is C607, wherein the numbering of the amino acid position corresponds to the amino acid position with the UniProt Identifier Q13191.

16. The protein-probe adduct of claim 1, wherein the protein is Kelch repeat and BTB domain-containing protein 4 and the cysteine residue is 68, wherein the numbering of the amino acid position corresponds to the amino acid position with the UniProt Identifier Q9NVX7.

17. The protein-probe adduct of claim 1, wherein the protein is Kelch-like ECH-associated protein 1 and the cysteine residue is C23, C38, C151, C241, C257, C288, C297, C319, C613, or C624, wherein the numbering of the amino acid position corresponds to the amino acid position with the UniProt Identifier Q14145.

18. The protein-probe adduct of claim 1, wherein the protein is E3 ubiquitin-protein ligase pellino homolog 1 and the cysteine residue is C282, wherein the numbering of the amino acid position corresponds to the amino acid position with the UniProt Identifier Q96FA3.

19. The protein-probe adduct of claim 1, wherein the protein is E3 ubiquitin-protein ligase RNF128 and the cysteine residue is C317, wherein the numbering of the amino acid position corresponds to the amino acid position with the UniProt Identifier Q8TEB7.

20. The protein-probe adduct of claim 1, wherein the protein is (E3-independent) E2 ubiquitin-conjugating enzyme and the cysteine residue is C370, C400, C910, or C913, wherein the numbering of the amino acid position corresponds to the amino acid position with the UniProt Identifier Q9C0C9.
